(19) United States
(12) Patent Application Publication
(10) Pub. No.: US 2023/0295639 A1
(43) Pub. Date: Sep. 21, 2023

(54) De novo designed NTF2-like scaffolds for de novo design of enzymes and small molecule binders

(71) Applicant: University of Washington, Seattle, WA (US)

(72) Inventors: Christoffer NORN, Seattle, WA (US); Ivan ANISHCHANKA, Seattle, WA (US); David BAKER, Seattle, WA (US); Sam PELLOCK, Seattle, WA (US); Douglas TISCHER, Seattle, WA (US); Hsien-Wei YEH, Seattle, WA (US)

(21) Appl. No.: 18/155,179

(22) Filed: Jan. 17, 2023

Related U.S. Application Data
(60) Provisional application No. 63/300,178, filed on Jan. 17, 2022.

(51) Int. Cl. C12N 15/62; C12N 15/63; C12N 15/10

(52) U.S. Cl. CPC C12N 15/62; C12N 15/1044; C12N 15/63

(57) ABSTRACT

Polypeptide are disclosed that comprise an amino acid sequence at least 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-1615, not including any functional domains added fused to the protein (whether N-terminal, C-terminal, or internal), and wherein the 1, 2, 3, 4, or 5 N-terminal and/or C-terminal amino acid residues may be present or absent when considering the percent identity.

### RELATED APPLICATIONS

This application claims priority to U.S. Provisional Application 63/300,178 filed Jan. 17, 2022, incorporated by reference herein in its entirety.

### FEDERAL FUNDING STATEMENT

This invention was made with government support under Grant No. BB/S017135/1, awarded by the National Science Foundation. The government has certain rights in the invention.

### REFERENCE TO AN ELECTRONIC SEQUENCE LISTING

A computer readable form of the Sequence Listing is filed with this application by electronic submission and is hereby incorporated by reference in its entirety. The Sequence Listing is contained in the XML file created on Jan. 11, 2023 having the name “21-1623-US.xml” and is 1,589,305 bytes in size

### BACKGROUND

Computational enzyme design generally starts from an ideal active site or theozyme, consisting of protein functional groups surrounding the reaction transition state that is then matched into a set of existing scaffolds - typically, native proteins. However, de novo enzyme design has been limited by a lack of suitable protein structures and the complexity of native protein sequence-structure relationships.

### SUMMARY

In one aspect, the disclosure provides polypeptides comprising an amino acid sequence at least 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-1615, not including any functional domains added fused to the protein (whether N-terminal, C-terminal, or internal), and wherein the 1, 2, 3, 4, or 5 N-terminal and/or C-terminal amino acid residues may be present or absent when considering the percent identity. In one embodiment, the polypeptide comprises the secondary structure arrangement H1-L1-H2-L2-E1-L3-E2-L4-H3-L5-E3-L6-E4-L7-E5-L8-E6, wherein “H” is a helical domain, “L” is a loop domain, and “E” is a beta strand domain. In another embodiment, the disclosure provides fusion proteins comprising a polypeptide of the disclosure fused to one or more functional polypeptides.

In one embodiment, the disclosure provides nucleic acids encoding the protein or fusion protein of any embodiment disclosed herein. In another embodiment, the disclosure provides expression vectors comprising the nucleic acid of any embodiment operatively linked to a suitable control sequence. In a further embodiment, the disclosure provides host cells comprising the polypeptide, fusion protein, nucleic acid, and/or expression vector of any embodiment herein. In another embodiment, the disclosure provides libraries comprising 5, 10, 35, 50, 100, 250, 500, 750, 1000, or more different polypeptides, fusion proteins, nucleic acids, expression vectors, and/or host cells of any embodiment herein. In one embodiment, the disclosure provides methods for use of the polypeptides, fusion proteins, nucleic acids, expression vectors, host cells, and/or libraries of any embodiment herein, including but not limited to as specific protein and small molecule binders, and to identify specific protein and small molecule binders.

### DESCRIPTION OF FIGURE

FIG. 1A-FIG. 1G: Generation of idealized scaffolds and computational design of de novo luciferases. (FIG. 1A) Family-wide hallucination. Sequences encoding proteins with the desired topology are optimized by Monte Carlo sampling with a multicomponent loss function. Structurally conserved regions are evaluated based on consistency with input residue-residue distance and orientation distributions obtained from 85 experimental structures of NTF2-like proteins, while variable non-ideal regions are evaluated based on the confidence of predicted inter-residue geometries calculated as the KL-divergence between network predictions and the background distribution. The sequence-space MCMC-sampling incorporates both sequence changes and insertions/deletions (see Methods) to guide the hallucinated sequence towards encoding structures with the desired folds. Hydrogen-bonding networks are incorporated into the designed structures to increase structural specificity. (FIG. 1B-FIG. 1D) The design of luciferase active sites. (FIG. 1B) Generation of DTZ conformers using AIMNet. (FIG. 1C) Generation of a rotamer interaction field (RIF) to stabilize the anionic DTZ and form hydrophobic packing interactions around the DTZ conformers. (FIG. 1D) Docking of the RIF into the hallucinated scaffolds, and optimization of substrate-scaffold interactions using position-specific score matrices (PSSM)-biased sequence design. (FIG. 1E) Selection of the NTF2 topology. The RIF was docked into 4000 native small molecule binding proteins, excluding proteins that bind the luciferin substrate using more than 5 loop residues. Most of the top hits were from the NTF2-like protein superfamily. Using the family-wide hallucination scaffold generation protocol, we generated 1615 scaffolds and found that these yielded better predicted RIF binding energies than the native proteins. (FIG. 1F) Scaffolds generated with family-wide hallucination sample more within the space of the native structures than previous blueprint generated scaffolds, and (FIG. 1G) have stronger sequence to structure relationships than native or blueprint de novo NTF2 scaffolds.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M.P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R.I. Freshney. 1987. Liss, Inc. New York, NY), Gene Transfer and Expression Protocols, pp. 109-128, ed. E.J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In all embodiments of polypeptides disclosed herein, any N-terminal methionine residues are optional (i.e.: the N-terminal methionine residue may be present or may be absent).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In one aspect, the disclosure provides a non-naturally occurring polypeptide comprising an amino acid sequence at least 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-1615, not including any functional domains added fused to the protein (whether N-terminal, C-terminal, or internal), and wherein the 1, 2, 3, 4, or 5 N-terminal and/or C-terminal amino acid residues may be present or absent when considering the percent identity. The amino acid sequence of SEQ ID NO:1-1615 are shown in Table 1.

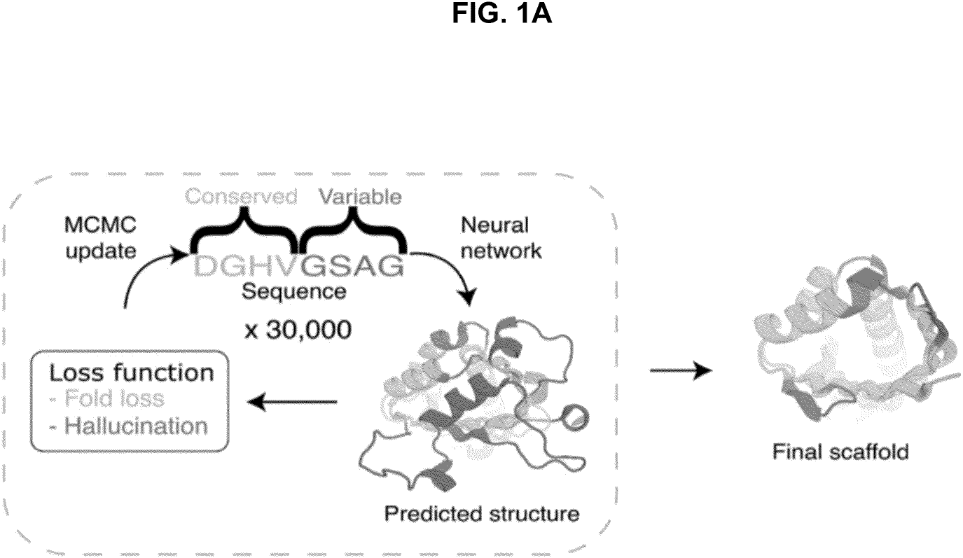

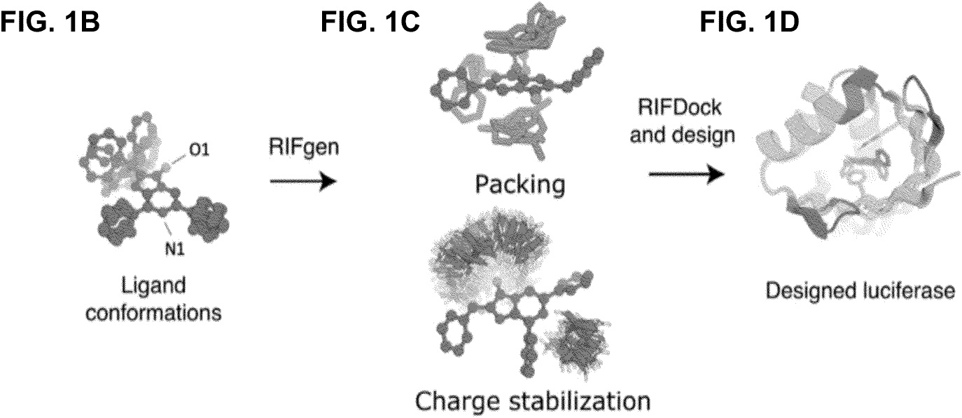

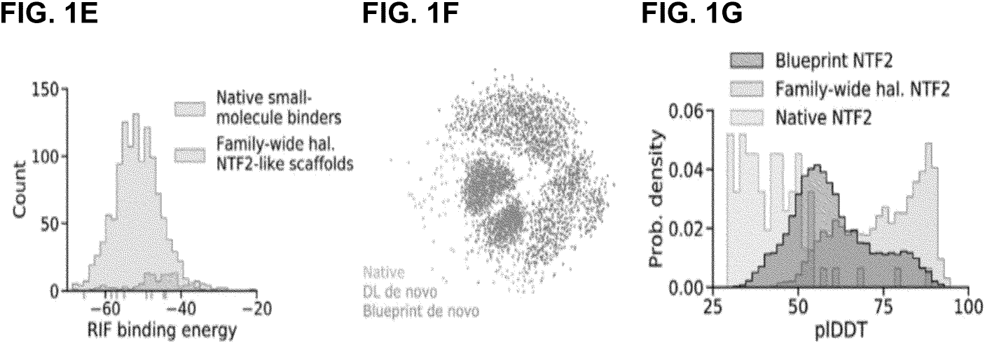

As disclosed in detail in the examples, the inventors designed the polypeptides of SEQ ID NO:1-1615, which serve as scaffolds with pockets of the appropriate size and shape for binding to a target of interest (exemplified in the examples by diphenylterazine (DTZ)). The scaffolds are based on the NTF2-like superfamily as a target topology. The inventors implemented a steered hallucination method to generate idealized structures (FIG. 1A). As seeds for diversification and functionalization, trRosetta was used to generate a diverse set of structural models for 2k naturally occurring NTF2s and used them as seeds. Starting from each native seed structure, steered hallucination of structurally conserved regions was conducted by minimizing an empirical topology-specific loss function over residue pair geometries, while structurally diverse regions (long flexible loops, etc.) were modified both in length and sequence to maximize the confidence of neural network predictions. To further encode structural specificity of the backbone as well as of the binding pocket side chains, the inventors identified buried long-range hydrogen bonding networks in native structures and incorporated these into the idealized scaffolds while designing the sequence constrained by sequence bias of the fold family.

The polypeptides can be used, for example, as specific protein and small molecule binders, and libraries of the polypeptides can be used, for example, to identify specific protein and small molecule binders.

Any N-terminal methionine residues are optional and may be present or absent in the polypeptide and when determining percent identity.

In one embodiment, the percent identity relative to the reference sequence is carried out by sequence alignment with the Needleman-Wunsch algorithm, a common sequence alignment tool for those of skill in the art, which allows for insertions and deletions. In another embodiment, 1, 2, 3, 4, or 5 N-terminal and/or C-terminal amino acid residues are absent and are not included when determining the percent identity relative to the reference amino acid sequence. In another embodiment, all residues in the reference amino acid sequence are included when determining the percent identity relative to the reference amino acid sequence.

In one embodiment, amino acid substitutions relative to the reference amino acid sequence are conservative amino acid substitutions. As used herein, “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Proteins comprising conservative amino acid substitutions can be tested in any one of the assays described herein to confirm that a desired activity, is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In another embodiment, the polypeptide comprises the secondary structure arrangement H1-L1-H2-L2-E1-L3-E2-L4-H3-L5-E3-L6-E4-L7-E5-L8-E6, wherein “H” is a helical domain, “L” is a loop domain, and “E” is a beta strand domain. In some embodiments, domain H1 is 19 amino acids in length, domain H2 is 7 amino acids in length, domain H3 is 14 amino acids in length, domain E1 is 4 amino acids in length, domain E2 is 4 amino acids in length, domain E3 is 10 amino acids in length, domain E4 is 12 amino acids in length, domain E5 is 14 amino acids in length, and domain E6 is 13 amino acids in length.

The loop domains may be of any length and may include insertions, relative to the amino acid sequence of SEQ ID NO:1-1615, of any residues or functional domains as deemed appropriate, including but not limited to metal binding domains, drug binding domains, GPCR receptors, protein switches, and small molecule binding domains.

Thus, in one embodiment, the disclosure provides fusion proteins comprising the polypeptide of any embodiment herein fused to one or more functional polypeptides. Any suitable functional polypeptide may be used, including but not limited to a therapeutic polypeptide, diagnostic polypeptide, targeting polypeptide, scaffold polypeptide, or polypeptide that confers stability on the fusion protein. In one embodiment, the one or more functional polypeptides are fused to the N-terminus and/or C-terminus of the protein. In another embodiment, the one or more functional polypeptides include ones inserted internally into the polypeptide of any one of SEQ ID NO:1-1615. In a further embodiment, the fusion protein comprises the secondary structure arrangement H1-L1-H2-L2-E1-L3-E2-L4-H3-L5-E3-L6-E4-L7-E5-L8-E6, wherein “H” is a helical domain, “L” is a loop domain, and “E” is a beta strand domain, and wherein one or more functional polypeptide is inserted into a loop domain.

In another aspect the disclosure provides nucleic acids encoding the polypeptide or fusion protein of any embodiment or combination of embodiments of the disclosure. The nucleic acid sequence may comprise single stranded or double stranded RNA (such as an mRNA) or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the nucleic acids, expression vectors (i.e., episomal or chromosomally integrated), non-naturally occurring polypeptides, or fusion proteins disclosed herein, wherein the host cells can be either prokaryotic or eukaryotic. The cells can be transiently or stably engineered to incorporate the nucleic acids or expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In another embodiment, the disclosure provides libraries comprising 5, 10, 35, 50, 100, 250, 500, 750, 1000, or more different polypeptides, fusion proteins, nucleic acids, expression vectors, and/or host cells according to any embodiment or combination of embodiments herein. Such libraries can be used, for example, to identify specific protein and small molecule binders.

The disclosure further comprises methods for use of the polypeptide, fusion protein, nucleic acid, expression vector, host, and/or library of embodiment herein for any suitable use as disclosed herein, including but not limited to identifying or designing enzymes or small molecule binders.

In another aspect, the disclosure provides methods for de novo enzyme design, comprising methods according to any steps or combination of steps as described herein.

### EXAMPLES

### Summary

De novo enzyme design has sought to introduce active sites and substrate binding pockets predicted to catalyze a reaction of interest into geometrically compatible native scaffolds, but has been limited by a lack of suitable protein structures and the complexity of native protein sequence-structure relationships. Here we describe a deep-learning based “family-wide hallucination” approach that generates large numbers of idealized protein structures containing diverse pocket shapes and designed sequences that encode them. The design of highly active and specific biocatalysts from scratch with broad applications in biomedicine is an important milestone for computational enzyme design, and our approach should enable the design of a wide range of new and useful luciferases and other enzymes.

### Study

Bioluminescent light produced by the enzymatic oxidation of a luciferin substrate is widely used for bioassays and imaging in biomedical research. Because no excitation light source is needed, luminescent photons are produced in the dark which results in higher sensitivity than fluorescence imaging in live animal models and in biological samples where autofluorescence or phototoxicity is a concern^{4,5}. However, the development of luciferases as molecular probes has lagged behind that of well-developed fluorescent protein toolkits for a number of reasons: (i) very few native luciferases have been identified; (ii) many of those that have been identified require multiple disulfide bonds to stabilize the structure and are therefore prone to misfolding in mammalian cells; (iii) most native luciferases do not recognize synthetic luciferins with more desirable photophysical properties; and (iv) multiplexed imaging to follow multiple processes in parallel using mutually orthogonal luciferase-luciferin pairs has been limited by the low substrate specificity of native luciferases.

We sought to use de novo protein design to create new luciferases that are small, highly stable, well-expressed in cells, specific for one substrate, and need no cofactors to function. As we are not constrained to natural luciferase substrates, we chose a synthetic luciferin, Diphenylterazine (DTZ) as the target substrate duo to its good quantum yield, red-shifted emission³, favorable in vivo pharmacokinetics^{14,15}, and lack of required cofactors for emission. Previous computational enzyme design studies have primarily repurposed native protein scaffolds in the PDB, but there are few native structures with binding pockets appropriate for DTZ, and the effects of sequence changes on native proteins can be unpredictable. To circumvent these limitations, we set out to generate large numbers of ideal protein scaffolds with pockets of the appropriate size and shape for DTZ, and with clear sequence-structure relationships to facilitate subsequent active site incorporation. To identify protein folds capable of hosting such pockets, we first docked DTZ into 4000 native small molecule binding proteins. We found that many NTF2 (nuclear transport factor 2)-like folds have binding pockets with appropriate shape-complementary and size for DTZ placement (FIG. 1e), and hence selected the NTF2-like superfamily as the target topology.

### Family-Wide Hallucination

Native NTF2 structures have a range of pocket sizes and shapes but also contain non-ideal features such as long loops which compromise stability. To create large numbers of ideal NTF2-like structures, we developed a deep-learning based “family-wide hallucination” approach that integrates unconstrained de novo design^{19,20} and fixed backbone sequence design approaches²¹ to enable the generation of an essentially unlimited number of proteins having a desired fold (FIG. 1a). The family-wide hallucination approach utilizes the de novo sequence and structure discovery capability of unconstrained protein hallucination^{19,20} for loop and variable regions, and structure-guided sequence optimization for core regions. We employed the trRosetta™ structure prediction neural network²², which is effective in identifying experimentally successful de novo designed proteins and hallucinating new globular proteins of diverse topologies. Starting from sequences and predicted structures of 2,000 naturally occurring NTF2s, we used trRosetta™ to optimize the amino sequence of conserved core and variable loop regions. Protein core idealization was carried out with a topology-specific loss function over core residue pair geometries (see Methods) and variable loop optimization, by optimizing sequence length and identity to maximize the confidence of the neural network in the predicted structure. To further encode structural specificity, we incorporated buried, long-range hydrogen-bonding networks. The resulting 1615 family-wide hallucinated NTF2 scaffolds (SEQ ID NO:1-1615) provided more shape complementary binding pockets for DTZ than native small-molecule protein binding proteins (FIG. 1e). This approach samples protein backbones closer to native NTF2-like proteins (FIG. 1f) and with better scaffold quality metrics than a previous non deep-learning approach²³ (FIG. 1g).

### De Novo Design of Luciferases for DTZ

Standard computational enzyme design generally starts from an ideal active site or theozyme consisting of protein functional groups surrounding the reaction transition state that is then extrapolated into a set of existing scaffolds^{1,2}. However, the detailed mechanism of native marine luciferases is not well defined as only a handful of apo-structures and no holo-structures have been solved^{24,25} (excluding calcium-regulated photoproteins). Both quantum chemistry calculations^{26,27} and experimental data^{28,29} suggest that the chemiluminescent reaction proceeds through an anionic species and that the polarity of the surroundings can substantially alter the free energy of the subsequent single electron transfer (SET) process with triplet molecular oxygen (³O₂). Guided by these data, we sought to design a shape complementary catalytic site that stabilizes the anionic state of DTZ and lowers the SET energy barrier, assuming that the downstream dioxetane light emitter thermolysis steps are spontaneous. To stabilize the anionic species of DTZ, we focused on the placement of the positively charged guanidinium group of an arginine residue to interact with the anionic imidazopyrazinone core.

To computationally design such active sites into large numbers of hallucinated NTF2 scaffolds, we first used AIMNet³⁰ to generate an ensemble of anionic DTZ conformers (FIG. 1b). Next, around each conformer, we used the RIFgen method^{31,32} to enumerate Rotamer Interaction Fields (RIFs) on 3D grids consisting of millions of placements of amino acid sidechains making hydrogen bonding and nonpolar interactions with DTZ (FIG. 1c). Additionally, we included an arginine guanidinium group near the deprotonation site at the nitrogen of imidazopyrazinone (N1 atom) in the RIF. RIFdock was then used to dock each DTZ conformer and associated RIF in the central cavity of each scaffold to maximize protein-DTZ interactions. An average of eight sidechain rotamers including an arginine to stabilize the anionic imidazopyrazinone core were positioned in each pocket (FIG. 1d). For the top 50,000 docks with the most favorable sidechain-DTZ interactions, we optimized the remainder of the sequence using RosettaDesign™ (FIG. 1d) for high-affinity binding to DTZ with a bias towards the naturally observed sequence variation to ensure foldability. During the design process, pre-defined hydrogen bond networks (HBNets) in the scaffolds were kept intact for structural specificity and stability, and interactions of these HBNet side chains with DTZ were explicitly required in the RIFdock step to ensure preorganization of residues essential for catalysis. In the first sequence design phase, the identities of all RIF and HBNet residues were kept fixed, and the surrounding residues were optimized to hold the sidechain-DTZ interactions in place and maintain structural specificity. In the second sequence design step, the RIF residue identities (except the arginine) were also allowed to vary, to identify apolar and aromatic packing interactions missed in the RIF due to binning effects. During sequence design, the scaffold backbone, sidechains, and DTZ substrate were allowed to relax in Cartesian space. Following sequence optimization, the designs were filtered based on ligand-binding energy, protein-ligand hydrogen bonds, shape complementarity, and contact molecular surface.

### Conclusion

Computational enzyme design to date has been constrained by the number of available scaffolds, which limits the extent to which catalytic configurations and enzyme-substrate shape complementarity can be achieved¹⁶⁻¹⁸. The use of deep-learning to generate large numbers of de novo designed scaffolds here eliminates this restriction; moving forward, the more accurate RoseTTAfold^{TM39} and AlphaFold2^{TM33} should enable still more effective protein scaffold generation by leveraging family-wide hallucination abilities. The diversity of scaffold pocket shapes and sizes enabled the exploration of a range of catalytic geometries and the maximization of substrate-enzyme shape complementarity. The family-wide hallucination method opens up an almost unlimited number of new scaffold possibilities for substrate binding and catalytic residue placement, which is particularly important in cases where the reaction mechanism, and how to promote it, are not completely understood: many structural and catalytic hypotheses can be readily enumerated with different catalytic residue placements in shape and chemically complementary binding pockets. While luciferases are unique in catalyzing the emission of light, the chemical transformation of substrates into products is common to all enzymes, and the approach developed here extends to a wide variety of chemical reactions.

### Methods

### 1. Materials and General Methods

Synthetic genes and oligonucleotides were purchased from Integrated DNA Technologies or GenScript. The synthetic gene was inserted between NdeI and XhoI sites of a pET29b+ vector, containing an N-terminal hexahistidine tag followed by a TEV protease cleavage site and a C-terminal stop codon. Restriction endonucleases, Q5 PCR polymerase, and T4 ligase were purchased from NEB. Plasmid DNA, PCR products, or digested fragments were purified by Qiagen DNA purification kits. DNA sequences were analyzed by Genewiz. All other chemicals were purchased from Sigma-Aldrich or Fisher Scientific and used without further purification. To identify the molecular mass of each protein, intact mass spectra were obtained via reverse-phase LC/MS on an Agilent 6230B TOF on an AdvanceBio RP-Desalting column and subsequently deconvoluted by Bioconfirm software using a total entropy algorithm. ÄKTA pure M with UNICORN 6.3.2 Workstation control (GE Healthcare) coupled with a Superdex™ 75 Increase 10/300 GL column was used for size exclusion chromatography. DNA and protein concentrations were determined by a NanoDrop™ small-volume 8 channel UV/vis spectrometer. CD spectra and CD melting experiments were performed by the default setting on a J-1500 Circular Dichroism Spectropolarimeter (Jasco). SDS PAGE and luminescence images were captured by a Bio-Rad ChemiDoc™ XRS+. Images were analyzed using the Fiji image analysis software.

### 2. General Procedures for Protein Production and Purification

Lemo21(DE3) strain was used for transformation with the pET29b+ plasmid encoding the gene of interest. Transformed cells were grown for 12 h in TB medium supplemented with kanamycin. Cells were inoculated at 1:50 ratio in 100 mL fresh TB medium, grown at 37° C. for 4 h, and then induced by IPTG for an additional 18 h at 16° C. Cells were harvested by centrifugation at 4,000 g for 10 min and resuspended in 30 mL lysis buffer (20 mM Tris-HCl pH 8.0, 300 mM NaCl, 30 mM imidazole, and Pierce™ Protease Inhibitor Tablets). Cell resuspensions were lysed by sonication for 5 min (10 s per cycle). Lysates were clarified by centrifugation at 24,000 g at 12° C. for 40 min and pre-equilibrated with 1 mL of Ni-NTA nickel agarose at 4° C. for 1 h. The resin was washed twice with 10 mL wash buffer and then eluted in 1 mL elution buffer (20 mM Tris-HCl pH 8.0, 300 mM NaCl, 300 mM imidazole). The eluted proteins were purified by size exclusion chromatography in PBS. Fractions were collected based on A280 trace, snap-frozen in liquid nitrogen, and stored at -80° C.,

### 3. Computational Design of Idealized Scaffolds

Our generation of idealized NTF2-scaffolds can be divided into four parts: (3.1) Generation of seed-structures, (3.2) optimization of backbone geometries using trRosetta™-based hallucination, (3.3) generation of structure-conditioned sequence models to bias design, (3.4) design and filtering.

### 3.1 Generation of Seed Structures

We thought to increase the set of NTF2 structures by complementing experimentally resolved structures from the PDB with highly accurate models generated by trRosetta^{TM22}. To achieve this, we first collected 85 NTF2-like protein structures from the PDB based on SCOPe annotation (d. 17.4 SCOPe v2.05). Corresponding sequences were then used as queries to collect sequence homologs from UniProt™ by performing 8 iterations of hhblits at 1e-20 e-value cutoff against uniclust30_2018_08 database; default filtering cutoffs were relieved (-maxfilt 100000000 -neffmax 20 -nodiff -realign _max 10000000) to maximize the number of the output hits. All the hits were redundancy reduced using cd-hit⁴⁶ with a sequence identity cutoff of 60% yielding a set of 7,573 candidates for modeling.

To generate inputs for structure modeling with trRosetta™, we built multiple sequence alignments (MSAs) for each of the 7,573 selected sequences with hhblits using a more conservative e-value cutoff of 1e-50; the resulting MSAs were also complemented by hits from hmmsearch against uniref100 (release-2019_11) with the bit-score threshold of 115 (i.e. ~1bit per position). After joining the above two sets of alignments and filtering them at 90% sequence identity and 75% coverage cutoffs, only sequences with more than 50 homologs in the corresponding MSAs were retained for modeling (2,005 sequences). The filtered MSAs along with information on the top 25 putative structural homologs as identified by hhsearch against the PDB100 database of templates were used as inputs to the template-aware version of trRosetta^{™47} to predict residue pair distances and orientations. Network predictions were then used to reconstruct full atom 3D structure models using a Rosetta™-based folding protocol described previously²².

### 3.2 Hallucination of Idealized NTF2s

Seeking to idealize the native structure seeds, we reasoned that trRosetta™, a convolutional residual neural network, which predicts residue-residue orientations and distances from sequence, could serve as a key component in a protein idealizer. Previously, this network has been used to generate diverse proteins that resemble the “ideal” structures of de novo designed proteins by changing the protein sequence to optimize the contrast (KL-divergence) between the predicted geometry and that of randomly generated sequences¹⁹.

For our purpose, the desired fold-space is not diverse but instead focused on the NTF2-like topology. To guarantee generation of ideal structures within this fold-space, we implemented a new fold-specific loss-function, which biased hallucinations based on observed geometries in native crystal structures. As many experimentally characterized NTF2s contain non-ideal regions, we began by creating a set (χ) of trimmed but ideal NTF2s by manually removing non-ideal structural elements such as kinked helices, and long or rarely observed loops. For each seed structure, we then used a structure-based sequence alignment method (see 3.3) to find equivalent positions between the seed structure and χ. Residue pairs were considered to be in a conserved tertiary motif (TERM) if there were 5 or more equivalent positions in χ. The smooth probability distributions based on observed geometries in χ were then computed. For distances we used a Gaussian distribution with mean equal to the true distance denoted by D and standard deviation denoted by σ equal to 0.5 Å. The probability density function for distances d is given by:

$$f \left(d ; D , σ\right) = \frac{1}{\sqrt{2 π {σ}^{2}} exp \left(- \frac{{\left(d - D\right)}^{2}}{2 {σ}^{2}}\right)}$$

Using this density function one can construct a categorical distribution for binned distances by evaluating this function at the centers of the bins and then normalizing by a sum of all values in different bins. Similarly, a von Mises distribution was used for omega angle smoothing with probability density function given by ƒ(ω; Ω, κ) = N(κ) exp[κ cos(ω - Ω)] where N(κ) is a normalizing constant, Ω is the crystal value, κ is the inverse variance chosen to be 100, and ω is the smoothed angle. For phi and theta angles a von Mises-Fisher blur is given by ƒ(x; µ, κ) = N(κ) exp[κ µ^{T}x]where N(κ) is a normalizing constant, µ is a unit vector on a 3D sphere corresponding to the phi and theta angles from the crystal structure, x is a smoothed unit vector, and κ is the inverse variance chosen to be 100.

Next, we converted those probability distributions to energy landscapes (ie - negative log likelihoods) and sought to minimize the expected energy. This soft restraint encouraged the network to seek out the consensus structure, while still allowing deviations where needed. Specifically, we formulated the fold-specific loss as:

$$\begin{matrix}{L}_{f o l d} = {\sum }_{x ∈ \left\{d , \text{ω,θ,ϕ}\right\}} \left[{\sum }_{i , j = 1}^{L} {\sum }_{k = 1}^{{N}_{x}} - {m}_{i j} {p}_{x , i j k} l n \left({s}_{x , i j k}\right)\right] / {\sum }_{i , j = 1}^{L} {m}_{i j} \\ {m}_{i j} = \left\{1 i f i a n d j a r e i n a T E R M ; e l s e 0\right)\end{matrix}$$

where p is the network prediction and s is the smoothed probability distribution of the conserved residue pairs. For the second part of the loss function and similar to previous work¹⁹, we sought to maximize the Kullback-Leibler (KL) divergence between the predicted probability distribution and a background distribution for all i,j residue pairs not in a TERM.

$$\begin{matrix}{L}_{h a l l} = \\ - {\sum }_{x ∈ \left\{d , \text{ω,θ,ϕ}\right\}} \left[{\sum }_{i , j = 1}^{L} {\sum }_{k = 1}^{{N}_{x}} \left(1 - {m}_{i j}\right) {p}_{x , i j k} l n \left({p}_{x , i j k} / {b}_{x , i j k}\right)\right] / {\sum }_{i , j = 1}^{L} \left(1 - {m}_{i j}\right)\end{matrix}$$

where b is the background distribution and Nₓ is the number of bins in each probability distribution (N_{d} = 37, N_{ω,θ}, = 25, N_{φ} = 13). Briefly, b is calculated by a network of similar architecture to trRosetta™ trained on the same training data, except it is never given sequence information as an input. The final loss is given by:

$$L = {L}_{f o l d} + {L}_{h a l l}$$

We used a Markov Chain Monte Carlo (MCMC) procedure to search for sequences that trRosetta™ predicted to fold into structures that minimize this loss function. We allowed four types of moves with different sampling probabilities: mutations (p=0.55), insertions (p=0.15), deletions (p=0.15), and moving segments (p=0.15). Mutations randomly changed one amino acid to another, with an equal transition probability for all 20 amino acids. Insertions inserted a new amino acid (all equally likely) into a random location subject to the KL-divergence loss. Deletions deleted a random residue from the same locations. Finally, we also allowed “segments” to move, cutting and pasting themselves from one part of the sequence to another, while maintaining the same overall segment order. Here, a “segment” is a continuous stretch of amino acids all subject to fold specific loss, often composed of a single strand or helix. Starting from a random sequence of an initial length (typically 120 amino acids), we used the standard Metropolis criteria to accept or reject moves:

$${A}_{i} = m i n \left[1 , e x p \left(- \left({L}_{i} - {L}_{i - 1}\right) / T\right)\right]$$

where Aᵢ is the chance of accepting the move at step i, Lᵢ is the loss at the current step, Lᵢ₋₁ is the loss at the previous step and T is the temperature. The temperature started at 0.2 and was reduced by half every 5 k steps. Generally, it took 30 k steps to converge.

### 3.3 Structure-Conditioned Multiple Sequence Alignment

Given the complexity of the NTF2-like protein fold, we hypothesized that it was necessary to impose sequence design rules to disfavor alternative states (negative design). Towards this end, we computed a structure-conditioned multiple sequence alignment based on native NTF2-like proteins. Specifically, we used TMalign⁴⁸ to superimpose each of the 2005 predicted native structures (from 3.1) onto each hallucinated backbone (from 3.2). Next, to find structurally corresponding positions, we implemented a structure-based dynamic programming algorithm, similar to the Needleman-Wunsch algorithm⁴⁹. However, instead of using the amino acid similarity as the scoring metric, we used a tunable structure-based score function. After aligning the two structures, we scored the structural similarity of any two residues by empirically weighting several metrics: (1) Distance between Ca atoms, (2) differences between backbone torsion angles (phi and psi) backbone torsion angles and (3) the angle (degrees) between the vectors pointing from Cα to Cβ in each residue. To calculate the unweighted score for each component, we normalized each by a maximum possible value (180 degrees for angles and 10 Å for distances) and included a “set point” that approximately delineated when we judged a metric to indicate two residues to be more similar than not. Values above this setpoint are positive, indicating two residues are similar and values below the set point indicated two residues are dissimilar.

$$S c o r {e}_{u n w e i g h t e d} = \left(s e t \_ p o i n t - v a l u e\right) / m a x \_ v a l u e$$

Each value was scaled by its normalized weight and summed to give an overall similarity score between any two amino acids.

These similarity scores were used as the similarity metric in our dynamic programming algorithm, in place of the typical BLOSUM62 similarity metric. We used a gap penalty of 0.1 and an extension penalty of 0.0. Finally, after concatenating all the structure-conditioned aligned sequences, we used PSI-BLAST-exB^{50,51} to compute sequence redundancy weighted log-odds scores for each amino acid at each position (position-specific scoring matrices, PSSMs).

### 3.4 Design

To design the resulting backbones, we sought, in addition to the sequence patterns captured in the PSSM (3.3), to further specify the backbone conformation and functionalize the pocket, by installing entire hydrogen bonding networks from native NTF2-like proteins. We compiled two sets of hydrogen bonding networks: a set for the cavity containing 85 networks and another set of networks connecting the C-terminal region of the first helix with the third beta-strand containing 25 networks. In 20 independent attempts for each backbone, we randomly grafted a network from each set, fixed the identities of hydrogen bonding residues, and designed the sequences for all other positions under PSSM constraints. The resulting models were filtered for various backbone quality metrics and for maintenance of hydrogen bonding networks in the absence of constraints, resulting in a total of 1615 idealized scaffolds.

### 4 RIFdock Tuning Files

The hierarchical search framework of RifDock is a powerful way to search through 6-dimensional rigid body orientations. While originally designed to work with physics-based forcefields, the scoring machinery can easily be modified to do other things. A system was added called “Tuning Files” that allows one to tune the energetics of rifdock by “requiring” specific interactions. Specified interactions can range from specific hydrogen bonds, to specific bidentates, and even to specific hydrophobic interactions. The specifics are that during the RifGen stage, each stored rotamer is compared against a list of definitions in the Tuning File. If the rotamer satisfies a definition, it is stored into the RIF with a “Requirement Number”. Later during RifDock, these Requirement Numbers are available during scoring and the presence or absence of certain rotameric interactions may be used to penalize or even completely discard dock solutions. In this work, the Tuning Files were used to require the specific hydrogen bond interactions between the arginine and the secondary amine in the pyrazine ring of the colenterazine-like substrate.

### 5 Designing Theozyme Architectures Into De Novo NTF2 Scaffolds

De novo design of luciferases can be divided into three main steps - scaffold construction, substrate placement with required interactions, and sequence design. With the idealized NTF2-like scaffolds in hand, we selected 5 diverse rotamers from AIMNet and used the Rotamer Interaction Field (RIF) docking method³¹ to exhaustively search a large space of interacting side chains to the anionic form of DTZ. Chemically, deprotonation of N1 hydrogen is the first step to forming an anionic species. We first generated RIF using RifGen³¹ to guide placement in the protein scaffolds. We required the placement of a positively charged Arginine sidechain by a tuning file (see below) to stabilize the formation of negative charge N1 atom where the deprotonation occurs initially and enumerated large numbers of possible sidechain interactions with the rest of DTZ.

HBOND_DEFINITION
N1 1 ARG
END_HBOND_DEFINITION
REQUIREMENT DEFINITION
1 HBOND N1 1
END_REQUIREMENT_DEFINITION

Rifdock was then used to hierarchically search for the best combination of RIF to place on the input backbone. Although the negative charge can move to another electronegative atom O1 via resonance of the imidazopyrazinone core, it is unclear which anionic species is more critical for the luciferase-catalyzed luminescence emission. Thus, we let RIFdock place the polar rotamers on the basis of hydrogen-bond geometry to O1 and apolar rotamers to DTZ without specific requirements. In the next docking step, we parsed the -scaffold_res argument with a list of residue numbers as scaffold backbone positions that were annotated as pocket residues to allow a hierarchical search of RIF placement. We only allowed the RIF placements in the pocket residues and left pre-defined hydrogen bond networks (HBNets) intact. After RIFdock, we continued for Rosetta™ sequence design where the score function was reweighted for higher buried_unsat_penalty⁵² and the amino acid selection was biased by giving a pre-generated PSSM file via SeqprofConsensus task operation. This would minimize buried unsatisfied residues and increase pre-organized architectures in the core that are known to be beneficial for a catalytic pocket⁵³. Two rounds of FastDesign calculation were included: we restricted the RIF rotamers and core HBNets to repacking in the first round while we allowed other residues for re-design based on PSSM during the Monte Carlo simulated annealing procedure. After the surrounding residues were optimized to retain the RIF interactions, we allowed the re-design of RIF rotamers, to find efficient aromatic and hydrophobic packing around DTZ while catalytic residues (the N1 requirement) were still limited to only repacking. The final set of designs was obtained after filtering by ligand-binding interface energy, shape complementarity, contact molecular surface, number of HbondsToResidue, and the presence of N1_hbond

TABLE 1

| SEQ ID NO: | SEQUENCE |
|---|---|
| 1 | PSEEEIKEFLRRFYEAINRGDADAVKELLTPGWTYHHASGQTFTDQEEFREWVEKMFKAGTRLQIDEIRIDGKTVTVHVTMHQTRGNQDHTIRTTDHHRFHDDGKLVETRAHYNHE |
| 2 | ASEEEIRELIRRFYEAFNKGDMEKLKEILAPGAQLHISNGQTFNDLEEITKFFQTMLSQGTKIEIKSIEVRGDTVTVHVTFHTTHGNQDHTVDVTDHYHFDKDGKIRRLQSHFNPR |
| 3 | PSEEEIRRMVRRFFEAFNRNDADALARLFHPGAQMHVSNGQTFNDREEIKEWFERLLSQDVQIEIHSIEVRGNTVTVHWTGHMNQGNQTKTVTATTHFHLVKDGKIVEMRIHINQQ |
| 4 | MSEEEIKEILHALYEAFNRGDMDAFAEYFAPGAQMHISNGQTFNDREEIKEFFAQLFKQGVQIEIKEIRIDGDTATVHVEVHRTHGNQDHTYTVTHHWVFRKDGKVVELRQHVNPQ |
| 5 | DFKEEVKRVLKEFYEALDKKDIDTAASFFTDDSTMHVQEGTTFKNKEEIKKWFKEFLDTFPRMHREIKSIHITGDKVTIHVTIHVTHGDRDYTYHITEHHRLDKDGRIKETHIHTNQQN |
| 6 | DFQEQVKEWIKRFVEALDKGDVEKVVSLFTDDWTFHLYQGQTFTNPEEFKEWVERFLRTFKRMHREIKEIRVEGDRATVHVTVHLTQGDRDYTVHVTEHHKFDKDGRITHTQMHENEQK |
| 7 | PDEEAIREWLRQFFRALDEGDMDKAMSLLSDDVTVHFYEGYTATTKDEIKEWFARWYSKFPQMHRQIDSIEITGKTVHVHWTIHLHQGDQTYTYRATTTFHFDHDGRIKEIRITIREEK |
| 8 | PISEEEIKDWVRRFIEAFNKRDADALKSLYSDDSTIHFGDGRTFTNPEEFAKWVKEYLRHSDTRLEIKSIEIHGNTVTVHVQMHTTHPSGQTYTYRVTRHITFDHDGKIKEDHIQTDQLH |
| 9 | SLSEEQIKELLKRFYEAFNRNDADALAELLHPDATMHLSDGSTFHNVEEILKWFREFMKKSDVQIDVKEIEIRGDTVTVHYTMTVNHPSGQTHTVDGRDHFTFDHNGKIVEVRSHYQPQS |
| 10 | PSAEEIIRELKKRLVEAMNKGDMDKLRELVHDDAQFHFNGQTFHDKEEFLEWFAQTSPVKIQIDELHIQQNDDGTYTVTVHVTMHNDDEGKTVRVTAHDRYDMRHNKLVETRFHTDQG |
| 11 | PSDEEIVRELVRRFYEALNNGDADTAKSLLHDDWRLHVDGRTFNSPDEWIEWVKRVPQLHHEIKSLEVRVNHDGTVTVTVHVTVHDSSKNTTHRFTDHHHVDSRHNKIVRTQVHTDNG |
| 12 | PSDEEVARELVRRFIEAWNKKDLDTMFSLLHPDWQYTQNGRTFHSRDEFKEWVRTHPDLKVEVDSIEVRTNPDGTVTVHVEVTARDDTTNQDIKFHEEHHVDSRHNKIVETRQHEDQG |
| 13 | PSDEEIVRELIRRLYEAINRGDMKEIFDLLHPDWQFTNNGTTAHSREEFIERLKKRDQFHVHVDDLHVQINHDGTVTVEVHVTVTDSKTNRHFKVRDEHHFDSRHNKLVETRVHTDPG |
| 14 | PSDEEIIKELVRQFYEAVNNNDMDTLRELLHPDATFTFNGRTFHDVDEFLKWVQDIPQTHHEIKSIEVRVHDDGTITVTVHVTTTDESTKTTVTVHDHYTVDKRHWKIVRVQSHEDKG |
| 15 | PSDEEIARELVKRFYEAFDKKNMDELHEMLHPDWQYTQDGNTWHSREEFLKWASNVPQLRHEIKELHVQTNPDGTITVTVHVEVHDSSTNRTVNVTDHHRIDKRTNKIVETRSHTDNT |
| 16 | PMSKEEVKEVLRRFFEAINKNDRDTMKELLDDDWEFHSSSGRTFHGADEFLEWITQWASTAQIEIKSIEIEIRGDTIHVTVHVTVHETHGNQTHTVDVTEHHRLDARNNKIVETRVHFNQR |
| 17 | VSDEQQERAKQAIERFIEAMNKDPEEVYKLLAPGWRYTDSSTGRTFDKDEFKEMVEKLFKQNFHFQIESIRVELNPDGTITAHVTVQVTNDSTGSTVTVHETHHLDSRTGQIVETRVTPTG |
| 18 | PSDEQQIRDWLKKFYEAWNNNDLDALVELLTPDWTYHHSSGQTYTTLDEFREWVERMLSQDTQINMEEMRIEFDGDTATVTVHYTATNGSTKTTYKATETHHFERDKDGWKIVETRVHHHQT |
| 19 | PSDEEAIRELARRFYEAFNTKDADALVEMYDPNWTYHHSSGRTFTTLDEFKEWVERFLSSDVRLEIKSMEVEVRGDEATVTVEVDVTQRQTGRTFHAHDTHHFRRTKDGWKLIETRTHTHQA |
| 20 | PSDEEAIRELKRRFIEAFNNKDADALARLFHPDAQMHTSNGQTFNTKDEIKKFLEDLLKQDIRIELKSVEIEVRGDTIHVTFHVTVTNSSTGRTWDGTQHDEYRKEPGGWKLVRTEERLNQK |
| 21 | PSDEQIIRDWVRRWVELLNRGDADAVASLYAPDATVHHHDGRTLTSREEIKEWVEEMFSRQVEVHLDSLEIEFRGDKATVHAHLTFTEQRTKRTFKGTATFHFQRTKDGWQLKEQHVHIHQT |
| 22 | PSDEEQIRELVKRLYDALNNGDWEAFASLFHPDAQFHHASGRTLHTLEEIKEWAKQFLSNNIRIEIKEIRIEINGNTITVTVHVTVTNAKTKETFEVTETHHFRKDEGGWYLVRTEVHIRKL |
| 23 | PEVEEAVRELKKRFVEAFNKGDLEALERMFHDDATMHVSNGQEYHSKEEIKEWIKRFLSNDARVEVSIEVQIVNGDEATATSHFHVTYRNGQTYTGTAHDFYRRKDGTWKIIRVELQWHQN |
| 24 | HEIEERIREIIKRLIEAIDRGDLDALEEIYDDDAQMHISDGITFNNKEEIKEWLKRYSSFRTAHEVHVEVHIRNDSHVTVHVTVHYTHENGQHFTVHRTFHFQFENGQVKLKEDRIQRDRQ |
| 25 | MSEQEIREAIREWLRRYYEAFDNGDWEKLSRLMAPDIRMEYNGRTFTNLEEILRFLREFWEKFNTAHEVHIEIHIDDKDTTTVTVEVHVTLHHNGKTFSTTAQHHWHVEFHGRQLRFKEVRTQTHESPQ |
| 26 | MSEEEIREAIKETLRRFYEALDNNDLDTLRSILHPDWQMHYNGEQFTNREEFAEWVDKFWKNWDSRHDVEIHIDMSDDSTTTATVHVHVTFHHNGQQFHVTVDDTHHFRMEGNELKMIETRSQTRENKD |
| 27 | ASEEEIRQIIKRLARALDEGDWDTLAKLYSDDSRIEENQDNGTTIEFTTREEFIRWVREFKKNAEVHREVHIHVDKSTNTATAHMKVHVTHGNKSITMDVTSHFHFTDGKLKEEQVHIRAQE |
| 28 | MSKEEIKRILKQFFEAMDKGDADKLKKLLHPGATFHLANGQTFTDVDEFVKWVEDFFNNVQLHREVHIHIHPNGRTVHVEWTLHLRHGDTKYTIHGRTTFHFKDGKIAEVRFHWDTQET |
| 29 | PDEEKIRQIVEALVEAINHGDLDALESFYTDDSTMHFNEGETLTNREEIKRWFEEFIKKFNVRIEIKSVHVDGKTITVNVTVHLTHNGRTVTYHVQRHFRVEGDKLTDDHIHRNQTE |
| 30 | SSEEEIREILRRLFEAFDKGDLDELKELFHDGWTFHDPSGQTFTTKDEFKEWVKRLLESLSDIKRGDIEIRVQDDGTVTVHVTVHVTDSRTGRTVTIDYTEHHHFRNGKLTETHVHSHQKS |
| 31 | DDREEIKDIIRKFFEAFENGDADKLKELLAEDATFHLYQGITFKSLDEIREWAERLFSTAEFRFDSIEIHIHDDGTATVHWTMTVHHDNQTFHFNATTHFVFENGKIKEVRIHADNTD |
| 32 | DRREEIKDWLRRFYEAFEKGDWEKLARLMDDDATFHLYQGITFTDKEEFIRWFREFISNAEVRFDEIRFHINDDGTATVHYTVTVHHDNKTIHANATDHFVFSDGKVTEIRSHADNQE |
| 33 | DDREKIKEFIKEFFEAFQNGDADRLKELMAEDATFHFSNGTTFTNPEEFREFVERFFSRMDIKIDRIEIHINDDGTATAHVEITVTHDDRTHHVTVTVHDRYVDDKLKERRFHYDNKN |
| 34 | PFREEIKDWKRRFIEAFEKGDMDKLKELFAEDATVHFWNGETATTLDEIKEWFTRLWSTAEFRFEEVRIHIRDDGTVTVHMRVTVRHDNEDRTARYTVHDRYRDGKLTETQFHTDQDR |
| 35 | PDEEEIRDTVRRYYEAFEKGDAEALAELFHDDATFHVSEGITFTTKDEFKEYVSQLLRNAEYRFDSIEIHINDDGTVTAHYTVTVHHSDRTIHINATDHFHFSDKYIQELRSHFDRQE |
| 36 | DDRERIKEWLRRYFEAFEKGDADALKSLFTDDWTFHHSSGRTFTSRDEFKEWFKEIMKNAEYRIDEIRIHINDDGTVTVHVKITVTHDNKTVTMDVTEHHRFTNDRIVETRVHIDNKQ |
| 37 | PWEERIKELLKRFYEALQNGDADALAELMSDDVQFHHSSGTTFDSKEQFREWVERLFSSADFKIDSIEIRFNDDGTVTVHVTVTVHHDNKTIHINATHHWVFRDGKVTRVEARLDNTE |
| 38 | PSEEQLRELLKRFYTAINNGDLDALKELLHDGWQFHSSSGRTFHTKEEFAEWVERMLSNNTRLQIDSMNADQTEPRATVHVTFHTNEGNQTKTVHVTEHHRFKDGKIVETRQHHHTG |
| 39 | PTEEEIKEWVKQYFEAMQNKDVDKLVSLYDPGVQVHFWSGQTFNDREELKEWYERWFSQFEDAQMEIHVEIRGDRVTVHIHVTNPSTKTTTTYTFHFQFSDGRITEVHIHRNDHE |
| 40 | QDEDEKRIKDFIRRFAEAVDKGDWETLRRLLHDDWRFHHSSNIQFDNPEEFVRWYIERFRKEFDEVHRQIDSIEIHGQTATVHMTAHFTRGNKTFTVHVTEHHHFRDGRLVETHVHYDEKET |
| 41 | PDENEEAIKELVRRFFEALDNNDRDTFKSLFHDDAEFHLSDNITFSNADEFVEFFFEKFAREFERVRHEIHSIEIRGNRVRVEWTAHFTRGHKDVTIHGTTTFQVRDGKITRVEIHFDSKDT |
| 42 | PKEDEEAIKEFKRQLVEAFNHKDMDTIKELLTPDIEVHVSDGITFTNLDEFVKWFADKFLKHSDFRMQITEIRIHGHKVTVHVEVHWSHNGSSRTVRVTVHDRYRNGKITSTEFHWHNEN |
| 43 | MSEEEIKETIKRLMEAFDKGDMDTVASLFHPDAQFHISEGITFDNLEEFVEWWFQKFARKITRAHREIKSIEIRGDTVTVRWTVTVTNKDTNTTRHIEGRTTFHFKDGKITEVHFHEKQD |
| 44 | MSEEQIKEWLKRFYEAMDNHDWEALKRLYAEDATFHVSEGITFSNRDEFIEWWFRKFASTIDKSQHDIDSIHIDGDTVTVHVTQHVHHKDTGETRTVKITQHFRFKDGKLKEDRIHHDQS |
| 45 | TSEEQIEELARRMYEAFDKGDWDALLKLVHPDATFHVSEGITFDNAEEFVKWWFEKWRRHVSDMKREIKSIEIRGDRVTVHVEVHVTHKDTGTTVTIRATHHWVFKDGKIVEVHMHYDQS |
| 46 | PSDEEQLKEFKKRFIEALNRGDWKAVAELMSEDFTYYDARTGQTYDYEQFVKMVREFLSKAKDLRIEIKEIRIHGDTVTVHVEFHATHNGETRTVDVTTHDHYKDGKLKETRFHHNHQ |
| 47 | PSDKEQIKEWLRRFYEAFDHGDVEAVAELFSEDAQFTDARRQQTYDYEEFKEMVERMLSQMKDVRHQIDSIHIHGHTVTVHVTVHFTHNGKKVTVDVTEHHEFRNGRIVRTQTHHHPR |
| 48 | TDESEIRELVKRYFEALNKGDLDTVVSLFTPDATIHIYNGETITSLEEFREWVRKFLEDAKDVRFEIKEIRVDGDTVTVHWTVHMTHGNRTYTYRATTHFVFVNGKIKEMRIHMEQQH |
| 49 | PEEEELRKQVEEWLRQFYEAINKGDLEKLKELIDPEQSTFHFNGQTYNFEQFIEIVEKLFRNDIKLEVKQIDIEINDNGTITVTVHVTTHHKDGKTSDSTDHYHFRVENGQLKLVSVESHRQPT |
| 50 | PEEDERKEQVKELVRRLVEAFNRGDMDALKELFTDDDWSFHQNGQQFDADEFIKMLKELFDNGVRIEVKSIEIRVHPNGEVTVTVHVEITFSDGSHLDATEHHHFRKENGQIKLIETRVTHQPK |
| 51 | ASKHEIEEIARRFYEAFDKGDLDKLSELLHPGVRIHFEEEQITFTDKEEFQEWYKEWKKRFQEAHREIKRIEINDGTVTVHVTVHVRHGDETYTYHVTHHWHLKDHKIVEVRIRVDQT |
| 52 | MSEEDIREWKKRFVEALDKGDWEKLHELLHPGARIHYEKYQITFTDREEFIRWYKRLYSQFEEVRREIKEIRVDGGRVTVHLQFHVRHGDETKTYRVTVHDEYRGKKLVSSRFRWDDT |
| 53 | MSEEEIRQIIKRFVEAFNNGDIDTAVDLLYTPDAEVHHSNGQTFHTRESIKKFLKELYKQGTHIEIKDLHVDGDTVTVHVQLHTHHDGQTRTVRVTSHFHFVNKRVTHEQQHYDSN |
| 54 | ASKEQIEEFLKTYYELFNRGDYEAAAEFIYTKDWRFHNNNGETLNTKESVKEWLRKLLSRGTQIQIDSLEVRGDTVTVHLTFTTHDDGETHHVRVTEHHTFQGNRIVRTQQHSDTG |
| 55 | PGEEAIREWIKRYVEAFNNNDLDALASLYAPGATVTFSARGETATDQEEIKKWYEEFLSQDIRVEVKSIHITDDTVTVHVTFQKRDSHQTKTVDVTVHFRFENTKLTEVRYHFNEQ |
| 56 | TDEEQIKDFIKKFYEAFNDWEKFREFIDDNVTFHFEQHQQTFTDKEEVVDYIRKWLESVKSFRLEIKSLEIRGDTVTTHVTIHVNHKDQTRTFHVTEHHTFKDGKLVETRQHVHQT |
| 57 | ASEEEIRRWIKRFYEAFDRGDADALKEMLAEGWQMHQTRNITFDTLDEFKEWVERFQRHTDAHREIKSIRVDGQTVEVHVTMHTRHGDQPKTISVTDTHHFNGRKITSTRAHFDGND |
| 58 | PSEEQLREWVKRFVEAFNNNDLDALRELFDDGVQVHFSKGITFHTREEFAEWVRELLEKTNMRIEIKSIRVDGQTVHVHWTAHWTHGDQPYTSHGTTTFHFGGQKLVEVRFNVDDEE |
| 59 | PEQEEKIRELIKRFAEAFQKGDMDALFELFHPDATYHDSNGQTFHSREEIKEWYERWFSQDWVVEIKEIRINHENNTVTVHMQYHLRRNNQTVNVTYHDRYRGDKLVERQTHRDEGG |
| 60 | TSEEEIREILRRFYEALDKGDLDALEELLTPDWQFHSSNSTLHNIEEFKEWVERWFKSVGEVRREIEEIRITGNHVTVHVTVHVTHNGKEVTVRVTDHHHFKDGKLVETRAHYDNHEK |
| 61 | SSEEQIREFLKRFYEAVDRGDMDKLRELLTPDIQVTFNTETLHNVEEVVDWFREMFERLSDVHREIKRIEVSDGEVTAHVQVHVNHNGKTVTMDVTDHYRFKDGKLVSMRSHTDNRES |
| 62 | PVSEEQLRDWVRRFFEALNKRDLDTLVSLFTPDWQFHHISGVTIHSLEELREWVERWLRTFSDFRIEIDSIEIHGDTIHVHVTIHFTDDRTGRTFTVRITETHHFDKGGKLKETHVHEDRQPK |
| 63 | ADREKIKELLKRFFEALDKGDFDTLKSLMHDDWTVHITHNGQTFTITTREEFEEYYRQLFESVKDVQRHIHSIEIHGDTVTVHFTVTQRHGDKTYHIDITEHHRFEGDKLVETHVHVDHKQ |
| 64 | PLSQEQIKEWLKRFYEALDRGDADAVAELVTDDWTMHINNGITITTKEEFKEWVERRLSQFSDIKRTLEEIRIDDRTVHVHLTVHTTHNGKKVTIHTTDTHHFTDNRLTETHFTEDQKDT |
| 65 | DWREEAEELLRRLYEALDKGDRDKIREILHPDVQYHDSNGITLHTLEEFIEYVTRLLSRFDFQRHIHEIRFSDDGTVTVHYTMHFTHGDQTYTATVTDHFHFTNGKITEVRSHVDQHE |
| 66 | DFREEIKDLIKKFYEAIDHRDRDKLREIVHPDATFHLSEGITFTNREEFEEWVEQVQSHFDTRHQIDEIRIDDSGTVTVHVTVTRHHGDKTQHIRVTEHHRFTDGKIAETRVHFHEER |
| 67 | PQPDQERIEEIVRQLYEAFDKGDLDTIASLLHPDVQVHINRDDETITFNDLEELKEWIERWRSNFDVQRHIDRIEIRGNTVTVHVTIHTTHNGQNYTIHATHHWTFKDGRITRVEAHFRTQE |
| 68 | PSEEEIKEFKRRLIEELWSTRDASAAERYMHPDATYHNSTQTFTNKEEIREFIQQLLERYKRIHVQIDEIRIDGDTATFHVTLQFHHKDGTVHVHVTYHDRYEGKRLKESRAHEDQHD |
| 69 | MSEEEIEEWKRRVVDAFFRNQDAESWAQYYHPDARYSYNNRTFTNMEEIKRWLEEFLSRVKRAHVQLDQVDVDGDTATFHFTAHFHHDNGTVTVHYTVHDRYEGNKLTETQTHTDQHD |
| 70 | MSEEEIKEWVRRMIEAVWSTKDWNAFLSFYTPDATMHYNDQTFTTLEEIRRFVEQVLKEAKQTHVQFDKMDVDGDTVTVHVTIHVHHDNGTVTIHVTRHFEFRGNKLKRDQSHEDQRD |
| 71 | MSQEEIEEWKRRVMDIVFRTKDADAFAELYHPDATVHNGDTTYTDPDEIIKFVREFMSRAKQVHVQIDRVEIDGDTATFHVTITVHHKDGTVHIRVTAHDRYKDNRITETQYDYDQKD |
| 72 | PSEEEIKEWVTRMVEAVFKRNDYSIWLSLVSDDWQLHVNTQTFTTKDEVREWLKKWISDVKQVHIQLDEVRVDGDTATFHFTVHVHHDNGTVTFDQTEHHRFEGNRITETRQHTDQKN |
| 73 | PSREEIKEFKRRAIEAFWSNRNADIWLELYDKDATMHVDNRTFTDKDEIKEWLERMLSRHKRAHVQIDEIRVHGDTATVHVTLHFHHDNGTVTTRATYHDRYEGKKITESREHRDTHE |
| 74 | PTDRSEIKEIIEREIEAFNRRDLDKWASLFHPDATMHLYNGRTFSTLDEIKEWFEKMMSSMRDFRIEVKSIEIHGDTAHVTMTMHVRNDSKDVTINYHVTIHFRNGKIVEKHIRADHQD |
| 75 | PGEEAIKEIITRWYEAFDKTGDAETLAQLLHPDWQFHYNGQRFTNLEEFKEWVERFLKRFPDSEHRIERIEVHGDTVTVHLTVHVRHGDQTVRLPTTDTHRLKDGKIVETHVHTGGDH |
| 76 | MSQEEIKELDRRRAEEAFQKKDPDAFISYFHPGATMHINNGQTFTTLEEIKEWFERFLKEMQDAQIEIKSIEVRGQRVTVHGTITVHRDNKTVTIHMTYTDEYRDGKLTHSEIHIDEQPT |
| 77 | PSQEEIKEWLRRYIEYVFQTGDADAAAKMIHPGWTFHHSSGQTFTDLEEFKEWVKRYQESMQDVQVQIDSIEVHGDRATVHMTLTVHTPTQTFQIHVTEHHQFKDGKITETHQHEDNQPT |
| 78 | DEKDEQALKEFKERFIREVWEKKDADVFVKYIDKDAQFHFSSQTFHNQEEIKEWVQRWFEERVKDAQVEIKRIEFHGKRVTVHVEVHVNHDDGTVTYRYTYHDEYQDGRLKHTTVHEHQHD |
| 79 | PEKDQRAIEEWVRRWYERVWRTGDWDQLAKMLTPDWTVHYNDRTFHDRDEFAKWWREMFEKTFKRVQVQIDRVEIHGDRVTVHVTVHRTSDSGDYTIHTTDHHQFRNGRIVETHIHTDDRS |
| 80 | LDPSQEEQIRQWFRRYYEAMNKGDLETLERLFHDDWTQHFSNGQTITTKEQLKEYIKQITDRNVQIEVHVEIHFHGTDHATVTVHITLHENGSTVHVTEHHHFEFTNGSLKIKETRTTIHGE |
| 81 | PDPSQQEAIKELKTRLVEALNKGDWETFRSLFHPDATMHLSNGRTFTTLDEIVDWFRKMTSNDVKIEVHINIDAIGRDTVQVHVTLHLHYNGQTFTVHATDHYQFTNGQLRIIETQFNYHGS |
| 82 | ADPSQQEQIKEWIRRFVEAFNKGDMETIKELYHDDATVHNYNGRTFTTKEEIVEYIKQLTDQDMKIEVHIEIHFHGNTHATVHVHLTMHARGQQYHFQSTFFFEFTDGKLFLKEERVTTRGS |
| 83 | VDEEQEKQVREWIRRFYEALNTGDMDALKSLYDDDATMHIANGRTFTNPEEIVKWFKEFSDQNWQLHVEVHIEMDGTDTVTVTAHVQAHGNDQTFNITQHFHFEVTDGRLFLKEDRIHRHGS |
| 84 | PMTAEQVVRAFWEAFQKGDWKKLLDMITPGWTLHISQGTTYHSLEEMRRWFEEFQKNNEQMHIQIHRVEVHGDTVTVHMTIHVTHNGETRTYHVTEHHTLSKDGKIVETRQQHDST |
| 85 | PVSAEQVAKRFAEAIAKKDLDELASIYADGATFHVNNGITYKSKEEIKKWWEEMLSNFPELHIQIDRIEVHGDTITVHVTMHTTHNGQQVTFHITSHFTFDKDGRLKSEHFHMNPT |
| 86 | PMTAEQVARFYWEAFQKRDLDAFAEIFHPGATLHIWKGITYHNPEEIKRAFEQWMKEFPQFHIQIEEIRVHGDTVTVHWTVTVHHNGQQYHFHATTHIRLDHNGKIKETRFHVHST |
| 87 | PETAEQVVRKVEEAFNKGDRELFDKMLHPGWQLHHNSGHTWTSKEEVKEWFDRWLKNHPRFHIQVDRVEVHGDTVTVHMTVTTHHNGQQYDFSVTEHHHLSKDGKLTETRQQRDPT |
| 88 | PMTAEQFARAFADALNRGDLDALRRIYHPGATLHIYQGITFHNLDEIKDWFKKWFSQFEEVHFEIKRIEVDGDTVTVHFTVHVTHNGETRTIHVTSHFELSKDGKLVSEHIHENST |
| 89 | PITVEQFVRTLEEALNKGDLDALARLYHPGATMHINNGETYHNLEEIKKWFKEFFESFQETHFQVDEVRVEGDTVTVHVTVHATRNGKTVTMHATSHIEFSKDGKIVSEEIRIHGS |
| 90 | PSEEELKEIKKRFAEEAFNTGQWEKFAEFFHPDATMHINNGTTYTDKEEITRWFQELMSNFPDVRVEIKSIEVRGDTVTSHMTIHVNRNGQTFTFSVTTTDHYKDGKLTETHIHIHGD |
| 91 | PSEEELKTAAKEAVEREVEAFNKGDMDAWKELFHPDATMHNSNGQTYTTLDELKRWVKKLFDNNIRVEFEVEVHIVDSKEVEVHVHFHFHNPDGTTMTVTATDRFRREDSQYFIVERHIHHYE |
| 92 | DKEEEIKQWIKEWYERWITAVNQKDVDKALSLFHEDVTIHYQNGQTYTTKDEVKEWLTQFISRVERIELKSMEIRIDGDTVTVHVHITFTNRQTGRTFHVDITHHWQFKDGKIVSVHEKRDRTDTD |
| 93 | DKEEEMKRLIRELIERYVDALNRKDRDAAAEMFHPDWQFQEASGQTFHSLEEWKEWVTQLMSEVTTIQIRHIEIHIDGDTATVRVTIEITHSKTGRTTRFRQDEHHEFRNGKIHRTRTKRDPTRTD |
| 94 | TSEEQIEEWLRQYYTAINNGDMDAVRELVHPDATFHESNGQQFDTRDQIVEFIERFWKTFADVHIDMKSFEITDNHVTVHYQVHTHHQDGKRVTFDGTDHFQFKDGKLTRLESNRDQKD |
| 95 | MSEEEIERAVREWLREFYRAINQNDLEALRELVHDDWTMHWDGRTFTTKDEVKDWFRKMLENSDITVHFEVHVEYKRDGRVTVTVHAHITHKETGTTTQVDEEHHFQFKDGTVKLLETHARTQPT |
| 96 | SSEEEIREAVKEIIRRFAEALNNNDMDTLRELYAPDATMHIDGQTFTTLDEIVDWFRKFLESFDITIEFEVEIRITKDGSVTVHVHFTVHFNDTGTTTKFTSTFHIQQVNGTFKIKEEHLTTQPT |
| 97 | PDERKIKDWIRRYLEALDRKDLDELKELFTKDWTFHLTGGVTFTNIEQFIEWVERWLSSFPRSRHQVDSIEIHGDTVTVHVTVTLTHPDGKTVHATYTEHHRFAPDGKITETRVHIDQKDT |
| 98 | PDAEEIKEWVERWFEEVFNTGDADRAKELLTPDWQYHLYAEQRTFHTLEEFREWFTEFIKTFQEIHVEWKSVEVRGDTVTFHIEMHTTHNGKKVTFTITEHHRFKDGKLVETHVRIHQRN |
| 99 | PDEEIVRWVREMFERAWNTGDADQAASYYWPDWRVVHNGNKENRDLEEYKEFWERMLRSASDVKFQFDSIEVHGNTVTVHVTVTFHHKDGETVHVHVTETHHFDKDGRLVETRQHSDQRDQK |
| 100 | PDERLIEFYLRYFDYVFNTNDADAMKSAYHPDARFTFDGNTSQISLDEFKEWFKKWVESVKRFHVKWDRVEADGNRVTFHVTIHFHHDSGKTTTVHITFHVVFDKDGQIVEERIHRDTHEHE |
| 101 | MDQSEAEEMIRQFYKRFAELLNRGDIDVLAQLFTPDATFHISNGQTFNSKDEFAEWLEELFRQNWQFEFHLEFKEFNGDHVTVEVHVTMRHSNGKTVHVTATHHWRFGDGELKLVEVRMHFNET |
| 102 | LSEEEIKEILKRFYEALDKGDADKLRELLAPDWRYHNAAENRTWDDKEEWIEEYIKSLRRHAEWQRQIESIEIRGDTVTVHYTVHWHHGDQTRTIRVTDTHHFRGKRIQETQAHWNQT |
| 103 | MSEEEVKEIARQIIKRWFEAVDKGDVDALMELYDKDRARMHINGQTFTTMEEIKRWFEKWASQNIKHDFEVHSQIEGSNVVTTHVTVHVTFSDGSTMQIHTTFFMEYSDSSWKILEQRTNNQNE |
| 104 | MSEKEVKEQIKEFKRRLVEAFNNGDLDAIAELVDKDESTFHFDGQTYTTLEEFLKWVKELLDQNVRLEVEIHIEIEGSNQVHVEVHITFHFSDGRTVHVTSHDRYDLSDSKLKLREMRVHNQGE |
| 105 | NSEEEIQRKIEEWLRQFYEAFNKGDADHMARLVDKDMSTMHSNGQTFTDIEEIKEWLKQLFDQGIRIEFEIHIQINGSSEATVTVHVRFHHPDGSTINVTEHHRFRADDSSLRLVETHQNWQNE |
| 106 | PSEKEIREMVKEWIRQWAEAFNKGDWERLAELYDDTNVEMHISGQTYTTLEEIIKWIQEFLSRDVQVHIEIHIQIHGSNTVTVHVHFTFHFKDGTTVHSTSTFHFDASDSSLKLTSQHVTNQTE |
| 107 | ASEEEIRETLKRFFEAINKKDVDKIASLFHPDATVHFYQGITVTTKDELKEFLEKWFKRLRDVKVQVESIHIDPTGHTVTVHWTMHVNKDDTTITFHATTHFHMKDGKIVEMRIHVEQRD |
| 108 | TSEEEIRRFLRRYYDAFNKKDVDKLEELLHPDWQYHNNTGQTLTNMEEFREWVKELLSRLKDVKIQIEEIHIDSNGHTVTVHVTVHVNNGDRTVTIHTTDHHHFKDGRLVETRVHAQQTS |
| 109 | PEWEEVVRELKRRLIEAMNSRDWSKLAKLIEEIFHDDATLQVNNQPFTTKEEIKEWFRRVASNDIDIEVDTEVQPKDGKYVTVHTTYHMTDKSTGTTTTFHATDHYQLEGNKIVRTTMHSRST |
| 110 | PSKEEIKDWVTKWYQALNHKDLDKVVELFHPDATIHTSTLNITWTTKDEFRKFVESWLRQFPDHRIQIDEIRIDDTSATATVHVTVHLTQNGETYTIKYTEHHTFDSTGQVVETHQTIDPK |
| 111 | PSKEEIKETVKRWFEAFDKHDLDALVELYHDDATITDYSNNTTFTTKEEYRRHVESFLRTFPRMQHDIHEIRIDETNYTVTVHVTTHLTRNGKTKTVDVTFHFTLDHDGKIKEQHTRFNDR |
| 112 | DWEEEIREIIQRFFEAIDNNDRDTIRELLHPDARMEVSEGYTMTNLDEIVKWVEDFFKNWHRMRHEIEEIEFNGKTVTVHWTAHFTHKSGQNYTIHGTTHIHFHDNGKIARLEFHLDQRD |
| 113 | TSEEDERAIKEWVRRFYEALNTDPDAVKEFYSDDAQFHAPDGTQFDTKDEIAEWLRQVASNTDITLHSIEVRTDEDGTIHVTVHVQVTNRTNNQSFTVTQHMTFKSDTYQIKRDELRVDDN |
| 114 | ASEEDKKRIEELIRRFYEALDKGDWKAIRDLFHDDARYHDSSKGRTFDKEELLRMVKEHSKHSSQHHIKSIEIEKRGDTIHVTVHVEVHNNSTGTTFSVTEHHTFVRHGDDWRIVETRQHIQPT |
| 115 | PSKKDEEDIRDMIRKYVEALNNNDLDAVREFYDPDATVHFNSEQRTLSWEDVIRWYEEMLKFRFRIEIKDLHIEQRGDTYTVHVHMQVHFEDTGTTVKVTATFHFEKRGDEWKLVEVHFTFQPE |
| 116 | PSEKEEEDIRQLIKHYADAINNKDVEAIASFYTDDATVRFASEQRDFDAEEVKRWYEEMLKNDHRIQIKSIEIRKSGDTVTVTVHMDVTYSSTGRTVKVTATFHFELRGDRWYLVSVHFHFQPT |
| 117 | ASKKDQEDIKEWLRRFYEAFNRNDLDAIMELVTDDATFRFSRHQKDFDKEEFREWVERFLSQDSRIQIKSIEIRQDGDEYTVHVEVEIHNSSTGQTFRVTHTWRFRKEGDRWKLIEVHFHWQPL |
| 118 | PSEKQQQDIEELLRRFYEAFDKGDLDALRELFHDDVTFHFHSEQRTYDKEEFLEWVERRTRFSTKHQIDSIEFRLHGDEVTVTVHVTTHDETTGTTFRSTHHWRVRQHGDEWRILEVHFTLQPT |
| 119 | PSDEEQIRDWFRRWIEAFNKHDMDTLRELYDPNATFHDSRTGQTYDYEEYIKMVKTTLDRFPDVRMELKSMEIEKRGDEITVHLHFQFHYKKTQTTQHVTQTFHFRREGDRWKLKEQRSREDG |
| 120 | PSDEEAIREIIRQLIEAFDKKDLDALMSYYDEDATFEDASTGQRYNYEQIRELIERWLRRFPNTAHQLDSLEIRKHGDTVTVHVHVTVTYKDTKETHTFTRTVHFVRKGDRFFITRDRTQSDN |
| 121 | PSDQEQVRDIIRRFYEAFDNNDLEKIKELLHPDATFQDSSTGQEYNWEQYVKRVKELLERFPNTAHHIDEMHIEVRGNEATVHAHVRVTYAETQTTTTADHTWFLRRHGDRWFVIRVTTTTRN |
| 122 | PMSEEQIRELLKRYYEAMNKGDVDTIAELFHDDARFHHSNGQTFDKEEYRKWVEEYLKKFDMRIQVKSIHIEIHGDTVRVTVHVEFHNPKTGRTFSITHHWHFDASTNKIKRVREQRDDQE |
| 123 | PASEEQIRDLVRRYVEAMNTKDVDKLVEFYHDDARYIHSNGQEYDKEEIKEWYERFQKRYDAELRIKEIRIQVRGDTVTVQVHVEVHFPKTGRTVTVTAHFFVDARHNKIVRVREQRDDQE |
| 124 | PLSEEQIRELVKAYYEAWNKRDLDALAELLAPDATFEHSNGQQYDYEEFREWVEEFMKKFDAQLRVKEIRIEIRGDTVTVTVHVEVHFSKTGQTYTVTHHWFIDARHNKFKRVQEHTDTQY |
| 125 | PDDKEAAEEFVRRYYEALDNGDLDAIKELFHPDAKFHSSSNQTYDREDFIEMIRQWMSQAKDSKHDIDSIHFDKQPDHITVTVHVTVHNSTEGQTYHFTHHWFFRLTDGSWQLVELREQTDGG |
| 126 | PSDEEAIREVDTQRLEAIDRGDLERLKELVHPDARFHTANHSTYDRDEWLELVKQFVERFPDSKHEITGMEIEIKPNEVTVHARVTVHVDQTGQTFHVQYTIHYRKTDGGWKIVEVHVQHDGG |
| 127 | MNDEQAIKEFKQRYVEALNNGDLDAIRSLFHPDVTFTSADNQTYDWEDFARMIEEWIKTFPDAQIQILSMEIEKTDSEIRVTVHIQVHYPTTGTTHHFTQHDRYRRTDGDWKLVESREQTDNG |
| 128 | PVSQEQVKELLQRMYEAFNNGDMDKLRELFHPDAQFTDSNGQTYNAEEMLEIIEEILRQFPDTQIEVKSIHIQVHGDEVTVTIHYTIRRNGQQFDATDHIRFRRTGDKVQIIEMRSDNSEVKK |
| 129 | PLSEEQIRQWLRRWFEAWNHGDRDAFRELYHPDSTYEDSDGQEYNRDEYLEMVERTLKRFPDARFEIKEIRIQQDGDTYTVTVHFQVTLNGQTYTVTQHFHFRADGSKLQLARQQSDHSNDSN |
| 130 | PMSQEQVKELIKRLFEAFNKRDLEKLERIYHDDATFRDQNGQEYDKEEFKDMVEKFMKTFPDFQLHIDSVEIRIHGDTATVHVHVTAHLRGQEFRVTSTIHLDAQDSEIKIISETVNRNEVKK |
| 131 | PVSKEQIKETDKRRFKSFDDGDIEALKELFHPDARYTDQHGQDYDKEEFIRWFKEWKERFPNTAHEIDEMHIQQDGDDYTVHAHVTVHINGQTYHVTYTVIYRYDGDELKIVRVQTHTDDTKS |
| 132 | PEEEEQEEAVRDLIRRYAEAFNRGDAETAARLFDEDWTYYDPSGQTYTTREEFKEWVKTLFSRNLRMEVEVHIQINPDGTVTAEVHFTFHHQSGQTYHVTETHHIRQENGTFKITRTEAHLQPS |
| 133 | PISEEQIKEFLKEYYTAIDNNDVDKIASLYHPDATIHFPNGQTLTNPEEIREWYERWLSQFPGTAHHIESIHIDKTSNTVTVHVTTHFNHNGEKVTVHYTATYHFKDGKLVSYQDNSGDTTG |
| 134 | PDEERIKEILERFFEALQNNDLEKLKELVHDDATFHFDSEKITFTTKEEFIRWVKEFWEKFDMKVEIKSIEIRGDTVHVTWEAHLTKDGQTYTFEGHTTFTFKDGKIVEVRFHVRRT |
| 135 | DQEEEIRRIIKREVEAFQKKDIDTWLSLFHDDARVHYTRHGETYTTKEELREFVERVLRHFDVSVEIKEIRISGQTVTVHVTVHETHNGQKYTFNVTVHDRIKDGKITERRVHWNPT |
| 136 | ASKEEIKEWLERFYEAFNRGDMDALKRIVDPGVTMHVSNGITFHDPEEIKEHFARMFESSKNTRLQIDSIEFQENGDTVTVHVTAHETKPDGKNVTAHITHHWRFKDGKLVEVHEHVDEQPT |
| 137 | MSREEIKDFKRRMVEAFNHKDLDALASLVHDDAQFHSSKGETIHDKEEFKEWVKKTLSVPMRVEIKSIHIDDKDATVTVHLEIHRTHGDKKVTTTATAHDRYTDGRLTETQYNFDDS |
| 138 | PSKEEAEEIARRFYEALNKGDWETIHELFHDDAQMHLPNGQTFHTKDEITEWVKRLISVPMRIHIEKIDFNDSEATVTFHVTMTFTHGDEERHIRATHHWRFTDGKVTELREHIDNS |
| 139 | PEEEEIKEVIKRFFEAIDKRDADKLRELIHPDARMQLNGEQLTNLEEIVKVYFERFVKNFQQAHHEIKSIHIDSNGTVTVHWQLHVTRNGKDVTINATTHIRFRNGKITEMRFHVDNQEN |
| 140 | PLEEEIKELIRRLFEALNKNDWEKYKRLFHPDATQHFNGITLTNLEEIVEWTYKWWVSQYDEWHFEIKSIHIDHDGTVTVHWTVHVTRNGKSITIHATTHIRVRNGKITESENHLDNQEN |
| 141 | PSEEQLKELKKEFIDAFNKGDADKLKSLLTPGSTWETNGNETITTLDEFKEWVERVFSNNTQIEIKEIHIDGDHVTVHVTIHTHHPTRTKTQQATQHDTYDKDNRITNTRENTDPKD |
| 142 | SSEEEVKEIKRRLIEAFNNKDVDALRELFHPDSTFHSDGRQYDNKEEFVDKFFEELVRRYPRAHFQLDHIHIDGNTVTFHMTVQFHQGDQTYHYRITVHDEYRNGRLANTQIHTDNHG |
| 143 | TSEEEIKELVRRFFEALDNRDRDTLKELYSDDAQFHVNGETYHDADEWLKRWFEEFVKKFPSTAHEIKEIRIHGDTVTVHFTVHVTQGDQDYTFHATSHFEFKDGKIKSEQEHMDQTD |
| 144 | MSEEQIRDIVKKFFDAVDKGDLETLKRLLHPDATFHLNGRQFDTLDEFLKKYIEEFQRRFPETHRQIDRIEIDGNTVTVHWTMHVTQGDKHITFHGTTHIHFKDGKITRVQIHIDNTD |
| 145 | TDPRIKEWVERFKTALFEAFNKKDADKFVSIFHPDIQFSDSDGSTIHTLEELKEWFEKFISQFDRIQVDDQEIRVDGDTVTVTTHVTFHRGHKTVTITYHDHYEFRDGRLVNVHTQTDPTK |
| 146 | TDPSIKEIIREFYTRLLEAFNKGDWETVVSIFHPDHTFHFEDGSSITTLDEWREWVERFMSQADRIKVDSLEVRQDGDTVTVHVTVTITHGDETYTFHATYHHVIKDGRFVETHQHNQPEQ |
| 147 | TDPRLRKWLEDFYREFAKALNNKDADLAASVLHPDVTIHLENGETLTTLDEFKEWFTKMASTMDRVQFDEIRIHQHGDTVTVHVTVTITHGDETYTFHATHHWVFKDGRLVEVHISTQPQR |
| 148 | TDESIKEFIKRFYEAVFDAFNKKDWKKFVDLFHPDVTLYDTDGRRYTTRDELREFFERLASRFDRIQLDSIEIRQDGDTVTVHIHVTFHHGHKTVHQTITHHWRFDDGRVTEIRQNSDPKD |
| 149 | ASKEQQEQIKDWLRRYFEALNKGDWETLKELFDDDWTVTDASTNTTYDAEEWLRMYRTLLSQGFEFRIEVKSVEIRLNPDGTVTVTVHFTMHVTHNGRTVTLHVTEHHHIRFKDGSLQVVHTQVHTDERHP |
| 150 | PSKEDQEAIEEFLRRFYEALQKKDLDALEELLTPDWRYYDARTNRTYDLEEFKEMVERRIQNGFDVKVEIKSIEFQKNPDGSYTVHVHLQFHFTHNGKTVTVHTTDTHRFEKHDGRWRLIETRFHYDNRHP |
| 151 | PSEEDKKKIEEFVRRFYDALNKNDLDKLKEVLDDDWTYYDARTNERFDLEEYLRRLEELIRQGWETHIEIKSIEIRKNPDGSYTVTAHVTLHFTHNGKHITVHATDHHHIQKKDGTLKFKETRAHYDSKHP |
| 152 | ADPSQQEAIKQIIERYYKALNEGDVDTMVDEIYHPDGRFTYSGADVEQFKEWVKKWLSTFNVKIDITSLEIQGKDNGQVTVTVHVRFQVHQGDKTYTFHVTQHFVFEFTKDGVRIQNDQINRENEG |
| 153 | DEREELKEAARQIAERFYEAFNRGDLDTLYELMAPGWTFHHQGGETYDRDQFREMVEKWLRQKIHLHIKSIEVEVKDDGTVTVTVHVEARQEDTGRTYPFTDHHTVQFTKDSWRIVETHQHRDN |
| 154 | DERKEIEEAIREMIRRYVEAFNRGDADALYELFDDDWRYHHQGGETYDLDQFREMVERIFSQGVRLQLDSLEIEVKDNGTATVTVHLTMTEAQTGRTTHFTEHHHFRQKKDGWRIQRTHQHTSG |
| 155 | DERQEIEEKVRELKKRFIEALNSGDADTVASLFHPGAQFHHDGGETYDLEQFKEMVERFFSAKIEIRLDSVEIRVNDDGTATAHFHFTFHETDTQRTFHGTQTDFYRQTKDGLYIVEVRSHRDG |
| 156 | HDEEAEEKIRQWLTEYYRAIDNGDWEKIAKLVTDDSKFTHRDNGRNETYDKEDLEERIKELANYESQHNLKEIRVQINPGGSSVTVTVHVEVHFHHDTGTTTFHATHHWHFRFENGELKLVEVRVQIDNS |
| 157 | MSEEEVRQAVREFKKRFFEAIDKGDLEKLKSLIHDDARFHHARGQTYNKEEFIEMVEKWHRQDTAHQIDSIEIQFHDDGSVTVTVHVTVHVNHKDKTVTVHVTYHDHYRYGSPGWKIIRMQVHRDHT |
| 158 | TSTEEAKEDIRQFVEEEFKAFNNGDLDKWAELVHPDATFHTDEGTTYDVDQMLEMIEKAVKNNTHVTIDSIEIRQHDNGEFTVHVHITVHTNRDGSTQTVHVTQTDRFDQSSPGWKLIERHTQRDNT |
| 159 | ASKEDQEAIKELLKRFFEAMDKGDLEALKRLVDEDWRYYDASRNQRFDADEFLEMVERQARQDTKRELKSIEIQPTDEGHITATVHVQVHVTRNGETRTYTVTEHHVFVKKNGSWKIVETQVHTDSN |
| 160 | ASPEQQEQIRQLVKRFFDSMDKGDRDAIREFYDDDATYRDARRNQEYDKEQIVEMIERIFRNDTRHEIKEIRIQPTDEGQITVTVHVEVHQTHNGKQVTFTVTSHFRFEFRNGEVYLVSEEVHTDDN |
| 161 | ASEEQQEKIKQWKRRFFEAINNGDLDALRELVHPDAQFTDASTQQTFDVEEYLKRIEELLKQDFQIEIKSIEIRPTDEGRVTATIHVQVHENHNGRTKTVDVTQHDLYQFENGSVKLVSTQTHRDET |
| 162 | ASEELRQAAEDWVRRYAEAWNKGDVDALASLYHDDARYTDDSTQRTWDKEEFRERFERLMSQDMRMEIKRIEFHPTDEGEITVTVHVTIHANHNGRTHTVDVTRHFRFRQQDGEVYLVSDQSTRDSK |
| 163 | ADKEQQEAIKDFKRQYVEAYNKGDMDAIKSLFHPDAQFHDASRNQTYDLDEFIERLERILRQDSRMEIKSIRIEPTDEGEVTATVHVTVTHNHNGKQHHITYTQHDRYVFQDGSVYIVETQIHTDSN |
| 164 | TSKEQREKLEEVIRRFYEAFNRGDLDALKRIVDPDATFHDDSKQQTFDLEEFIKRVEEILSSDVKIEIKSIEFHPTDEGEITVTVHVQVHQNKDGRTQTFTVTNHDVFREENGSFKIVEHQSRTDDN |
| 165 | DSPDQQQIKEIIKRFAEAFDKGDADTVLELVAPDWRYTDARSNTTYDAEEFAEMVKRLASQDLQRQIHSLEIEFRGDTATVHVHVTVHHNKGDRTQTVSVTETHHLQKRNGQWYLVRTEVTHHQT |
| 166 | DSEDQQAIKELMKRLYDAINKGDMKALKDLLHPDVQFTDSRSNTTYDLEEYIDMVKKMLSRDVQVHIESIEVRIHGDEVTVTVHVTFHEHHGDKTHTVRVTHHWRIRKKDGQFYVIEVRVTHNPS |
| 167 | DSEDQQQIKRLKKELIEAFNRGDMDRFKELIHPDAQFHHYYSNTTYDVDEFIEMLEKMLSTDVRVQLDEIHIQQDGDEYRVEVHVTVTWHKDDKTQHVHFTATDRYRKINGQWYIVEHRVRRHQS |
| 168 | PEEEELQEAIKEIIHRFYEALNKGDLDAVMSYYDDDARMHHASGQTYSKEEIRRWFEELLKQGVQFEIKEIRIQVDKDGEVTVTVHVTLHHTGTGKTVDVTQHFRFRRENGSFKLLEDQVHEHDK |
| 169 | PEEEELEEKARRAVKRFFDALNNGDIDAVLSLFDDDWTYHHSSGQTYSKEQFKEMVERFQSQGFEWHIESLEAEVKKDGTVTVHVHVTARQRGTGQEFKITETHHFRFENGSVKIVETHVHTHDT |
| 170 | PQEKELEEQVRDVKRRMFEAFNKGDWQTLSELIHDDATFHHASGQTYDKEQFIRMMEEMFKQGWQLHVDSLRIEIKKDGEVTVEVHVTAHQPGTGRTTHFTSHDRYQQKDGSVKLTETHTHEHST |
| 171 | PKEKEDRDEIKEIKQRLAEALDKGDWKTFRELFHPDAQFHDDRTGQTYDLDEMLEMIQRQASIPARREIKEIRIQFIDNGTATVQVHVQLHQTRGDVDYTTDVTTHDHYTAKSDEWKITETKITVDDKDG |
| 172 | PWKKEAEEQARELVRRLYEALDKGDIDAIREMLHPDFTYHNPSTGQTYDKEQFLEMVERLASIPMQRDIESIRVEIKDDGTVTVEVHVTIHETHGDQDYTVHVTDHYHFRSDSDELKLVELHSTTDTKHD |
| 173 | PEEEELKEQVKEWLEREVEALNKKDLDTWVSLFHDDFRYHDPRTGRTYDKEEMRRMVEEFLKFDFRIEIKEIRFEFLPDGTIRVTVHVQVHVTRGDQDYTVHITYHLQFRRDSDELKITSKQSHSDTKDD |
| 174 | PWEEEMKEQIEKIVRRLVEAWNKRDIDKIVSLYHDDWQYHNEKTGQTYDKEQFKKMVEELLQIPFHIQITSIEFRTHKDGEITVTVHVQVHRHHGDKTYTIDYTEHHRFRAESKEVKIQETHVRTDEKRE |
| 175 | PAGEEDVRTWLKRFYDALNNKDADKVASLYADGAQITFSESNRTLDKEEFKRWVEQMASTDAQFEIKSIEIRFGDDGSVTVTVHVQVHENHGDQTKTVDVTQHFHMRYTDGQWKVTRDEIRFHED |
| 176 | AVGEEKIKEVKRRLAEAFNKKDVDALLRLFHPGAQFHFAESQTTYDQEEFKEWFERYASTDVEMRIDSVEIRVNPDGTVTVTVHMTLHMTHGDQTQTFHVTAHDEYRETQDDLKVAETRFHMHQD |
| 177 | SGGEEEIRRLVKRFADAFNNKDLDAILSLFTDDWRITDARRNQTYNLEQFKEMFERIASQDVQVQIDSIEVRHNDDGTITVTVHLTYHERRGDKNDTFHVTEHHHFQLTQDGMKLVETRVHHRGE |
| 178 | TSEEEQKEQIKELIERLVEAFNKGDRDKFESIYDDDATIRDASDPTDFTKEQFLELVKQFHSQNFHLQITSIEIRINPDGTVTVHVHVQVHLHQGDETKTFNVTSTFFFTQENGSWKIKEERIQFNTHE |
| 179 | DSEEEVREKIKETVKRLFEAIDKGDLDKVRELIHPDATFHHDSGETYSLEEFLEMVEKLARNNSRHHIESLEIRQVDDTTFRVTVHWTLTDDRTGSTLRVTTHIQVRFENGELKITRVEERTDPT |
| 180 | DSEEEAREKIKDFKRRLVEAFNKGDIDALRELFHPDVTFHTSDGQQYDLEEFLQMMEELFRQGVQMHVRSMEIRQVDDTTFTATVHVTLHNESTGTTVDVTFHDHYRFQDGELYLIRSEARTDSK |
| 181 | DSEEEIREKIKELIKRLVEAINRGDADAVASLFDDDWRFHQDQGTTYDREEFKKWVEELNKANVHIEIKSIEIRIVDHNEVTVTVHLQFHNDTTGTTVRVTEHHRFVRKNGQWRVVETRMHTDPL |
| 182 | DSEKEQEEKIREFIKRFYEAIDKGDLDELRELLHDDWRYHHARGQTYDKEEFLKMVEEMHSNDLKHQIDSLEIRVDSENTVTVHVHVTVHNSSTGKTFRFDDTHHFRFQDGELYLTRTETHTDNS |
| 183 | DSEEEMKEKVKEVIERFYEAINKGDLDALRSYMTPDWRYYHWSGQTYDRDEFLKMVKEMNDAGVRVQIDDMEIRIDDHNQVTVTVHLTLHNQSTGTTVKTTDHHTFRFQNGQVYIVSTHVQEDHR |
| 184 | PGQQEDQRAIKEWKERYIEALNKKDWKAVADLVSPDAKFTNNSEQQTFDREEYIKRVREYLEKFDTRFEVKEIRIEIKPGNETFTVHVHVQVHVTHGDETRTFDVTQTDHYTKKNSQWYIVETRWTTREVK |
| 185 | PGEEEARRQAEEIARRFYEALNNKDWEAIARLVDDNAKFTLTATQRTYDRDEMIEMIRRMLEQFDIRVEIKSIEFHIDPGGDSITVTVHGTITFNRGDKQKHVDVTHHWHFKQGDSQLKLTHLQETRRDLE |
| 186 | PGDEESEKEIREFIEREFEAFNKKDWEKWARLVSDDAQFHMASEQRTYDAEEMKEWVRRTLEQFDIKIEVKSIEIRFHPGGDHVTVHVHFTVHETHGDETRTFHVTVTLHFERRNDQWYLVSKHIHIQPQE |
| 187 | MSEKEAEEAIRDTIRRWVEAINKGDADTAKELYTPDATIVHDGQQFDRSLDEIKEFIKKLFDNNVKLHVRSIEIHVDPNGGDVTVEVHVQLTDDRTGKTVDVTFHAHLTINGDEVRIKRQHEHRSDT |
| 188 | PSEKELEEAAREVVKAFYEALNRGDADRLAELLAPDVTFVHSGNQFDLSLEEFKKWVEEFTKAGFTLHVHDMEVEIHESGGTVTVHVNFQLTDHRTGTTYTVRDTYFLQFHGDEVKIKRVESQTDQQ |
| 189 | TSSDEAREAIKEIFRRLAEALNRGDLDTIKELYDDDVTYTNNGETQKRTRDEYKKFAKEFFDNNIKIQIHDMEIHQDPRGGTFRVTVNFTVTNDRTKTTRRFQSHMVLQFHGDEVRIKEEEERTDQL |
| 190 | PSKEDEEQLREFDKRRFEAINRGDWEAVERLMHPDATIHVASRQRTYNLEEFIRMMQELFRQNVQLEIKSIEVRQLSKDTFQVHGHLTVTNRTTGTTFHVDYTVFYRKGSGDWKIIEVRFRTDEEK |
| 191 | ASKEQQEQIREWLKRFYRAFNQGDADALMELLHPDWTYTNESEQRTYSKEEFREMVKKLFDAGVQIKVDSIEIQPDSKDEITVTVHVTVTQKTTGRTWHVTEHHHFRHGSGDMKLIRTRFRMHQQN |
| 192 | PPPEDEEAVRQVIRRYYEAWNNKDADKIMSLYHPDARFTDESKGQTYSYEDYKEMVERILRNSDMRIEIKSLEVRKVSDGRITATVHVQFHHTHGDEHHTVRVTQHFFFQKKDGEFYIVEDRVRIDNN |
| 193 | PPPEDQEQVEETDRRRIEAFNHGDVEAIASLFHPDATYEDSRTGQQFDREEFKEMVERFFKSVDFRIEIKSLEVQLYDEGHIEVTVHVQVHVRHGDEHYTMHVTYTVRYRKVNGTWKIVEMRTTSNDS |
| 194 | DEREESKEAIKEWKRQYVEAWNKGDLDALRELFHPDVTWDGHPYDYEQWVERVERFLRQFDTQLHMDSMEIRIDSDNTTVEVRVTITVHNHRGDRTETYTFHYTDVYDSRHNQLVKSTTHTDT |
| 195 | PHEEEIKEIVKEIVRRWVEAYQNRDMDTLRELYAPDVEFEGLPVDYEEYLEWVERVLKRADVRVEIKSIEIRIDHDNTTVTVEVHVEVHERHGDQDHTYRATYEFVIDRRHNKIKRQREQRDT |
| 196 | DEREEIERKIREFKRRYLEAWDNGDLDKLDELLHPDATWDGNDIDWEQWKEIVRSLLENSDTAHHLKEIHIEINDDNTTVTVTAHVETHHHHGDRTETYRVTYHDHYDRRHDKLTRSTTRRDE |
| 197 | PKAQEDIRQLVKRYFEAMNHGDMDALRSMIHPDATFHFADEQKTFDREEYLERWKQSFDNVDFRIEIKSIRVEQNDNGTYTVTAHWTVTFHHDHRTHTYDGSTTFFVVKDHNGEWKFVEVRENMNQNQ |
| 198 | PEAEEKIREWIKRFYEAFNKRDMEKLKKMIHPDATFHFEDEGRTYDAEEFLEMIEQFLKKFDARIQVDEIEIRIDEDGTITVTVHVTLEVTDSSQTYTHRATHHWFFRQDHDGSIKLVEVRINTDQEQ |
| 199 | PKDEEDIRDIDRKRLEAIDKGDLDKLRSLFHPDATFHFADYGRTYDKEQFLRYMEELTKKVDMRREIKEVHVEKKPSGEITVTVHVTVQFHHPDRTIHRDITYTVFYRKDHNGEWKIIRFHINETQKN |
| 200 | PKWQEDVERLVRRFFEALNKNDMDALDELFAPDAQFTFEDTQQTYDKEEFKEMIEKMLSNVDMRIEIKSIEVRLNDDGTVTVTVHWTATRHHDNSTFTHHGTTTFFFQPDHDGSWKIKELRINSTNEN |
| 201 | MSDEELKEAVEEFDRRRFEAFNRGDLDKLRELYHPDATFHFASEGQTYNLDQFIEMVRELMSRDVKIQIKRIEIQIDSRKDYITVTATFEMHEHRDSRNKTVQYHYTVVYRKGSFKIIEVRVQSDEDS |
| 202 | MSEEELKEVIRQLVRRFYEAMNKGDLDTLKELFHPDAQFHHAARQQTYDLEEFLEWVKEMFERNVRIQVHQIDVQINPNKDTITVTVHVTFHNHETSSNKTVRVRDHYTFQRGHFKILRVQSHVDDDS |
| 203 | PDPSEIQEQVKDFIRRYVEALNKNDLEELRRLFHDDWTYHDTRTGQDYDRDQFLEMVKRRIDAGNRAEFHVDRIEIDMNDGDDVTVTVHMTVTFHHGDQTRDYRVTEHHTFRRGTNSLKIVRTQTTTHNTQ |
| 204 | SDKSEAREIVKDWFRKFYEALDRGDMDAVRELLHPDVTFHNDSTGQTYNKEDFIRRWEEMLRQGNDAQRQLDSVEFDDNETDEITVTFHVTVHWHHNDHTQTVKITNHDRFRRGTNQFYIIEHRSRRNNTR |
| 205 | MDTSEAQEAIKEWKRQYIEAWNKNDLETLKKLIHPDVTFHDSSQGQTYSADDFLERFERMVRAGVRVQVQLDSMEIDQNHSDTYKVTVHLTVHQHHGDKTTTFSVTQHDRYDQSTNDFKVKEVHIHTDQTE |
| 206 | MDESEAKEAIKEWLRRYYEAWNKGDADALRELLHPDVTFHDSSTGQTYTLDDFLERFERLLKSGTRFQIEIKEIRIDPQETDDYRVTVHYTMTVHHGDQTYTFTATDHFRFRYGTNEFKLVEVHSTVDNTS |
| 207 | SDKSERQEEVKEWVKQFYEAWQNNDADALRELIHPDVTFHNDSTGQTYTADDFLRRFEELVKNGARVKVDIHSLEIDDNETDDVTVTVHYTVHRHHGDETQTQRATDHFTFRYGDNGMRLTSVRSTTDDTE |
| 208 | PQEKEQEEAVRDLIRRMFEAFNKKDVDAVLSLYAEDATYHDSSTGRTFSYEQFKEMLKRLSQTDTRLEVDSLEIRFHDDGTVTVEVHVTMHTRSNSQPQTVKVTSQFHIRQENGSFKIKEEHVTHDST |
| 209 | PEEEEFRKAVEELKRRFVEALNKGDWETIARIFTPDSTMTDSNNQTFNLEEIRERVKQLFSNDTKVEIKSIEIRFDPSGRTVRVTVHITVTTTHNGQKKTFDVTAHDVYVQDGDTLKIVHTQFRRRDT |
| 210 | PEEEEMKKAAEEVVRRFYEAFNNGDADALKELVSDDSQFHDSSGQTYNAEEFKEMVEKLLSANVKLEIKSIEFRIHKDGDEITVHVHVQMHQTTNGNTKTFDGTNTDRLRKDGDSFKIVEHRSHWRNT |
| 211 | PTDEELEEAARQWVRRWVEAFNHKDLDALRELYHDDFRYTDSRTQRTYDKEQFEEMLKEQMKFDFRIEVKSIEVRIDSRKNHVTVRVNMQVHETHGDQDHTVDATYEFHFRFDGTSVYLIEQRVHYNPQ |
| 212 | PSDEEIKKQIEEVVRRYVEALNKRDLDTILSLLHDDWTYRDEKTQRDYSKEQFKKMVEEAAESEFRVEMKSLEIRVDHNKEEVTVQVHLQFHHHHGDRTQTIDVTEHHRLRYDGTSWKIIETRVHIREQ |
| 213 | PEEKEMEEQIREALRRFYEAFNNKDMDKIRKLIHPDATFRFEETQRDYDYEQFIKWVEELLSFDTKIEIKSMEVRIHPNGREVTVTVNVQVHVHRGDKTTTQDVQHHWHFRNDGTDWKVISLQIRIRTT |
| 214 | PSKEEIRKQIEEIIRREFEALNKGDLDTWLSLFHPDAKYHDASKQRTFDREQLKEMFERLVKSDFRVEVKSLRIEVDDSGTEVHATVHFQVHFNHGDETHTFDVTTHITFVRDGTSWKIKSKQTHRHSQ |
| 215 | PEKEEIKKQIKEIIERMFEALNRGDMDTAASYFHDDWQFHNNKEGQTYDKEQWKKMVEELLKNDVRMEIKSIEVRVDDSGREVTVTVHVQMHFNHGDRTQTVDITEHHRFVNDGTDWKIIETHVTINEK |
| 216 | PEEEEMEKQVREVIRRLFEALNKGDLDTADSIYHPDATFRFASTQKDYDYEEFKEWVKRLVETSAQLQIHSMEIRIDPSGREVTVHVHFTVHRNHGDTTHTFRVTSTFHLRNDGTGWKIVSEQIQTDST |
| 217 | PEEEEMEKAVREVIRRYFEAWNKGDMDKIEELYHPDATVRLERYQRDFDYEEFVEWVRRVLSFSLQIDIHSLEIHIDPSGTEATVTVHYTVHYNHGDRTKTIHVTSHFHLRKDGTSWKITSEQAHFRET |
| 218 | PSEEDEKRIKEWKRREVEAFNKGDMDKLEELFDPGVTFVHGNETQDRDKDAFKEFVKQLLSQNIQIEIDSIEIHKSSSDGDLTVTVHVTFHNKSTGYTYHITYHDRYRVDGKEVRLVESQERRDNGR |
| 219 | PSKEDQEQIKEIVKQFYEAFNKGDYDKLSRLLDDDHTVHISNGQTITSREEFEKWVREFKSKFNVHIEIKSIEVRQHDDGTFTVTVHVEVHETHNGETRTYKVTTHHEIRKTKDGWQMVRTEVHWNNTGS |
| 220 | MSDDEAKEAIKEWVRRYVEALNKNDLDALDELFDEDWEYHNSAEPRTYDKEEFKKMVEELIRNRLEFRIEIKSIEIRRTNGDDYTVHVHVQVHTHTDGQQHTMNVTETHHFRFKDGELKLKETRENINPT |
| 221 | TSEEEAQEAIKEMVHRFFEALDKGDWKKLADLFHDDGKFHDSSNPTTYDKEEYIRRVQSRVKANIQSHHEIKSIEIRRTNGDEYTVTVHWTLHEHHDGQTRTRNATTHLRFRFENGQVYLIEMRTTWHST |
| 222 | MSEEEQRQQAKEWLERYFEALNKKDADAVASLFDDDWTFYDSTNQQYDKEEFKEMVERMLRHFDAQFDPKEIRFQIHGDNHITVTVHVTVHFHTDGRQHTFHVTEHHHFRFKDGGIKILETRLHRNPS |
| 223 | MSEKEMEEEARRVFEQLMRAFNTGDADAALSLYHDDARFHDSDGRTFSKEEFKEWFKRWADENNAQLHIHDLEVQIHGNSEVTVHVNINVTFHTDGRQFHITFRSTFRLEFHNGELYIVEEHLHTHST |
| 224 | PSDEQEEQIRQTKKRFIEAFNSGDIDQFKELVHPGATFHFSDTGRTFDKEEFIEMIKKLLSQDTKIEVKSIEIRLKDGTEVTVTVHVTFHTHRGNKTQTFDVTFHDHYRQTKDGWKIIEVHITTNPT |
| 225 | PSKEQEEAIREILKKFAEAFNKNDLDKLFSMLHDDWHYHDASTNQTYSREEFREMVERLLSTDVKIEIKEIRIELKDGEEVTVEIHVTYHFHHGNKTKTVDVTEEHHFRFTKDGVRIVETRVTSNER |
| 226 | PSEEQEKQIRELVQRFYEAFNNKDMDKMSELVHDGAQFHFSNTNQTYDKEEFLDMLRKLLSQDVQVEIKEIRVQLFDGEEITVTVHVQMHRNPGNQTQTYSATHHWRFRLTKDGWRIIEVHITTNPT |
| 227 | PSEEAQEQIKQIKDRFIEALNKKDMDAIDELFHPGAQYYNSDTGTQYDKEEFKDMVKKRLSRDVRLEVKEIRITEHDGDHYTVTVHVEVHFHQGNETQTYKVTFHDRYRKTKDGLYIVSTEVTTRPT |
| 228 | ASPEDQSKIEEWKRRYIEAMNRGDLKTVIELFESSPDARVTENGQTYNPEQFKKMVEEFLKKFDFRIEIKSMEIRQDKDGEIHVTVHYTVHVTHNGETQTYHYTSHDRYRKSNGDWKLVRHQTHTQPS |
| 229 | TDPEDQRKIKEWKQRYIEAMNHRDLDALRELVQSSPDATFYLNGQQFDIDQYIEWMQRLLENFDIRLQLDSMEIRTDEDGTIHVTVHITVHVTHNGKTDTYTVTQHDRYEKHNGSLKLVSSRAHTQPT |
| 230 | MSEEEIKEWVKRWAEAFDKGDVDALASLYDDDAEIHYSDGITITSREEFKEWLKKFMRHADVEHKEIHIHIDKRTGTVRVEFHAHETRRGTGETYDATFSRTFHFDDGRLKRDQAHSDQKKT |
| 231 | TSEEEIKEWVQRWLEAFDKGDWKKLVDMLDEDWTYHNSNGRTITTLEQFRRWFEEFKKNADTQHKEYEIRIDSSTGTVTVHYTVEWTQKGTGEKYTTHVTEHHTFTDGKIQETRTHEDTKKT |
| 232 | MSEEEIREWLKQYYEAIDNKDRDKLDSLVTPDVTFHYSDGITLTTKDQYREFVTRVLSNMDVRHTEVEIHVDSTSGTVTVHVHVKVTHPGTGETYDATVTDTYHFSDGRLTEVRSHRDTKHT |
| 233 | STTEEIIEIIRRFLEAFDKGDMDALAELYAEHATMHTPNGRTFTTRDEIREWFRELFEKAEFHRTEVEIHVDKDTATVHVHVTVTHGDETYHARVTSHFRFKDGKLTEERIQVDSGD |
| 234 | MTSSEIVEIDRQRFEAFDKGDMDRLLELMHDDSTMHTPGGRTFTTKEEIKEWLERLLSNSDIKHTELHIHTTKDTVTATVHVTRTHGDQDYHVTFHYHLTYKDGKLTEVHIHSDENS |
| 235 | MDTEAAIQIVRRLIEAFDKGDVDKLLSLYAEDVTIHHPGGETLTTKEEFREWFKQVKENAEFHHETVEIHVDKDHATVHFTVHVTHNNETITINVTSHFRFKDGKIKEEHVHEDENR |
| 236 | MTTEEIIRIVRRLLEAFDKGDVDALLSIYADDVTLHTPNGRTFTTKEEIEEWLRRFQSNAEVHRTEVEIRVDKDHVTVHVTVTVTHGDQTFHFNVTSHFRFKDGKITEERIHIDQGD |
| 237 | PGEEKIREWLKRYFTAFNNKDLDAVDSLVSDDATFHFSQGQTFHDREEFREWVERLLKTFDFQIQITELRADIDGERITVHWTVHFTNSTSTTTIDVTTHFRFDGDKLVEVHFHLNQRD |
| 238 | PGEEKVKQLIERLFEALDKKDIDAFMSLYDDDATMHYADGITFTSKEEIKKWFEEVLSRFDFRHQITNLRLDLDGETVTVHVKVTFTNPTSEHHFDQTRHFRFRGDKLVEDRIQTHQTN |
| 239 | PSDEEIIRELKRQYIRALNTDIDAFARMWHPDATFHYGDQTFTNKDEVREWFREWLKNFHLEIKSIEIHVYDDGRIEVHAHVQAHTPSSTYDFRATDHYRKTKDGQFRIVETRITWDE |
| 240 | PDDEEEIKEWLKRFYEALDRDADQARELLHEHSTMHVYGTGRTFKNREEILEYLRKQAQHYDHEIKSIEVQPHDNGQVTATVHITIHTPTQTFQVTETHRFQKEGDTWKLVETQIRIQPT |
| 241 | PSDEQQIEELLREYYRAINTDMDEFKKLVSDDSTFHFDYFKITFKNPDEVVDWFAKFASKWQFFIESIEIQPHPDGTATVTVHYTVHTDNQTVRATDHFHFEKRGDQWYLIEVRSHFQDS |
| 242 | DAEEEIKELVKREAEALDKGDVDAWASLFHPDARVEHSNGEEYTNLEELKEAIRRFQKNSEVHREIKEIRIDSSTGRVTVHMTMHVTHGDQDHTFHYTVHIQFDKDGKIVEKQVHWDQQD |
| 243 | PDKDEIEELLRRFYEAWNNGDFDKLRSILHPDVQYHHSSGQTFNNIDEITEWIRRFLSNADAQIEIKSIEVDKSRQTVTVHYTMHVTHGDQTHTIHATDHYTFDKDGKITRMQSHFNQQD |
| 244 | DAEEEIKELLKRLYEALDKGDLDKIREILHPDVTVHYSDGTTLTNIDEVAEYVERFKRNAETHRQIDSIHIDSSTGRVTVHVTVHTTHGDSDHTFHATHHWQFDKDGKITEMRVQVHQQD |
| 245 | PSREEIKELIERWYDAFDKGDVDKLLELMHPDATIHFSNGETITTLEEFREWFERWFSQFERMHRQFHSIEFHGKTATVHSTLHLTRPDGRTFTIHQTNHDTFVDDGKITEHRAHLDEKPT |
| 246 | PSEEEIKELLKRFYEAFDKGDWEALLRIIHDDATFHFSKGETITSKEEFREWFERVMSQMSRIRREIKSIHIDGDRVTVHVTLHVTNDDGSHTTIHVTDTYQFDKDGKITRVESHINEQPT |
| 247 | ASPEQQQRLRETVKRFYDYLNNGDIDAVMSLIADDATFHFDGRTFTTREEIKEFFEKWFKNFDVQIQIESIEIHGDTVTVHVTVTMTNDSETQHHRYTETHHLKKDGRIAQTHIQTDN |
| 248 | TDEEQQRQLKTFARRFYEAFDKGDLDKLISLFADDATMHWNGQTFTTLEEIKKWLKEVMDNVDTAHEIKSIEIHGDTITVHVTVHHHHDHETHTVHVTHTWHFDKDGKIKELRAQTNN |
| 249 | ADEEFQRAVKETKRRLAEALDNGDIDTFISLIHDDSTMHVNGQTFTTKDEIREFYERWLSNADVKHDIDSIEVRGDTVHVHVTVTVTHDHETRTYHVTYTDHYHKDNKLVETRIHTNN |
| 250 | ASEEQIREIIKQFAEAFNNKDLDKMESLYDDDATLHISDGTTFTTPEEFRRWLEEILKRFKQLHMEVKDLHVEGDTVHVHVELHFTHDGQQVTVHATSTFHFDHNNKIKEERIHHDSS |
| 251 | PSEEEIKDLLKKFYEAMDKGDMDLLRELLHPDVQVHISSDPKTFNSLEAFLEWVERWNKNVRVRREIDSIEIRGDTVTVTVTMHVTHKNGQNYTFTIHDHYHFDKDGKIVRIESQVHEHD |
| 252 | PSQEELEEIDRRRLEALDKGDLDTFKKLVHPDATFHISEYQVTFTTADEYIEWWARLYSNFEVHREIKEMHITDREVTAHVTVHVTHRDGRTQTIHMTYHVRYDENGKLVELRVQVHHTG |
| 253 | PSEEEIRKIVKRFYEAMDRGDMETLKRLFHPDAQMHIEKHGITFHDRDEIIDWIAKLISNSDVHREIKDQHVTSNTVTVHVTVHWHHKDGKTDTVHVTHHWHFDHDGKIKSVRITLHDTG |
| 254 | PSEEEIREIDTRRIEAFNRGDMDKLAEIFHPDATIHFESYQITFHSLEEFKEWLRRWVEQIDYKIDIESMEITDRTVTVHITVHLHHKDGRTQTVHITYTIHYDHDGKIKHVRFHSDSRG |
| 255 | PSEEELEEAVRQILQRLYEAIDHGDADTIRSLLDDDWRYTNNNHNQTYSRDEFLDMVKKMVSTDVRHEIKSIEIQIHPNGRTVTVTVHVTVHHTHNGSKRTVDVTDHHHFEYKGNDVKIVHTQATHRQT |
| 256 | MSEEEIREAIRQAVRRFIELFNKGDLDQIKSLFDDDWRYTDARHGQTYDRDEFIEMMKELFDRDVKIELKSIEIRINPDGTTVTVTVHVEVHEIHNGQQRTVTVTEHHHFRFSGDTWRIQRTQVHFRQQ |
| 257 | TSSKEAEEAIRDMLRRYYEAFNKGDMEKLKSLVDPDASFHDARTNQQYDKEQWLEYVKELLKQDAKVEVKSIHIQQQPRGDHVTVTVHVEMHVNRNGQTHTYTITDHYHFVRKGDSWKITRVQSHTHQQ |
| 258 | MSEQELKEAIKEFVERYVELLNRGDADTLAELIDEDWRLHDARTNTTYDKEQFKEWVEKTFKNDTKVELKSIHIQVDPNGNTVTVTVHLTYHTTKDGETRTVDVTEHHHFTFEGDSVKLKRTQVHHHQR |
| 259 | MSEEEIKSAIEELVRRFYEAMNKGDMETIERLVHDDATFHNPDTGQTFDKEEFKEWVETLFRRDTKLEIKGIEIHVDPSGKTVTVTVHVHMTTNKDGQTHHYTVQHHWVFRYKDNSFYIVHVTVRTEQQ |
| 260 | PSDEELEKAVREWIKREAEAFNKGDMETWYSLFHPDARFTDARSNTTYDLEEVKRMIKEMVERNTRMQIDSIEVRVDHDRRTVTVTVHVTFQTTRNGKQQTVHVTYHLHFRYEDNTLKIIHKRAHHNQS |
| 261 | MDEEEVKEAIKDTKRRLIEAMNTGDMDKLDEIYHPDAQFTDNNTGQTFDKEEFKEWVKEQHKMDAKIEVKEIRVQQNDNGETVHVTVHVQIHTTHNGENRTFNVTTHDRYQFKDDTVKIVESRYTTNQT |
| 262 | MSDEEFERAIREYKRRLIEAFNKGDMEALRRLYHPDVTFHDARTNQEYDLDEWLEMVKELLRQDAKIELKEVHIQVHDNRKTVTVTVHVQVHVNRNGQTHTYDVTYHDHYRFKDDTVRIVRVQVHHRQT |
| 263 | ADEEELKEQAKELIRRYYEAWNNNDADTIADLLDEDVTFHNHGQTYRLSRDEVKEMVERLASQDYQLHMRSLEARVGDSNTITVTVHVTMHSRHDTGTRTTHFTDHYVFEYKNGEMKIVEVHSSRETRQT |
| 264 | TSEEEVREAVKELLRQYFEAFNKGDWRTIADIVDENVTYVHNGQTYNLNKEQIVEWVKEMKKNNYKLDIESIEIRVGDSKNATVHVTWTMHVTHDTGTRTIRATTTFHFRQSEGPWKIVSVQTDYEERQN |
| 265 | PSEDELKEQAKELIERFFEALNKGDWDTLAKLFTDDVTFVHNGQQYDLNKEQFIEYVKELFKADTKFHVESIEFQIGDSNTITVHVRWTAHTTHDTGTRTVHANTTFHFVANDGPMKIKRVEVDWEERQN |
| 266 | PDEKEMEEEVREFDKRRVEAINNRDLEKVKSLVHPDATFRFAKTQKDYDKEEFAEWVERMLRNDTQIDIDEVHVEIKHKTQATVTGHITFHTTHGNRTITFHVTYHVHYRRGTHGWKLIRLQLHFNDS |
| 267 | PDDKKAEEDLREIKQRLLEALDKGDWDAVRKIVHPDATFHFESHQQTYDLEQFVEWVKQMASQGTQRHIHRIEIEKKDEHEYTVTVHVTAHHTHGTKNVTVRFTFHDRYRRDTHGWKIHEVRVESDDS |
| 268 | SDLQEQIKELIERWVEAWNRKDLEAIDSLYHPGAQVDDNQIDKEQFKKRVKEILDNTDTQLHIDEIRIQIGDDTRITVEVHVTVHRNEGDRTYTFHITTTFHFRLINGQWYLIEQKTHTNST |
| 269 | PEWEEAVREIIRREVEALNRKDLDKWASLYHPGATDHTQNRDLEEMKDFWRKRLENSNIHIEIKEIRVQKGDDTEVTVEVHVTFHWDHGDRRETYHVTYTMFVQLRNGQWKIVRKQEHHNST |
| 270 | PEEEEIRQAVEEIVRRFYDAFNKNDLDAMASLFDDDWRFHNDSTQTTYSRDEFKDMMKKLLEQGWQLHIKSIEIRINPDGSVTVTVHVQAHNQTSNQTVDVTDHHHLRRDGDEWRIVRTQSQSHQT |
| 271 | PEEDEAKRDIKDIARRFYEALNNKDLDKAASLFHDDAQFHFDNTQRTYDKEQFREWVKKMLDQDWQMHIDSIETRVDNDGTYRTTVHVTIQNSTSNQTQHVTHHWVFRKEGNKFKVVELQERTDST |
| 272 | PSKKDEEDIRTWVRRFYAALNNKDLDAVMSLYTPGSQFHFESTNRTFDREEFRKWVEEAMRRDQEFHIKNIRVERDDDDHFTAEVHVEVHNRKTKVTFTVTQHFHFRKSDGRWRLTEDRIRTHEHPK |
| 273 | PSKEDQEAIEEWYRREEEAFNKGDLDTWASLFHDGSTYHDETRNQTYNKEQLREMFKKLFDQGWQMHIKHIEIRLEDKDTFTVRVHFEFHNKSTKNTFHVDATDRFTKSDGRWKLTERRERRHEQPT |
| 274 | PSEEAQKEIERIIREMYEAFNKGDLEAIYSLLAPDWTFHNESNNQTYNREQFKEMVEKLIKAGFKIDVESIEIRAPDKDNYTARVHVTVTNKQTKDTFKTTDTHHFRRSEGTFYLLETRVTMDRHPT |
| 275 | PSDEDEREIREWYKREAEALNSGDVDAWKELFHPGAQFHDQTRNQTFDLDEMREWVEKMIRAGFRFHITSIEIRVDDKDHITVHVHFQVHNEKTGDEFRFDSTDHFRKSHGRLRLTERQIRTEEYPS |
| 276 | PSEEQQEAIKEFKKRFYEALNKGDLETIKSLMHPGATFHFESRGQTYDREEFIEIVEKLIRANFKFQLDSVEIRKDDEDNITVHSTVTMHNDSTQRTFHVDATDHYTYSNGELRLTHSRFRTHEQPT |
| 277 | TDKEQEEKIREWLNRFYEALNKGDLDAIDSLLTPGWTYHDKSRNQTYDKEQFRDMVEKMMRNNLRIEVTSIEIRSDDDTTATVTVHVHVTNDQTGTQFDVTDHHHFRLSTGELRLVETHFETHSKPT |
| 278 | ADPSDQEIVKQLVERLFEAINKGDRDTIKSIYAPDAQMHISQEQVTFDTLEEIAEWLVKWSSRYDIQIDSLRIELHGDYVHVTVHVTIRQRGTGKTIRVTSHITFDKNGWKIKREHVHFDNGE |
| 279 | MSEDEMREAIKEVKRQLIEAMNTGDLDTLRSLFHPDATFEHSSGQQYDLEEFLEMVKKLNENNVRIELKEMHIQIDSDGRHVHVTVHVQFHNATTKQTYKVTAHDRYRFDGDTVKIVETTFHHHPT |
| 280 | TDDEEAEKKVRDWFREYARAFNEKDVDALASFYHPDARLRDPNGRDYSYEEIREWYERVLSNDAQITIHSMEIRFDSDGHHVTVRVTMTFHNSRTGETYTVHAHFHFRFNGDTLKLVHVQYHHHST |
| 281 | PGEEEAEKAVREVLRRLFEALDNKDWKTLSELYDKDATFHHTDHGETQTYTKEQFIRRVRSNKDIQTAHHMKSIEIRIDSDGHNVTVTVHVEVHETRGDKTDTITVTRHFHLRLEGDSWKVKRDTSHRDSS |
| 282 | PGEEEIKEAAKELLRRFYEAVDKNDIDTLRSLLDKDWKYHETRDGKTETYDADQFLERIARNKDVQVHREIKSIEIRVDSDGRTVTATVHVQVHEHHGDKTHTVDVTDTHHLRYEGDSFKIVETHAHYDSG |
| 283 | PQEEEIKEAVKQIVERLIEAINNNDWEKFASLYTDGSTFYLSQTGQHYDLEQFIEWLKQMASTGFQIEIKSIEIRINGDSREATVRVHFTLHQTRNGQDYTIQVTSEIRIRQDDSTFKILSERVHFSDS |
| 284 | MSEEELKRAVREVDERRFEAFNRGDMDTIRDIVHPGAQYHDASKQRTFDKEEYLKMLEELLSQDVKIQIKEIHIEVKDDGTVTVTATVEVHRNHDNETRTVEVHYTIFYRLQDGQWKIVEMRESTDSGE |
| 285 | MSEKELEEAVRDALRKFYEALNNGDWEKVARLVHPGAQIHFASTQRTYDVEEMIEWVKKMFSNDTEIHIDSIEIEIKDNGEVTVHVHGQMHTNHDNRTITARVTDTYHFRLSNGRWKIVELHSTIDNGS |
| 286 | MSEKEFKEQIREWVQRWVELFNQGDADAMKSLYADGATIHFSATQQDYSKDQFREWVREMFEADTKMQVDSIEIHIYDDGTVTVHVHMTLHTHHPDRTQTVQATFTFHFRFENGSLKLTRQEVTYDSRS |
| 287 | MSEEELKEAIKEIKERLARAMDSGDADTFASLVHPGAQFHDERTQQTFSIEEMREMIKEIAKSDTKRQIDRIEIQIYKDGEVEVRVHVTVHTHHDNETKTQWATYTDRYVFQNGQVYLTESRVTYDSQS |
| 288 | ASEEEQREAIKEMIKRLVEAFNKGDADALKRLFHPGWTYHNNAEQRTFDLEEFREWVRKVFSQDTKVQITSIEIEVKDDGRVTVHVHVKYTTHHSDQTRHVDVTETHHVEFHNGQLRFVETHVTWDSGS |
| 289 | ASEEEQREQIKEWIKRFLEAFNNGDLDTLKKLFHPGIQFHFSSEQRTYDLEEFIKMIEELLSQDTKVHLHQVDIEVRDDGTVHVTAHWEFRTHHDNKTKTVRVTTTFVFEFHNGSMRLVEVRITHDDGS |
| 290 | DEEEIKKQIKEILEQFYEAFNKGDMDRIRELVHDDATFHNQSDPKTYNKEQFLRMLEELFKQGVTMEIKSIHIDVKSDDAEATVHVQFHRTHGDETQTYDVTHHWRFRRFGDEWKIVEVHERTHQT |
| 291 | DEEEDKKEIKEWLERYYEAFNKGDMDALRELLSDDAEFHNQSDPKTYDKEEWLEMLEKLWKQGTRMQIDSMEIDPGSDTLTVTVHVTVTTHRGDKTRHTTFTHHWHFRLEGDSWKLVRVEERTHQR |
| 292 | TPEEIEKAMRDIKRRYVEAFNKGDMDTISSLLSDDAQFTFSSDNRTYDKEEYLEMIKKLLSNDAKVEIKEQHIDVKSDTVTVETHVQIHVTHGDETRTYDITTHDHYERRGDQWYIVHTTITTRQT |
| 293 | DEEEAREAAERIIRALVEAMNKGDLEAIMSYYDDDATFTNASNNTTFDKEEMKKWFKDFFSSGTQVQIDSIEIHVHSDEVRATVHLTMHTHHGDKTRTVHYTSHFVLVRKGDEFKIVHEKLQSDQA |
| 294 | TPEEIQEQIKEAVRRMFEAINNGDADAVMELVHPDATFHHESNSTTYDKEEFRDWVEKFFKAGMRIEVKSLEVDVKSDTATVTVHWTVHQHNGDKTRTVNATTHIRFTRFGDEWKIIEVRIRTHQT |
| 295 | PDPSDQEAIRQWFRREVEAINKGDIDAWASLFHPDSKYHHQSGQTYDYEQFIEMFERLMKTVDVKIEIKSLEIRQTKDGNEIEVHVHVTVHENHPNKTRTVQVTQTDRFEKRGDQFYLAERHTHTDGE |
| 296 | IDPSDEEAIRQLIERFYEAFNRKDWKAVADLVHPDATYHNSDGQTYDWEQFVEMVRRRIERFDIKMEIKEIRFQPTPDGNEITVTVHVQVHVTHDNETRTFQATHHWFFRKIGNQWYIIRLEERRNGT |
| 297 | ADPSDQEDIKRIKRRLAEAFNKKDWEAIARIFHPDATFHHENGQTYDWEQYVEMVKRFLEKFDARFEIDHIEIRLDKDGNDFHVTVHVTVHTTNDNQTRTVHITQHDRYRKEGDQWYIIESQTHTSGR |
| 298 | PHEEEQKDIKEWFKRYAEAWNKKDWETLARLFHPDATFHHQSGQTYNWEQYVEMVRRFLSTTDSKIQIHSLEIRLDKDGNKITVHVTWTMHTHHDNQTRTVTGHTTFFFVQEGNQWYVKEVRTTTDGS |
| 299 | PHEEDQRELEEIIRRYYESFNQKDWQAAADIVHPDVKYHHQSGQTYDYEQLVEMIKRLFENADVHIEIKHIEFRQHKDGGRATVQVHVQVHVRHDHKDTTVDVQHHWVFEKRGNTWYIVSVEERTDGS |
| 300 | PEEKEIEEAAREVIKRFYEAFNKRDMDALEELLHPDARFHTYQGETYDVKQFKEMVERLLSLDIKIDIKSIQADTFKDGTATITVHVEVHVRHDTSTHTVRVTHTWHVELQPNGELKIKSLQENRDND |
| 301 | PESEEIQEQIKETIKREAEALNKRDIDAWASLFHPDARYTENNGRTYDYEQMKEMVERWLSTDVQVEVKSIRIDDTKDGTGTGTVHITFTQHHDTETRTVRVTYHIHMERDHNGEVYIKEKHARRHDS |
| 302 | PESEEIEKQIREWLRRFYEAFNKRDLDAIASLFDDDWRYYNHSGQTYDKEQFREMVEKLLSTDAQIDIDSIQVDDTKDGTATVTVHLTVHERHDTEQRTVRTTDHHHFRLQPDGQLKITHTHEHRDND |
| 303 | PESEEIQEKIRQWLRRFYEAFNKGDWEALASLYAPDARMEHNNGQTYDYEQIVEMIKRLLSVPIKMEIKRIEIESTKDGTGTVTVHVQVHTHHPTSTHTVDVTQHIHFEVQPNGELKIKRDRESSHNS |
| 304 | PEEKEIEDLVREWLRRFFEAFNKRDLDKLKEMFDDDWRFHNANNQTYDRDEFKDWVRKLLSVPIRVQVDSIEVKDVKDGTATVTVHVTVHEHTPTKTKTVRVTEHHHFRVQPDGSIKITRTEVRHHDS |
| 305 | PEDEEIKEAVKQVVERFFEAMNKGDLDELKSLFDEDWRYHHASGQTYDLEQFLKMVEEFHSVPTRIQVKELRIDDTKDGTATVTVHVEVHETHKDSHHTQRFTEHHHFERHDSGEFRIVETRTQTDDS |
| 306 | PEDDELRQAAREIIKRLYEAMNNNDLDTLRSLFAPDATFHHNKGETYDLEQFLEMVERLASIDTRIQVDSMEVKDTKDGTATVEVHVTITQRHDTSTHTVRFHHEWHVRRRPNGQVYVKELRIQSNDD |
| 307 | PREEELRRQIREFIKRLVEALNKRDVDHWLSMFSDDWTYHDSRDKKTFDKEEFAEMLKELLKFPFQIQITSIEIRIYPDWRTATVHVTVTLTVHRGDETRHVNATETHHFRFTENDLRIKETRVNYSGEQ |
| 308 | PEEKEIEEEIRDVDKKRVEAFNNRDMDTWKSLFHPDATFHFYEDKKTFNLEEVVKMIKELLSFEMRVEIKEVHIEFEPDKSRVTVTARVQFHVTRGDKTVTVHVDYTFHYQRSKDNWKIVEVRIRTSGDQ |
| 309 | PSKKQQEDIRDIVRALFEAFNKKDWEKMASYYDEDARITFTSTNQTYDKEQFIRIIQELLKQDYHLHVHSIEIRLGSDNNTVTVRVHFTMTQQTTGTTFEATSEIHITREGDQWKIKEEHITMRSQPT |
| 310 | PSKEQQEQIKELKERFIEALNKGDWKAIAELMHPDAKYTDNSTNKTFDREEIEELVKKQFNAKLHFQLDGIEIRLDPSNTDIRVTVHLTVTQADTKTTVKATTHDRYVRFGDEWKIVETQTTRHEQPT |
| 311 | PKEEELKRQVREFDERRIEAINRKDLDKVKKLFDDDVTFHFAATQQTYSAEEFLEMVKEWLDNDVKIDIRSIHIQVVGDGTVHVTVHVTMHVTRNGKTDTVHVTYTITYQLKDGSWKILRMQVHIHPT |
| 312 | VSEEQVEEWVRRFFEAVNKNDRETLKKLFHPDVTFHFNEGDRTITYTTLEEFVEWLEKFAQNYHFEIKSIEIETKGDEVTVHVTWEVTNRTQGHTYEGHTTVRFDKDGRVVEVRFTFDST |
| 313 | MSEEQVKEWVKREVEALNNGDLDAWASMFTPDVTVHFFHGDTTYTFKTLEELKEWLEKWLRNFQIHIKSIEVQTDDSSVTVTVHLQVTERRTGRTFDVTFHDHFDKDGQLVERHVHFNHS |
| 314 | PSKEQIKEFVKREVEIFNQGDLEAWASLYAPDATVHFYHGDETYTFTTREELKEWAEKFWRSHTVQLDSIEIRHDSDTATVHVHLTVHNRDTGKTAKVDSTVHFDHDGKLKEKHVRFRDT |
| 315 | PSQEEIKELVKRFYDALDSGDMDTFDSLIAPDWTYHYYQDNTSITFKTREEFKKWVNEWASFVHRTFRSMEVRKDGESVTVHVEVQVTDSRTKQTFDTRDTHHFDHDGKLRETRSQYHST |
| 316 | PSKEQIEEWLRRFYEAFNKGDADAAKELMDPDSQMHVQEGDETHTMTTQEEIKKWIEEFAKDHEIEIHSIEIRHEDSSVTVTSHVTVHNRKEKQTYEATETHHFDKDGRLVETHTHFHPT |
| 317 | DTPERQEAVRQLIRRYFEAFNSGDWEKLASLYTPDSRVHNPATGTTLNYEQVVRWYQKQMSRDAKMELKSVEIRINDDGTVTVRVTFTMHFTQDGQKYTTHIHATFHVERGDSSWRFKEVRFQRDTRG |
| 318 | DDEKNEEDVRELLKRFYDAINKGDLDKIRSLMDEDWRYTDASRQQTFDLDEFLDMIKKMHSNDTKVQIHSIEVEIHDDGKVTVTVHVTFHSNHNGQTYTTHVTDTHHVDYGDSSWKFVRTQTHRDTRG |
| 319 | DDEEAEKAVKDWLRRFAEAFNKGDMDTLKELFTDDWRFTDARTGTTIDLDQFMDYLRKLLSNNARMQIDSVEIHINDDGTVTVHVHVTIHSTHNGQKYTFHVTETHHFRRGDDDWKVVRTQAHTESRG |
| 320 | DDKDDEEAVRQTIRRWAEAFNKGDADALYELYDEDSTIHDPSTQQTYNVDQFKEMVKKLQSQDVKLQVHSVEVEIHDDGSVTVHVTYTVHSTHNGQPYTVEFHTTFHVEKSEDRYRIVEQRDSNDQRG |
| 321 | MSEEELKRAIKELKKRLVEAMDSGDWETVKKLFDDDVTFHDPSKNQTYSVEEFVRMMTKLASRGIHRQIDSIHIEFSDDGTAHVTVHVTVHERHGDKTHTFTATAHDTYRFENGSLRILSSTFNTDS |
| 322 | DKEEQKKQVEEIVRAFYEAFNKKDLEKIRRMLHPDATYHQQRGNQEETYDYEEFIDMVRERLKFDFRIEIKEIRIQVLDDTQIEVEVHVTFHWTHNGKTKTVDVTHHWVFRLTDTGWYLIELHARHTEK |
| 323 | DFPKAEEDIKRLKKRFVEAMNHNDWEKLARMFHPDAQFVFINGNQREQYDWEEYVRMVQELAKFPFRIEVKSIEVRKVDDTTYTATVHMTVHFNHNGKTNTVTVTQHDVYRQEKDGWKVLETQEQHDQE |
| 324 | DEPEQQEQVERVIRRFYEALNHKDLDTVRELIHPDATFHTFRGDEQQTYDYEQYIRRIEEMAKFDFRIEIKSIEIRVDDKNRVTVTVHVQVHVNRNGQTHTVNVTHHWTLQQTSTGWYIIEVRSRFDNS |
| 325 | DKPKAESDVRETVRRFYEAMNKRDLDKLRELLHPDAKVTQYEGDQQKDFDYEEFLKMVKELASIDFRIEIKRIEVEQKSDNEFTVRVHVELHFNHNGETHTVTATHEWHLRRTSTGWYIIEVRERRDQQ |
| 326 | DEPEVREQVKEFKRRLLEAIDKGDWEKFARLVHPDAQFTHTHGDQSQTFDREEVIRMIQEQSKFPTAHHMKSIEIRKVDEHEFEVRVHVEVHENRNGQQHTVTVTYHDRYRRDSTGVKLIETQTRFTNQ |
| 327 | DQPEQIEHVLEIVRRFYEALNHKDLDKIRELIHPDATFHHTHGDKTETYDYEEFLKMVREMMKFDFRIEIKSIEVRIDSKNEITVTVHVEVHETHNGEKRTVTVTHHWRLRRTKDGWYIIEVRETRDQQ |
| 328 | MSEEEQKEAIKEFKKRVIEAFNKGDWEAVRRLFTDDVTFHHDSGETYDLDQFAEMIKKQKSTDAKMELKEVRIQVDENNTVHATFHVEVHRRHGDKTTTIEYTSHDRYRFENGQMKIVETHIHSRQRQ |
| 329 | MSEQELEEAVREWLERYYEAYNKGDVDALRELIHDDWRNTEENGTTYDKEEFLKMVEEQMSNNTQLDVKSIEVRVDEDGTVTATVHVQMHTTRGDKTHTVDVTETHHFRFKDGQLRLVETRFTHDDKS |
| 330 | TSEKEAEEEIREILRRFYEAFDRGDADALMELLHDDVRFHHESGETVDKEEFRERVERIASTDTAHKIDSMEIRFDSDGRATVTVHVTVHQRHGDKTVTRQVTHHWFFRKKDGEYYIVEVRIHTRQED |
| 331 | MSEEELKEAAKEFVRREVEAFNQGDADAWASLFHDDVQFDHARGTTYDKEQMKEMIERLLSNDTKVQIDSIEVRIDGDGTVHVTVHVTVHTTHGDQDHTYHVTEHITFRLENGRWKVVRKEIHTNQTQ |
| 332 | MSEEELRRAVEEWLRRFYEALNRGDWEAVAKLVTDDATFHFDEGQTYDKEQFVEMIRQMLSFNVQIQIDSIEIRVDSDGTVHVTVHVTVHETRGDKTVTVHITETHTFRFENGRLKLVETHIHTDNTK |
| 333 | PSSEELKEAAREVIERFYEAVNRNDLDKLRELIHPDATFTHESNPQTYSAEEWLKRFEERVRAGAQLHLQIHHIEFQTDDNGEITVTVHVTVTNSQRNKTVEVTHHWFLRNINGQMYIVHVRERTDNKE |
| 334 | PSEEELRELVKEWVRRYFEAWNKNDWEKLKSLFDDDWRFHDSDENTTYDLEEWVERFRQYVSSGNDTQLQITSIEIEVKPGGEVTVTVHYKMTNKKTGTTYDVTEHHRFRNENGSLKLKETRTTTDRKE |
| 335 | PEKKKIKEDIKELIRRYFEALDRGDWEELEKLMDDDWRYHQNYENQTYDKEEFLRWVEKMASQDTAHHIKSMEIRVNDDNTHVTVHVHVEVHHNRNGKTKTVRVTETHHFQRKGDTWKIVETRVTHDDT |
| 336 | PEEKEIREQLKEIVEKFFEAFDKGDVDKLKSLMAPDAQFHFSGENQTYDLEEFLERVQRFASQGTAHELESIEIHVNSDGTHATVHVHWTLHVTRNGETRTFRGTTTFHFQRRGDSWRVVEVEFRVQPT |
| 337 | PEDRETIKELIKRFFDAINNGDRDTLKELLHDDAQMHVAAEQRTFDTKEEIVRWLVEMASTMHIEIKSIEIRKDEGDQWKVTIHWTGRRRNEPSDFDATTHMRFDKSGKIVELRFHVHQT |
| 338 | PEQEERIKEIIERFYEAINHGDMDTLRELLAPDWQFHDNANQITFTTLEEFLQWIAEWAKNFRIEIKDLHIQPGDGDTVTVEVHYTIHNKTNPTTYTATDTHHFDKNGKIKETRSTVHQN |
| 339 | PSEEQQEKIKELKKRLVEAFNKGDWKALRDLFHPDVTFHISDNKTIKTLDEFIEWVKKFFQNIQLHIKSIEIELKDDTTVTVTVHVEIHRTRGDKDDTATVTFHDVYRFENGQLKITESEARFQPS |
| 340 | PKEKEQIEAIREILRQYYEAWDKKDLDKLRELLHPDWTYYDSRTGQQYDREEYIKMVEEMLSKFKDVRHEIDSIEFEINGDEVTVHVHVTVTFNTGNQQHHVQVTETHHFRQEGDRWKIVRTQTTTNNTR |
| 341 | PKEKKMEEQVRDWVRREIEALNKGDMDTWASIFDPDVTFHFSDTGTTYDRDEVKEMFERFLKRFKDTRIEIKSIEIHIDGDHVTVHVTVQVHVNQGDRTYTNHYHQTIHFRFEGDQLKITEKHVTTDNSD |
| 342 | PKEQELEEAAREWARRFYDAIQKHDLDAIASLYAEDSRMHNSDTGRTFDREEIIEMVKQFLERFKDTRVEIKSMEIEIRGDTVTVTVHVTITFNRGNQQYHVNFTQHFHFQREGDEFKLKHDEASTDNSD |
| 343 | PGEESAKEAVKQFFTRFVEAVNKGDMDKVRSLFHPGSTFHHDRKGQTYDLDEFIDIVEKLAKAGMQIQIESIHVEQLPNGQYRATVHWTATVHHNGRTYTFHGTTTVRLEYHGNEWRVVELTVNIDST |
| 344 | PGDETAKEEIKRFLKQLYDAFNTGDMDKIKELLHPGWKYYDATKNQTYDKEQFLEMVKKLLSQGVQLQIKSIEIDKDKDGGWTVHVHVTFTVTHNGENKHFDATETHFFRFEGDTLKLVETRVNEQPS |
| 345 | PSQEQQEAIKDFKRRYVEAFNNGDLEKLKELFHPGSQFTHESHQQTFDREEFIEMLEEMLRHNTKIQIDEIRIELKGTHEVTVTVHMTVTTHHDTETRHVRITQHDHYRLENGTWKLVRTHVETHEQPS |
| 346 | PSKEAEEAIREWIKREVEAFNKGDLDAWVELFHDGAQLHVEATQTTYDKEEMRDYIRKLLSQDTKIQITSIEITQHGEDEYRVTVHVQVHVHHDHKTVTTDVTATFFIKQQDGEFKIVSKHVERDTKDT |
| 347 | PPEEEIAEAARQIVRRLVEAFNNNDMDALEELYHDDARIHQPNGETYDKEEFKEWVREFIEKNNAKVEIKSIQVQSIDSNGEITVEVHVTVTVHRNGQQFHSDVTSQFRFRKKDGSFKILSEHIHTDDKD |
| 348 | PEEEEAKEAAKDTVRRWAEAFNNNDLDKLEELYHDDARIHHASGQTYDKEEWKRWVEEFISKFDTKIEIHSIHIVQIFSDGTITVQVHVTLHANHNGQTYTVHFTYHFRLVKINGDWKILRQEERRDDID |
| 349 | MSKEQIKELLKRLYEAFNKNDLDKLRELFHPDATIHFSRYGITFHDLEEFLEWVERFIKTFNVHIQITEIRIDDTELRVTVHVEVHFHHPGETRTVRVTEHHTFRDGRLVETHTHFQPQE |
| 350 | MSEEEIRELKKRLAEAIDKGDWETVARLMTPGATFHFQSENYTATDLEEFIRWVQEWFKRFPDVQRTLESIEVNGHTVTVHMTVHFHHKDETRTVRVTVHDHYRGNRITESRFHFNEEK |
| 351 | TGKDHIKEILRRFYEAINRNDLDALAELFHPGAQIHFWNGTTLHDLEEMRRFFKKLFEDLEDVKIQIEEIHFDGSTATVHYTVHVHHSSGKTVTFRGTDHFRFRNGKIQELRSHVDSTHGS |
| 352 | PSEEVKEIIRRAYEAFDKGDWETLRRLFTPDAQFTTHNSDGSTIHVTTQDEFREWFERVKSTFTEMHREIKSVDATGDTVTVHGTVHFTRPDGQTYTIHVTHHWTFENGKIKEVHEHIDYGG |
| 353 | ASQEEIKEFLKRFYEAVNRGDLDTAHEMLHPGWTFHHASGETLKSRDEVRKWLEDFSKTYQLEVTSIEVHQNPDGTYTAHVHVNVTNKNTQTTITFSDTHHFDRNGKLVETRTQETG |
| 354 | TSKEEIEEIIRQMYEALNTGDRDAIKEYFHPGAQLHIWNGQTFDGLDEIAEFLVQFASEYQMQIDSIEIHKNPDGTYTVHVHVTVTNKKTQQTYRQHHTWHFDHNNKVVEVRVQMTG |
| 355 | TSEEEIRRWLKEFYEALDNGDLDRLLELIHPGATFHITDGETLTTKDEIKEFLREFFERFSDLKHDITQIDIDGNTVTVHVQVHVHHPNKTVTIDITEHHHFKDGKLVETHVHIDQKDN |
| 356 | PISEEQIREVVKRLFEALDKGDWETFERIYTDDSTIHIDDRTFTDKEEFLRWFEQFHKTFKDVQHDIESIEIRGDTVTVRGTMTVTHGDKTQHYTVNSHFHFKDGKLKSERIHRHRQPR |
| 357 | PTEEEIKQLVEQFYESLNRRDLEKVASYFHDDATMHIDQGTTFTTLEEIKEWFERFLKRFPRFRIEIESIEFRGDTVTVHVTVQHHRRDTGKTFHTRVTHHWRFDKDGKIVELHIHSDTKES |
| 358 | PASEEIIRELVKRYYEEFWNTGDADRAAELLHPDATIHYYNGTTLTTMDEIKKYLEEFLKRFSRVHVEIESMEVRGDEVTVHVRMTTHHGDQTYHYRITHHWRFKDGKIVEVHIHENQE |
| 359 | DELKEIINRILKAIYDSKDPKAARDYFHPDATITFKSEQQTYDLDQYIKWFEEFMRTFPDATFQIDHIRVDGDTVTVHWTVHVTDQRTGETYTTHATTTFHFKDGKITETHYQLHTQKT |
| 360 | PSRERIKELIKRYYDAYNNRDLEALKEYLTPDATMHIDATKITFTNLEEIEEWIRRFLSRYKDFRFEIDRIEIDGDTVTVHVTVHFTHDNNQTFTYEVTEHHHFSDDKIVETRTHVHERK |
| 361 | PEEEEIKEWVKKYIEAIFNTKDIEVARQFLTPDWKFHNNSEQRTYDREEFAEMLEQFQKKFPDAKVQIHSIEIRGDTVTAHITVHVTDKDKDVTIHVTEHHHFKDGKITETRVQEHTQE |
| 362 | PSEEEIKELLKRFYEAFNKGDWETLLDLFSEDSTIHFSNGETLTDLEEIRRFFERVLRNRVQIEVKSIHIEPGAGRVTVHVKFHQNRDGRTYTFDVTEHHQIRGKKIVETHFNWDNHP |
| 363 | TSQEEIKDLIKRYYEAFNRKDLDALSELMTPDGQLHIEYQGITFHSAEEIREWAKRFIEKHSRFHLEIKSIEVRGDTATVHVTAHFTDDDTPTDVTIHITEHHHFKDGKLKETHVRSDTQQN |
| 364 | MSEEEIRQWVRRAVEAFNRKDFDALKEIYDDDWQFHYAGEQRTFHDIEEFLDWLRKFFSEFSEVHMEIKDLHIDGDTVTVHITVHVHHEEEPKTTTIHQTEHHHFRNGKITETEVHFDEQET |
| 365 | MSQEQAEEIVRRLLEALDKGDFEALKEIYDEDAQFHFAARGETLHDLDEFIEWVRRVKSQFSRTAHTPESIEVNGNTVTVHVHAQFHHSEEPKTVDVTMTRHFRFRNGKLVEDRVQMHEQPT |
| 366 | PSEEEIKEWKKQFVEAFDNGDMDALARLMHPDATLDVSNGEQLTTLEEIREWFERFFKTFTSQHTEIEIHFHDDGRVTVHLHVHFTQNGKNITIDVTVHDRYEGKKLTESRFHFHQRD |
| 367 | ASEEEIRELLKRFYEAIDKGDVDAIMRILHPDATFHDSNGETLTTKEELKEWFRRFFESTGMQRTKLEIHIDDNGTATVHVTITTTHNGQTFHIDFTEHHRFKDKKIVETRQHSHQKD |
| 368 | MSEEQLREFKKRFMEAFQRGDLDALAELVHDDATFTDQQTNTTFTSKEEFKKWVEEFLKNSEFRVEIHSIEIRGDTVTVHFTVHVTHGDEHRTIRFTTHDRYTDNKLTETHFHFDNGG |
| 369 | PSEEELRELAKRFYEALNKGDLDALAELYSDDATLHAQQDNITFTTKDEMKEWFKKFMESVDFRIEVKEIEARGDTVTVHVTIHFTHGDKHQTIQFTHHWHFSDGKIKETQESVDTGG |
| 370 | TDEEQIREFLKRFYRAFAEERDANTVASLLDEDSTFHVQNGITFNNQDSIREWFERLIKSVERFRVEIKSIEVRGDTATVHVTIEMHHGDETVTVHVTHHWRFKDGRVVEVHVSTDERR |
| 371 | ANREEIERLKKKAIEAFWRHKDANIFADLMTDDVTFHISNGTTLTTREEVVEWFQRWAETNQRFHIEIKEQHIHGNHATTHVEIHVTRGDQDYTIHVTQHDRYKDGKVTESHIHIDERE |
| 372 | MSEEQIREFLRRMYEAMDKGDLDALRELVHPDATFHFDSGETYNDREEFIEWVRRWQSQFPDVRRQIHSIHIDGDTATVHVTLHVNTPGKTKTVTATHTWRFRDDKIVEVHFHSDDQK |
| 373 | PSQEEIEELVKRLAEAFDKGDAEAVRRLLHPDVTIHFSDGETLTSWEDIKRFVEKFKSQFPDVKRDIESIEVNGDTVTVHWTLHVNTKDSTITNTGTTHIRFKDKRITEIRFHVDKQN |
| 374 | PSREQIEELLRRFYESLDKKDADAIKELFHPGATIHFSDGVTLTDPDEIKEWFAKWLSKFPDSRHEMKSVEFHGDEVTVHVQVHLRDDSRDQTIDVTHHWRFTDNKITRLEARFDRKTS |
| 375 | ASEEEIRQWKRRWVEAFNNNDLDELKSLWHPGATMHISRGITVTDLEEFREWYERFAKQFPDFHIEIKSLEVRGDTATVHLTLTLNHDNQTIHVHFRVHDRYTDKRLTESHFHYDEQRT |
| 376 | MDESELREIDKRRIEAWNNRDPDKLKEHFHPGATVHFSEGVTLTDRDEIKRFFATLLERFKDSRIQIDEQHFDGDTVEVHMTLHFTHDNQTITVHATYHVHYKNKRLVRMQVHRDNRTS |
| 377 | DDESQIRELVKRFYEAFQKGDLDALSEIVHDDAQMHISQENTTFNNKDEIRKWLERVLRELKDVRVEIKSIEIRGDTVTVHVQFHVTHGDETVTVDVTDHYRFKDGKIVELRSHVNQRRG |
| 378 | DDEEEIKETKKRAIDAIQRGDWEALRKLIHPDARFHFASEQITFTTLDEFIEWFRQWFSSVKDVRAEIKEIRIDGDTATVHVQFHVNHGDKTTTIDATFHDRYKDGKLVESQVHFDQKDD |
| 379 | DDESQLRELVKRFYEALEKGDLDKLSKLVHDDATFHFSSEQITLTTKEEFQEWVERWIKRFKDFRFEIKEIRVHGDTVTVHVQVHFHHGDETVTVDITDHYTFKDGKIVEVRSHVDEKSG |
| 380 | DDESQIRELVKRFYEAFAKGDMDKLRELIHDDATMHLSQKQITAKTKEEILKLLEELIRSLKDFRVEIKSIEVRGDTVTVHVQFHVTHNTKDVTIDVTDHYRFKDGKITELRSHSDTKTD |
| 381 | SVSEEQIREFVKALMEAFDKNDWERLARLMHPDATFHHSDQTYPLSKEEIEEWIKRFLKSIKDSHHQIESLEVRGDTVHIHWTVELHDDSRTVTVHATTTIHFKGDKVTEIRTHYDEQPT |
| 382 | PDEEKIEEILRRFYEALDKGDWEKLADLLSDDWKYTNGNETRPGDRDEFIEWVKRFKSQFPDVKRDIRRIEIRGDTVTVHVTIHVNHKDKTKTVHVTDTHHFEGDRIRETHGQTDNQ |
| 383 | PDEEDIKQILKRFYDAMDSGDAEAAASLFSDDVTFYNGNQTFNLGKEEFKEWVERWFSQFQDTHREIKSIHIDGDTVTVEYTVHFNHKDETQTYRYQDHFRFEGKKIKEVRSRRDEQ |
| 384 | MSIEQFVRQWFEAFDKGDVDALLSLYHEGVQIHVTHSSGNTITFDDKDEFREWYTQWIQRWDDMSRQIESIEVRGDTVTVHATVHMTHKTGQRYTVHVTAHFHFRNGKLVEQRTTFDEKKT |
| 385 | PSQEEIKEIVIRMIEAVWKTGDWRIIERYYHPGATVHVHHSDGTTITFTDLEELLEWWERMMRSLKRVHVHIDSVEVRGKTVTVHVTIHVHQGDQTFTFHVTSTFHFDDNGKITSEEVHINQQD |
| 386 | TSEEEIRELLKRFYEAWDKGDWETLVSLLDDDATYHHSNGQTFHNAEEFTEWYKQMMSNMEIHREIHSIEIDGDRATVHFTVHVTHDNEDRTIQGTEHHQFKDGKIVETHVHLDTKNG |
| 387 | PGEETIKELVKRFYEAVDNGDWDAVAKLLTDDATFQVAKEGRQFNDKEEIKEWFRKYWENYDVQRHIHKIDIDGDTVTVHYTVHRTHNGETQTYTATDHFRFRNGKITRVTSHITG |
| 388 | ASEEDQEQAKQMIDTYYEAINTGNAEEAVRKLFDDDATMHNDGQQFNDKDEMIEYIKSIGEVHIEIKEIRITVNPDGTYTVTVHVTFHHGDNTVRVTHHWFLEKENGSWKIKELREHRDQE |
| 389 | SEDEEAAREVVRQYFEALNHNDVDALASLYHDDATMHISEGRTFTTREEIKRWYERFLKESDIRLEVKEIEIRIHDNGEITVHVHVQVTHRETGRTVTVTFTFFLVKIGNRWYIKEVRAHFHQN |
| 390 | PSDEEAIRELKKRFIEAFDKGDLDAIAELLHPDATMHIQNGRTFTTLEEIKEWFKRLMDNMTTAHQIDSIEIRHNDDGSITVTVHVTVTFPETGTTVRVTFHDFYDKRGFKIVETHITWDQNE |
| 391 | PSDEEVIRDWYRQEAEALNNRDVDTWASLFHDDATVHFSNGQTFTTKEEYKEWARELLEKFDFRIQIDSLEVRTHDDGSITVTVHMTVTYNKTGTTYHFTYHDHFDKRGFKLVERHEQVHQRN |
| 392 | MSDEEVIRDLKRRFVEAFNHGDADTMASLFAPDATMHIQNGITFTTREEIREWIEKLLKRFDARMQLDSIEIRFYSNGTVEVTVHVTVTRNETGIDYTFTFHDHYDRSSFQIKRTQVTWRQHD |
| 393 | PFEEQIKEFKKRFIEAFNKGDWKALLDMIHDDARFHFSQENVTFTTKDEFREWFERFISNSDVKIDLKEIHIDGQTATVHFTMHVNHDSRTFTFEVTVHDRYKDGKLVESQFHIDEK |
| 394 | PTEEKIRELVKRFFEAMDKGDMDALKSLLHPGATMHIDEGSTAHDLEEIKEYIARWLSDFRDIRRHLESIEIRGDTVHVHWTATFHHKTGNTYEFRGTTTFHMRGDKIAEIRFHTNQQPT |
| 395 | DQEEIIRQLLRRFFEAINHGDMDTIESLMAPDWTFHYFSGQTFTSKDEFKEWFRRFFESFKDMRIEIKSMEINGDTVTVHVEVHVTHPTGKKITYHVTEHHHFRGNRIVHTQVHERQQET |
| 396 | MSEEEAREAVKEMLRRYYTAWNNNDMDKLEELITPDARFQDARTGREFDLEEWKEMLERLYKTFPGVQIQIDEIRIEIKDGTITVEVHVTMHYQSKKQTTHYTHFWHIELTDGGWRITRVTVQSDSG |
| 397 | PSEEEVREAVKELIRRFIEAINKGDLDALFELYHPDARLHISETGTTYTREEIKEWFKRWFETFPDTQVELKSVEIHVKDGTGTVTIHVEVTYRRDPKTVKFDSHIRIVYTDGTFKIVEEHIHTDSG |
| 398 | ASEQELKEQIKEIKKRFAEALDNGDWDTVRKLFHPDATLHISDGRTFHSLESIVRYIQELASSNLTSRHEIKSMEIEVKSKDHVEVHVHVQVHYDQQPRTVHITFTDVYRYTDGGMKIVETRVHTDH |
| 399 | MSEEEIEREIRETLKRYFEAINKNDVDKLFSLLDEDWTYHHSSGETITTREEFVRLFRQLATRNNDTRIQIDSMEVRIDSPDHVTVHVHVTMTNKYEKRTYHVTETHHFRRTDGGFKIVETRAHFDH |
| 400 | MSEEELEEAARQIVRRYIEAINRNDLDAIASLYHDDARVHYWNGQTFTTREEIKEWFERQIKSGAQVHLQITDMEIRIVSKDRVTVHVHVQIHYEQNPRTYRVTSTFHIVLTDGQWYIIEERIHLDH |
| 401 | ASEKEAEEAAREVIRRYVRAINQKDLDTLKELYAPDATMHHANGRTFTTLEEIIDFFKKQIDNNNDTKVEIKSLEVRVRSKDTITVTVNMDLTNKLNKHTIHVQSHFHMERTQDDFKIKEEHVHIDH |
| 402 | TSEEEAEEAIRRLLREYYRAIDQGDWETLAKLFHDDATYHHANGQTFNSKEEIIRFFKSLFDSGLRTEHQIDSIEVEQKSKDEWTVTVHVTVHIDKESSTYRVTEHHHFRQTDGDFKIVETRVHIDH |
| 403 | PSEKEIEEQVREVIRRYFEAWNNNDFDTLHKLLHDDWTYHHSSGETITNREEFIKRIRELIKNGFQIQLEVKSLEIRVESKDTVTVHVHVTLHFEKHPRTVHVTETHHLRLTDGGWKIVETRAHFDH |
| 404 | MSEEEIKEAAKEVIRRWIEALNKKDLDTLRELYHPGATMHQSNGQTFHDVEEIRKWFEEFMKKFDFQLHLTGIEIEVKDGRTVTVHVHIQFTDSKTGTTVSVTSTFHLEVKNGQLYITEQRNHRHQT |
| 405 | TSEEEIREVLKRFYEAFQEGDVEKLLRLMHPDAQMHVSEGITFNNWEEIKEWLERFFKRFGDMRVEIEEIRISNGTVTVHVTFHFNHKDGTVTIRVTDHYHFKDGKITRIESHHHPQD |
| 406 | PKEEKLIEIAKEIIRRFYEAFNRHDFDEIIELLHPDWTYHHSSGRTFTDPEEVKEWIRRFLSNDNTHIEIKSIEFRTGGDTITVHVHGTLHEHRKDHTQTTHFTETHHFQQSGNDVKLVETRTHWNSDGS |
| 407 | PKEKEMEEAVREFLQREFEALDNKDWEAWAKLLDDSTIHFADSNTTFTSKDEITEFFRQSLEQFDSEHRVKSMHIEIDGDTATVEVHVEVHFKGEGQTFRFTATFHFTFENGELKLKEKQIHTDT |
| 408 | PKEKEMEEKVREIKKQLIEALNKNDWDTFKKLMDDSTMHNPDRGRTFTSPDEIAEWFRRFVETFDVTIEIKSIEIRISGDTVTVHVTVQVHFKGEGQTYTFHFTDHYEVKDGEIKLKSSTIQTQD |
| 409 | PKEEEAREQAKEWLKEFYRAINENDMDALKRLMDDATMHIAQFQITFTTPDEIKKWFEEFIKKFRHRVEVKEIRIEVRGDTVTVQVHVQVHFEGSGQTFRFTDTYHFEVQDGQMKIKELRSQIHS |
| 410 | PSEEDEEDIRQLIERFYKAFNEGDLDALLSLLHDDWTFHEYNGRTLTTREEFKEWVKQWLSNSARIHITEIHIENKDGDTATVEVHVTVHFNTNNKTKTVHTRDTHHFVRINGQWYIVETRVQVDETGD |
| 411 | PSKKQQEDIRDIIRRFAEALNKGDLDALESIYHPHAQMHIYRGITMNSREEIKRWFKEWLSQPFRIHVHSIEIEDKDGDTITAHAEMTVTFTDNGKQKDVTVRRTFHFTLINGRWFLIEDHEHVDNTGT |
| 412 | ASKEQQEKIKEIKQRFFEALDNGDWETLAKLVHDDAQFHFAKGETLTSKEEFKEWVKRFASTPTAHHITEIHIENKHGSEIEVTAHVTFHVTHDGRDLTFHVTTHDRYVEENGELKILETRFNFDDTGD |
| 413 | PSEEAKEAIKELIRRWVEALNNNDRDTLRDLYHDDAQYHDASGQTFDDREEYVQWMTEWFQKFNVKVTILSMEIRKDSEHQYTVHVTFQFHRHTPSSTHTVNVHSTFRVERHGDEWRIVEQHVQRDST |
| 414 | DKREEMEERIRQLKRQLIEAMNNGDADTFISLFHPDATYHHQSGRTFTTKDELKKWFEEFWKKHPTKLENQSMEIQITSEGRATVTVHIQATDDNTGKTVTFTYHDFYQFFGDELYVVRVETQTDE |
| 415 | DERKKAEELIRETKRRFVEAVNNKDVDAIASLFHPDATMHHSSGRTFTTLEEIKDWVEKFLKTFDVHLNVDSIEFQPTTPGRMTVTVHITVTDNDTKTTVHITFTDFYQFDGDTVKIVRTEIYVHE |
| 416 | MSEEEAREAIKEIIRREVEAFNKRDLDAWASLFAPGATIHFANGQTLTDLEEIKEWFKRFLDHFPSRMEVKSLEIRIVDSTTVQVHVTLEVHVHHDNETKTFTVHATDRFELDDTGFKIVERHVHMEEQ |
| 417 | TSEEQIKQWVRRYLEALDRHDIDELAELFSKDWTYHNSRGITFSTLEEFIEWVERWLSQAEDSRHELKSLEIHGDTVTVHVTQHLRHGDKHKTIDITEHHTFKNGRLVRTQTHFDEKD |
| 418 | SSEEKIKEDIKEMLKREFEAIDNHDWEAWKSLFHPDAKQTNSDEKRTYDLEEIVEMIRRWLSTAPDSRHDLHTIHIEIRGDQVHVTVHVHVTVHRNGKQYHQTVTQHVVFRRSNSEWKIISKHVTTRQE |
| 419 | SSEKENKDAVEEIKRQILDAINKGDRDLFKELFHPDATMHISNGRTFNNLEEILEWFKQMTENNFHFELKSFRIEVHGDTVHATFHVDVTERQTNRTFHVQFHDTYVVFNGSWKIVHTQIHYDNS |
| 420 | MSEEEAQEAIRQWKERFVEAINRGDMDALREMVHPDATFHFSDGRTFTNIEEVVEWFKQMFENGIELEIKSIEIHTDGDEYRVTVHITLTERSNNKTVTVTFHDRYRLENGSFKLHESQIHYNKD |
| 421 | TSEEEAKEDIKEWFRRFYEAFNKGDWHKLKSFYHPDATMHLSDGRTFTNRDEIVEWFRRLWSNGVTTHIHSIEVEIRGDQVHVTVHVTLTEHTNNKTVRVTQHVTFDLENGSWKLKRDEIRHDKS |
| 422 | TSEKEIEEAIREFLRRFYEAFNKGDMDALKSFYAPDATFHVSNGRTFHNPDEIKEWFKKLFDQNVEMEVKSVRIEVRGDTATVTVHFHLTNRTNRQTITITQHFHFRFENGELKLKRDEIRIDNS |
| 423 | MSEEEMKEAIKELKRRLVEAMNKNDADKLKEIFHDDATFHGDGRTLKNKEEIAEWFARFISTFRFKIQIDDMHIQFGDGEVTVTVHMTVTFDDTGRNVSVTAHDRYRFTKDDVRIVSTEFRFTN |
| 424 | PSEEDEKQIRELIERFYEALNKGDAELIARLLHDDAQLTVNGRTFNSKEEVKEWVKRFFSHSTHIQVESIEITYDKDHITVTVHVTMHDNQTGKTYHVTDHYFFRKTKDGWKIIRVQSHTQQQD |
| 425 | PSDEEEIREWLKEFYEAFNQGDLDKMAELVSPDATMHVYNGQTFNSLEEIKEYLERVFKTFPTMQIHVRIVRIEIRGDHATVEVEVTAHVTHDGRHTTYHVREEHHFRKTKDGWKLTETHIHRNQD |
| 426 | PSDEEQIREWKKRFIEAFNKKDLDKLRELVHPDATMHVSEGITFTTLEEIVEWLERFFRRFKDVKVSVQIDEIQIHGDHITVHVTVHVTFNHDGEQKHVTVHATDHYRKTKDGWKLVRSQFNWHQQ |
| 427 | SDKEAEEKIREWKKRFIEAFNHKDLDKLRSLLHPDVKYHDTEPQTFDYEELVERIEEFLKKIDVRIEIKSIEIEIHPNGHTYHVTVHITVHMNRGDRTQTYHVTQHDRYELRNGQFYLAETETRTNGG |
| 428 | PKEEKARQQIREFVERLYDLMNKGDADAVASLYTDDATFHFSDTQRTYSKEEFKRMVEKMFRAGVQFHVKSIEIQFDPNDNHVTVHVHVDVTNESTGQTYTFTQTFFFRFEGDELKLTEDRFHLDNS |
| 429 | PSDEEAVREMVRRYVEAMNNNDLDKIKSLYDDDSKYHDASQQRTFDAEELKEWYERFISHFDFRIEIKSIEVRVDDEHTITVHVNLHVTVTHNGETRHHTATYTFFLEKHNGSWKIVEVREHRDDE |
| 430 | DDEEMKEQIKEWLKREMEAWDRGDVDAWAELVSDDAEFHENDTNRTFDKEEVRERIERFVRNNIDAQRQIDSLEIQIHGRTHATVHVHVTIHEQGDNKTRHFTTTDHFEFEDNGVKLKERRTTTYHD |
| 431 | DDKELEDQIREWLRQYFEAFDSGDIDKLRELVTDDATFHNTSTNQTYNKEQFIEMVERFIRSGIDSSHEIHSIEIRINGTEHVTVHVHWTLHRRGGNRTFHGTTTFFFRFEPNGMKITRVQTTTQPD |
| 432 | PDEEMQEAAKQWIERFYDALNKNDLETLYSLFDEDWRYHETDTNQTYDLDQWKEMVERLIKSGIRFRVEIDEIRFETKGHTHLRATVHVTVQTEGGDTRQRFRDTHTFRQEKDGWKVVRTQTTTYHE |
| 433 | PDEEMEEEVRRLVRRYYEAIDSGNLEEVYELLHDDWTYHDASRNQTYDRDQFKDMVRKFLENDLKVQRHLKSIEVRINGTTEVTVHVHVEVHRSRGNTTYNVTDTHHFRRYKDGMKFVETRTHTQPT |
| 434 | PDKEFEEQIREWIQREFEAWDSGDLETWASLYHDDSTYHNTNHQQDYNKEQLKEMLERFFKAGNEAHREIKSIEIQKYGTHTFRATVHVTAQHNGGNQKVNFTYHFFFRFEKDGMKLVEKRETTQPS |
| 435 | PEDEEAIKQFLKRYYEALNRGDMDTIRKLVDDDSTFHDATRNQTYDWEEWLEMLKKQTDNDTKFEIKSIEVETKSSNTITVHSHVQVHTTHNGQPYTFQVTETHHFTLEGDSWKLTETHTHSDSS |
| 436 | PEDEEAIRQIVKRYFEAVDKGDADAVFSLYDEDARWHDSTTGRTFNAEELRERYRKLAERDSRHDIKSMEIRVVDSTTITVTVHVQVTFNQDGQSRTYSVTYHFHITKHGDSWKIVEVHTTRDSS |
| 437 | PKFEEAIKRWLERYYTALNNGDADTVAELLTEDWRYYDSSRNQTYSRDQFKKMVEELLSHDTKHEIKSLEIRVNSKDTATAHVEVQVHRTHNGQQHTFDTHDTHHFEFRGDRIYITHTHFHMDSH |
| 438 | PEMEEQIRQIVKRLYDAMDNGDADAVKSYYSDDSRFTDENHQRTYSAEEFKEMVKQQASNDVRREIKSIEVRINDSRSITVHVTFQLHENHNGQTRTITFHQTLHFVLIGDQWYIKHDHTQTDDS |
| 439 | VDEEDAKEIKEIIERLVEAFNKGDMKTFRDLFHPDWTYYQSDGSTEYDLDQFIEWLERLLSQGVRVEIERIQFDSQDGDTVHVTVHVTYHVTRNGNTRTIHATEHHVFTRFGDEWKIVRTQVHHDERQQ |
| 440 | SPDEDQEQIKEVLRRFYEAWNKGDRDKLRELLDDDWRYHDSNGRQTYNKEEFLEMVEKALSRGFKLEVKRIQIDDQKGNTIHVTVEVHMTWTHNGRRFEATVRDHHTFVKKGDSWKMVRTQSHWDNHET |
| 441 | PEEEDKEKIKEWYKREIEALNGRDLDAWKELVHPDATFHDASRQQTYDVEEVRKMIEELQSHGFRIELKSMEIRFDGDHVTVHAHFTVHFTHGDRHVTTHYTVTDHFQKKDGELKLIERQEHHDTQED |
| 442 | MSEKELEEQIRDFVRRLVEAFNKGDLDALERLYHDDATITFASTGQTYSKEEFKRWVEEWWKSVKGAQVQIDSIEVRVHDDGSVTATVHVTVHQRHGDRTHTFTVTSHFHFRFEGDQLYLIEEHVHIHHQ |
| 443 | SSEKEAEEAARELIRQYYEAVDKGDWEAVKRLFHPDATFHDESRQQTFNLEEFLKMLREWWESVKGVQHDIHSIEIQFHDDGSVTVHVHGTVHVTQGNRTKTYHFTDTYHFRLEGNRWKIVEYRSRRHQA |
| 444 | PSEQEAEEAIREWLRRYYEALDNGDWEALKRMFHPDWTYHNSDTGQTYDLDEFIEMVRQTLRRIQDSRHQIDSIRFEFDDSGQVTVEVHVTVHFNRGDQTHTYHITEEHHFRYEGNDFKIVETHVTTHQS |
| 445 | MSEKEMEEAAREIIRRLAEALNNGDFDTIKELYHPDATITFPSTGSTVDQDQFLRWVREMMERMKGFQIKIDSIEFEFKKDGELTVTVHVTVTVHQGNQTYTFHVTSHIHIRFEGDTVKIVREHITTHQS |
| 446 | NSEEELKEQIKEFIKREMEAIDKGDADAWLSLFDEDVTITQNGNTRPMHAEEFKEMIEQIASNNSNHHVDSIEIQIRGTDEVTVTVHFTVHFTHNGKNVTIHITAHFHFQVRNGDLKVKSKEEHTDNRP |
| 447 | MSEEEMKEAIKQWVREYFEAFQNNDWDKMLKLFTDDWTFTQDGQKQDRTPDEIREWVEEQFKNDAKVEIKSIEIQIEGTTHATVHVHVTFHHTHNGQQVTIDVTETHHFQFENGSVKLVHTRQHRDNKE |
| 448 | MSEEEMKEAAKQVIEQYYEAFQKNDLDKLKSYITDDWTYTNNGQTENRSPEEFKEFVEQQLSQNTTVEIKSIEFRIVGTTHITVHVHVTYHTTKNGETRTFDVTDTHFFQFENGSVKIVHTETRTDNKE |
| 449 | MSEKEIEEQAREIIKRFYEAIDKGDADAVASLYSDDSTFTHSGNTKDVSKDEIKEWVKKFASNNSQHHIKEIHFQVVGKDSVTVTVHVEIRMHHNGQTKTVTVTQHFFFEFHNGSLRIKRDEEHRDEHS |
| 450 | ASEEEARQDAIEILQRYYDALNKGDWEAVLRIVTDDWRYHENNGITLTTREQIRRWVEELFKKFNFQIQIDEIRAQIHDDGSATVTVHVTVTFHDGDKTVTTHTTDHHHFQLRDSRYQLIETHVHRDTKEN |
| 451 | SGEETLREAAKELVRRLFEAFNTGDMDTLRSLYHPGARFDVDEGRTYDKEEYLEMVKKWNSQDWQLQIDSIEIRIIDSTHVTVTVHVTLHNRSTGTTIHITRHITVELRDTQVFVIRDEVHTNS |
| 452 | MGEETLREAVKETIRRLAEAFNKGDLDKLRELYHPDATFHHYSGETYDLEEFLKMVEEWNSNDWHVEIKSIHIQIVDETTVTVTVHFTFTQPKTGRTQHFTSHFHIRFEDSELKITEEHIQEHS |
| 453 | AGEETLEKQARELVQRLVELMNNGDLDALRELFHPDWTYHTNNGETYSLEEFLEMVEELFRQQWRLQVDSLEVRINDENSVTVHVHVTFTQEQTGRTRHVTETHHFRYKDSTLKLTRTHAHRNS |
| 454 | NGEETARRAAEEVVRRFYEAINKGDLDEIRELLHPGWRFHQDQGTTYSIEEFLEMVKELFDQDWRLQIDSIEFRIVDENEITVTVHVTMTNRSTKETRHVTDHHTIRFQSSEWKIVTTHTHTNS |
| 455 | SDSSELQEAVRQVVKRMIEAINKGDRDTLRELYHPDARMHHANGQTFDYEEIIQFMAKLASADIKIDVESIEIKETKSNGEVTVEVHYTVRNKRTKQDVTVTRHFRFQQQPDGSFKLIEDRVQHDDR |
| 456 | PDPSEIEEKIKQWLRRMYEAINKGDIDTLRELFDDDWTYHQQNGQTYDKEEFLKMMEELFRRNVRIEIKSIEIKEFDSRGEATVEVHVQFHNASTGTTVTVTETHRFRFEPNGQIKLVETHQHRDSN |
| 457 | TSDEAEEEIREFDRRRFESFDRGDADAVFELVHPGAQFTDEREQRTYDQEEFKEWVKRVASNNVRHEIKSVEVQQTGENEFTVTAHVTVHNNSTGTTVDVTYTLHYRRQGDRWYLVRVREYWHQR |
| 458 | TSDEIKQAVEEWLKRWFEAFNRGDVDAVASLYHPGATMHLGPTSRDYDYEEIKRFIKEWLDQDWKLEIKRIEIQQVDKDEVTVTVHFQATNKSTGTTVDVTRHLTFRRYGDDWKLVEDRADFRQS |
| 459 | PEEKEAREIVKEIIEREVEAFNKGDMDAWKSLFAPGATFHHSNGTTYDLDEVKKMLEERWRAGVHIEIKSIEIRVKDGETMHVTVHLQMHDRRTGQTFDMTSHIRLERTQDDIKIVEKHSHTDSGS |
| 460 | DDKTAEEIIREVVERYIDAFNKGDWKTALELLDDDWQFHHSSGQTIHTKEEFREWFEKLASDAKIEIKSIRVEKKDSNTYTVEVHVTVTHDSTGQTFTVTEEHHLRREGDDFKIVETRVHVNPN |
| 461 | DEKTQEEQIRQIIREFYDALNKGDADAVMSLFHPDATMHIWNGQTFKTREEIKEWIEKWASNYKIHIKEIRIQFEDHNRITVEVHFTATNDSTGTDIDFTETHHLELHGDDWYIIETRTTSHQQ |
| 462 | DEDTAREAIKETKKQFVEAFNKGDADRLLELLHPDATVHISNGETITTMEEFKEWVERLASYVEIHLKSVEIEQKDHNEFEVHAHMTFTDKRTKQTYHVTATDRYRFSGDSVKIIESRFHFHQT |
| 463 | PSAEEIIRDLVRRLVEAIDNGDWEALSELYAPDARFTEGPKKQPLDREEFRKWVEEFSKHPSTREIKEIRIQQHGTHTYTVHVHVQLHDQSTGRTFDGTSQLFIVRKDGSWKFKREQDDFTNS |
| 464 | PSMEEAVRDWLRRYFEALNNNDLDALEELFDDDWRFHLAETQKTFDKEEFKEWARTLMSFKSQVQIDEIRIQIYGETEATVEVHVTITRDDTNQTTKFTEEHHFRRKDGSYKLVETHVQRDQA |
| 465 | DNSEIEDWIRKYFAAINHNDLDAIASLYTDDSTYYDAQPRTFHDKEEIKRWYEEFMKRFKDVRFEIKEIRVHGQTVTVHVEVHATHGDRDYTFKVTSHFVFKDGKLKEVRDHMQSTD |
| 466 | PASSEEIKDWIKKYYEAYNNKDVDALVELVDDNAQIHDQHGETLTDKEEFREWVEKWLKEVKDVKFQIDSIDTTDSTVEVHSTVHTTQDGKTVTIHVTETHHFRNGKLTSTHIHSDPDDG |
| 467 | PSQEEIKELDERRIEAFLKQQDADTLAKLMTPDSTFHTYNGETLNDQEEIKKWFKRYFEENFDEMHFQIDHMHIEPNHITVHVTVTFTRNGQTYHMRVTYTLTYRDNRLAHMRIHVDTKHS |
| 468 | PSEEEIREIVKRLMEAFDKGDADAVMSYYHPDATMHVSDGVTFTDLEEIKRWIEQWFKSFDDVKRQIESIEIRGDTVHVHVTVHATQSNGKTITFHVTSTFHFNKDKRIVEERIHIDQRE |
| 469 | MSEEEIRQFLRRFYEAFDKGDVDTFASLFHPGVTIHVWQGITFTSREELREWVERFLRNFKDMQREILSLEVRGDTVEVHVQVHTTHNGQKYTFDVTHHWHFRGHRVTEIRVHVNPT |
| 470 | DSMEEIAKKVVERFAEAFNRKDLDAFAEIFHPGATVHFWEGVTITDQEELKKWFEEFMKRFKDVKVQIEHVDVDQDHVTVHWTMTVHHDDRTIQFHVTTHFRFKDGKLTEVRFHMDWQEHD |
| 471 | TSKEEAKEILKRMYEEFAKERDYDKWVAYFHPDATMHIYQGTTFTTQDEIRKFLKEFLERFKDMKIQIERIEVENGRVTVHYTAHFTHGDETRTYRFTDHFVFKDGKITEERSQIDSL |
| 472 | PSKEEIEEFLRRMFEYWAKTGDVDAYMSLFHPDATFHISDGETFTTPEEVKRWFREFLKHAKDVKIQIERIEIENGRATVHWTMHVTHGDQTHTVRVTTHFQFKDGRITEVHIHRDDT |
| 473 | PSKEEIKEFLKRWFEYFNKTGDADAAASLLHDDVTVHLSNGTTFTDKEEIKEWLRRWLKSAKDVHIQIERIEVQDGRATVHWTVHTTHGDQKYTFRATTHFVFKDGKIVEVRHHYDST |
| 474 | PSKEELIQLVQRWYEEFFKTGDVDKASSFFHDDATIHNASGITFTTKEEFKKFMEEWFKKAEDVKIQIERIEVEGGTVTIHYTVTVTHGDETKHTRFTDHFRFENGKLTEVHSHYDDT |
| 475 | DDREDIERLVRRFIEALNKKDLDTLADLFHDDATMHLYNGITFTDKEEIKQWFEEWMKRFKQVHIELKSMEFRGHTVHVEWTIHATHGDKDITFHAHTTFHVRGKKIIEMRIHVDTDNN |
| 476 | PGEQAIKEWVERWVEAYNKRDADALASLYTDDVTFVHGDQPYQLSREQYKEWVERFLSHFKDSKLDLKSIHIDGDTVTVHMEMHVNTNSQTYTFEITVHLRFKDGKLKEQRDNTDSQS |
| 477 | MSEEQAKELVKRFMEAVDTGDLDALKELFHDDATLHLSDGITFTTKEEIIEWFERWLSQLQDVKRQIESIHVTDDTVTVHWTMHVTHGDQDFTFHGTTHIRFKDGKIVEMREHVDQDDGG |
| 478 | PSWEEIKEWVRRMVEEVFNTGDWEKAKSLLHPDWQFHHSSGTTFHNREEFVEWFQRFLKRFPDTRVDVSHIEITSEDVTVTFHFTFHARHGDQKFTYRVTEHHKFRDGKLVETRVHWHQT |
| 479 | PEEEIREWKRRLFEALDHKDWEKLSELFKSSKDIKMTTQNGRTFDAEEIIEWFRRFVESFADMRHEIKQIDFEGDTVTFHVQVHFTRPDGSTKTIDFTFHDRYDKNGKIVSTNITWDEKD |
| 480 | PEEQIREFVRRLIEAFNKHDIDKLKEIYDSSKDVKFHQDSGETYDKDEFLKQVKEILERWASVHIEIKRIEFDGDTVTVHVQVHLTRPDGQTHTFDVTSHFHFDKDGRVTSEEIRTRKVD |
| 481 | PEEEIKEFFRRLLEALDKKDWEKLKEIYSESKDARITEDSGQTFDKDEFVKMLREFLERWAQTAHQIDRIEFHGDTVTFHVTVHLTHQNGQNVTTHVTSHLHFDKDGRLTHEQIHRDKRD |
| 482 | PMSEEEIKQFVKRAYDALNNHDWETFASLFHPDARVEFNDGTTLTTLEEIKEFIKQLLEKLGRMHIKIDEVHIEKHGDTITVTVHVTVHVHTPDKEITFRATHHWHFTRGRLISLQEHMNQDNK |
| 483 | PDEEEVRRWLREYYDAWNRNDLDALASLLSPDWTLHNGQQSREDFIKWVEEFLKRFKRMRVQIDEIRIERHGDEVHVTVHVTIHFTSSNSTDEVHTTDHHTFRNGKIVETRTHTDQRS |
| 484 | PAEEEIRQWIRRYVEAWNNNDWETIVSLYAPDATFHFGGMSREEFERWVKEWLSRAKDIKIQIDSLEIRQHGDEYTVHVHMTMHITHDNKKTTFRFTSTFHFRNGRLKREHFHSSTES |
| 485 | PDEERAKDWVRRFYEAFNQNDLDAILSLYSPDATFHMNGMSYEDFKRWVEEFLKKFKDVHVQIESIEIRVSGDTVTVTVHVTVHVTLTDGEVTFKATQHFHFKDGKLKEDEMHWRTES |
| 486 | PAKEEVKEWLQRFYEAWDNNDADKLKSLVHPDATFHDGGYDYEQWKEMVEEWLSRVKQSSHHIKEVHIEIHGDEITVTVHVEVHLTHDDRTVTVDVTDHYRFRNGRLVEVRSHRRTHS |
| 487 | MSEEEIREWVKRFFEAINKGDMDKLRELLDDDWRYHDTDRQQTYDRDEFLKMVEEFLKKFPGAQFEIKSIEIRGDTVHVHVQVHMTHDDGSTQTIDITETHHFDSDGKLQRTEVTSDPKD |
| 488 | MSEEEIREIDKQRIEAINNGDMDKVKEIFHPDATFHFNDTQTTYNLEEFIEWVERFLRRFKDVQIEIKSMEVRGDTVTAHSEVHVTHPDGSTQTIRVTYTIHYDKDGKIKRMEINTDPKD |
| 489 | PSREEIEQLDTERIEAFNKGDAEKLKELMHDDAQYHNNDEQQTYDKEEFIEWFEQFMKKFKGVRIQIEELHVHGNRVTAEGTVTFTHDDGSQTHIRFRYTVEYDHNGKIKRVEESTDPSD |
| 490 | ASEEQIEDILRKMYEALNRNDLDAFLSFFHPDATFHVYQGITFTDLEEIKRFFKEFLERMKDVHIEVTRIEFHGDRVTVHYTVHVNHPGETKTYHATDHFQFKNGKLTEVRSTIRQED |
| 491 | TSEEEIREWVKRWAEAFNNNDLDKLVSLFHPDAQIHFYNGQTANSLEEFKKYLERFFRHAKDVRFEIKNIEVHGDTVTVHWTITVNYPGKTVHFHGTTHFRFRNGKLTEVHFHVNQED |
| 492 | PSEEEIKEFLKQLYETIDHRDADAFVSLFHPDATLHISEGITFTTLEEIKRWFEQWLDRVTDVRHDIKRIEIHGDTVTSHVQTHVTHPGQQTTINVTEHHRFENGKLVETHTHWNQER |
| 493 | TSEEEIREIIKTFFEAVDRGDWKKIKDLYAPDATLHHSSGRTFHSLEEIVRWFQQVFSQLKNSSHQITGIEIRGDTATVHVEVHVNNDGRTITYRFTSHFHFSDGKIKEEHVHVHQEG |
| 494 | PSEEEMERAVREVMKRYYRAINEGDLEALKELFHDDARFQSSPMNKEQFIEWVKKANEAGTQLHVHDMEVQIDGDEVTVHVNVTMTVTRGDETRHTTFHETHHLRFTDDGLKIISTRVHFQTHQ |
| 495 | PSEEEQKREIKEFYKREAEAFNKGDLDAWDSLFDDDVKNSSNPADKEEIKEWIKKAFDNNTQMHITHMEIEVNGDTVTVHITVTVHTHRGDRTTTTDYHTTDELRIDSDGWKFVDRQTHFQTHH |
| 496 | MSEEEIKRAVEEFVRREIEAINRGDLEAWKELVDDDARVESEPANKEQMAKRIEELFRAGTQIELKSIEIHVDGDTVTVHVHIQFHTHHGDRTTTVDVTATDVIEIDHDGFRIVSRRFQRHQHT |
| 497 | SDSEDEEAAKEWVRRWAEAFQKGDLDTLSSLYHPGATIHHSDGETYDRDEYLDWLGKYHKKFDTSVTIHEIRVEINGDEIRVHVNMTLTHHHGDQTYHFRVTYTFRFEKKKDGLYLVEQHTHTDDT |
| 498 | DSSEAQEEIKQWFRREFEAMDNKDRDTWASLFHPGSTVHHSNGQTYDREELIERFGSFQSRFDAKHEVKSIEIQQHGDHITVTVHVTVHLTHGDKDKTIDVTYHDRFRKTKDGWKLVERHVHTDEK |
| 499 | SSEEEQKEEIKKWKERFIEAFNKGDVDTIFELVHPDATFYDPNGQSYNIEEFRDMIEKIVSQGYQIRLVSLEIQIVPNPGEVTVHVTVQIHQPGANKTYTAHFTDRYRQKDGQFYLVESHYRRDQEE |
| 500 | SSEEELREKAKQLVERFYEAFNRGDMEAMKRLIHDDARIHHDNGETYDKDQMIDMLKKILDNDVRLEIKSIEIQVSSVPGQVTVTVHVQVHFDKEGKTYKVTHHWVFQVKDGDMKIVNLHEQTHDTS |
| 501 | PSEEELKEQVKEVLERYYEAINRGDMDTVASLFHDDATFRHSNGEQINKEQFVKWLKELFENRTRIQVDEVHIQIDEEPSTVQAQVHVTMHTDGSGKTFHVDESHRLRNQDGEVKFVSTRGHTRDSS |
| 502 | MSEKEQEEAIREVIRRFYEAINRGDMDALEELLHDDWRYHHPSGQTFSKEEFKDMVRKLVENGAKFHIDSLEIQIDPSPGDVTVTVHVTVHHDGKNETYRATDHHHLRLSNGDWKIVETQVRTDNTT |
| 503 | PVSDKEAEEDIREIVRRYVEALNKGDIEAIASLYHDGARIEFTAEQRTVDKEEFKRWVEEWKRQFKDATIQIDSLEVETKDDGTYTVTVNMTITDPSTGHTVHVQSHMHFKAKTKQLYIASEQIHFQSS |
| 504 | MSDDELKEAVKDWLRRFYEALNHGDLDTLKSLFAPDWTFYNVNNPQTYDLDEFIEMVKELLEKFDYRINIEEIRIEIQGRNTVTVHVTFTVQFHHGDETQHYQFHETHHFRFEGDELKLKETRTQTDPT |
| 505 | MSDKELKEAVEEWIRREAEIFNKGDLDAWASLFHPDAQYHDSSDPKTYDYEQMREMFERLQRRFDARMQITEIRIQIFGRNEVRVTVRMEVTFNHGDRQEHYTYHYHFVFRFDGDELQLVEKHTTTQPS |
| 506 | MSDEELKRAVKDWIRRFVEAFNNKDLDALDELLDDDWTYYDSSRKTQFSKEEYRKMVKELMDKFDFRIQIHEIRIQIFGRTTVTVHVHMTVHLTHGDETHTFHVTETHHFTFEGDELKISETRTTTQPS |
| 507 | ASEEEQREAIKEFKTRFIQAVNNGDLDTVRSLVHDDATYHHKGGQSYDKEELLKMIKEMFDNNIQIDVDKIDIQSNGTNSATVTVHVTLHNRTTGQTYETEQHDHYEFRGKRVFLTETHVNTDQT |
| 508 | PSEQEFKEAVKEWKERFIEAVNKGDMDALRELFHDDATMHHSGGQTLDKEEIIQWFAEAFKNQTKWDLKEVHIQIDSSDTVTATFHVETTNQRTGRSFDNTQHDHYRFEGDTVKLVETHTHTHES |
| 509 | PWRKELEEQVREFVRRYIEALNKGDLDALARHFDDDWRFHDARTGKQYSRDEFKKMVEEMFKANVRVQLDSVEIRVNDDGTVTVTVHVTYQDSRTGRTFKVTEHHHFRREDGTMKVVRTQVHFQELGS |
| 510 | PEEKEIEEKIREWVQREIEAWNKGDLETWARLLHDDVRYQNNDTGQEYNKEQMKKMFKEFFDNNIEVQLDEMHVQINPDGSVTVEVHVTFHDRRTGRTFKVTYHLFFRFQDGQLYIVRKRATRREVGS |
| 511 | DRREEVERKIREWIKREAEALNTGDVDAWKSLFTDDFQYTNSDTGRTFDLEEVREMVKKLFDAGVRVQVDSMEIRFKPDGTIEVHVTMTFQDSRTGRTRQAHYTDHVRFENGELKVTSRRATTQPLGT |
| 512 | PEEKEIEEKIRDWLRRWAEAFNNGDLEKLASLYDDDARYHDSRTGRTFDKDQIKKMLEELLRKNVQIEIKEVHIQINPDGTATVEVHATVHNNSTGNTVDVTYTFHFRFEDGELKLKSQQARFHNRGK |
| 513 | PDEEEAKEQLKEWYRREAEAFNKGDVDAWASLFHDDATFRDSRNNQDYDKEEVRRMFEKMSSQGWRVQMDSIEVRIIPNGEVEVTVHFTFTDQRTGRTFKVTAHDRVRKENGTFKFLERQVTTQPTGS |
| 514 | PSEEEIREWVKEFLRKMYEAINRGDVDAVASFYHPDATFEHSNGETYDYEQFREMIKRLFEQNTRVEIDSLEIQIDSSRSVTATVHVTMHTNRGDKTQTFHVTQHYHFEYTNGEWKLKRDEVHTEQRTG |
| 515 | TSEDEQKKKAKEWLERFYEAINRGDLDALRELLDDDWRYHDASRGQTYDREEFLDMVKKWNDNNWQLDIRSIEFQVQPKDHITVTVHVQFTNRTNDRTIDVREHHHFRFKDGSIKLTETHSHFDSG |
| 516 | MDEEELKQIIKEVIERYYDSFNKGDMDKLRELIHPDAQFTNADTGRTFDKEEFLDWAKKATDNNIKIEIHSIRIEVKDKDHVTAEVHVTFKNDKSPQQFDVTNQDFFRFKDGSVKIVEHRTNSDSK |
| 517 | PSEEEIERQVRDWLKKFYEALNKDWREVKKLVDPGVTFHFNNTTFTTQDEIAEWVRQFISNATVQVDSIEIHIHDDGSATAHVTVTVTINGQTFTVHETHHFQVDHNGEIRLVETRTHFDE |
| 518 | PGEEAIREWVRRYIEAMNRRDVDALASLYHEDATMHVSSGQTFHNIEEIREWYQRFFETFKDVKIEVKSIEIRGDTVTVHMDVTVHRGDQQYHFKVTVHFHFKDGKLKEVQINRDPT |
| 519 | PDKEEIKEILRQFYEAIDTGDADKIRELVHEHATMHIYQGRTFTNREEIVEWFERWTSRTRSHHQVDEIHIDDTSKTVEVHVTVTHQETGTTFTFTDEYHFKDGKIVEMRSHEDSN |
| 520 | DDREEIKEFLRRFAEAFNKGDLDTMAELVHPDATVHFSSGETLTTRDEIRKWLEEFFKKNDSKVHLKEIHIDSSSQTVHVHWEVTHDSTQQTFTVTTTFHFQDGRLVEVRFHHDST |
| 521 | PSEEEVRELIRRYYEAFDKGDADALEELLTPDFQYHDSRNPTTFDLEEFKEMVERFFKTFRDMSREIKEIRVDGGHATVHYTVHVTDYRTGRTVTINATDHMRFKDGRIVSIRSTFDDQD |
| 522 | ASEEEAEEFVRKFIQMAFRTGDADAMASMLAPGWTFHDSSGVTYTTREEFKKWFEDMLKRTDEIKVQIDEVRVSDNTITVHVTIHFHHKDDDKTIRFTETHHFRNGQLKETRLHVDNEG |
| 523 | PSDEEIIEEAVRRFYEAFNQRDVDALAEILHPGVTFHSAEGVTLKTKDSIKEWMTQFLQRFKDIKVQIDEIRIHGDTVTVHVTVHVTHDGNTHTYEFTHHWVFKDGRIVEVHEQMRDDR |
| 524 | ASEEQIREWVKRFYELWAKRNDADTAADLMDDDSTLHVWTGETFTDKEEIKEFAKRWLDTFSSFRVEIKSIEIRGNTVTVHVTIHVTNKDTGTTVTIRYTEHHTFENGRLKETRIHADNQQ |
| 525 | DDKREIKEWKERYLQRFFNTRDWRVIRELVHPGVTMHVESQNTTMTDVEEIVRWWKEWLERFPDSKVQVDSIEFDGDTVTVHVTIHTNHKDKTITFTVTYHDKYKDGRLVETHITIRQHR |
| 526 | DKREKLRQIAEEFIKTFTEAVNKNDLDTAVEMYHPDAKYTNASEQRTYDREQFREWFKEYVERVGRMQVQIKHIHVDGDTVTVHVEVHYHHNGKTVTTRVTRHFRIKGDRVTRDEVHTDNQQ |
| 527 | DKREKLKELIREFFERYVEALNKGDWEKVASLYAPDATYWDARRQQQYSREQFIEWVKEFAKRTGDIEVHIHEIRVHGDTVTVHFTVHYHHNGKTVTTRVTRHFTFKGDKIAEDRQHTDEQQ |
| 528 | DKREKLKRIIEEFFERYVKAFNSGDVDAIASMYHPDARFWYSKTQTTYDLEQFREIIERLIKRWGRIQVKIDEIRVHGDTVTVHFTQTWHSNGKTVTARVTRHFTFRGDKIVEDRQHVDDQQ |
| 529 | DKREKLKQLMEEFLERYVRAFNEGDADTVKSMYHPDARFTHSKSQQTYDLEQFAKMFEELARRTGRLQVHITRIEVSGDEVTVHYTVHYHTTSRTTTVNVTSHFRFRDDRITEERVHEDDQQ |
| 530 | PQEEILKELIRRFIETYVEALNKGDVDKLMSLFSPDWKYHLDKTKETFDREEFREWFQRLSSQVRRMQVQIHEIRFDGNTVTVHVTVHIHHDGETVTSRQTEHHTFEGNKIVETHVHADDQR |
| 531 | TSEDQKKAKDILRRFFEAWDKKDLDKLASLFHPDWTNHVSYEQITFTTLEEFKEWVKKWFDRGFDKSRHQIDEIRISSHTVTVHVTIHQTRNGQEFTYHVTEHHRFKDDRITETHQHMNQED |
| 532 | SSEDQQKIKEIKKRMLEAMDNKDADTFASLFHPDAQMHIEQYNITFHNREEIKEFFEQLIKNGFDRVHHQIDEVHISTHTVTWHITAHVTRNGETYTYHFTAHDRYEGDKLTETHYHSRQQD |
| 533 | PEEEHIRTLLERFYRALDEGDLDAIRELFDDDAEFHNKSTGQTYDLEEFLRMIEERLKRNKDSRHEIKEVRITGNHATVHVTVTFHHDGRTHTYHVTHHWHFRDGKIARLQEQSHDAG |
| 534 | PDEEEIKEILERYYEALDKGDWKAIEDLIHDDARYHNASTGQTYDREEFLDMIRKWLSTFQDSRHELKEIRFTGKHVTVHVQVHVNTPGSTQTYDVTHHWRFKDGKIVRIEEHRRDTG |
| 535 | PEEEEIKEFKKRFIEAFDKGDWEAIEELVDDDVTFHFASTGRTYDRDEFIEWIRQWLRHFKDVKRQIHEIRITGGHATFHVTVTVHHDGSTRTYRVTVHDRYRDGKLTETRFHVDDAG |
| 536 | ASEEEIRQALRRFYEAIDKGDLETLAQLFHPGAQIHFSDGVTLHDQEEFKEWMTKFLSQVSDIKHEIEEIRIDGQTVTAHVTVHVNQGDKTYTMKVTEHHHFKDGKITETHFHFDRKNT |
| 537 | TSEEEIREVLKEFYRAIDQGDWETLRSLLHPGARIENSNGIEFTDQEEFIRWVREFFRQVTNVHRQIKRIEIDGDTVTVHVEVHVTQGDRSYTQTVTEEHHFKDGKIVETRIHVDTKQN |
| 538 | PSEEEIKEVLRRYYEAFDNGDWETVRRLFHPGATIHISQGVTITDLDELRRFFEELFKKLTDVKHDIKRIEIHGDTVTVHVTVHVNQGDQTFTIDVQEHHRFKDGKITETRTHIDTKRN |
| 539 | PSEEEIKRIKKRFIEAFNTKDLDELKRLFHPDATMHIWNGQTFTTLDEIKDWFRKFMESVKRFHMQLDRVEIDGETATFHVTVHVHTKDKTVTIRVTVHDRYENGKLIESQFHEDQKDT |
| 540 | SSEEEIRQWVRRYVEAFNNGDLDALKSIFHPDWTYHDASGQTFTTRDEFKKWVKELMEKVKSYRLEIDSMEIRGNTVTVHGTLHVNHDGKTVTIHVTEHHHFRNGKLTETRVHRNTQPT |
| 541 | PSEEEIKRFLKRLYEAFDSGDADALFNLFHDDWTFHSSDGRTFNDREEFREWVERFFKRFGDTAHQIDEIRIDGDTVTVHVTAHFRHPGKTITVHVTDTHRFRNGKLTETRVHVDQQET |
| 542 | PSKEEIKELKERLLEALDKGDMEKLKELVHDDATFHFDNQTFTSKEEFLEWVERFFKSIKDTHREIKEIRIDQDTVTVHVTQHTTHGDKTITVHVTTHDTYTNGKLTESRIHRDTQPT |
| 543 | ASQEEIEEWLRRFYEALNRKDLDAVRELIDEDSTFHISDGSTLTTADEVKEWVKQMVERFNRFRIEVKSIEIRGDTVHVHYQVHFDSNSQTITVDATDTFHFKDGKLTEVHSHWDRKE |
| 544 | MSEEEIKEWLKRFYEAFQRKDLDALRELIDDDSQFHWSNGETIHSADEFKEWVKKFAEDFPDFRVEIKRIEINGDTVTVHVQFHFTNNSQQQTFDVTDHYHFKDGRLVQVHSHINQKD |
| 545 | TSEEQAKELKERLVEAFNKNDMEKLDELVHPGAQFHHNGQQYDKEEYKDWVRRLLERFDVQIEIKSIEIRGNTVTVHLTAHFHTNGKTVTFHFTVHDRYKDNRLTRSEEHTNNAND |
| 546 | MSEEEIKEFKKRLLDAFDNGDMETLDRMFHPGAQFYHNGQQFSKEEYKEMVERMLSRWKFDHQIDSIEINGDTVTVHVTVHRTTNGKTVTVHFTAHDRYTDGQLTESRFHEDNRND |
| 547 | MSEEEIREWLKRYYEAWNNKDLDKIKSLLAPDWTYHHSSGRTFHDPEEFAKWVQELMETFKEFRIDIKEIRIDRSHSRVTVHIEVHWTQDGKHQTTTTTDHHQFDHDGQLKETRVHWHQA |
| 548 | MSEEEIREIDKRRIEAFNHGDLEKLERIVHPDAQFHFSDGETIHNLEEFKEWVERVLRRFPDTKIEVKEIHIDSSTNRVEVHMTVHHTHNGKTTTVDVTYTVRYDHDGKIVEVRETRNQR |
| 549 | MSQEEIKELKKRWIEAWNKKDRDKLRDLYHPDAQFHYSNGRTFHNLEEYLKWFSEFLKKFPRTHLELKSIHIDDSTNRVTVHIQMHLHHKDETRTVDVTQHDRYAPDGKIIESRLHFNPK |
| 550 | PSEEEIREWIKRYYEALDRKDLEALQRLITDDVTFHYSNGTQINTKEEFTEWVERWMKTWAEVHHEIKEIRIEGDTATVHYTVHLRHKNGQTYTIDATDHFHFDKNGKVKRVQSHSNQRD |
| 551 | PSEEEIKEWLERFYDALDRKDREALDRLFHDDVTMHVDEGTTYTTKDEIKEFLERWLETWDSTAHEIKRIEIEGDTVTVHYTTHLHHKSGQTFTQDATDHFEFDKDGRIVRVESHTDQKD |
| 552 | PSEEEIKEIKKRLAEAFNRGDVDTVASLFHDNVTMHTYNGQTFHSKEGIRKWFERFIREFPEIHMQIERIEFNGDEVTFHFTVHATHPDGKTVTIHITVHDRYDKDGKIVESEFRVHQQD |
| 553 | MSDEEIKEWKVKAEFEAVNRNDADAFAELFHDDSTVHFDEVKITFTNKEEIRDWYKKWAERVGQVHLQVDEVRVEDSDTTVRVHMTIHLTHGDKTQTIHVTQTDRYQDNFLKEQHITVRST |
| 554 | PSEEEVKEFVVRAFLEVFNTNDMDKLKSYFHDDATVHFESEQRTLTTKDEIKEWFKQWFERVGDFKVQIDSVRVTDSIITVHVTVTIHFNHGDQTRTVTIHFTDRYVGNKLVERRVRVDQQ |
| 555 | PDEEKAKELLKQFYEAFDKGDWEKLVSLVHPDAQFHFNDGSTIHDQEEFRKWYEEWKSRFPEVHRQIHHMRIEDDDATVHVTVTLTFTHNGQEYHVEVHETHRFRGDKIVETHVHENEQK |
| 556 | SGREATKEWKKRFVEALNQGDLDAIMRLFHEDATLHVSKGETYTDKSEIREWFERWLKSLKDVKIQIDSIEIRGNTVTVHMTMHVNRGDQTYTFHITTHDHYKDGKLTETHFHEDSRD |
| 557 | TSEEQIRETLKRFYDAFNRKDLDALREIVAPGVTFHFSKGITVTDREEFLDWVKKFLEKVKQMHIQIHSIEIRQNEDDTITVTVHVTVTLHHDNKTITMTVTDHYRFKDGKLVEVHSNIDQEDS |
| 558 | ASEEEIKEFVRRFVEGFNNRDADKIASLYTDGATLHVSEGRTFNSRDEIRKWAKEFLDKVKQVHIEIKSIEIQQDEDKQVHVHVHIEVHLHHDNKTTTVHITSTMTFRDGQLVEEHIHVDSEDR |
| 559 | TSEEEIKEFIRQFLEAFNKKDLDLFASLFDDGAQLHISNGVTFNSQEEIKKFLKELLDAYEELHMHLTSVEIRQNEDETVTVTVHWEVHVRQNNKDITMTITTHFTFKDGKLKRVKIDIDQEPK |
| 560 | PSEEEIREFVKRFLEALNKRDLDKVRELFTPGVTLHINNGVTYTTQDEITKFFKEFLDSFKDIKIQVTEIRIEQKDDSTVTVTVHWEVHAHHDNKTVTLHMTTHFHFKDGKLVEVRIHIDSEDT |
| 561 | PSEEEIKRLIKRFAEAFDNGDMEKLASFYDDDATMHVSSGYTATTREEIKEWFERFFKNFPNTRHEIKSIEIRGDTVTVHVTVTVHHPGKTITIHVTRHITFDKDKKIQRDQIHLDERD |
| 562 | PSEEELKEFLEKFYEALDKGDLDTLRELLHPDVTFHFRDGETITTMEEFEEWFKKLQSRFKDSHHEIESIEFSDGTVTVHYTAHFHHKDETVTVHATDHYEFDHDGKVKRVKSDYDERR |
| 563 | PSEEEIREFKKRLVELFDNRDLEALAQLYHDDATFHYADGVTVTTKEEFKEWVKRVLDRFKNARHKIDRIEVSDGTATFHITAHFTHDGETVTIHATQHDHYRKDGRLTRSEDNSEERR |
| 564 | PSQEEIREILRQMYEAFNNNDLDAIASYFHPDATYHIWNGRTITTLEEFKEWLEEVMKKFKDLRIEIDSIHVSDGTATVHVTFHVHHDNETVTIHATETHHFDHNGKIVETHNNADEHR |
| 565 | PSEEEIKQWKRRFLEALDRNDRDELKSLFHPDATMHLANGQTFTNLEEILKWLKERMEKFQNTAHQIEEIRVSDGTATVHVTVHVHHKDKTITFHFTQHDRYDKDGKLVETHDNTNQHD |
| 566 | PSEEEIREMLKRFYEALNHNDLDTIEELLHPDATFHHASGTTFTTLEEIKEWVTRMLRRFKDLRIQIHEIRVSDGTVTVHVTIHVHHDNKTVTIHATHHWHLDKDGKVTEVHDNSDQRD |
| 567 | PSEEEIKEWIRRYMEALDRNDVDAVASLYDDDATMHIYNGQTFTTKEEIREFYERWMKSVRDTAHEVKKIDVSDGTVTVHVEVHLHHDGETVTIHATVHLRFDKDGKLKELRDNAESRR |
| 568 | ASEEQIKEWLRQWYEAFNTGNWEEAIKRLVDKDWTYHYNNQTFTDLDEVLKWFERMFKSGAHDIKRQVDEIRIDGDTVTSHVTQHVTSNGEHYTYHITEHHRFEGDKITETRVHEHST |
| 569 | MSEEQIKEILRKFYEAMDTGNVEDAIREVFHDDAQMHVNNQTFTSKEEIVEFFKRQWSSGNDEVHREIHSIEIRGDTVTVHVTVHFHSKGETQTYHVTHHWVFKDDRVTEVRQHTNST |
| 570 | TSEEQIKRLVQKFLEAFDTGNVEDAIREIYHPDAQLHVDNQTMHNLEEIVEFFKRLFASGQKEVHRQVDRIEIDGDTVHVHVTVHFHHDGETQTYHQTSTFHFKDDKLTEEHIHTNST |
| 571 | AHEEEIREWIKRWVEAFNKGDADTLAELYDEDSTLHDSNGQTFTNKDEIKKWAEEFFKRADDTKVEIKSIHIDGDTVTVHVTVHVNHNGQKYTFHFTSHLRFKDGKLKEQHIRTYQT |
| 572 | THEEEIRQWVRRYAEAFNKGDMDTLKSLYSDDSTYHNSNGITATTKEEIVKFIKEWLEKVGDSRFQIDSIEIRGDTVTVHGTVTAHTNGQQYHQTITRHFEFKDGQLKRDEARIHQQ |
| 573 | TSEEQLKELLRRLFEAFDKGDWETIAKLLHPGATMHIWDGYTATDLEEIIEWVRRWFKSVKRVHREIKEIRIDGDTVTVHWQMHVHHDNRTITRDGTTHFHFENGKIKEVRFHVDQRD |
| 574 | PDEEKIRQWVQRFFEEVDKTGDWRAFASYYHPDATVHYSEGKTFTTLEELARWLATWVRNVPDFRVEIEEIRIDGKTVTVHVTIHVHHKDKTTTVRQTSHFHFRGDKLTREHVHRNNSD |
| 575 | PSKEEIEEFLRRFYEEIFKTKDPDAFMSYFHDDATYHYNGRTFKTKEEIKDWFEKYMSSFERLHVQVDEIRIQDGHATVHVTVTAHQGHETIHARYTHHWEFRGNRVASLREHSDQQD |
| 576 | MSQEEIKEFLRQLYERLWNTKNADVFLEYVHDDATYHNNGQTYKDKEEIKEFFQRWFAHFPELHVQIEEIRIENGRATVHVTIHATQGDEDVTFHQTHHWVFKDKRITEVRVHTDTQR |
| 577 | SSQEEIEEILRRFFEEVWNTNDREAVKSYFHPGATYTDARTNTTYPADAEELAEWFSEMFERIKDLHVQIKEIRVSDNTVTIHWTVHTNHGDQTHTFHATTTFHFKDGKIVEVRTNMNQTD |
| 578 | ASEEEIRELIKRFFELFDRGDLDALASLMHPDWQFHFGDETAHNLEEFKDWVREWLSTYGESHHDIKRIEIDGNTVTVHVQVHFHSPGGKTITVDVTEHHHFVKDGKITETRVHYDQKE |
| 579 | PSEEQIRELVRRMIEAFNKRDLDALFELYHPDATFHYNNRTFNSREEIKEWFKRFFEKFGEVHVQIEEIRIHGKTVTVHVTVTVHHPGGKTVHIRVTSHFHVHDDGKITEERVHSDQRE |
| 580 | PSEEEIREFLRRFYEAIDKGDLDTLKELFHPDARMHINNRTFDSLDEIKEWFERMLSRFKDTAHQIDRIEIDGNTVTVHVTVHFTHDGGKHVTVHVTHHWHFVKDGKVTRLQVHENQQD |
| 581 | PSEEEIRQIKRRFIEALNKGDLDALEELFHPDARFEWGDQPFDSKDEIKEWFKRWMESVGRYEIRITHMEVEGKTATVHVQVHVHHPGGKTTTIDFRAHDHYHDNGKITETRITWDTKE |
| 582 | VSEEEVKEWVKRFFEAFQKRDLDTLAELLTDEWQYHHYSGNRTETLTTKDEMREWFKRFLSSFDVKIDEIEIHIHDDGTVTVHVHITFRSGDKQFTMTVTETHHFKDGQIKETHQHVNIQDH |
| 583 | ASEEEIKEWVRRFIEAFEKRDFETLKELLTEDWTMHSQSGNTSETATTLEEFEKWVREFLSKIDLQFDELHIHVHDDGTVTVHMTVTVTSGDQQFTVHVTEHHHFRNGKITETHITFNYQPH |
| 584 | ASEEEIKEILRQFYEAFQKGDLDKLLELMDKDATLHFHQGNETYTLTTKDEFREWFREFKEKFDIQFDELEIRVESNGTVTVHVKVTVRSGDKTYHMDVTETHHFKDGKITETRTHIRVQPT |
| 585 | PSEEEIREWKKRFFEALQKGDLDALLEIVDPDVQFHFYEGNRTYTATTKDEFKEFMRQAFEKFRFQIDEIEIHIDDSGHVTVHVTITVDSGDQKFTVHVTQHDRYENGKLTESREHRNWQPN |
| 586 | PSEEEIREWKRRFIQEFWNTRDYHAVERFFHDDAEMHVYRGETFTTKEEIVRWFRQVLSQFPEAHVQIDSIEIRGDTVTVHVTFHLTRPDGKTVTYHITYHDHYKDGKLTHTRFHVDTKET |
| 587 | PSEEEIREFLKRWYEEFVNEKDADKAASMIHPDATFHSDNTNLEQWKERVERTFRHTIDEIHVEIKSMEVRGNRITVHVTIHVHHKNGQTYTFDATHHWVFRNGKITHVNEHTRQHP |
| 588 | PDQEKIKDIKTKFFEAFDRKDLDTMVSLFHPGATVHFSDGITHHNLEEFKEYIKRLLDTFKETAHHMKRMHINGDTVEVHVEAHFKSDNQPRTFRVTVHDRYKDDRITESHFHWDEEN |
| 589 | ASEEELKEILKRWYEEVWNSGDWNAAASYIHDDATMHIWNGQTFHNRDEITEWIRRFLSSMKDVRFEIKRIEVHGDTVTLHGQLHVNHDDRTQTIRYTHHWTFQDGRIVRVEFHNDTGSS |
| 590 | ASEEEIRSWAQRYYEEADNSRDAEAIASLLHPDAQVHFARYQRQYDREEFREWLERWFRSVGDAQREIESIEVRGNTVTVHVTMHVTHNGKNVTIRATHHWHFRNGKLVEVRVREHTHES |
| 591 | PMDRSQIEEWVRRFIEALNHKDMDKLRELYHPDATLRYSGDHGLDAVMEWLKQWLEKFNNTRIEIKSIHVHGDTVTVHVTVHVRRGDEDQTFQFTSHFRFESNNRLVSEQIHTQPVD |
| 592 | PTDQSQIRDLIKKFIESFNNKDVDLVRELVHPDARLDASQSDGLDEIVEWFKRALEQFKNAQIQIHSIHIHGDTAHVHWTMHTHHGDQTYTYHGTTTFHFVKDNKITSIRIHVRQKD |
| 593 | TSEEEIRELLKEFYRALDEGDLDKARSIIHPDATMHISEGTTFTTQDEITKWVEEWIKKFSNVQRHIHGMEINGDTVTVHSRVTFTHNGKNKTVDVTDHYRFKDGKITNFTSHFNDT |
| 594 | PSKEEVEEWLRRFYTAWNNKDIDALAELLHDDWTYHNNQEKVTLTTKEEFRKWVKEFLDKAKEVHIQIDSIEIEVRGDEVHATVHVTIHVHHDDRTTTIHATDHHRFRNGKLIETHSTLETRE |
| 595 | PSKEEIEQWIRQYYDAFNKGDLDRLAELVHPDVTFHFYEEQITLTTLDEFREWLERFMRTFKEVHVEIHSIEIRVDGNEIEVTVNYTMHVHHDDRTITVHGQDHFRFRNGKLVEVRSRLETRE |
| 596 | ASEEEIREWVKRFFDAFNNKDLEALQRLVHDDVRMHVAKEQITFTTKEEITEWVKEFMKRFKRVHIQIESMRIETHGNEYTVHVHWTMHVHHDDRTVTLHGTTTFHFRNGKLVEVTFHIEERE |
| 597 | PSQEEVRQLVERMVEAFNNKDVEALKSLYDDDATYHDSSSNETLHTKEEFERWVRQWMSTFKDTRMQIDEMHIEVRGNEVTVHVHVTVHVNHDDKTVTLHITSTIRVRNGKIVEEHTHVDERE |
| 598 | ASEEEIRELVKRMIDAFNHKDLDTLASLYHDDATMHVSDGYTANDKEEIKEWFERVMRTFPDHRLEIKSIEIDGQTVHVHVQMHTTHDNKTVTMHVTSTFHFTDGKLIEERIHVDNRD |
| 599 | PDEEQIREFVKREVEIWNNRDLEKWKELFDPDVKWTNAATGQTYDREELIEMIKRILETFKQIQIEIKDLHISDDSYKVTVHVTVHFHRGDRTTTIDYTHHFHFRNGRLVRKQTHEDSK |
| 600 | PDEEIAREVAKRYYEAWDKGDWEALEELVHDDATFHEAETGNTYDKEEWIERYKSFLSQFPRIQRQIHDMHVSDDSNTVTVHGTVHFHRGDRTHTIRFTHHWHFRNGKIARMEEHVDTG |
| 601 | PEQEEIKEWIRQFLDQMFRALNQGDRDTIKSLYSDDATMHIYQGITFTTKDEILEWYERISSRFPRIHWEIKRIEVSDGTATVHVRVTFTHPTGEQRTYQITSHFHFDKDGKLTEEHIHTQPVPS |
| 602 | PKQEELEEIVRQFIERFVRALNNGDLDAVASLYHPDATMHIWEGITFTTPEEIREWIKRWAERFPEIHVQITEIRVSDGKVTVHVTVTFTHPSGKRHTYQVTRHFRFNKDRQLVEDRIHMQPVPN |
| 603 | PSQEEIKETKKRFVELFNRGDVEAAVRELFDDDSEFTHSETNTTKTASREEVIERFKRWFDSWPEVHIQIDSMEITSGDATVHVHVTIHATRNGKTTTFHVTVTDHYKDGKLKRSQEDTGST |
| 604 | DEREEIKEWIKRFFEAFDKHDLDKLISLLHDDATVHFSSHNVTLTDKEEFKEFIERMLSRFKDSRHEIESIEFRGDTVTVHWTAEFHHGDQTYTVHGTTHIRFKDGRVIEVRFHVDETG |
| 605 | DEREEIEKFVRRLFEAFDKNDWEEIARLLHDDATIHFASEQITLTTKEEIKEWLKEWIKKVKDTAHQIDSIEFRGDTATFHWQVHVTHGDKTQTNDATTHFRFKDGRLIEVRVHIDSTD |
| 606 | DLREEIKELVERWFEAFNRKDLEALAELVHDDARIHFASEQVTMTTREEYKKWLEEWFKRVEEIHFQIDEIRFHGQTVTVHWTAHVTRGDKTVTYHGTTHFVVQDGKIVEIRVHVDQTD |
| 607 | PEREEIKKWVERFFEALNKKDLEALAELFHDHATVHFSMTQITAHTRDEIKKWFEEFFKKAEEVHVQIKEIRFHGDTVTVHWEMHVTHGDQPYTFEGTTHFRFKDGRLVEVRFHVRQTN |
| 608 | DDREAIKEFKERLLEAIDKGDLDTFISLLHEDVTFHFASEQITLTTKEEIKDWFEKWLKKVKQTAHELHSVEFHGDTATFHFTVHVNHDTKTVTIHATSHDRYKDGRLIETRIHENTTE |
| 609 | SSEEEAKEAARELVERFYEAFNKGDLDLLKELMHPDATIHFSEGRTYHDLDEFIEAVKEFLKRNIKLNITSLHAEVDGDRVTVHVTVTMKDEVHHTWHFQRDHDGDFKVVEVREHTTG |
| 610 | PPEDEEKIRQWKKEFLDALNKGDADRLLEIIHPGSTFTFNGQTFTTKDEFKEWVERMKRRDTKFQLDELHIEFDKDHITVRVHVTIHTNDNSQPYTVHVTARDHYRKINGQWYLVRTEFHFHDSR |
| 611 | PREDEEHIREWIKREVEAFNKGDWEAWLRLVHPGSTFTYNGQTYTTKDELREWFEHAQKRNTQIHIKEIRIQFTQDHATVEVHFQMTVNDNSQQYHVEVTQTDHFRKINGQWYLIRREVHSDDKR |
| 612 | PEEDEEHIRQWYRREAEALNKNDLDAWASLFHPGSTVHFDGRTFTDQEELREFFKELASRNFRIHIESLEIHIDKDHATATVHFTVHWNTTSQPYTVKYHATDHFRKKDGKWYITERHVHTDDKR |
| 613 | PEEDQEKIKEYKKRLIEAFNKKDVDALAELVAPGATFHFNGQTFTTKEEFKEWVKQMASNDFQIHIDSMEIQFTKDTITVHVHYTVTFHESSQTYTVHATATDHYQKRDGRLYLVETRSQFHDTR |
| 614 | PPEDEEKIRQIVEEEFEAFNRGDLDRWLELWHPGSTLHVNGQTFTSKDELKEWMRNLLRNNVQMEIKEIRIEFSDDHATVEVHFTMTMNRDSQQYHVDATATIHLRKVNGDWKILEKHVQYNDKR |
| 615 | PPEDQEAVKEVVERFLEALNKGDADAVASLFHPGAEFHWNGQTFTTKDEIRKWVKEFNDRRIRVEVKSIEIHVDENHATVHVHWTMTVNDSSQPYDVDVHTTFHLQKRNGQFYILEVHFHVDDKR |
| 616 | TPASEEQIKEIIKRLAEAFNKGDMDAIKSLYDDDATMHIWNGQTFTSKDEIKRWFTELHKNDIEVHVQVVEIRVHGKTVHVTANFEFRDRRTGRTVRITRHITIRVTDNGELRIVEDQFHSHND |
| 617 | PSQEQVKEWANRFYEAFNKKDLDALYSLYHDDATFRDARTQQDYDRDEFREMVRQMLSRDIQLHWEIKEIQISDNHATVTVEVHMTDQTTGKTVTATQHFEFRRDHDGRVYLLRDEVHYQE |
| 618 | ISEEQIRETIKREFEAFNRGDLEAWASLFHDDATLRDARTNQDYDKEEMREMIERMLSNNVQLHVEIKEIRVTGNHATVHVEVHVTDSRTGTTVTFTTTIHFRLDHNGQVYIVEKRLHFQS |
| 619 | PSQEEVKEILRQFYEAINKGDVDAVASFYHDDATIHFSQTNQDYDRDEFREWVERMIRANVQIHFEIKRIEVSDNEATVTVEVTVHDSRTGTQVHFTQHIRFVKDHDGRIKIVEDRIHFNT |
| 620 | PSEEQVRELKKRLVDAFNRGDMETFDRLVHPDATFEDASQGRTYDREEFKKMLEEMWKAGIRLEFHIEEIEFSDNEATVHVRFTMTDSRTGRHIDARATDHYRLDHDGQLYLVRTQFHWQT |
| 621 | PSEEEIREIIRRLYEAINNGNWEEVARLFHDDWRYHNAKTQTTYDLEEWRRWVERFHKNNIQIEVEIKEIQFSDNEATAHVRVTVHDRRTGQTFHATDTHFFRRDHDGRIFIIRTQVHYQD |
| 622 | PSQEEIRQFVNRFYTAINRKDADEVAELFHPDGTFTFASTQRTYSLEEFIEWVRQAASNDFRFEWKIDEIQISDNEATVHVTVTVHDSRTGTTYQVHETHFFRKDHDGRIKIVRTEVTHQS |
| 623 | PSWEEAVKEWVKREVHAFNSGDMDAWLSLFHDDVTMHDSNGQTFNDLDEMKKWAEELWRQGAEVQIDSIEIKEITETEITVRVEITFRKTNTTVTFHITFRLDHDGSWKVVEKHVRTDN |
| 624 | PSDEEAVREWIRRFYDAFNKGDADALKSLYDPDAQMHISNGRTFNSRDSIKRWFTELFKSGVEIQIESIQVDEVTEKEVHVTVHFTMKQHNITVTQHITFRKDDNGQWRVFRDEIRFHN |
| 625 | PSAEEAAKELIRRFVEAFNKGDADAAKELYDDDAQMHIWNGQTFNSKDEIKKWFEEFHKAGFSIRILSIQVDEVTETEIHVTVHIQVDQYNIRFTSHVTVRRDHDGRWKIFEERIQNNN |
| 626 | PSDEEAIKEWVEREIDAYNKGDLDAWASLFTDDVTVHDANGQQFNSKEELKRWVKEMIEQGREIQVDSIRIDEITEKQVTVTVHITDKSQNITFTMHLRFEKQDDGSWRVIEKRMRNDS |
| 627 | PSEEEIRQVVRRFFEAFAKRDMDTLRDLYHDDATFHYNGRTFKDKEEILEWFKRLLDNIQRMKIDVEVHVDGKTVTVHSTVHRTHNGQDRTQHVTSHFHLDDDGKIVEERVHSNTQE |
| 628 | PSEEEIREAVKRFYEAFNKRDADALMRLMHPDATFTYNGRTFKTPEEIKDWFEKVLKNIEELHVEIHIEVRGDTVTVHYQVHFTHNGQTYTVDATDHFHFDKNGKIKRIQSHTRQTE |
| 629 | PSEEEIREFKKRFFEIVANGDMDAFKELVHPDSTFHWNGTTYKDKEEVAKMFAEFASTLQDAKFDIEIHVDGDTVEVHITVHMTRNGQTFTFHITVHDRYDKNGQLVESRFHENQQR |
| 630 | PSEEEIRETVKRWFEAFQNRDLDALADLYHDDATVTIDGQTFKDRSEIKEWAERVLKSIQRLKWDTEIHVDGDTVTVHSTFHTTHNGQDFTFHVTVHLRLDKNGKIVEQHTHTNEQK |
| 631 | PSEEEIREILRQFYEAFAKGDWKKLSEIVHDDATMHVDGRTFKTKEEIVRWFREVWKKMSRAKFDVEIHVDGDTATVHVTVHTTHNGQDYTFHVTDHYHFHKNGKITRIQSHTNQER |
| 632 | DLEEQVKEWLRQFYEALDKNDLDTAMRLLHPDATFTVDGQTFKTKDEIRKWFEEQLRSMRDIKREVSIYSITEREVTVRYTVHVNHPSGQTFTIDAQDHFRFDKDGRITEVRSHRDQKD |
| 633 | DFKEIAKEFLKRLYEAFDRGDVDAFAELIAPGWTMTVNGQQFDTPEEVRTFIERVLKSLKHSRREVQVIEATDRTVTVESTMHTTHDNGQDFTFHAHDTHRFDKDNRLVETRSHTDPKE |
| 634 | SSQEEIKEVLRRFYEALNKGDWKKIADLIHPDATFHHSNGQTYDYEQFVEMIKKYASDSKTRLEIEEMHIHGDTATVHVTIHQQRDTSRHTTHVTHHWHLKDGKVTELREHTNNT |
| 635 | TDESQIRDWVRRYIEILNNGDIDKLKELFDEDWTYHDIARQQTYNLEQFLEMVAQFMSRASTASLEIKRIEIGGDTATVHVTDHTKDKTFHYTEHHHFKDGKLTETRQQTDDG |
| 636 | TDESQIREWVKRWIEAFNRRDVDAIASLYHDDARITFEENNQTYDKEEFREWVERFMRTFPDFQLELKEVRIGGDTVHVHITFHHNNRTVHVTSTFHFKDGRLTEQRIRTDST |
| 637 | TSEEEIKDIKKKLVEAFNKGDADTFAELVHPDATFHFAQGITLTSKESVRKWFEEWVERNKDYRIEIDEIRFDGDTVTVHLTVHLGDTTVKVTTHDRYKDGKLTESRSHFQPT |
| 638 | SESEEAIRDWIRRWVEAFNKRDVDALASLYHDDATVHFTQGETFTTKEELRRFIERYLRTFKDVKMDVEEIRVDGHRVTVHMTVTVHHDNSTQTYHYTVTLEFNGDKLQEQHVHIHHS |
| 639 | MSEEEIKEWKRRMVEAFNNGDLDALADLFDEDSTFEVNKGEQFTDKEEIKEWFERLLKTFSDSQITIDEIRIDGNTVTVHSTQHTPQYNQTFRVTTHDTYKDGKLVSSRFHRDEG |
| 640 | DEEEDREAVKEWVHRYVEALNKKDRDALQELYTKDDSTFHVWNGQTLTDYEEIVKWLTEFASTVESVELDSIEVHVDGDTVTVHVHITFKSSNQTFTRTFHFEKTKDGFKLKEDRVQRHS |
| 641 | PEEKRKEEIKELVERMVEAFNKKDIEKLKSVYTKDDSTIHIWNGQTLTTQEEIERWIKEFLKTAEHIEIKSLRIEIHGDRVTVQVHLTLRSNNVTYTSTLHFELTKDGWKLVEEHIQNDT |
| 642 | PSDEEQIRELVRRLYTALNTDPDKFAELIHDDATFHISNGQTFTTKDEVKDWVEKFASNTSIEIKSIEVQIYKDGRVTVTVHFTVHTRTSTVDVTDHYHFQKTGSEWKLLEVRSHYDNR |
| 643 | PSEEAEKAVRELIEREIEALNKGDWEAWVRIIHMEDITVHINGQTFKNPEEIIRWFREFISNVQLQIDSIEIHQNDNGEVTVTVHITIHTKDKTIHVTFHLHFRKENGSWKLTSKQAHFDNKE |
| 644 | NDPNDVEAVQEIIRREIEALNKGDMDAWASLLDKDNSTLHFNGTTFETMDEMKDFMAKYVSNAQIRITSIEIHVNPNGTVTVHVHITIHHKDKTVHVTSTLHMRKKDGSWQIAEKHTHEDQKD |
| 645 | PHDQEEVEEFLRRLYEAFNKGDIDKLRELFHPDWQYHDAATQRTYDLEEFLERVEKLTKDIHIEIKSIHIETRGDEIHVTVHVTVHNPSTKTTVTVTDHHRFERGSEQWYLVETHSQSHNE |
| 646 | PSDQEAVEEMIRRMFDAMNKGDLDKLESLLDEDWTYHLEATNRTYDKDEFLEWVEKFMSHSHLEIKEIRIEIRGDTATVTVHVTVHDKRTGTTVNVTEHHHFNKKGEEWRILETRAHFNNT |
| 647 | PSDEEAIRETKKRLIEAMNKGDWETLKKLFHPDAQFHLSAYQRTYDADEFIEWVRKFMKDWRITLHSMEIRQDGDEWHVTVHVQMQDRRTGTSYSVTAHDRYVRNGEEWKILESRFHFNQS |
| 648 | PSDEEAIRELKKRFIEAINKNDMDTLKKLFHPDATFHFAAHQRTYDLDQFLKWVAERASHLHFQLDSIEIEIRGDQVHVTAHVTVHDDRTGRTFHVTSHDRYRKDGDEWKVIESQFHFNET |
| 649 | MSDEEIIRDLLRRLFEAMDKGDMDTLRELYAPDATFHIEERNRTFDLEEFLEFFAKRTKDFQRHIESMEIQQHGDDFTITVHVTVHNRSTGKTYKYTSHFHVRKSSSQWKFKEEHLQTRET |
| 650 | AHKEQQEKIKEWAKRFYEAINRKDPDAVKSLLHPDWKYHDSRTNTTYDLDEFKEIVEELAKNDVRLEVRSIEITASGDELTATVHVHFHNNSTKVTYTVTDHHRFREENGRIKIVETHSTMQSQDQ |
| 651 | VDESAEEEIRRWLRRFYEAINRGDLEAVYRLLHPDWTVEFSSEPREFNLDEFKEWVEKMIRAGFKFEVKSIEIHQDGDTATVTVHVDLHNDQTKDTFKVTEHHHFALRNGTWYLVRTQIHLQPVNS |
| 652 | TDPEDEKKIRDIKRRFVEAFNKGDWETIRRLFHPDAQFHFSSEPTTFNLDEFIRWVKEMIKRNVELRLDETHIEQHGDTIHVTSHLTFHFQEEQVTYRVTAHDVYQKQNGDWKIVEVHIHWDNSNQ |
| 653 | AHKEDQEEIKRWLKEFYEAIDNGDADAVYSLYHPDARIDFASEPRTFNIDEFREWVERLASQGTAHELKSIEIHKSGNTIHVTVHVTTHIKSTGNTYHVTQHFQFELINGQWFLTKDEIHINNENK |
| 654 | TDPSDQEAVRQIVRRFYEAFNNKDLDAIASLYHPDATFHFAEDPTDYDLDQFREWVKKFLDADIRIQIHHIEVESSGDEITVTVHVTMHNQRTGNTYHVTQHMHLRKKDGEWKVLRDEIHRDNANK |
| 655 | PDPSAQEAITEWLKRWVEAVDKGDADAVASLYHPDATFHDSRNPRDYDLEEFRRWVERFARNDVQRELKSVEIHQDGDRFTVHVHFTLHNRRTGDTFHVTATFFFEKHNGQFYIVRQEVTTQQENS |
| 656 | PDPSAEERIRQILNRFYEAMNRRDRDTLRELIHPDATFHDSNGQRYDKEEFLERFERIPWYRIHIDDMHIEQHGDEATVTVHVTFHIKTDSTSYTVTHHWRFVRFGDDWKIVELHVHTHNNS |
| 657 | PNPEDIEAAKQIVQRLYEALNRGDWEEFKSLIHPDATFHDTNGQSYDLDEFIEMLRKRDITRFQIDSVEVRQEGDTVKVTVHGTVHRSSDPKTFTVTHHWVFVRHGDEWYVVEVHEHTHNRS |
| 658 | PEDEEAIKEWLRRYFEAMNKHDVDKLLSLYHPDAQYTDTSRNQTFDYEQMKEMYESQSGQQTHIHVTHIEIQKHGDEVTVTVHVHITAHTKDRTRHIDVTYHFRFRRDGDEWKVVHVQETRRQT |
| 659 | PEDKEQIKEFKKRFLEALDKGDWQKVKEMMHPDAQYTNTSTQTTYDLDQLVEMIKSNSGKETHRQVHEIHIQQESDDFTVTVHVTVHTHTPTETRTYHVTYHDRYRKKDNRWYIVEVHETTHQT |
| 660 | MDSHILEEQARQIVRRFAEAFNNKDWEKLASLYHPDATFEFEETGTEYDLEEFIRWFRRLAETADIEIHFSIDEIEIRGDEATVHVTFHLTDRRTGRTTTYHSTFHVRYQDSEVKILRERITTRQT |
| 661 | MSEEELKEAVKDVIRRYVEAFNNGDLDKIASLYHPDASYYDSNGQEYDKEQLIEMLEKLFRQNFEMHFQIKEIRVDGDTVTIHVEFHVRDYSRQLTFTVHRTFHLTFTDSKVYITRDEVHTENNE |
| 662 | MSEKEQKEAIEETKRQFAEAFNKGDWKRLAELLHPDVRFHDSNGQQYDLEQFIRRVQKLFSQDVKMEFSIKEIRFDGDHAEVHVEFTVKHSQTGTTRWFDQTDFYQFEDSEVKIIEVRVRTVNGE |
| 663 | MDEKEMTEQIKEVVQREVEAFNNGDADTWASLFHPDARFHHSNGQTYDLEQVKEMIKELVKNNVRIEFSIKEIRFDGDEAEVHVEFHYHDARTGTTQTFDQTIRMRFEDSQVKIVSKRVHTKNGS |
| 664 | MSEEEQREAVKDMVRRWVEAWNNGDWEKLASLYHDDATYTDSNGQNYNKEQYIEMVKKQASQNLKLEFSIKEIRVDGDTITIHVEFHITDDRTGTTVTFTSTFHLTFYDSQVKITEQRVHTHNGQ |
| 665 | SEAEEAVRDMIRRWVEAWNTKDVDKLSELYHPDATFHHQNNQTYNKEDFRRRFEQLASSDFKIEVKSIEIQIDNKDEVTVTVHFTVTYEDTQTTTNITSTFFLRRENGRWKIVEERESRNQQQ |
| 666 | PNLEEAVRQVLREYFEAINNGDVDRVMSLYADDARWHTNNGETYDKEDLKRWYEEFASIPFHIEVKSIHIQINDDTHVTVTVHVQITHPETGRTTDVTATFHMQFQNGSVKIKEVHTHHNDHS |
| 667 | SSAEEDIRQVMRRYFEALNKRDADAVASLLDDDWTYHNSNGETYDKEQFREMVERYASIRFKIQITSMEIQINDKDSATVHVHATVTEEDTGRTTDITETHHLVRKNGSWKIIETHVHRNDRQ |
| 668 | SEMQEAVREWLKRYYELFNQRDWEKVASLYDDDARFHHNNGETYNKEDYERRLQSLASLDFTLQVHEIRVQIIDDTQATATVHVTYTNHHTGETVRITQHFFFRVENGQWKIKRDEEHRNRQE |
| 669 | SEMEEAIREVMKQLYTAFNTGDAELVDRLLHDDWEYHHQSGNTYDKEDFIKMFEEMASIPFRIQIDEMHIQIIDDTRATVEVHATITFESTGTTQHFTEEHHLRRKDGSFKVVETRVHTNDQQ |
| 670 | SDDEDAAEELARRFYESFNKRDWEKFARLVHDDATFTQNNGQTYDKEQLIEMFKSMASSRFHVQIDSLEVRIIDDTRVQVTVHVTVTFEDTGTTTHATHHWFFVKENGEWYLVHLHERRNDRQ |
| 671 | SEAEEAIRDVIRRLVEAFNNRDAEKVKDLYHPDATVHHNNGETYDKEQFIERFKELASFNFQIQILSQEIHIHNDTEATVHVQVQITEDDTGRTTDVHSTFHLRVFNGQWKIVEEHINRNDQS |
| 672 | SELQEKIKEFKQRMVEAFNNGDLDKISELVHPDVTFHHSNGQTYDREQFIKMVERMLSSRFHIQIKSMEIRIEDDTHVTVTVHLEVHFRDTGTTVEVTAHDHYRFENGSLKLVEVQIHRRDRS |
| 673 | PHDEEEIREWVKRFIEALNRGDLETLDRMLHPDWRFHNNKTGRTFSREEFKEMVERMKSNNTRVELKELHVEIKDGHVTVTVHLQFHTNHNGQTKTVDVTEHHHFRRSNGEWKLVETHATFHEH |
| 674 | PSDEEEIREWVRRFVEALNKGDMDKLKSMLHPDWRYVDDRTGQTYDLEEFAEMVKKLKSHGMRIEIKSLEVEVKDGTATVTVTVQVHTTHNGQQYTTTFHERHHFEKRNGNWYLIRTEVTVDHH |
| 675 | PSDQEKIKEWLRQFFEAFNRGDMDKLKEMLHPDWRYHNSNEGRTYDLDEFKEMMRQMFSNGIRLEVKSIEIHIHNGTATVHVEVDAHQTHNGQNKTVKFHETHHFDKKDGRWFLIRTEVTTHET |
| 676 | PSDEEDVRDIVRRLIEAFNKGDLDALRELYHPDARFHNDKTGQTYDLDEFIEMVEKLASNGTQIELKSLEVEVKDGHVTVHVHVQFHTTHNGQTKTVDVTRTIHFRRSNGEWKIIRDKATFNER |
| 677 | DEEEIKKEIKKLIERFVEAMNRGDMDAIDSIYDDDATYTHASGQTYGKEAFKRWVKEFLEKVEHIQVRSLEIEIRGDTATVHVHMEVTFKEDNKTYHFTSTFFFRKKNSTWYLSSERVTTQQDDG |
| 678 | AGKEAEEAIRDIIRREAEALNNKDYDTWKSLFHPGAQFHISATGQTYDLDEFVELMRKVFDPKRMEIKSIEIQTDGDEATVHVHFQVTHRKHNTTVTVTATIHVRRSNGQWYIVEKHIHFQPT |
| 679 | AGEEAEEAVRQWLRRFYEAINNKDMDKMKSLYTPGSTIWISDTGTSYDVDEFIEFFEKFFTAKRLEIKDMHIEVSGNTATVTVHVQVHLESTQQTYNVTQHFHFRREDGTWRLTEDHIHVQPT |
| 680 | AGDELKEAVKEWLERFYEALNKNDMDALRELFHPGATIYDSSTGTSYDIEEFLERFEKIFKMTRIEIKSIEIHTSGDTATVTVHVEVHFSRSNQTYRFDHTWHFERKDGTVRLVELRERMHST |
| 681 | SGEEAEEWVREWVKRFFEALNNKDMDALKKLFHPGAQFHDASTGTTYDLDEFLEMLEKIYQFYRIEVKSLEIEFDGDTVTVHVHWTGTREQSNQTYDATTTFVFERKDGTWRIVEVHIHFQPT |
| 682 | PEDQEEIKELKKRFLEAFDKGDMDTLRSLIHPGAQFHDSTTGNTYDKEEFLQRVAEFTSNVEIHRQVDRTEIQQHGDEYKVTTHVTVHFTHNTKTQTQHVTSHDRYRKDGDEWKLLESRFHFQQT |
| 683 | PHPEDETKIREIKKRIAEAMNRGDMDAIKELFDDDVTFTHESGQDFDLEEFLDWVKKLTSTADRIEVEIKRIEQHGDHVTVHVTVTVTNSSTGTTTDFHATDHYQKKDGQFRITRTHFTTRDT |
| 684 | TDPDAQEAVREWIKQFVEAFNNNDMEKLKSLYAEDATIEHSGGQRFDYEEFIKRVERRIRNSTFHLEFEIKHIQETTDEVTVHVQFHMHEEDTGRTFTYHSTFHFRKTDGGYKLKSEQIHRHQT |
| 685 | DEKQEELVRQALTREIEAFNTNDMDKWASLFAEDARFHNYTGQTFDLEEVKEMIKQMLSNTRLRFELHIISIEFRGDTVTVHTTLHVTDESTGRTTTIHQTIFLKIDDDDVRIKEKHVQEDST |
| 686 | PVDPTDEEAIRQWVRRFMEALNNNDLDKIRSLYAPDAQYHNASTQRTYNLEQFLEYLEKFLPGTHFEVKSLRIELRGDEITVEVHVTVTVHHKDKTVDIDVTRHFHFRRENGSYKLVRDQAHWDSKEK |
| 687 | PSMKELVEEWVRRFVEAINKNDMDALKRLYTPSDSRVHFDNRTFETPDEIKEWFRQHAENAKLEIKSIEIHTSDNEVTVHVHVTVTNDRTGKTVDVTRTLRFRIEDGSVKIKEDRVHRHDT |
| 688 | SDDEAREAVKRLLRRYYEAMDNGDWETLLRLLDDDWRYHDESGETITTREQFRERVERQAQHPTHRQIDSIEIHQDGDQVHVTVHVTQHHEETGTTVKVTDHHTFTRKNGQWRITRTQSHRDPRQS |
| 689 | DEEKEAKEWAEEWITRYYDLLDKGDWEAVLSLYDDDATIHVYNGVTYTSKESIEEFIRRWISESQNARHEIKRIEVEVRNGTVHVTVHVQVHNSSTGQTFDVTQHFEFRKEGDNFKLKRDEIHRRDTT |
| 690 | DEEDESREAIKEIVHREAEAFNKGDWETWLSLFHDDATIHIQDGETLTTKEQIRRWFEEVRKQAQFSSIEIKSIHIEVHNGTYTVTVHFQVTDSRTKRTYDFTQHIVFVRHGDEWKIVEKHIHRRDTS |
| 691 | PEEEEDKEQIKEWVKRWVEAMDKGDFDTLRDLYHDDATIHIFNGETVTTKEQLEEWLRRVLSQSQFSKREIKSIHVEIHNGTATVHVHGEVTDSRTGKTFKVTSTFHFEKHGDRWYLVSQHLTMHDTS |
| 692 | PVNEEEAKEQIRDWVRRYIEAINHGDLEAIASLYHDDVHWWARNQNKEELKRWYERFLRRFPDVRIEIKDLHIEIRNGTATVHVHVQVHFPGQNQTYDVTTTFFFEFQNGQIRLVEVRTDNQPT |
| 693 | PINDEEVEEALRQLYRRELEAFQKGDIDTWASLFHPDATFEGRKLDYEQMVEWFERILRTFPTMKVELKDMHIEVKDGEFRVEVHVQFHVEGTNRTFNVTFHDFIRVRNGEFQIVNRQMHQNDT |
| 694 | PINEEEQREQIKEWVERFVRAFNSGDLETLDSLYHDDATFDGTGTSKEEFKRWIEEFLRRFPDARMQVDHLEIRFKDGEATVEVHFTVHVPGKNITFHFTSTFFFKFENGQMKLTNEEVTRDTS |
| 695 | PSPEDEKTIREWVRRFIEAFNNKDLDTLVSLFDEDWTFHDANGQTFTTLDEFREWMERVLRTFQDAKMQLDDLEIRFDKDHATVTVHITIHDSDSNTTYHVTEHHHFRKDTSGWKLLETHVHTDQQPT |
| 696 | ASEQEIREAVKEFKRRLVEAINKGDMDTIRELFDSTDSEMHIWQGRTYHDQDEIAKWFEEFRKKFEVEIHITNIEIHIRNNTEVTVHVHFHVTDRRTGKTFTYHATDHYRFEGDTIKLKESRFTRDEHR |
| 697 | AHPEQQEKIRQIIQRYFEAMNNKDADKLISLIHDDWTYHDSSGRTFHNKEEFKEWVEEFLSKFDVRFEVKSIEIRLDDSTQITVTVHVDVTHTGTNQTFKVTEHHHFRLKDGDWKMVRTEVRTDG |
| 698 | PEEEEIKEIIKELKKRLVEAFNKGDLKTLRDIFHPDATFHDSNGETLTSLDEFIEWVKEWLRRFDTNLQITEIRIEIKSKDEATATVHITVHFEDGTTYTFTFHDRYRRHGDEIKIVESEIRTNNTG |
| 699 | PREEELKEQVEEIIRREFEAFDKGDWETWLSLFHPDATLHDSDGTTFTTREELRRWFESLRKRTDTAHQIDEIRFEIKSKDTITVEVHVTVHTSEGKTYTFRAEIHLTRHGDEVYIIEKRLHTHNTG |
| 700 | PKEEELKEQVKEVLRRFYEAFNKGDVDTLAELVHPHATFHFSNGETIHSRDEFREWFKEFKKRLDFRIEIKEIRFEIKDETNITVTVHVQFHRQNGQTFQVTEHHHLRRHGDEVKIIETEFRFNNTG |
| 701 | DDDQQAVKKLKKRFVEAFNKDPDAVAKLWHDDSQMHDSNGSTFNSQEEFKEWVTRLARHAQIHVDSIEVHSRPDGTIEVHVHMTIHRENDTYTVTATDVYRKKDGSYQIVESRFRVHQT |
| 702 | PSDEEAIREVIRRFYEAFQTDIDDLASLYHDDATMHLWNGRTFTTKEEIKEWFERLARTTDVHIDSIEVQTNPDGSVTATVHVTTHITNSTFHVTQHIHFRVQDGDWKIIRDHIHEQPS |
| 703 | MSEEQIKEWVKRYFEAYDKGDWETLVRLVDDDWQFHQNSGETIHSREQFREWVKEFLERFQDTAHQIDDLEIRGNTVTVHVTVHVNMNGQTFTIHVTEHHRFEGNKLKETHQHTNQQD |
| 704 | PDPRAIEQIKEWLKRFYTIINTGDWEKLAEILDEDWTYHDSSGQTYHDLEEFAEWVKEFNKQNFHMEIKSIEFRQDSRPGYYHVTVHVTVTQEGTGRKFDTTDHHTFRFQDGSIQLVSTQVHWRET |
| 705 | PEKELQEKAKEIKRQLVEAFNKGDWELVARLVHPDATFHHSDGRTFTTLEEFIEWVKQWSSQNWEIEIKSLEARVLPDEREVTVTVHYEVRNQTDPKTFRATTHDRYVFKDGQVYLIESRTQWHPL |
| 706 | DEPEAQEEIKEIVKQLLEILDTGDWEKLSSYYHEDATFYYNGRTFHTREEFTEWVRQFASNQSHHQIHSIEIHKDGDEYEVHVHVTVHFRRDPKTFKVTSTFRFRQTKDGLKLLEERVHRDDGQ |
| 707 | MSEEEMREALKEWFQRWAEAFNRGDMDALKSLYDKDAQMHISGQTFDNPDEIKEWFERLFRQNVKIEIKSLEIRMTPDSTTATVTVHAQMHNETDSTTWHFTAHFEFTFEGDTLKLKRQQTHIHQDDK |
| 708 | PSEKEIEEAIRELLKRFYEAINKGDWEKVARLVDKDWTFHYNGRTFTSWEEFVEWVKEFFKQKFRIQIDSIEIEIDSDQNNATVHVHLTVHNEDNPVTRHTTDTHHIRYEGDSFKFVETRVQMHQDDT |
| 709 | TSTKEAEEAIREFKKRYVEAFNNGDMEKLERLIHPDATMHVSGQTFTSRDEIKEWLEEMIRQQWQMHVDSLEIRQEPDPTHFTVHVHVTFHLDRNPTTVHATQTDHYRLKDDSFRIIRMQVHFHQDDT |
| 710 | MSEKEQEEAIREWKKRFVDAVNTGDMDKIRELVHPDARFEVDGQEFNNIDEFLEFIKQFFREQWRIHIKSLRIEFDEDSREVTVEVHLEMRNERNPTTVEATFEDHYRFKDDTLKLIETRVQRHQKDT |
| 711 | MSEEEMIEAVRQWLRRFYEAIDRKDLDALARLFAPDATMHIAREQREFDWEEIVRWFERFLKTFPRTAHQIDEQEFHIHGDTITVHVRVTVHFQDTGTTVEVRDTYFFEFENGELKLRRRESTFHQK |
| 712 | MDRSEQEEQIRQFKQRWAEAWNKGDLDTILSLLHPDVTFHFASYQRTYSYEEYREFFERFQKRFPNARLEIKRMEIEFHGNEVTVHVHFTVTYPDTNTTVDYTTTDRYRFENGELRLLESRTQAHQN |
| 713 | DEEEDREEIKEWKERFLEAIDKNDMDKLRELLHPDATWYNGKHPQESTLEEVLEWLRQFWETFKDTAHQLDSLEIRFSDNTATVHAHVTVHFHHNGQRYTYRVTATDHYVKRGDEWKLVRSEEDRRQS |
| 714 | TSEEVKEHITEVVRREIEALNNGDMDTWRSLFHPDATVTFKSEQRTYHLDQVVEMIKEFLERYPDTHMEIKDLHIEQDGNTVTVHVHVTIHVRHKDGTITIHVTATIRFRLKDGDWQIVHKTVERHQEDH |
| 715 | ASEEDQREVREIIEREVEAFNKRDLDAWMSLFHPDARMTVAAHQQTYNWEQMRRWFKTFLDQYPNTHMEIKDLHIDQSGDEITAEVHVTLHITNKDGQVTIHVTATIHFRKINGQWFIIRKEVHMRQEDT |
| 716 | PSKEDQEKIKEIIQRELEALDNKDLDAWFELFHPDAQVTDSRTGTTYSAEELREMVEEWLKKAKDTAHELKSLEIEQDGDTYHVTVHVTAHVHHKDGTVTYHFTAHLTFEKRNGQFYIISKHVTTRREET |
| 717 | ASKEDQEDIRELINRFFEAINRNDVDAVKSYLHPDATFHFASEQQTYDLEQFRKWLEEFMSHFKDTRIEIKSMEIQQSGNEATVHVHWTLHAHTKDGTFTIHGTTTLRLRKENGKWKILEVRISIDRQKT |
| 718 | ASEEDKEEIRQIKQRLFELFDNNDADALRSLFHPDATFEFEESQTKYDLDQFKEWVKQFFRTFQDIQHEIKSMEIEQHGNEIHVTVHVTLHITFPNGHVTIHATYHDRYRLINGNWYIVETTVTFRQEDS |
| 719 | THEEAERQARETVRREFEALNHRDIDDWASLVHDDAEFHYESSPQTYNKDEFREWVERQLKSWSSFRIEIKSLHVEQRGDEYTVHVHVTVHFDETKTTTHFDFTIHMRRKDGEVYILEKHIRTHHE |
| 720 | TDPEQEEKIKEWLRQFYEAWNRRDLERLAELLAPDWTYHSNDGNTTYDKDQFKEMVEEFMRKFSRFRLEIKEVHFEQRGDHVTVHVHAQVHNPQTGTTTHTTDTHHFRFENGQLRLTHTHSTTYHS |
| 721 | THKEAYEEIKEIVEALAHAFQTRDLEKLKSYYTDDAQITFSDGNQTYNKEEFARIVERRLRELSDFRVHLTSLEVEQRGDEFTVHVRFQMHNNSTGETYEFRSTIHIRRENGRWKILSEKITTHKT |
| 722 | TDPSAREKIKEWLERFYTAWQEGDLDKLESLLHPDAKFHSGNGNQTYNKEEYKEMVERFMKSVRSYRVQIDSIEVHQDGDEFHVTIHVTFHNDDTGTTVRVTNHDRFRFKDGDLKLVEHRSTHHHE |
| 723 | PSDEEAAREIVKRFAEAFNTDPDAVAELIHPDATVHIYNGTTLTTKEEIKEWFTRLISNTRIEIKSIEVHKNPNGTVTVRVEWTMHRKDQTYRGTTTFFIEKRDSEWYILEVRIHVQPT |
| 724 | GDPSDEEEIREILKRFYEAMDKGDMDLIRSLLHPDVTMHLPNGQTFTNLEEVIEWIERFHRNFPHHRSLDSIEIEIRGNTATVTVHVTIHFPGENQTFHVTHTWFFRKKDDTWRIVEMREHRDDKG |
| 725 | MDKDDKEEVKQFIERYYEAYNKGDIDALAELVHDDAQFHYNNGETLNSKDDFREWVTQLLSKFPSQVHIHSIEFRASGDEITVTVHVTVHHDGSGQTVHVTDHYIFRKEGDRWKIVQVHSHRDTRD |
| 726 | PSKETEEALREWIRQYIRAINERDVDAIHSLYHDDSTLTNQSEGRTYDAEEIREMYERFLKTFPRTTLELKDLHIEQRGDTATVTVHITIRFWGEGRHVDVTYHFHFRRSDGPFKLVHVQVRADDKHS |
| 727 | ASKEDQEEIREILEREFRIWNNHDLEAWEELLDKDVTVQYSDTGTRYSADEVKEMIEEILRRFPDARIQIDDMEIRQDGDEYTVHVTYTVTFTGTGQNITFHATFFFRKSDGPWKVISKQVRVDRRTQ |
| 728 | PSDKAEEDVRQVVRRLVEAFNNHDLDALDEIYAEDVTWHDSRTGQQYSKDEFRERVERIMKTFPSMHIEVKDLHIEQRGDEVTVTVHYTQTFTGTGRQSNITSHFHLRQKDGEWKIVREHVTTDDSQT |
| 729 | PSKEDEERIREWVRRFYDAFNKHDFDKIAELFHPDSTYHDSRTGTTYDYEEFLEMVERFLRTFPDFRMEIESLRIEVRGDHYTVEVHFTVTFSGHGDNVRVTDTYFFRKKDGPFKLVEMRSNIDNVRT |
| 730 | AGEEDREAIKDLLRRFYEAIQKNDMDKLRELLHPDARFHDARTGQTYDKEEFLQRVQEFWERFQDINVEIKSIEIDADGDTITVHVHVQFHVNTPTRTQTFNVTETHRIEKHSNDYKIVETHIHTNQT |
| 731 | SSEEEMREKIKDVKTKFIEAMNKNDLEALAELVHPHITLNSANLHTRDEFKKWMKEWLEKFSDAQIQVDEMHIQINSKDTATVTVHVTVHNKSTGVTFKFTAHDVYRFEDGSVKIVSSQIHTDNGS |
| 732 | SSEEEMKEAIKEFKKRLAEAFNNNDLDKIRELFAPDVTLNSANAKNLDQFVEWVERWLSDWKDFNIQITSIEIRIDSKDTATVHVTFQVTDARHNVTFTFHATDVYVFKDGSVKIKSVEIHTDTRS |
| 733 | MSEKEMEDQIREVLKRFYEALDKHDLDTLDELLHPDWQLDSQNLTTREEVQRWFTKWLSEFKDSNHQIDEIRIEIKSKDTATAHVHVTVHDNSTKQTFHVTETHFMVFKDGSVKIVRTRVHRDSGS |
| 734 | MSEEEMKEAIKQVIERFYEAFDRGDMETIARLVDPHVTNRSRGIKNREELIEHLKRLVEEFKDVNHEIKEVRIQIDSKDTATATFHVQQHDSRTNTTYRVTDHYFLEFKDGTVKIVSVQSHTDTGS |
| 735 | MSEEEMERAIRETIKRFYDALQNNDRDKLRKLVSDDITLRSSGATTKEEFIKWFEEWLKTFKDFHVHIESLHIQINSSTSATVTVHVTFHNSSTKVTYHITDHYFVRFKDGSIKITHVQSHTDSQS |
| 736 | PDPRDEEAVRDMVRRMYTALNNKDADAVRELVHPDSQFHIYNGRTFNNAEEFTEWVKEWLKNDARVEIKSIEVRQDDDTRITVHVTVEIHYKHGPHTFTFHHTWFFVKINGSWKIKEVREQRDSS |
| 737 | PEEKEIEEAIRRLLERYYRAMNNGDHDALKKMLHDDWQMHIDGQTYNSKEEFEEWLRRMLSQGVRLEIKSIEIHVKDGTATVHVHVQFHNSSRPQTFTVTETHHFRRINGQWYLIRTEIHRDSN |
| 738 | PEEEEAKEKIKEFKHRVIEAINTGDWEAIARLFHPDAQMEVNGQQYDDLEEIIRWIQRLHSQNVQIEIDSIEIETRNGQYHVTVHVTVHRRNNPQTYTVTFHDVYRFKDGSLKVIRTEVRTDSN |
| 739 | PEEEEAKRAAKEILERFFEAMNKGDWEALERLFHPDAQMHVDGQTFNSQEEIRRWFERWFSADVQIQIKSIEFEIKNGTITVRVTWDAHNSRKPQTFTFHTTVHLEQQNGSFKITRMEIHTDDN |
| 740 | MSEKEMEEAIRDWLRRWFEAWNKKDMDTLKELYAPDATFQHSNGRTFRTLEEIMEWFKQMLDQDVKVTLHSVEFRMHDETHATVTVHMTFTNDQTNTDVKATAHFHFQFDSTSVKLKRQQVHEDST |
| 741 | YSEEEAKELIKEVIKREFELLDSGDLDAWLSLIHPDATFHDSNGITFTTLEEIAEWVKQMFEQNWRHRITSIEIRVDSKDEVTVHVHVKVQQESTGRTRDVTFTDRFRFDKDSVKIIERQVHYDST |
| 742 | TDESDIREWLKRFYESINKGDLDAIEELFHPDATMHLSNGRTFTTLEEIKEWFERLAKRFDIKVEIKEIRIDGQTVTVHVTVTRTHRSGKTQHMTVTDHYHFEGKKLVEMRSHFDTKDQT |
| 743 | AHEEEIRELLRRYYDAWNKKDVDALAEIFHPDATFHHSNGQTFTNREEFREWAERFMKRFDMQIEIKEIRVDGQEATVHVTMHFTHSDGSKQTARVTEHHRFRGKRIVETHQHIDMRDVT |
| 744 | ADESDAREWIKRFFEAFDKGDLDALAELVHPDAEIHVPEGITFTTKDEFKEWVERLFKRFEFHHEIKEIRADGNTVTVHWTLHVTHSNGETYTATGTTHLRFEGKKLVEMHIHVDMKKVT |
| 745 | MDEDELKEAVERTMRRFYKAFDEGDADTAASLVHDDWTYHQSNGETLTTREELKQWVEEWFKQFPDTRREIDSIEIRVGDETEVTVTAHFTVHFTHSNQTRHTTDTHRFQFQNGSVKVIETRSHENG |
| 746 | MSEQEAREDIKDIIRRFIEAINKGDMDKVRELYHPGATLHIDSGVTYTTLDEIIKWVAELASNNLRIEIKDMRIEQNGDRATATVHVKVTNESDKRTVDVTSHIHFERTNNGFKIVEEHVNTQN |
| 747 | YSEKEAREQIEEIKRRIIDALNKGDMDAIKSYFHPGATMHIWNGQTFTDLDEIKRFFKEMHDNNFKFQLDEIHIEFKGDEATVTVHVTVHNDSNPQTYTVTAHDRYVYTNGQWYLVESRFHMQD |
| 748 | MSEQEIREDIEDIIRKFYEAINKGDMDKLRSLFHPGATFHDANGTTFHDLEEIVRWLKQIFDQGWELEVKSLEIEIRGDTVTVRVEVTMRNRSDKQTYEVHDTYFFHRSENGYKIVRVESRIQD |
| 749 | PGEEEIKEAIKEFLRRFYEAFNKGDLDALMRLVHPGVTFTQDNGRTFKTMEELREYFQKLIENNFRLHIHEIRIEIKDGTATVTVHVTIQQQTTGKTVRVDHHWFFRVEGNRVKLVEVRSRTDDDN |
| 750 | DKEKKTREQIEDFKRKEVEAINRKDLDKIKEIIKDAQYTNSDTNTTYDADEFVEMVEEWARRFPNAEIHIDSIEIHIDPKGDHATVTVHVTIHYKDTGETRHVTYHDHYRQEGDELKLVESRFTTTNG |
| 751 | PDEEEQREQIKQWIERFYEAMDRGDWQTLKELIGDATFHNTDTQQTFSADEFIERVRQFQSQFPNVHRTMVSIEIRFDPRGNHITVHVTVEVHIGETKQTHRFDNTDFFRLEGSQWKLVHHRFTTRGG |
| 752 | DTEKMEQDIRELIRRFFEAFNNKDADAMASLLHDDAQIHFEEDKKTYDKEQFREWVERVLRRWPDTKVQIDDLHIEITKDHVTVEVHWTVTFNRNGKTHHYHGTTTFFFRLKDGQWRIVELRFHLESRD |
| 753 | DGGEAQEQIKELVERFYEAFNRKDVDKAMSLVHPDATFHFEEDPKTFDKEEFREWLERFIRTFPSHQLDIDSIEIHIDKDHATVHVHVTVHVNKDGKTYTYHFEETHHFRLVNGQWYLVETHIRYEERG |
| 754 | DSGKAEEDIRQIKRRFIEALNRRDVDAFMELVHPDAQFHFSSDPKTYDKEQYREWLERFFRTFPRVHIEIKDMEIRVDKDHVTVTVHVQVHVTQNGKHETYHFTYHDHYRLKDGTWQFIESRVTTDQTD |
| 755 | DSGEMQEAAKEVVKRLVEIFNRNDADAFASLIAPDWRYYDSRDPKTYSIEEFREMVKEFASRFPDTKVQIKDMHAEVTKDHVHVTVHVEVHLNQNGKTTTTHVTEHHTLRLENGSWKVVSTRTTMETRE |
| 756 | DSGEMQSAARELINRFYEALNNKDWKKIADLIHDDFTFEDSRDPKTYSREEFIEWVQRWMKQFPDARLQIDSQEVRVDKDHVTVTVHVTMHTNHNGSTITFHITHHWFFRLENGQWYIVRLRETMESRH |
| 757 | PSEEDERAIREIKKQLVEALNNGDADRFASYYHPDATVHTSEGRTFTTREEIKEWAKQLFSNNIRIEIKSIHIDRDGDRATVQVHVQFHNSNTGTTVDFTAEDHYQKKDGDFRIVEVHIHRDNGG |
| 758 | PSEEDQRAAKDIKRRLVDALNKGDADTFASLFHPDARIHFSDGTTLTSRDEIREWVKKFFSQGWHIEIKSIHVDQDGDEFTVTVHVHLHQERTGRTFTVTAHDRYRVKDGEWYIIESRFHFDNGG |
| 759 | PSEEDEKEIRDIIRREVEALNKGDADAWASLFHPDATYHLYQGRTFTTREELKEWIERFTSQGVHIEIKSIHITQDGDRLTVEVHVTYTNDSTGQTFHMTFTDHFQLQNGEWKIVERHVHVDSGG |
| 760 | PSEEDQRAIKEILKRLLEALNKGDADALLSLFHDDWTLHINNGETYTTKDEFREWVERFFSQNFEVQLDSVEIRQDGSTFTVHAHFTVHNRNTGQTFRVTETHHFQKKDGQFYIIETHTTHDSGG |
| 761 | PSKEDEEAIRDLLKKFYDALNNNDLDKLKELLHPDWTYHSSDGRTFTTKEEFAKWVEDLWSQNFKFDIDEIRITQDGDHFTVHVHVTMHDSRTGRTVHATDTHFFRKKDGQFYLVETRTHWDNGG |
| 762 | PSEEQKEKIKQWYERELDAFNKGDADAWASLFAPDATMHLWNGETFHDREELKRWFEQMISNDIKIQLDSVEIHVDGDEVTVHVHFTFHNDRTGQTFHVDATDRFRFENGQLKLIERHVRRDNGG |
| 763 | PSKEDEEAIREILKQLAEAFNKGDADAFASYFHPDATFFDHDGTQHNSREEIKEAVKKLFSQQFRVQLDSVHIDLDGDHFTATAHWTFHQERTGRTFTVTTHVRFRKKDGQWYIVEFRMHLDTGG |
| 764 | PSEEDERQIREFDRQRLEAFDKGDADKVKELFHPDARIHFYDGRTITNAEELAEWVKQFASNDVKRIIHGQRIEQDGDEFHVTGHFSLTDSRTGKTVRFTYHITYRKKDGTWKIIRMEVRVDEGG |
| 765 | PDESQIRDFKRKEVEAFNNGDMDTLRSLYDEDARVHTSQEQVTYTTRDEIVKHFKELLEKFPDMRIEIKEIHVDQDHATVHVQVHTTQDGKPVTFRVTVHDRYKDGKLKETQFDIDNHPD |
| 766 | PDESEIRDWLRKYYEAIDNGDWETVASLFHDDATVHYSKYQVTLTTKEEITRFIRRFFSRFRNSRHEIEEIRFDQDTVEVHYTATYHQDGKTTHRRATDTFHFKDGRLVEVRSHWDDVPD |
| 767 | DKEDEAREAVKEWVRRFVEAINNKDVDAIASLYAPDATMHINGQTFNDWEEIRKWFSQWASRADLQIHVDEQEIHITDNTVTVHVRMTIRNKSDPTDYEYHSTFHFTKEGNSWKLKEEHVHSRQL |
| 768 | DEDEEIQRAVKEIIKRLYDAFNNKDLDALRELITPDWQNHTNGQTFTDLEEVVEWIERYLKTFDTRLQIDSMEVETSDNSATVHVHVTAHHPEDPKTFTITETHHIEKNGSQWRLVETHVQSHEQ |
| 769 | DEEEMRQKVKEMVERHIEAFNKGDLDKLESLYHPGAQFHDSRDPKTYDLEQYKEMVKELFKANVKMQLDSIEIRINGSTEATVTVHVTVTNDSTGTTYRATRHIRFRFKDGGLKIIEDRATFREQKS |
| 770 | DEREMEEAARQTIEREIRAFNRGDLDTWASLFHPGAQFDDSRDPKTYDLEQMKEMIKELIKANFRLELKSLEVRIVGRTEATITVHVTITQDQTKRTYQATFHFHIRFKDGGWKIVKKEARYREQKS |
| 771 | PEEELKKIAKEAVEREFEAFNKKDMDTWRSLIHPGAQYHDSRDPKTYDKEQLTEMVKELLKADFQLEIKSITIEIEGRTHVTVHVHFHMTQPQTGRTYTVTTTIRFEVRNGSVKIVEKRTTTRQQDS |
| 772 | DEEEAKKEIERIVRAFYEAFNKGDLEAAASYLDDDWTLHVDRGRTITTKEEMREYLKQLLDNGIQLHIESIEVRVHPDKTHITVHVRVQVTNRSTGSTVTADDTHHFRRENGDFKMVETHITHREE |
| 773 | DDKELEDKIREVLERLYRALNNGDMDTFMSFFDDDWTFHDSSGQTFTTKDEFRRWVERFLKAGWKIEVKSIEIHIHDNGEEVTVTVEVHIHNDQTGQQYTIHDHHFIRFENGSLKFVETRVQYRNE |
| 774 | DEEEIRQRIEEIIRRLYEAINKGDLDKLRELIHPDATFHFSNGQTITNIDEFLDWVKKFTESGFKMQIHSIEIRIHPNKDEATGTVHFTLQNDRTGTHYRVTDHYFLRRKDGDWKVVRVQSNIHSE |
| 775 | PEEEQKKAAEEAIRRLYEAFNKGDVDAMLSLFDDDWTFHDASGQTYTDREEFKRWVERLLSNNIHLQIRSIEARIDKDGSTVHVTVHAEMTNSTTGVTYTVTEHHTLRFENGSVYMVETHIHIHSE |
| 776 | PLSEKELKEQVKELVKRYYDALNTGDLDTLLSLLDDDWRMHQKTQTWNSKEEFKDYVTKFKSNTNAEIQIRSMEIQIHGDTATVRVHVDVTIRNTGTTITYRDTHHITAGSNQAKITETKSDEDN |
| 777 | PETSPEDREEIKRWVERYAEAFNKGDVEALASLFDDDWTFHDSSTGTTLTTKEEFKEWVKKLLSNDVKVEIKSIEIRTVPNGTIQVHVHVTYHSKDTTVTVHETHHFQKEGDTWKIKETQVHFHQE |
| 778 | NPTSEEDKKQIKEIDQRRIEALNNGDRDALEELFHDDSTMHDESTGQDFTTKDEIKKWYSEVFDGDFKMQLDHLEIQSTDNGTVEVEGHATLHTKNSTVHVRYTVRYQKRGDTWYIIHVRYNFNQS |
| 779 | SQTSPEDQKEIKDWIRRFFEAINKGDADAVASIFDDDWTYHNKSTGQTYTTRDEFKKWVEELFSNDAKIEIKRLEIQSNDDGTATVHVHYTMHVKDSTFTVTEQHHFEKEGDRWKIKETHTQIDQN |
| 780 | SSEEETKEAVRQWKKRFVEAFNRGDADELRELFSPDVQMHFSNGRTFHNREEIVQFFTKQTQNGTKLTLKHMEIQIHSENRVTVTAHFEVHDDRDNKTYDMTTHDRYELRGDDWYLVETHIHIHQQ |
| 781 | YSEEEIKEIVKDVVRRWAEAFNRGDWETLLDLYHDDATMHISNGQTFNDPDEIARYLTSLASQDRKMDITHIEIQIESENHATVHVHFEVHDSQDNKTYTFTSTFFIVFRGDGWKIVEQRIHTHQT |
| 782 | PEDQEDIETWIRRFYTAFQHNDLDKLSELLTDDWTFHYVRDDERQTYDRDQFREWVERIAKTFKDIRVQLDSLRIEIRGDHATVEVHFTATFHQDGKTHTVHTTDTHHFRRTNGGWQIKETQFNLEQRSD |
| 783 | DEEEAREQIKELVERWAEAFNKGDLDALAELLTDATVHYSYDKKTLTTKESIKEYYERVFRTFPGSQIELESIEIHFKDGTAEVHVHWTQTIPQTKEHYTGTTTFHVEFHGDDLKIREVRITVDSN |
| 784 | DEEEAEKEIRELKHRFVEAFNKGDLDALLEMLHPDATYHDSSGRTFTTPEQFEEWMRRFLSFKVQIHIDSIEIHFSGDTATVHVHLTVHRDGTGQTYKYTATDHYVRFGDTWKIVRVQVHTQPT |
| 785 | DEEEIKSQVEEFDRQRLESFDKGDWEAILRMFHPDATFHDHNGETLTSWEDFREWVKKFASNQTHHEIKEIRIEVRGNEVTVHVHVSVRYSKTNQQYKMTYTIRYRLEGDEWKIVEFRAHLQPT |
| 786 | DEEEQRRQVKEWVHREIEALQKKDIDTWLSLFHDDFTLHISNGRTFTTKEQLEEFFRELASRDFRVEVKRIEIEVNGNEVTVTVHFTVHHDKENQTVDVTAHIRIRFEGKELKVTEKRMHFQPT |
| 787 | DEEEMKRQARDWLRRWFEAFNKKDVDKILSLYDDDATFHDGNGRTFNSRESIEEWIRQFLSRDARIEVESIEFRVDGDHITVHVHFTVHFSHTGKTVRFTATFHFRREGDELKLLRQQIHSQDT |
| 788 | ADPSAQEAVKQLVRRWVEAFNKGDLDALKELYDDDAQIHISNGQTFNDKEEYARWVEQLSQSAQLEIHGIEVRPTSKDYYTVHVNITMHVKDTGTTYHFTATFFVKRHDWKIVEQRTHFEQRG |
| 789 | LDPSQEEQARQLARRFYDAFDKGDADKLKSLVDENWRLHVSEGRTFQSKEEVAEWVQKFADGTAHKMDEIRVQLDSDKDITVTVHVTVHNKKTGTTVTVTDTHHVKADDPKIAETHMHYNQHG |
| 790 | LDPSQEEIIKELVERMVEAINKGDMEKLKELYHDDATFHAYNGQTFHDKEEFLDWVRKFASDAKLHITGIEVRPDSKDHITVTVHVEVRFDSTGTTVRFTSHFVIRNNDWKIQEEHLHRDNTG |
| 791 | TDPSQQEAIKELIRRFIEAFNKNDMDTLKELFDDDWQFHHASGQTFNDREEFLEWVEKFARDYKLQLHSIEIESVSKDTVTARVHVTVHQDSTGRTAHVTETHFFKNNDYKITETRVHYDTRG |
| 792 | ANDEDEEAVRQVLRRWFEAWNKGDWETLLSLYHDDARITHADTQTTYDKDQFREMYESQLSFDIKISVHSIQFDRASSHTITVTVHFQMHVNTPSRTHTVDVTTTFHLRREDGRWYIVEQRTRRDNKQ |
| 793 | PGDETEEAIRQWFRREVEAWNKGDWETWLSLFHPDVRYHNADTGTTYNAEDLRRMFEQIQSVPFRLELKSIQIDQFTETEITVTVHMQWHFHHKHSTHTVDVTYTDHFRREDGDWYLIERHTQTDNGQ |
| 794 | YDKSAQEAIKEWVEREAEAWNKGDWEAWASLFHPDVQYHNNDTQTTYNLEQLKEMFRQQQKIPHRVEVKHIEIKEMTDDEVKVEVHVQVHFHQKTREWTVDITATFVFRKDDGDYYLIEKHEQRDNKG |
| 795 | ADDEAERQIREWLKRFYEALNRGDIDAVREMLDDDWRYEDAQRGQTYDKDQFIEMVRRMQSIPFQIDITHIEFKRSSDTDITVTVHVQLHVHHKDSTHTVDATDHHHFRNEDGRWKIVRTQTHRDNKG |
| 796 | ADKEVQEAIKDWLKKWFEAFNRNDFDAIMSLYAPDATVENSGTNYEQFERWLREFLKNFQNSSLHVESVEIRLHPDKKHATVHVTVQVTEHHGDQTHHFTYHYTFHFQRTKDDWKLKRQQTQPDTG |
| 797 | PSEEQKKAMEEWVRRYIEALNKRDADKLVSLYDDDATIHVNSSNKEYSKEEFKEWVERMMSIPFRVQLDRIEVELKPGGEITVRVHITITFTRNGQNYTFHGTQTLHFRYDESSWKLVRHEITYNQD |
| 798 | PSPEDQEAIKQIKRRLVEAFNHKDIDKLSELVHDDSTFHFYTSGTTLDKEQFREWVERFMSFDFRIQIKSLEIRLDSGGEIRVTVHFDLTMHHNGQQHHVHVTAHDRYRRGDNGWKLVESRIRFDPD |
| 799 | PSDEEIAKELVRRYVEAFNKGDIDKLASLYHPDARMHDSSKGTTYDREEIIEMLKELIKRGVKIEVKSIEVQTHNGEVRVTVHVTLTDDTTGQTFHVTRHIRFRKDNGDWKIVEDTFTTDTG |
| 800 | PDDHEQVRETDERRIEALNKKDMEKIKDLIHPDATFTNSSEGQTYDREEYIERVKEMLKRGVKIELKEIHVEIRDGDATVHAHMQVTHDETGKTFNVTYTLRYRKSNDKWYLIEVRVTVDDG |
| 801 | PSDEEAVRETDRRRMRAFDEGDMKKVSELLHPDVTFHFDSENRTYSRDEFLEWAKQFISQGYHHEIKSIEIHVKDGTVRVHVEIDQHDRRTGRTFKIDYQVTYVKNNDEWKLVELRYRRDEG |
| 802 | PDDKEQIEEVDRRRLEAIDKGDIDAVASLYHPDATVHYSSQNRTYDKEEFLKMVEEMASQQTAHEIKQIDIQTKDGTVTVTVHVDVTFANTKTTVHVTYTIRYDKQNGEWKIVEVRITTDTG |
| 803 | ASEEEAKEQIRQFKRALVEAINKGDWEALERLITPGATFHHQSGETYDAEEWLEMVRQTIEKFPHRVEIKELHIEIHPSGTEAHVTVHLQVHFTHNGKHETYTFTYHDRYRFKDGRIYLVSSQVHEDSN |
| 804 | MSEKELEEAVRETLRRYYEALDRGDMDELRDLVDDGWTYHHASGETYSAEEFLEMVRQYTETFPTAHQIDSIEIHIDPSGTEVTVHVHVTVHTTHNGRTRTVRVTDTHHLRFKDGSVKIVETHTQEDST |
| 805 | SSEKEIREAIRDIIRRLIEALDKGDWEALDELYADGATFHHWTGETYDKEEFIEMVRRFTERVETAHQLDSLEIHIHDNNRDVTVEVHVTVHENHNGQTHTRTYTSTFHLRVFNGEWRITSEHIQTDST |
| 806 | MSEEEIKEAIKEILRRFFEAWDNKDMDTLRELFAPGATFHEYNGQTYDYEEWVERVEQTLRNVDTAHQIESVEIHIHPNNTTVHVTVHWTAHRHHNGQTHTVRGTTHFTLRRENGDLKIVEMRTQEDST |
| 807 | MSDEEAKEQIKRFKEQWVEAWNKKDIDAIKELVTPGAQFHHQSGQQYDAEEYIEMVEEILKKFDATLEIKSMEIQKNDNNKEYHVTAHIQAHFTHNGSHETYDFTYHDVYRFENGQLKLVRTETHTDST |
| 808 | TSDKEAEEAIRELVKRYAEAMNKGDIDTISSIYAPGAQYHNWSGQTYDYEEYLEWVKEFRSKFDFRLEIKSLEVHIHPNKRTVTVHVHITVHHTHNGEQHTTHFTSTMHFRNQDGEFKIVEEHEQTDSG |
| 809 | PSKKDQEDIKEWIRQFYEALNKGDADAVAELYADDAKYTNNDTQRTFDKEEIREMVEELFKNNTHFEVKSIEIRMQDENTFEVTVHVTVHTNHGNQTHTNQYTQHFRFEKHNGRWYLTSDQTQSDGE |
| 810 | PSKEDEEEVRELIRRFYEAINKGDMDKVRELLTPDIRFHNRSTQQDYDLEQFLKHIEELIKNDTKMEIKSLEIRQDDKDQITVTVNVTATTNHGDETKHRHVQHHWFLRRENGSWKIVEVQVQTDGS |
| 811 | PSKEQQEKIKEWLDRFYEAFNKGDMDKIRELVTPDVKFTDEATQRTFDYEDWLKRIEELHRSDVHIEIKSIEFRVDSKDEVTVHVHLTVHQNRGDQTHTNHVTHTWFFRFENGSLKLVEVRVQFDGT |
| 812 | PSKKDEEDIRDFIRRYFEAFNKGDLDALYRLYAPDATLHIESEQRTYDQEEIREWYERVLRQDTKIDIKSIEIRKVDDHEFTVTVHVQAHTNRGDQTHTAHVTFHFFFRKENGEWKLVEVRIRTHGS |
| 813 | DDQEDKEAIEELIRRWFEAFNKKDWEKVASLYHDDARVHFASHGRTYDKEEFIEWVRTMLSFDVTLHVKSIEIHFSGDTVTVHVEGDATFHHNGQTYHVTVHSTFHFVKYGNQWYLTEQHIQFDQT |
| 814 | DEPEQRKKIEEVVRRYFEALNNKDVDKVLSLLDDDWRYHNESTQRTYSKEEFREMVETLLSLDSEHRIQSIEIQLSGDEATVHVHVQVHTNKDGTTHTVNVTETHHLRQEGSQWKIVETRVTYNPT |
| 815 | DTPEDQEAAEELIRRFYEAFNRGDVDTAASLMDDDWRYHNNSEGRTYDKEEFKEWVEQLLSQDTHLHLKSIEIQTSGDTYHVTSHWDATFNRNGQTFHVSTTDHHRLEREGDQWKIVETRVTYRQT |
| 816 | PSDEEAVREVIRRFYDALNRGDWEELRRLVDKDNWTMHNNNQTFKTWEEFREWVERWFKNAQVEAHIRRIEFNGSTATVEVEVTYHHGDQTYHSRDTHFLQKRGDTWKMLETRIQYQE |
| 817 | NDAEEQIREFIEREAELLNRGDIDAWADFLFDDDKSTFHYNGQQFTTKDEIKEFIKQMVSNSHFELHIDRIEIKDKTTVTVEVTIHITHDNRHRTVHTTFFFRVENGSVKLTRKETTTHQQ |
| 818 | SDLQEAVKEFIKREAELLNRGDIDAWVEFVFHDDHSQFHVDGQTFNSKDEVKEWYERMVRNAEFEFHIDRIQVDDKDHVTVHVTVHVHTENSTFDVHVTIVLQFKDGDLKILSKHVHRQQQ |
| 819 | ANQEQIKEWKERFVEAFNKGDADKLAELFHPGVQVHFAQGRTFHDKDEIREYLERLARHFEMHMEEIRIQIDGDTARVEAHVRITDKRTGTTYDVTYHDVYRIENGQLKVVRSEERTRST |
| 820 | ADQEQIRQIVRNLIEAINKGDLDALMSYYHPGSEVHFSTGRTATDQEEIKDWFEKFARFVQLHLDSIEVHTHGDTATVHVHVTVHDRRTGNTYKFTSEIHFTRQDGTIRLTSEHVHMQPS |
| 821 | LDKKQIKEWVEQYIRAMNEGDADTLASLYTPGATVHFSSGRTFTDQEELRQWYENWARDFHVQLDSVEIHIHGDTATVHVHMTVTNDKTGTTVRVTMTIHFQVENGQLKVKEVRITFQPT |
| 822 | PDKEQVKEWVKQYIEALNNNDRDTLSELYDETKASMHVNGQQYDFDQIVEWYSRYLSQVTQFQLDSLEIRIKDGTVTAHAHVTVHVKHGNQTYRMTATFHFRYEGNTLKLIRVHYHHHQEN |
| 823 | PEDKEIAEEWIRRMIEALNKKDADAVASLYDSDEATMHIDGQTFTTHEEIKRWIQEWLSKFDVQIHMESLEIQIHGDTVTVHVTLHFTDNKTGITFTVHRTLHFERRNGTMYLVEDRLHRQSD |
| 824 | PEAEEQVKEAVERFFEAINKGDWEALERLFADNKATIHIDGQTFTTAEEFIRWFKEFRKKFDVTVQVDEIRIEIRGDTATVTVHWHMTDKSTGTTFRVTTHIRLENEHGTMYIVEMRIHTQDS |
| 825 | PEQEEKVRQLKKRLIEALNNKDWKTFADLFAKDEATFHFDGETLTSKEELIRWFRDFLEKFDVKITPEEIHIELDGDTVRVTVHVQFTDDSTGTTFTADQHDFYVNKNGTLYIVRVEETTKDS |
| 826 | PEFQEQVEEWKRRFIEAWNKRDLDTLADLFDKDKATLHWDGQTFTTKEEIKRFLEEFLKKFDTHIHMESFEIRLKGDTVEAHFHYQGTDNKTGVTFTFDTTDVYVNENGGMKILETRVHTKSD |
| 827 | PEDKEKIKEWIRREIEALNKKDLDAWASLFDKDESTVHIDGQTFTTKEELKRWWEEFLKKFDVHFHLESIHIEIRGDTIHVTVHVQVTDNSTGTTVTFTAHIRFERQNGGWKLVEKRVHTHNS |
| 828 | PEIKEQIKELKKKFVEAFNKNDVDALKELFDKDKAQFHFDGETIHTADEFREWVERFLRTFDVQMQMDGIEIHLDGDTATVHSTFQVTDNSTGTTFTVHATDHYVRENGTLKIVRTQFHTQSD |
| 829 | PDIEEAAREIVKRLFEAFNRGDWETLVRLYSDNKSQFHYNGRTFDTWEEFVKWVKEFASQWQLHVDSQEIEVKDKDSATVHVHVTMHHKTKTVKFTSQFHMRREGNEWKVVREHVQTDGT |
| 830 | PEEEMQKHIKEYVERYYTAINNNDLDTLESLIHPDWQLHNSNGRTWNDREEFKKTVEELFRRNIRIEVKSIEIHIHDDGSVTVHVQVTIHHEGHTTVQTRDTHHFQREGDKFKLVETHIQYIPT |
| 831 | PEKEAKEEIKKMIERFFEAFNKGDLEKLHRMLHDDWQFHLWNGQTFNDRHEFAEWVRQMHEAGVKMEVKSIEIETNDDGTYTVHVHVKVHTPGNKTYDVTETHHIERHGDQFRFRETRVHMTPT |
| 832 | DDEKEMEEAIRQWLRRYVEAFNNKDLDTIKELFDEDWTYHHTNGQTFTTLDEFEEWVERFLKRFDFEMHITQIDIQIFGKETVTVTMHVKVTFRETNRTFDITEHHHFERKGDEWKIKETREHRQPT |
| 833 | PEEEEMKKQIREVLRQFYEAFNNKDLDALSSLVHDDATFHFSDGSTLHTKESFRDWVEKFLKRFDFRIEITSIEIRIHGTHEVTVHVTVHVTFNKEKRTYTVHETHFFRRDSTEWKITRTQFNWQPD |
| 834 | PDPSAEEAAKQIVEELVRAYNEGDMDAFEEAYHPGATMHLPNGQTFTDLEEIKEWYERFKRTFPSRLEIKSIEIRQDSETTYTVHVHVQMHRSDGSTFNFTSTMVFVQKNGSWKIVEEHIHTQST |
| 835 | LDPEDEEAIRQMIRRFAEALNTRDREKLKDLYDDHFTMDLGNMDAEQIKKWLEEFLERFKTFRIQIDSMEFEKDGDEVTVHVNVTVTHGSTGRTFEVTRTFFFRKTPDGWKLTNDHVHTRERK |
| 836 | MSEEELREEVKEWLRRFYEAMNNNDLDKLRELLHPDWQHHDYSSGKTHHSREEFLEWVKQFLSQGWELEIKSIEIEVNGDEVTVHVTVQAKHPSGSTLTFHDTHRFEFRNGQMYLVETHTRVQQT |
| 837 | PTGEEEIEKIARRLYEALDKNDIDTFVSLFHPGATIHFSKGETLTDPDEIRKWFAEWLKSVKSTAHEIKSIEIRGDTVTVHVETHLNHGDKTHTISVTDHYHFVDKFLTNVQSHENPT |
| 838 | PSKDAQEAARQWVRRYFEAINKKDLDAFASFYHPDVRQHVNSEQRTFDREEIVERFKRYVETFSSFRIEIKHIEIEVRGNTVHVTVHVTVHHPQTGTTYDVTRHFTFELEGDSFKLVRDEVQTHQR |
| 839 | PSPEQEKAARELVQRFYEAINKGDWETAVRLLTDDWQFHDSSGQTLHSLEEFREWFEKWHRAGFHIDITTIEVRLIDKDEIQVHVHAQVHSRSGRTTFNTHDTHHLRLFDTDWRIVRTEVRMQPT |
| 840 | SDEEDERKIRDILRKYFEAFDKGDADKLKSLYDDDSTFHDANGQQFTDKDEFKEWYERWKSQFPTVHREIKSLEIRFDGDTVTVHVHVQVHVHHESSTVTIDVTATFHFERENGDWKIKEVRVHWDEKRQ |
| 841 | TGEETEEAIREFKRKFLEAFDNKDLDKLKEIIDPDSTFHFDSGFTATSLEEVVEFLERLLRTYKDTAHKIDSMEIQQDGDEVTVTVHVTVHVNNDTSTITIHITAHDRYRKKDGDYKLVETHFHFDQRQE |
| 842 | SGSEDEEKIREWKRRFIEAFNNGDLDKLAELVHDDATFHFSEGYTATNRDEVKEWFERVMRQFPTMRMEVDSQEIRVSGDEITVHTNVTVHVTNETSTTTIHVTTTDRYEKKDGDVKLTESRTHFDQRQS |
| 843 | PGEETMKREVKKWVERFYELFNRKDVDALASLYAPGAQFHDSSTGTQYDREEFRRWLEEFFRRFPNVQIDVQRIEIQIHSGNEATVTVHAQVHFKDSNYTVTVTQHFHFRYKDGTWKLTEDRMHFDQDRS |
| 844 | PGEETAKEEIKKILKRFYDLMDNGDMDAMASLYHPGARFHDDSKQQTYDLEQFREMVKRFLERFPDIQRHIESIEIQIDSGDEATATVHVTLTNKSNNQHVRVTQHFRFRYKDGTFKIVEDRIRTQNQNT |
| 845 | PGEETIKEVIKEWLRREFEALNNKDIDKWKSLVDPGVTFHDDSKKQTYDVDQVAEMIKQWLETFKDVRIQVDEIRIQIDSGDEAHVTVHVTFHYRSSSTQVHVTYHLTFRYKDGTWKLLSKRAHTDNQNS |
| 846 | PSEEEAKEQIEEWLRRFYEAFNQKDLEKLKSIVTPDATFHIYDGRTFTTLDEIAEYIKEMLSNESKIHIKSIEFHVNPDGSITVTVHVEVHTSNQNQTYRFTHHWHFKVDHDGQWKLVEVREHVDQHS |
| 847 | PSAEEAAKEIARRLYEAINRGDWEAVRSFYSDDATMHVSEGYTYKSWEQIVRWIQEWFKRDWQIQVHSIHVEVKHPDRFTVEVHAQATNSSTGSTVTITQTFHFQRKDGTWKIIHDHMHIHQQ |
| 848 | PSDEEIIREILERFYEALNKGDWETLSRLLDKDATVHISDGVTHTTLEQFREWVERFHSSGSKIHIHSIRIETKDKDSITVTVTVTVKNDQTGGTVTVTDHYHFRRENGSWKIVEVHSHFHQD |
| 849 | PDIEEQIREIKRRLIEAFNKGDLETLKKLFDKDATMEISDGVTFTSIEEILKWFEEMNRNNYKLEIKSIHIQVDSKDTVTVTVHVTLQQDDTGRTVDVTFHDHYVFENGELKILRSEIRFHQR |
| 850 | PDAQEAVKELVERFIEAFNKGDWETLKEIITDDWQNHDSSGITFTSAEEFREWVERILKDTRQITLKEIHIQVEHDDHVTVTVHIEQTNSKTGQTFTATEHHHIQRRNGSWKMTETHVQFHES |
| 851 | PSEEEIRELVRRFLEAFDKGDADALASLYHPDATLHVYNGRTFTTRDEIKEWIERWFKAFSDVHREIKSMEIRGDTVTVHVTVHAHHKDKTVTYNFTSHFHFKDGKLISEEVHVDNTES |
| 852 | PSIEEAVRDWIRRYFEAIDKKNLDELIEMFDEDWRYHLNGQRFSLEEFREMVERFLKSFPRVHHEIKEIEVHITKDHATVHVHVQVHLHQGDQTITIHVTETHHFRQENGSVKIQETHVDTKPS |
| 853 | ASEEEAREWVKRYIEAFFNTKDAETAASFYTDDAQVHHSNGQTFDNKDEFKEWIKEWAKRAKRVHFQINSIEVNGDTVTVHITVTFHHDDRTTTTDFTSHFHFKDGKLTSERVQTHTHNE |
| 854 | NPDEEEARELVKRWADALNRRDIDALKELIHPDATFHVDSETYDRDEYIEWFYKQFASHVTRVELKEIRVEKRGTTIHVEAHWTVHETHGDKDITVRVTTHFVFQKDHDGRWYLVELRIHFDHS |
| 855 | DPDKEQVREVKKRFIEALNNKDLDKVASLVHPDAQFHYDSETFNYEEFREWYFKMIASHAERIEIHEIRIHVSGDTVTVHAHYTVHFTHGDQKRTVHITSTDHYRRDHNGQFYIVRMETHADHS |
| 856 | DEKEARDAAREVVKRFAEALNHRDLEKLKSLYHEGATITFNGRTYRNPSREEIAQWFASLPIYRIQWQIDEIRVDGDEVTVHATVHVTHRNGNTVTFHSTFFLRRKDGEWKIVSEKVDRRQS |
| 857 | DEKEQEEQVREWIRRFAEAVNNKDLDTLAELLDKDWTVHYNGETYHTPSKEEFKEWLSSIEIKRLEVHIREIEIRGDHVTVHVELHMTTSSGKTVRFEETHHFTVKNGELRLTRTHITTYND |
| 858 | TSEEEAREIAKDIIRRLNEAINNNDMDALRELYAPDAQFTDASTGQTFDLEEFIKMIEEMLSSGTRIEFSIEEIRVSNNHVTVHITVHVHTGHTTQTVRRTIHFRVKDGEMKIKRDEINRNK |
| 859 | SSEEEAREAVKRLKTRFVDAINKGDLDAIRELFHPDVTFEFASEGQEYDLEEFLEWVEKMLSNGTRIEFEIKEIQLSNNHVTVHVTLHFHTKDRTQTVHQTDHYRLVNGEWKIVRVHIDRQP |
| 860 | MSEEEAKEAVKDWLRKYFEAINNNDLDALREMMAPDWRYHNADTKTTLDKEQFLKWVEDVFKNRTRFEFHVDEIRLSNNHVTVHVTVHLTTDDRDQTVHETHHLRYKDGTWKIVRTHVQTDQ |
| 861 | YSEEEAQRDVRELVERFYEAINKGDMDTMESLLTPDWRYTDDSTGQTYSRDEFKEMIEKMLSQGTKIEIHIREIHVGKDHVTVHVEVHFHTGDRTQTVHDTHHFRLVNGKFYLVETQTNRNT |
| 862 | SSEKETEEEVRRLINEFYDALNRGDIDALREILHDDVQFTNESYQRTFDKEEFLDWVRKMKSRNTRIEWEIKEIEISNNHATVTVEVHYHTNDQTYTFHDHYHFRLSNGEWKIVRVQSRSDQ |
| 863 | DDEEMKEQAKEIIRRFYEAINKGDLDKVREILDEGWRFHDASKQQTFNKEQFLEMVEEMVKNNIKIEIHFKEIQIDDKDEVTVHVEVHIGNVTTTDTHRLRRTKDGWKIVETRFQVHN |
| 864 | DKREDKEAIKEWVEREARAFNSGDMDAWYELFSPGAQFTNADTNQTYDIDQMKEILEKLWKAGFSLHFEIKEIEVKDDHEVTIHVEIHWRNQTVTYTDRFERDKDGWKITHRRIQTDN |
| 865 | TTEDEARKAVRELLNRMYEAMNQKDMDKIYSMYAPDVTFHHHNGQTYDLDQFKEWVEKFLRHFELHWEIKEIQVHNGTVTVHVHVTAHTNGRTLHITQTFHFEFRGDEVRVKEDHIHEQS |
| 866 | SSREEAEEIARQILNRFYDALNKGDMDKVKSLYHDDATFHHNKGETYDLEQFIKWVEEFIKHWELHWSIEEVRISNGEVTVHVHVQMHGDGETIDVTQTFFFRREGDEWKIVRDEIREQS |
| 867 | PSEEEAREIVKEFIKREAELFNKGDMDTWYSLFHDDFRLHHASGQTYNLDQVLEMIERLARNFKMEFSIKRIEVHNGEVTVHVEVHFHTNNKTVTVEFTDRLEMHGDTVQVTHRHVREQE |
| 868 | NSREDAEEAVRQLIEREVEAFNKGDMDTWEDLFHDDFRFHDSNGQTYDLEQLKKWVKELAENAELHFSITRIEISNNQVTVRVHMTFTTHNETRTITYHDHIRQEGDEWKIVRRQQQPDS |
| 869 | PEEEELREKVKEAIERMARAWQHQDLDKIKSFYDDDSTVWYNNIDAEEFERWVEEALRRFKDARVEFHIDEIRVTDPTHVTVHVTFHVTTPSGQTHTSTRTFHFRFEDGQIKLVEDHVYNQQT |
| 870 | DRREEIKEKVKEWLSRFYEALNRGDLEAIARMVDPDSTFHSDNKDWEQFVEWVERFLKRFPDVRFEWEIKHIQIETKDHVTVHVEVHVTHKDGSTQTSRETHTFEFKDGRLRLVRTKSDNQNT |
| 871 | PKEEELKEKVKEAIERMVRAFNHGDLDKILSFYSDDATVDTNGESLEQFKEWVERFLKTFKDGRMTFEIKHIQIEHETQVTIHVEFHFTTPSGQTQTSRRTFVFRFEDGQIKLARDKFYNQQS |
| 872 | PSEKELEEQVREWLRRFFEALNRKDVDEVASIFDEDWRVTHHTQTYNLEQFKEWVRQMLSQVESIEWQIDEIQFHGDTVTVHVTVHIHYTGKTVTVRETHFFTVENGSLKIKETRIQEDEVK |
| 873 | MSEKELKEAVRRWVERYARALNNKDLDALASLYDEDATMHINNQEYSYEEIIRWYKEMLSQARRFQVHIDRIEVNGDTVTVHVTMTIHYQDKTVHVHVTFFFKFENGELKLKRVEFHEHEQS |
| 874 | MSEKEMKEAVKEFINRYYDAFNKKDADAIASFYDDDATFHYNDTTYDKEEFKRWVTEFLKNVERVEWQIDEIEVNGNHVTVHVTATFHHPGKTVHVTQTFHFTFENGSMKLKEDRLHEQEQK |
| 875 | MSEEEAKRAARELIRREFEIFNSGDLEAWASYFHPDARMHIHDQTYSWEEMKEWFKEMQEKVERIEVEIKEIEVRGDTVTVHVEVTIHHPNKTVHAEVTIFLRIENGQFKIISKHIRTQDQS |
| 876 | PSAEEAARDLVRRWAEAFNNNDLDAVASLYHDDARVHHQNGQTFDKEEYKRWVEEMFRRAKQMEWEIKEIQVENGTTVTVHVTFHVRAYNITFHMTFHFRVENGQWYIIEQHISTEN |
| 877 | PSDEEQIRDFKRRYAEAINKGDLDALRELLHPDVQFHHSSGETYDLEQWKEWVRQFRSQAKELEVHIERIEIRNGTDVTVHVTVHAKATGTTFHATDFYRKKDGSWKIVEVRLHTQE |
| 878 | PSDEEAVREWKKRFAEAMNNNDLDALAELLHPDATLEHQNGQTSDREEIKEWAERLWRTATSIQVHIDEIQVDDGERITVHVTIHFKSRNVTVHATDHYEKHNGEYYLVRSQFHEQD |
| 879 | TSEEVERKIREWFRRFVEAFNKGDMDTLAELYAPDARMRAYNLDKDDIKRWFEELFKNDVKVEFEIKEIQQDGDTATVHVTFHFTDSRTGKTTTFHSTFFFEFQDGELKLKHEQVHFSDQ |
| 880 | PKDAEQKIREWLKRFYEAFNRGDADALLELLTSDSNVTFHEASGRTFTTLEEFREWVEEWLRQNWKTHIKSIEIRPDSKDEYTVHVHVEVRGTNQTIKVTETHHFRFENGSVKLVETRVRWDHG |
| 881 | PVSEEEVREAVKRFFDALNRGDWEELLKLVDESQWRFHNADGSTLTTKEEFRKWVERLFRENWQFEIKSIEVRITGEEVTVHVHVQVTQRDTGKTFRVTETHHFRRGKDGWKIIRTQVHVDPQ |
| 882 | PEEDEIRERVKEFIRRFYEAFNRGDVDALMSLIHPGATMHIDGQTMTSMEEIKEWFERFFKNGFKDAKLDIDEIRIQINSGDTVTVTVHVTITRQNGETYHVTNHDRFKYDKDGLKLVHHQAHTDN |
| 883 | PEEDEVREAVKEFLRKFYTAINNNDLDAIASVLTPGATMTLNGRTFTTKEEIKEWFRQFFERGFKDVKLQIDSIHIQINSGDTVTATVHVTVTQPDGSHIKVTHHWHFKYDKDGFRIVNVQEQSHN |
| 884 | PEEEEIKEAVKEIIKRFYEAIDNNDLDAIKRLYHDDATFHDSEPRTYHSKDEIKEWFKRWFESGWTDSRHEVHIREIRIDDKDQVTIHVTVHVTNADGSTTTVHQTFFIQRHNGEWKIIEDRVHTES |
| 885 | PEEDEIRTQVIEVIRRFYEALNHNDLEKIKRLMHPDATMHIFDGTTMTNLEEIIKWLEEWMRTFPDFRIEVHITEIEIRGQGTVTVHVTVHITNKDGSTQTFHHTWFLTQHNGQWYIVEVREHRDE |
| 886 | PQEEEMKEKVKDWVKKFAEAFNKGDADALASMYTDDSTMHISDSTTFNNREEIKRWFQKLMSQNIQMHVEIKHIQIHGRTHATVHVTFHLTHQNGQSVTIHSTFFFVFENGQLKLTNEQIHFQQN |
| 887 | MSEQEFEEIARDYVRRFAEAFNKGDFDKLKSLLDDDWTFHHSSGRTFTTKEEFIEWVRQFLSNGFQIHFEIKEAQLHGTNHVTVHVTFHFTSKDGQTHTFHETHFFRFEGDSVKIVETQLHTDH |
| 888 | ASEEEMKEAAKEIIRQIFRAFNEGDMDAIKSYYAPDATVHIWNGETLTTQEEIVKWFERLFSQNIEIEVHIRRIEINGTDTVTAHAEVHMHHPSGQTYTYHSTFHLELKDNRFFVKEEHLQRDH |
| 889 | MSEEEMAEAAKEIIKQIVDALNNGDMDAIASYYAPDAQLHIWNGQTFHTLEEIKRWFEELFKQNWEIEVHIREIRINGSTHVTVHVELTFHHPSGQTYRFESTIHFEVVGDRMYIKSEHIQSDH |
| 890 | PSEKEQEEAIREWLRRFYEAIDNGDWEAIRRLLSPDATMHVNGRTFTNLDEIVEYIRRWAENIQTAHEVHIREIQFHGNEVTVHVTVHATNRKTNSTITFHDTYHFRYRDNSWFITEVRSQETT |
| 891 | PVGREAAEKFVRRLYEAFDKGDRDTFDSLFDDDWTFHDSNGETITTKEEMKKWFDTMFKQLGDTAHEVHIKHVEEKDNGRVTVHVEVHQHIKKTGTTRTVTDTHHFQYKPTGVYVVETRSQWHN |
| 892 | SSEEEIKEAAKQIVRRLVEAFNHNDLDKLREIYHPDAQMHWNDQTFNSLDEIVEWIKQYLQRLRGSRIEFEIKEIRIHGTDEATITVQFHVHYSGENQTTTYHSHMRIRLEGNKWKILSEHVHEDST |
| 893 | ASEEDQQEIKEFIKREAELWNRGDLDTWKELFHPDATLHDSSGITFTDQEEIHKWLEEFLKDNFKDTKIEIKSIEIQADDKDQVTVHVHITVHINGQTFNVTQTDRFRKDGDRLKIVNRHVTIDNS |
| 894 | PDAEEDIKRLKEEFVRAFNTDLDTLKELFHPGVTFQSDNITFTDRDEIKKWFADLRSTMTVQVDRIEIQKNDDGTVTVTAHMTVHTPTQTFKVTAHDRYQLTDGRWRLVSSRFHRHDT |
| 895 | PSDEEDMRQLVRRLIEAFNKDVDALFEIFHDGWRFHWNTQTFTDREEFREWVKELQSTTHIEIKEIRVEKKDNGEVTVEVHVTFHTPTQTFRVTETHHVRKEDGRWRFIETRVQTHSS |
| 896 | PEEEEMKEKVKEWLERFYTALNRRDADAVASLYAPDSTFYVSRGETLTNPEEIKEWFKRFLDTFPDLKIHFTMEIHIHGRTTATVHVNMHVTHPSGQTYTFSQTFRFEFHNGRLKLTEDHLQTIQT |
| 897 | PQEKEAQEKAEEIIRRFAEAFNKGDLDAFRELYHPDATMHVWEGRTFNSIDEIIEFFERVLRTFKDLEMHVTIEAHQSGDTHYTVHANFTVHQPDGKQVHYHSTFRLRLKDGEIKILEEHISRHQT |
| 898 | PEEDEMKEAVKEILRRLFEALNRGDAEAIARLLDEDWTFHDAEPRTFTSRDEVKEWVKEMFKNDFKFHVESIEIQIYGRSTVTVHVHVTVTNKNGQTYHATETHHIRRKDGRFQIVETHNNWQPT |
| 899 | PYEEEMKEKIKEWVKREIEALNKGDMDTWKRLLDDDVQYHDSQPRTFNSADEMKEFFKQLHDNNFRLELKSLEVQIHGHSEVTVHVTVTVHHSSGQTYHNRYTDRFRFNNGELKLVERQAYWDST |
| 900 | DVSEEEAREAIKDWVRRYAEAFNRGDLDAFEELYHDDAQMHLSNGQTFNNKEEIKKWYEEFFRRFPGTQLEFQIDRIEVGHPGTYTVHVTFHFHHDSGKTTTVHATFHFEFRGDQMYLISVHYHTDNYP |
| 901 | TSEEEMKEMAKEVIDRLFEAFNKGDWEKVKSLLHPDWTYHHSNGQTFTTRDEFAEWIIKFWSQGWRIEWEIKRIQIDDGKEVTVTVQVHYHHQSDKTTDAQEHHRLRVDDDKEVKVVETRVHTDEKPK |
| 902 | MSEEEAKEIVKDLLRRMYEAINNGDMEELKRLFHPDWQYHQNKGRTFTTLDEFVEWAAQFWSNKWQLHWSIKEIRIHNGREIEVHVEVHITHESDRTITVRETHRFRVDDDHEVKIVETHTQIDEQPK |
| 903 | PGSEEEIKDLKRRFAEAFNNKDAETLASLYDEDVEFTEDKETYKLSRDEFKERWEEQMKKQFPDLQVQIDSIRIENKNGDEVTVHVHLTMHVSDGQYHFTQTDVYRYQDNTWKIVRTQSHTRGG |
| 904 | AGSKESIKEALKRYFEALDKKDADTLAELFDEDWTDTQDDETYHFSREEIREWFREFQSKHFPDSRHEIKEIRIQENDGERFTVTVHVDVHVKDGTYKVTEHHHVRVEGDKLKFKETRSHRQSS |
| 905 | PGSKESIKEWKKRYIEAFNKNDADKLADLVDEDATFTHSNETYKLSKEEYKERVERIFKTHFPTAQIEVKSIEIENKDGDQYTVTVHVTIHVKDGTIRATQHDVYRFEGDQLKLVRTQEQHHGS |
| 906 | DQHAEEAAREIIKRFYDAFNNGDMDTLKSLYHPDATFHDWNGQTFTSRDEIKEWLKQLFDNGWRIHFSIKEIQVHGTDEITVHVEVHFTNTNSGQTYTATQTFRFVLQDGKWYLVEDRLQRTG |
| 907 | DKDAEEAARELVERFYRAINNGDLDTMLSLLHPDWTYHDSSGRTFTSREEFAEWVEKLFRNDIKIEVHIKEIRVHGTDEATVHVEVHFHNTNSGQTRTTRDTHHIRLQNGQWKIVRTEVRWTD |
| 908 | DQSDEEAIREIIKRFYDAINNNDPDKLRSLYHDDATFHHANGSQLDDKEQIVQFFAELAKQGWQLEFSIKEIQFFGTDTVTVHVDVRMTQTNSGRTTTFHQTIVITKINGRFFIVRDQLQETR |
| 909 | DKSMEEKAREWKRRFVEAFNKGDMDKLKSLLHPDATMHNADGRTFTNPDEIVKWFKELHDNGWQIEFSIKEIQIHGRTHATVHVEVHFTNTNSGKTITVRVTDRYVFKDGQIYLTESRFHFTD |
| 910 | DQHDEEAVREVMDRLYEAINRRDLDKAASLFHDDWTLHHANGQTYTTIEEFKDWVKKMLDAGVEIRWKIDEIRVFGNTEVTVHVTVHMTHTNSGQTQTFHDTHHLRKINGQWYIIETRVQETG |
| 911 | TSEEEIREWKKRYATAFNNNDWDKLRELVHPDAQFHFDEGTTFDNAEEYIEWLRKFFTTKHQIRFQIHIEEIRIHGDTVTVHMTVHGTHKSGQNYTWRVTVHDVYKDGKLTSTELHTDQHD |
| 912 | MSEEEIKEILRRAFEALDKGDMDKIKSLYDKDAQFHFTDGITIDTLDEFIEWFVKFFLANSEAQYSHHVEEIRIDGDTVTVHFTVHVTHSSGQTITFRATSHFEFRNGKIQSEREQMHQED |
| 913 | PSEEEIKEFIKRWAEALDNGDRDKLKSLYDDDATLHVSEGYQMDNKEEIVKWLAEWYLKLFTDVKRQIDSIEIRGDTVTFHVTVHMTRPDGKTVTVHQTSHMHFRGGKLVEQHIQTDEDEG |
| 914 | PSEEEIKEWIRRYLEAIDNNDIDKLKELFTDDWEYHNYSGETVHNPDEFIKRWEEKFKKRVTRSKHDLDEVRIDGDTVTAHVTAHFTHKSGKDVTQHITEHHRFENGKLKETHETTHQDDG |
| 915 | SSEEEIREWLKRFYEAIDNNDMEKMLRLVDDDVTFHVYQGITFENAQEFIEWWAQSFRRRFTDSRHKIDEIRIHGQTVTVHVTVTLHHRSGRTFHRTVTEHHTFRNGKLVETHVHEDQKDN |
| 916 | ASEEQIKEFIKQYIELLNKKDIDAIASLYHPDAKLHDSDGSTYQNRDEIKEKWFKEFFESVDEMHIEIKSIHIDGDTATVHYQVHLTRGDRTHTYDVTDHFRFKDNKITEKESRMRDTTS |
| 917 | PGAEEAVRQVIRRYVEAFNNRDMDTLRDLYAPDATYHNAKRQQTYSYEEFLQMVREFLEKFDFHLTFRIDEIRVTGDEVTVHVTFHLRRNDHKTEHQVHSTFHLRRKDGTWKIKEEHAHFDDK |
| 918 | PDAEEAIRQLKRRFLEIYNTKDLDALRELYHDDVKFHNAKRNQTYDKEEYLQMVREFMEKFDFKIEVQIQEIEITGDEVRVTVEVHITREREGRTFTVQYHDRYRFENGTVKIISTQAHYDEQ |
| 919 | PDAEEQIKDFKRRLAEAFQNKDWEALASIFDDNVTFRFASTNKDYDKEEYIQMVREFLEKFDVEVEVHFQEIEITGDHIRVRVEVHFTIKKEKKTYTATYTDRYRVENGDVKLVSTEIREDSQ |
| 920 | PSDEEAIRQWIRREAEAFQRRDLETWASLFTDDSKVHDESTQTTFDKEELKDRVREFLEKFDFHVEVHIQEIRVTGDTITVTVEVHFTNNDEKKTHTVQYHDHFKKKDGQYYLKERHARWDSQ |
| 921 | PGEKTAKEAARELVERVAEAVDKGDRDTLRELYHPDATMHIWNGRTFKNLDEIIEWFTQFGSDLQRHFRVDEIEVNGDTVTVHVTFTVHHSDGKTIRFESTFFIERKDGSWKIVEERIHADSG |
| 922 | PGQETAERAVREWIRRFAEAINKNDMDALEELYHPDAQMHISDGRTFDSKDEIKEWFREMAENAQFHFSIDEIEVRGDTVTVHVTVQVHRRDGQTYTYHSTFFFRQEDGQFYLLEERIHNRSG |
| 923 | PGRETFERQVREWIRRFAEAFNKRDMDTLFSLFHPDWTFHDSSGRTFTSREEFKKWAEEFASHFQLHWEIKEIRVHGDEVTVRVEVHFTHRNGQDFTFEETHRFRLINGQLYLIETHLRQDGG |
| 924 | PGRKTMQEQVREWLDRFYTAMNNNDADALKSLYHDDATMHIANGRTFKSKDEIKEFLTRFASQFELHWSVEEIEVHGDTATVHVTVTAHHKSGQTYTIHQTFHFQFSDGRLYLTHDHIDNQSG |
| 925 | PSEEEIREDIKELLKREAEAFNKRDLDAWLSLFAPDATMHDQSGQTFTTLEELKEWVKQLLSQNWEVEVHIDEIEVRGDHVTVHVTAHFTNQDTKQTITVHFTIHFQLRGNEWKIVEKHISTRQT |
| 926 | VSEEEARERVKEWIRQWVEAFNKGDMDRLKELYHPDATMHLSNGQTFTTQDEIKEWYERLFRQNYRLEVHIKEIEIRGTTATVHVELHFTNESTGSTFTVHTTFHFQFEGDQVYLVEQRSHTDQT |
| 927 | PSEKEQEEQIREAYRRLAEAFNKGDMDAIKSLYHDDAQMHIYNGRTFNNKDEIKKWWAELFKQNWQLQIEIKQIDFNGDTVTVHVQFHFTNESTGTTMTVDATHRWVFEGKEVYIIEVHYHTRQN |
| 928 | PSDQEQIEELLRRFYERFAKQQDWKAFEEFFDPDAQYHNNNQTFHTLEEIVRFFKEMLERMQDVRFEVEEIEVRGDTATVHVTVHVTRNGKTITYRVTHHWVFKDGRVVEVREHTSEHD |
| 929 | SGEEDEEDIRQIIRRYAEALNRGDMDALESLYAPDATVHDSSGRTFTTKDELKRWYDEWLKKFPQRVEVHIKRIEFHGDTVTVHAEFHVTTKDGTQTTTATFFFVKDHNGQWYIKEVHWNSNG |
| 930 | AGEEDEKAVRDIVRRFYEALNKGDWEALASLYHPDAQMHLSNGQTFNSREEIVRWFREFLEKFPLQVHFEIKHIEFRGDHAHVTVEVHITTKDGTMTITQHITMTRDHDGSWKISEDHINFNG |
| 931 | SGEEQEEKVREVIRQYYRALNEGDMETLKELIHPDATFHFSNGETLTNLEEVIEWFKRVFERFPFRIHFEIKEIRFDGDTVQVHVEAHVTHKDGTQTFHDTYFFKFDSNGRVKIVEVHSNANG |
| 932 | ASEEEIRRAIEEMVRREAEAINKGDWEAWLSLFDDDATMHVSKGETFNSKDELRRWFEQLVKNNVKIEIHIIRIEIKDKDTATVTIEVHFTFTTYGRTITVNAHDHIVFEDGRFKITERHVTEQPN |
| 933 | DVSPEQYEKIKQFLRRFYEAVDKGDMDTFVRLFADDWTLHVDGRTFTTKEEVKEFFERFHRNADVERHIEDLHVEQKDGYVTVHVRVTVHYKESHQTHHYQETHHVRFDHNGQLYFVETHVHTEN |
| 934 | DASPEDEKEIREIIRRLYEALDRGDVDEFVSLFHPDAQFHSDGETIHTLEEYKEWVRRLHENADLRREIKRLEIEQKDGNATARVHVTVHYNESHQTHHYTHTWFFRRDHDGQWYIVEVRAQTHG |
| 935 | PSEEQIKDILKKFYEAIDNGDWKTLRELVHPDAQYHHADGTTFNNVEEMAREFAEKWFKNNDTAHELEEIRIDGDTVTVHYTLHVNRGDTTYTFRGTDHFRFRGDKITEIRSHIQQTD |
| 936 | ASEEEIRDLIRRFVEALNNKDRDKLKSLYHPDAEMHIYQGKTYTNLEEIITFFFEEFFSNNDAQIEIKEIRINGDTVTVHVTVHENHGDRTHTFDVTSHFHFRGKKLTEEHIHVQQTD |
| 937 | ADEEFQEHVKEFKKQLIEALNKKDVDKFVSLFSDDATVHYNNGETLTTKDEIREWVERWIKSFKDFKIEVKSIEIRGNTVTVHMTVHMTHDSSTHTVDVTSHDRYENGRLKEMEQRHNQR |
| 938 | TSEEEIREIKKRLLEALDNKDLDTFASLFHPGATVHFWDGYTAHDQEEIKEWFAQFFKKFDDDYRHEIKSMEVRGDTVTVHVEQHAHKRPQTFTITTHDRYENGKLKESRIHRNPQ |
| 939 | MSEEQIRDWKRRFFEAFDKRDLDALAELWHPGATVHFSDGTTVTDKEEFKEWFARFFSRFPHDFRHEIKSMEIRGETVTVHVEMHWTSHPKTQTITVHDHYKDGKITESRFHVNDQ |
| 940 | ASEEEIKEWVRRFVEAFDNGDLDTLVSLFDDDWTFHHSNGQTFTTKEEFKEWVKQFFEKFPQDLRHQIDSIEIRGDTVHVHVTQKHRDDKTTRKITETHHFKDGKLKETHQQHDDT |
| 941 | TSEEEIRELARRYYEAFDNKDMDALKEILDDDATFTNNSTQTTVDAEEYIEWMERWLRRFPRVRHEIKEIRIDGDTVTIHVQVHFTTRSSQTYTVDVTHHWHFRNGKVVRLQVQTHQRE |
| 942 | PISEEEIKEWLRQFFEAFNRGDLDTAVSLFHPDATVHVSDGITFTNLEELREYLKRLMDNNAQIEVKSIHISDGTVTVHWTMHMTHGDKTVTIRGTTHFHFRNGKLTEMHIHVDHNR |
| 943 | PSEEEIRDIKRKFAEAFNKGDLDTLKELFHPDATVHFYIHNTTAHSLDAFKEWVRRFLRIPFTVHIESVEINGSTATWHMTVTFHHNGKQHHIRYTVHDHYTDGRIRETHFHVDTG |
| 944 | PSEEEIRELKRRLVEAFNKNDVDKLAEIFHPDATIHFYQEPSTVHSKEAFREWVKKMARTPFRVEVKSVEFHGDTVTWHLQFTFTQDGKEVHIRVTVHDHYSDGQITETHFHFDEG |
| 945 | SDQEEIRQWVRRFIEALNNKDMDTIASLYDDDATLHVYNGQTWTTREEIKDWFRQLIEKMKDVRFEIDSIEVRGDTATVHVTVHVHHETSTHTFHITSQFHFKDGKLTEEHIHENDT |
| 946 | ASEEQIEELIRRFYRAMDEGDVEALAQLLHDDATIHFYNGITITSKEEFKEWVKRWQSQFQRIHRQIHSMEFHGDEVTVRVTLTVHRGNQTVHYRYQHHWRVQGNKIVEVEVRSDSQPT |
| 947 | AGTEKIKDLLKKMYEAFDNGDWEAVASYFHDDSEVHFQGGITIHSREEFEEFIRQVLSNIKSLQREIREIRIDPSTQRVTVHVELHWNLGRRTVTVTETHVVRGDQITHTHVQEDQQ |
| 948 | DSEFQQKVKDIKQKFIEALNNKDADKVASLFHPGAEIHVYQGITFTDVEEFRKWVEEFLRHWKDVKFQIERIEIDGNHVTVHVTVTFNHDHPGQLHFHFTFHDVYRNGRLVRTQINSQPKN |
| 949 | DREEQEEEVRKWKKRFVEAFNNRNWEEIASLIHPGSTFYHTGGQPSDKEQFVRWVKEMAERIDHVEVHIDEIQFHGDTITVHVTSHFHQGDTTFTVHQTDHYQKRGDTFRLIETHTHRDS |
| 950 | DKEEDKEAIKQWFRRYFEAMNNKDIEKIASLFSDGWTYYSPGGQPYDKEQFKEMVKKLLDQIKQIHWEIKEIRVDGNTITVHVESHIRINDKDITFHETHHFEKRDTQFYLIETHVQSKD |
| 951 | PSLEEQIREWLKRYYEAFNRGNLDELAELVDDDWQYHDEKTQRTFHTREEFREFVERVMRTFPTMQIEIKRIEINGNTVTVHVTVHVTHPNSHTTVQVTEHHRFENGKIKETHTREHQQD |
| 952 | PSWEEQIREWLKQFYEAIQNKDLDALARLIHDDARIHFESEQITLTTKEEIKEWFERFMKRFKNTHVEIKHIEIRGNTVTVHYHVTVHHDDRTVHFTVTDHFHFKDGKITEIRSRIEERR |
| 953 | DEEEETEEQARQVDTRRFEALNRGDRDTVRSLFHPDATYVDSNGQSYDYEEFIEWVKRHRSDQQHLEFTIKEVRINSENHVTVHGEVTDQRTGVSATYTAHYRKNGDEMKIIRLEVNNNN |
| 954 | DEEEKMKEIVRQIIRRELEAMNNNDLDAWLSLFHPDATYTQQNGQHYDREELKEMFERHRRYHSNIHIEIKEVHIDSEKHVTIHVQVTFNARGTTFTYTFFVRVGGDQAYIVRKHESNNN |
| 955 | DKDFEEAAKEVIKRMYEAMNKKDLDKVASLYHPDAQFHYNNQTFDYEEFREWVERMLSTVSNIRFSIKEIRVHGDEVHVTVEVHFDSDKRTFEQHITIVRTGSQWYIVEDRMQTIN |
| 956 | LNPEAEKAVRDTIRRYFEAFNKGNMDEIKSLFDEDWTVQDERTGTEYDLEEHLKRLKELLSNGTQMHVEIKEIRQSDSGEVTIHVTVHVTQDGRTFTVRETHHLRFEGETLKIVRTHLQFRST |
| 957 | PDPNDQEQIKDWLRRYYDALNKGDMDAIKSLFAPDWRDTDASTGQTYDLEEHLERMRQMTSRNVKVEVEIKEIQIDKDGRATVHVTVRMTENGQTFTFHETHHFQKHGDGFQLVETHVHSHGS |
| 958 | PNPEDIEAVIQLIKRMYDAFNAGDMDKLKSLFDDDWRWTDARTGQTFDLDEFLDMVRKMQSQDIKIEVHFKEVRQEKDGTIHVHVEVHVTRNGRTITYDDTHTFQKHGDSWKVVHTQTHERTN |
| 959 | NDPNDEEAVRQILRRLIEAMNKGDWETLASMFDEDWRFHNSKTGRTFDLEEFVRMVRTRHSRGVRIEASVERIEFDHNGRVTVHVTVHMTENGRTRTYHETHHIQKHGDSWKIVETRQTERGN |
| 960 | SDPEDLEQVKETDKRRVEAFNRGDMDTIKSLFHPDAQFTDDSTGQTYDLDEYIDRLRKLFSQGVQVEVHIHRVEQDKDGEVTITATVHWHRNGQTFTVRYTIRYRKTGDDWRIVEVRYQRHSS |
| 961 | TDPSAEEAIRQIIKRLAEAFNKGDLDKIKSYYHDDAQFHFSSTGQTYDLEEFLDMVREMTSRGVQVEVHVDEIRQQDQGEVTVHVTVHFTENGQPFTVQRTLHFRLDGDSWKIVSDHITHHST |
| 962 | PDPNDQEAVKQLIERFYEAINKGDLETLESLLTPDWRYTDASTGQTYDKDEFKDMVRKRFEQNIQIEIHFKRIEVEKDGTVQVHVEVHFTRNGQTFTTHDTHRFQKYGEGWYLVETRSHMRGG |
| 963 | PNPEDQEAIKQIVRRYFEAFNNNDIDAIYSLYAPDSQIHFESTGQQFDVDELREMIEKRLRRGTQTEFEVKEIQVYKDGTVTVHVQVHMTTDGQTFTVDQTITITKDGDDWKIVEHRVREHGS |
| 964 | TDPSVQEAVKELIRRFAEAFNNKDLEALKELYHDDARYRDSTGQDYNKEQIEEILERMMSRIQQMHFSIDEIQQHNDTEVTAHVTFHVTINGTTYTIHSTFFFVRTKDGWKIISEEVHERST |
| 965 | TDPSFQEAIKEFYKEEAEAINKGDVDRWMSLFHPDATYYSSNGQSYDQEELRRMFERMLSTVQRLEFSIDEIQQDDSTHVTVHVTFHFTVNGKTYTVHATDRVQFEKDGVKILERREHTHST |
| 966 | PDPEAQKAIKEAVRREAELLNKKDLDAWMSLFADDATFTDSTGQEYDKEEIREWVKQMLSRAQHFEFHINSIEQKDDTTYTVHVTVHVTIDGKTYTIHMTDFWVVTKDGWKIKRREVRTHST |
| 967 | TDPSDEEAVRQVVQRLVEAFNNGDFDALKELFHPDATYVDSNGQEYNQEQIEEWIRRAMSRIQQIEFSIKEIQQDDHNRVTVHVDFHITVDGEDYTFHMTDFYVKTKDGWKLVRREVHERST |
| 968 | TSEKEFEEMAREWLRRFYEAINKGDLDTLKSLFHPDWTYHNASTGQTYTVEQFLEMVKKMVENNVQIHWEIKRIEISGDEVTAHVEVHITTSSGRTFTVHDTHHFRVEGDRLKIVETRSQYQEN |
| 969 | PSEEEIRQLLRKYMEAIDSQNGHDAVRELYADDATFTYNGRTFKTKEEIAQWYREWLSQHPDMRREIKHLEIRGDTVHVEFTVHFNHNGQTYTVDFRATFRFDHNGKIKEVRIHTHDKR |
| 970 | PSEEEIRRLVKKLLEALDSGNGEDAIREIYTDDSTMHVNGQQFNSKEEIVEWFKRFLSQFPDIHRQVDSLEIRGDTVHVHVTMHVTHNGKKVTIHITSTFHFNKDGKLVEERIHIDNRD |
| 971 | PSEEEIREMLKRFYEAVDSGNGEDAVQKLFHDDATFESNGQQFNSLEEIKEFIQKWASQFPDLHRQIDSIEFDGDTVTVHVTVHFNHNGQTHTVTFHHHWRFDKNGRIVSLHEQEHDNS |
| 972 | SDEDQEEIKRVIEALARALDEGDADAFASFYSDDGRIHFADGSTLTTKEEIKEWVERFKSTNPDAQRHIHSIHFSDGEVTAHVTLHVTHGDKTVTIDVTRHFRFKDGRLKRDEIRWDEKPT |
| 973 | PDQEEIIRTYIRAYVEALNSGNVEEMIDKLYHDDATVHVNTQTFTNKEEVKEWYKRWLSNFKDVKVEPQTNVEVRGNTVTLHFTVHATHGDETKTFHFTYHVRFRGKKITEEHVHENRG |
| 974 | PKEEEIRRTKRKFFIEAFNRGDLERMVRELFHDDATFHYNRRTFKNPEEIVEWFRRFWESFPDATVEDNTHVHFDGDTVTFHFTVHLRHGDKDQTFHFTFHDRYKDNYLVEVRVQTDQG |
| 975 | PKAEELIEIKIKYFAEALNRGRAEDMVRELFHDDAQIHVNNQTFTNREEIIEWFKQWLRRFPDTHVDNDAEFHVDGDTMEAHFTVHVNIGDQTYTFRITVHDEYRDNYLYRTQMHFDTG |
| 976 | PKQEEIIRTFLDFFVRAFNTGNVDKMVDDLYDKDAQLHVNNQTFTTLEEIKKWFREFLKTFKDVQVHNESNVEVRGNTVTVHFTFHATHGDTTYTFTMTSHVTFRGDRIVEEHVHFDSG |
| 977 | PDQEEIIRILLRKLAEAYNTGNIERMLRELYDKDAQYHYNNQTFHTLEEIIKWFKEYFKRFPDVHVELESNVRVEGDTVEAHFTVHVRQGDKTITYRYTRHFRFEGKKITEDRSHEDKG |
| 978 | PDQEEIVKILLRKFVEALNSGNWEEYVDELYDKDVEVHFNNQTYTDKDEIKKWFTEWFSNFPDVKVEIHSNVEVRGNTVTIHFTIHVTLGDKTYTYRVTRHIEFRGKKIYRDQAHFDSG |
| 979 | PDQEEIVRTYLKLFVEAFNSGNWEEFFERLYHDDAQMHVNNRTFNDKEEIKKWFREWFERFPDTRLENKSNVEVRGNTVTFHFTFHVTHGDQDYTYHYTFHVTFRGKKITREQVHEYRG |
| 980 | PKQEEIVEEFLKKFAEAFNKGEIERFIREIYHDDAQFHYNRRTFNNPEEMLEWFRQWFERFPDVHVTLNSNVEVRGDTVTIHFEIHVNHGDRTYTFRYTSHITFKGDKITEEHMHTDSG |
| 981 | PDPKQIKKEMAELLNSGDWKKALEAFERYFTPDAEITFNGETITTLDELKEWIKKWFEDVKDAQVHVDENIEINGDHVTAHVHIHFSGENQTVTATQHDVYKDGKIKRSQDHSQPNN |
| 982 | PSEEELKERVKEWIHREVEALNKGDWEAWKRMFHPGAQFHDNTRQQTFDLDEVTDMIRKQQRHSDVHVEIKEIRIEIKSKDTVTVTVHVQVHEHHGDQTYTYSVTTHFHFEFRGDELYITEKQTQRDRT |
| 983 | PSEEEQERAARDWLRRWFEAMDKGDLDTLSELYAPGAQYTDATTQQTYDKEEFLEMVRQLFENSGVQHHVDSVEIEIKDETTVTVTVHVTVHWHHGDETRTVTITYHFHFRFEGDELKLTHQQTRRHQN |
| 984 | MSEKELEEIARDWVRRFFEAFDKGDMDKLRELYTDDAQYTDARTQRTYDKEQFLKMVEKYLRHTGTAHHLESIEVRIDDETHVTVHVHVTVHHHHGDETKTVHVTSTFHFRFEGDTIKLVEEHVQFNQS |
| 985 | MSEKEREEAIREWLRQFYEALNKKDLDAIRELFTPGAQFTFNGQTFNDAEEFEEWVKKWLSADFQIQIESIEIRINDDGSATVEVHVTVTNRKTGETIRFTDTYFFRFEGRQMKLVRVESRSDQLKD |
| 986 | ASEQEIKEAVRELIRQFYEALNKKDRDKVRELVAPGATMHVDGQQFDDLEEIEEWATELMRQDWELRVKSIEIRINDDGSVTATVHFTAHNRKTGTTVDVTDHYFFERRGDEFKLVRVESHRNQKKD |
| 987 | MSEEELKEAVRQFAQRMYEAFNKRDRDKLDELLTPGWRFESDGQQFDDKEEFKEWVDRMLSNKFHLHVKDMEVRVHSDGTVTVHVTVELRQDDTKRTWTTHDTHHFRFEGDQVKLVETRVSFQPNKD |
| 988 | ASEKEAEEAIREILRQYFEAVDKGDVDAVASYYHDDARYTQAGRNETYNKEQIKEWYERFLSHFDTQRHMKEIRIQFDSKDHITVTVHVEAHHNGSGQTVTFTYHFHMQRDEGEFKITRVQSHNTD |
| 989 | SSEEEAEEAVRRVVRRFFEAVDKGDMDTLRELLHDDWTYHQNDHNETYDKEEFLQMVEQWARRVRVHRQFHGLEIRQSDKDEYTVHVHVTVHHDGTNRTQNVTETHFFRRDRDEWKIIETRVTTTD |
| 990 | MSEKEIREAIKEWVRQWAEAFNNKDIDTIASLYHPDATVTHKGTNQTYDYEEYVRMVEEFLRKFDTRFEIKSIHIELSSEETVTVTVHVTATNTGEGQTFHFTYHFFFRRFDGQWYLLHQREDNTN |
| 991 | TSEEEVEEAVREFLKRWVEAFDKGDMDTLRELYHPDATVHFNDHGETYTYEEYLEWVARFLSNFNLERHIHEIHIENESSTSATVTIHMTVHRDGSGQTFHFTYHFRLRQDSGEWKIVEQHVTTTN |
| 992 | TSDKEIEEAVREWVKREIEILNNKDLDAWASLFAPDATYTQAGTNQDYDYDQMREMVRQYLEKFNAKIELKSLEVRSDSKDSATVTVHIDITEDKSNRTITFTYHFHFRQDDGEWKLKRKQTHNTN |
| 993 | TDKSVEEDIREILKRFYEIFDSGDADKVASLYDEDATFHDSKTGTTYDREEFKERVERQASNDSRHQIDDVRVEQNGDDVRVTVHVTVTFNKDNQQYHSEFTQHFRLRLENGQWKIKEDRLTTKDR |
| 994 | TDPNDEKAIRELKKRAIEAMNTGDWKALKEIFHDDATFHDARTGKTYNLEEYIRMFKELQKFDMRIEVKSQEIEQRGDQIHVTTHMEVHVNHGDQTKTTRVTTHDVYVKENGDWKIAETHYHTDDS |
| 995 | ASEEDQRAIKEVKKQLAEAFDRGDWERLRELLHDDVTFHNSKTGKTYNREEFREMVERFASIPAQRQIDELHIEQRGDDYHVTAHVTVHEHKDNQTKTYRVTFHDVYRKKDGTWKVVRSQEDIDDE |
| 996 | ADPSDQEAIKKILERFYRAMDEGDREALDRLLHDDVQFHDSNTGKTYSKEEFLEMVTEQAKFPLQRQIHGMEITQDGTTYTVHVTVTVHVNHNNQPRTNTFHDTYFFQKKDGQWKIVEVRSTSDHS |
| 997 | MSEKEAQEAAKEVIRQWLEAFNKGDMEALKELYDDGAQVHFASTQQTYDKEEFLEWVKKMFKQDTKMELKSIRIEIDDHNSVTVHATFQLHTNQGNTTITFNIHSTFHLTLENGKWKITEQHITMHQEQT |
| 998 | MSEEEMKEAVKEFKRQFVEAFNKGDFDRMRELIHPGAQFHFASTGQTYDVDEFLDWFRKFFENNTRVQIDQIDIEVKTPTDVTATVHITFHVTHGNTHTTFHVTFHDHYRFKDGQIYLIETHIRTHQRDN |
| 999 | ASEEELKEKVKEIVKRFIEAMNKGDMDAVKSLYHPGARVTFSNTNQTYDLEEFLEWVERLTKQGTKIDLESIHIEIKDETTVTVTVHVTFHTTHNTKHQTFHVTSHMHFQLRDGQLYIIEEHVTTETRDS |
| 1000 | DKREEFKEQIKQLVQRLVESFNTGDRDAFREIYHPGAKFHFSNGRTYDKEEFEEMVEEMHRRDIKLELESLEIHVSGDEATVRVSVTAHNSSDNKTTRFDSTFRFRVGDGEMKLTEEHIHRND |
| 1001 | PEEEEIKEKIKQFKRQLVEAFNKKDLEAIAELIHDGAQFHFYQTQRTYDKEEYKEWVERMLRNNTQIHIESIHIEIKDNGEVHVTVHFTAHRTQNGKNYTIHGTAHDRYRVKDGKLRLIESRIQLHEQPQ |
| 1002 | PEEEEMIRQVREFLRRLMEALDKGDADTIRSLMTDDWTFHDSSQQRTFSKDEFVEMVKKFASNDVKREVHSIRFEIKKDGELTVEVHVTVHATHNGKDYTQHITETHHFRFKDGELRLVRTQVTTHQQPN |
| 1003 | PEEEKMEQAIRELVRRLIEAFNKKDIDKIDSLYHPGAQFHFYSTQRTYDKEEFREWVERLLSFDTQFQVDEIRVQLYDDGTATVQVHVTVHTTHNGKRYQTHMTSHFFMRFKDGQLYILSERIQMHTQPT |
| 1004 | PSISEEQVREWVKKFFEALNNNDPDKLEELFHPGSTVTFPDTGTTLDQEEFKEWVERMLRSAKDSHFQIESLEVRIHGDTATVHVTWTVHFNHGDRTETYTGHTTFHFKRHGDEVRLVNMQVQTDQT |
| 1005 | DEETEIIEDAEEILRRYYDAFNKGDLDTMYELVHPDAQFHDSTGQTYDLDEWFEWLREILEKTDMKIEIKSIEFRIDEDSRTIHVTVHVTVHLTHNGETKTYTVTHHWVLQRRGDSVYVVSVEERRNSH |
| 1006 | PDTEEAKEDIRQIIRRFFEALDKGDMDTLRELVDEDWTWHDNDGQSYSAEEFLEMVERFNRNFNSNHHLESLEIRQKPDQREFTVHVHVKVRFTHNGKNVDVDITETHHLRKKDDEFKIVETHTHPRQT |
| 1007 | NDEEEAKEAAKQIIERLVEAWNKKDLETLDSLFAPDWKWTNANGQTYDREEWKEWVERWMRHFDTKIEIKSIEFRVQPDSRQITVHVTVQVTHTHNGQQRDVTFDETHHFRKFGDSWKVVETRTQPRQT |
| 1008 | PKEEEAKEIVKELLQRFYEAMDKGDMDKLRSLFHPDATFYDSTGQSYDLEEWLEMVRQSTENSDSRHHIKSIEIRQDSDERTFHVTVHVEVHFTHNGETQTYTFEHHWVFVKEGDSVKVIELREHRYNH |
| 1009 | PDEEEAREAVKQTIRRLIEAWNKKDWEALRSLFDEDWKWHNADGSTYDLDEFIDWFRKWQRHFDTHVELKEIHIQIQPDKRHITVTVHLTMHEHHNGQRQTYTYTEHHHLRRQGDDFRIVETHTRPINH |
| 1010 | PDEEEDREDIKEWLKRFYEAIDRGDADALREMIHPDATFHHASGQTYNLEQFLEMVERTRRHSNSNHHLESIEIRLDDDKRQITVTVHVTVHFHQDGRQYTFRFTEHHRFRKEGDSWKLVETHINSNQT |
| 1011 | ASEEEARKAIEEIIRRLIEAFNKGDLETFMSLFDEDWRYHNDSTKRTFDYEEVKEMVKRLLEAGFKLQVDSIEIRQDGNDEFTVTVHVTVTQTHPGTKTVHITEHHRFRYGESSWKIVETRVRRSET |
| 1012 | PSEEEIKEQIRQFDRRRLESFNSGDIDKAVRLVHPDATFRFDSTQKDYDLEEFKKMMEELFRSNTHLQLHGLEVQVHGRTHATATGHITVHFRHPGTRTTHFTYTVTYRYGDNDWKLTHMRVHREHR |
| 1013 | ASEEEIEEAVRQIVRRWVEAFNRGDADEMASLYTPDAEFTHQSHQQTFDKEEFREWVTRMFRAGVEVELKSVEIQIFGDDTVTVHVHVQLHFRHPGTKTVHVTFTFHLRYGESSFKIVRQEIRRHND |
| 1014 | PSTEEAVRELLRQFYESFNNKDWKKIEELFAPDARMHNQSTGRTWDYEEIVEWFKRFASNGHDVQIQIDSIEIQQGPEGHATVHVNVTFHFHHNDRTRTVQVHETHHFRLTKDGWKITRTQIHRVQK |
| 1015 | PSDEEAARELVRKFFEAWNNRDREKLEKLVHPDATFHFPSHQQTYDWEEFIEWLERSAGNNVDAHIEIKDLHVQSTDEGEFTVEVHWTVTFHHGDETKHVDATTQFHFRLTKDGWKIVEVHFTHDNQ |
| 1016 | PSTEEAIREWIKREIEAFNKQDLDTWISLFHPDATMHVSDTNETFDYEEIKEWFSRFAGNNVDVKIQVDSIEVRETDEGHVQVHVHVTIHFHNGDETRTLRFTFTDHFRLTKDGWKIVERHITTDGS |
| 1017 | NSEEESKEIAKELKERFIKAFNEGDADAAVSIVFTPDSQFHDKDGRTYNLEQFIEYLRELLKNDVKIHLHSMEIRVHGNGHVRVTVHVTLTSTHNGSKVHVTVTAHDFYQQKDGTMKIKRSQFHEDSN |
| 1018 | SSEEEFKEAVKETIQRMIEAFNTGDMDKIRELYHPDFRFHHSSGQTFDKEEFLDMIRKLLSQNVKIELKGIEFKEITDTEVHVEAHVTVTNNSTGTTYHFTSHFRIRQDGDEFKITEEHLRTIG |
| 1019 | TSEEEFRKAVEELLKQFYEALNKGDLDKLRELLHPDARFTHDQGTTYDREEFLEMVEELVKAGVRIRITGIEFKEITSEEVTVQVHVDVTNSSTGTTVHVTEHHHIRQEGDEFKIVSTEFRRDS |
| 1020 | YSEEEVKEAVKEWKKRYVDALNKGDMKEISELIHPDARFTHERGETYDRDEWLKMIEELLSQNYHMQIRSIEFKEITEREVTVTVHVEVTNASTGTTYKFTYHDRYRFEGDELKLVELREHTRD |
| 1021 | MSEEEFKEAVKEWIRRYVEAWNTKDLDKLHELYDDDATFHHQDGETYNRDEWLDRVRELLKQDYQIQLLSIEFKEITDTEVHVQVHFQVHNRKTGTTRHMQRHFRFRFEGDELKLQEDHERRDS |
| 1022 | PSEEEAKEAARQIIERLYDAFNKGDLDKLRELMHDDWRYYDSTGQTYSAEEFLDMVKKAKEADVHVQIDSIQVQEVSSHTITVEVHATVHNGSTGTTVHTTDQHHFQLFGDEWKIVETRVHTDG |
| 1023 | MSEEELKEAIEEWKRQFIEAINKGDLDKLRELVHPDARFHHASGNTYDREEFLDRVEKLNKNNFQFDMKSVEFKELTSHEVRVQVHVEVHNRNTGTTFHFTQEDRYRIEGDEVKLIETRETSHG |
| 1024 | PSEQEFEEAIREFKKQWAEAFNNKDMDKLSELIHPDARFDHASGQTYDKEEYLKMIEEMLSNDWNIEVEHLEIKEITERHVTVEVHMTFTNNRTGTQYKVTQTDFYRIKDHEIKLVKSQEHHDG |
| 1025 | TSEEQMEELARRYYTAFNNKDWKAFADLLDPDATVTIFHDGQQHDYDWEQMVEMVKRIAETFKRFRIQIDSIEFRGDTVTVHVTFHWTNDSEDTTVHATHHWVFKDGRLTELHAHYNRQR |
| 1026 | ADEEELRELVKRYAEAMDRGDWEAAARLMHDDWTFHLNNGQTFTTQEEFIRFVQQFFSNADSRHDIKEIRVHGDTVTVHVTVHVNHKTGKTYTIHATEHHEVRNGKIARTQVHEDTQPT |
| 1027 | DSEEQIKELIKEFYRAINERDLDALSELLHDDAQLHLSNGQTFDNKEQIIEWFKRMLDNSDMRVQIDEIRIDGNTATVHVTMHFTHDSGKTVTVHVTEHHRFKDGKIVRTQIHFDSQPN |
| 1028 | PDEEKLRQWKKKFIEAFERGDLEKLEKMIHPGARFHFAEQQITFTDREEFRQWVEEFLERFKNTHIEIKSLEVRGDTVTAHVTVHTTHGDKTQTYDYTTHDRYEGDKLVESRFTFDTKN |
| 1029 | PQEEKLEEWKRRFIELVNRGDLDALKEMIHPGATLHDENTQVTYHTLEEIKKHIEDWLSRFSDSRFQIDSIEIRGDTVTVHVTFHFRHGDKTTTIHMTQHDRYKDDKLTESHTHYDTKD |
| 1030 | PDEEKIKEWVKRFFEAFERGDLDALVELLHPGVQITFSQYQQTMHDLEEFKQWVEEQLKTLEDTKFQIDSMEVRGETVHIHWTVHTTHGDETRTVHVTTTFHFRGDRITEIRFHFDERR |
| 1031 | MSEEEIKDWVRQFMETLDEAFEKGDWKAVKEMFDPGVTIHDDSRKETFTDLEEFARKFFTKYFKNIKDMRREVKEIEIRGDTVHVHWTVHVTHNGKKITIHGTTTFHFKDGKLVEVHFHFHPT |
| 1032 | DSEEEIKEAVRRFYAAMNEAFSKGDADKFKSLFHPGATFHDDSTGTTFTDLDELAERWFREFFETFKDMEIQIDEIRVDGQTVTVHVTVHVTHNGQKITYHVTDHYTFKDGKLVSMRSQWHQT |
| 1033 | PSEEEIKEWLEKFYEALAKGDLDKMRELLHPDATMHIAQSQVTFTDLEEIMEYLKQWIEKAKDTQMDSMEIHFDGDTVTVHVTITVHTPEKTYTFHVTHHWRFKDGKVTEVRETTETRET |
| 1034 | PSEEEIKKFKKRLIEAFANGDLDALEELFTPDVTIHFSSEQVTFTTKDEFKEWVRQFLSQFKDIQLDNMTIHIDGDTVTVHVTITVHTDTQTYTFHWTTHDRYKDGKIVETQFHVETRET |
| 1035 | PDVSEEEIREFLRRFYEALDRGDVDAVVEILDDDWQFHDSSGVTLHTKEEVKEWVERWVKSLPDVRREIHDLHVDGTTVTVHLTVHVNHKDGTHTVHTTDTHHFRNGKLTETHVHWDSRD |
| 1036 | SDVSEEEVKEWFRREFEAFNHKDVDTWVDLFHDDAQIHFSDGETLNSREEIKQWFEKLIRELKDARIQVEDLHVDGDTVTVHYTLHVTHDNGQITVRVTTTVRFEDGKVKEKHVHYDRKE |
| 1037 | ASEEEIKELLKRFYEAIDKGDMDALDELIHPDWTYTDASKQRTFSKEEWKKMVEELLKRSKDTRHQIHEIEIRGQTVTVHVTVHHTHGDETRTIHFTDHHTFKDGKLAHTRTHWHPKD |
| 1038 | MSEEEIKEWLKRFYTAFDNGDADTVLSLLHPDWTYHNSNGITITSLEEFREWMKQWKEKAKRVHRQIDRIEIHGDTVTVHSTVHVHHDDRTVTIHVTDHHTFKNGKLTETRSHVDNGN |
| 1039 | MSEEEIKEWFERYFEALDKNDLDKVASLYADDATVHFQDGITLTNKEEIKRWYKEWMSKVKRTAHEIHEIRVHGDTVTVHVTTHVHHDDRTITVRVTVHVTFKNGKVKEVRFHSDKGS |
| 1040 | MSEEEIKEWLKRFYEAIDKGDLDALRELIHPDATFHFQDGSTITNADEFKKWVEEWFKQVKRVHREIHEIRIDGDTVTVTYTMHVHHDDRTQTRQATDTFHFKNGKLTEFRSHWNNQS |
| 1041 | PSEEEIREIKKQFFEAINNGDMDKVKDLFHPDAQFHIFEGITFDSREEFVTWFTKKIREHFNTKMHLREVHIDGDTATFHYEVTHHHDDRTRTVRVTTHDRYDKRTGKIVESEFRMDTGG |
| 1042 | DDREEIEKLDRQRLEALDRHDMDAFREYIHPDAEMHIHDSSGNTITFKTIDEIVEWFARVLSRYKDSRHEIKSVEFRNGTVTVHVTVTFNHDTGQVHIDVTYTMTYKDGKVTEVHFHYDNK |
| 1043 | PEEEEIKEWKRRFFEALDNHDLDALRELFHPDAQVHFNDSSGQTYTYDTIDEIVEFFARVLSTLKDSRHEIKRIEINNGTATVHVEVHVNHDNGTKTRRVTSHDEYENGRLKRTQFDFDDQ |
| 1044 | DWREELEELDRRRLEAIDRGDMDTLKELIHPDAEMHIEDQSGNQITFKTIEEIVDWFKKLWESLKRVHRQIDRIEFHNGTITVHVTVHVHHENGTRTITVTYTMTYKDGKVTEVHFHYRDS |
| 1045 | MSEEEVREFKKRLAEALDRGDVDAAASLFHPDARFHHNSGETLNDLEEIKRWFREWFSKFPNVHRQIDSMEVNGNTVTVHVTVTVHHPDRTLHYRFTVHDHYKDGKLVETHVHENPT |
| 1046 | PSEEEIKDWVQRFFEIINKTRDWDALKKLIHDDITMHIHNGITFTTRDEIADWWRKWFENNPEIHVEVHVHVDGDTVTVHWHVTVHRGDQQYHYTATTHIRFENGKIAEIRIHIRQWS |
| 1047 | PSEEEIKTWKKRYYEEFFKTRDAQAAAEYIHDDVTIHFNQGITLHNREEIREWFQRWLDNFPRVQVHVEIHIDGDRVEVHVTIHVTHGDRDITYHVTYEDRYENGRLVETRVTINQES |
| 1048 | PSEEEIKELDKQRIEALDNGDADTLAEIFHDGATFYHNDSQSRLNREQFREWVERWFKTFPRVHRQIDEIRVEGDTARAHVTVHLHNPTETITIHVTYEVHYKDGRLTELREHSDQKG |
| 1049 | TSEEEIRRIIKRLAEAFDNKDLDALEEIYHPGATFTSKTSTHDLDLKEWKKRVEEHLSRFKDSRHEIKSIHIDGDTATVHVQAHFHTDSEDITIDVTSHFHFRNGKLTREKQDRDQKG |
| 1050 | PSEEEIREILRRLFEAFDKGDADSFASYYDDGVEFTHNTHTQQFSKDELREWFERWHKTFKDVRHEIEEIRINGDTVTVHVTVTFHHDNRTTHQRVTRHFHFKDGKITHDREHRETRS |
| 1051 | MSQEEIEELLRRFYEAVDKGDLDAIREIMHENATITHDHSTKDYSLDEIVDMVRQFKEKFPNVHRQIDEIRIEGDRAEVHVTVHITTSHEDQTFHVTHHWVFKDGKVVEMREHFEQRG |
| 1052 | DQGTEAIEEWLRRFYTAINNKDADAIFELITPDATFHFHQPQTFTNAEEYKEWFERWVKSAGSYRVEIDSMEIRGDTVHTHVTIHVNHGDKTQTIHVTETHHFKDGKLVETHQHEDERPS |
| 1053 | PSQEEIKEWLKRMYDLLNKGDADALASLFAPDWTYHHASGRTFHSLEEWKKWFEETFKRFEFHIQITGIHIENGTVTVHVTITVTNKDGETRHVDVTDHHHFDKDGRLKETRSHLDEKPQK |
| 1054 | PSQEEIKEWLRQYYEAFNRRDLDKLEELLHPDATVHFSKGRTFHTKDEYKKWVEEFMKTFDFQIHIESIHIENGHVTVHYTVTVTRPNGETYHIRFTDHFRFDHDGRLVSVKSDSDERPQE |
| 1055 | PSQEEIKQWVERFFELFDRGDWEQLAKMLHPDWTLHHYSGQTFTTLEEFIRWYQSFFSNFEFHHELKELHVENGTVTAHVRVTMTSKDGETRHMRVTEHHRFDHDGKLKETHVHVDTKPQE |
| 1056 | PDEEEIRDIKRRYLEAIDHGDWEKAKKLFHPDATITIYHPDGKRETYDLEQMVEMIQRLLKQGDVRHEVKSVEFNGDTVTFHVTVTWHHNGQQHDSTVTYHDVYKNGKITETHIRRDDEG |
| 1057 | PDEEEIEEILRRLYTAINEGDLDKWDDLFHPDATFHEYKKDGSSSTYDLEQWKERWRELQKSGDLHIQIESIHFDGKTVHVTYTVTFHHNGQQYHITYHDTYRFRNGKITEVHSQEDTHG |
| 1058 | PDEEEIERILREHYDAFDKGDMDKVSKYVHPDATFHHYKSDGRTETYDYEQYMELIKQWMKRGNSSHEIKSIEFDGKTVTVHYTVHWTHNGQNHTVTATDHYHIKDGKITEVQSQEDSHG |
| 1059 | PDTEEIERFVRRFIEEVDKSGDADAAKSYYDKDARIDFSGGQTYSLDQFREWVERMFKQGWEVEIKSIEVRGDTVHVHVTMHNRNTGQQVDVTRTFHFRNGKLVKDEIYEDPS |
| 1060 | DEEELRERIKEIKTRFAEAMNKGDRDAIKELLHDDAQFTQSGNTFSLNLDQIAKWFEELFRQDVQVQVDEIHIEIKSKDEVTVTVHVTFHNKRTGSTVTATSHDRYQYTKDGVRLTESRMHSQQVT |
| 1061 | DEEEIKKEVRKILKRFFEAFNKGDWETIKRLMHPDATYVNNGQQYKLNQEQWAEWVKRMNSNNFRIEVKKIDIEIKSKDTVTVRVEWTMHQDRTGRTFHGTTTFHMRRTKDGWKITRVEEHVQEQK |
| 1062 | MSEEEIERIVRDWVRRFIEAMNRGDVDAVVELYHPDATFTDQNGQEYDLEEFRRWVEQMVRANVHIQLDKIDVEIKGTRHVTVTVHVTFHQEHGNQTYHVTSHFHFRLDDGRFYLTRERIETHST |
| 1063 | SDEEEMREHIKELVERFYDAINKGDLDKLREILHPDATFHHANGQTYDKEDFLKWVEELKKNNTHVQIDSIEIHIHGSTEVTVHVHVTFHTEKGNETYKVTETHRFRLTDGQFYIVETHVETHST |
| 1064 | TSEEEAKEEIRQWVRRWYEAINRGDMEKVKRMLHPDWTYHNQNGQTYDLDEFLKMIEELLRNDVRIQIDEIRVQIHGTTTVEVEVHVTFEFKNGGETYHTTDHHRFELKDGRFYLVETRVHMRST |
| 1065 | YSEKEMEEAIRDLVRRFVEALNNGDLDALDELYHPDATFHHASGQTYDKEEFKEMVKELIKRNAHFQLDSVHVQIYGTNEATVEVHVTVHFEHGNETYHFTRHLHFRLEDGRWYLLEDHLQRHST |
| 1066 | TSEKEIKEAVKDIVRALVEAFNKGDMDKIMSYYHPDATFHQYNGQTYDLEEFRQMVEKRLSQGVKVELKSIEIHVDGTTEVTVTVHVTFHFSNGNETYHATRHIRLRLEDGQFKIIEDQEHTRST |
| 1067 | PGEETAERAAREVLRRWFEAFNKGDLDTLRELYDEDAKFTNIKEQQTYDREEFEKMVEEMMRHPFHIEVKSIEFQFNSDDEITVTVHAQVHFHDDPKTYDVTFHFRLRFEGDSWKIVEQRTQRDPT |
| 1068 | PGEETLREEVKKVLKRYAEAMNTKDVDAIEELYDEDARLHIYATQRTFDKEEIKRWYEELMKHDFHVEIKKIDIQVDHNDQVTVTVHMTIHFRDDNKTVHVTAHFHMTYEGDRMKITEVELRHHPS |
| 1069 | PGEETLKRLAKEFLRRLYEAFNKGDLDTILSLFDEDWRYHDTSQQRTFDREEFKEWVERLLSFPFRIEIKSIHFEIKSGDEITVTVHATVHFAHDPKTTHTTDHHHFRYEGDSLKIVETQESRHPT |
| 1070 | PGEEEAEKAARELARRMYEAFNKKDLDKLLSLMDPNAQYTDANRNRTFDYEEFKEWVEEFLKYDFHIQIDSIEAQFHSDDEITVTVHVTIHFRTDPKTVHVTHHWHFRYEGDTWKIVRLQENRQPS |
| 1071 | PGDEEAKKAIKEWLKRFFDAFNNKDLEKIERLVDDDVQFHFAETQKTYDKEEFKKWVEEMLRHDFRIEVKSIEIQADHNDEYTVHVHWTVHFRDDPKTVHVTTTFVFRFEGDDFKLVELRMRSHET |
| 1072 | PGEETLKEQIKEVLNQFFRAVDEGDADTADSYLDEDWRFHNVSQQRTYDREEFREWVERFARIPFHRQIDSIEIQIDHTDEATVTVHVTVHESTDNRTYHVTEHHRMRYEGDTVKILRTHVNTQPD |
| 1073 | PGNETLEEAAKRIVKALIEAFNNKDLDTLSELYDEDAKFTNYSTNQTYDYEEFQDMVEKLMRYDFRIEVKSIHFHFHKDDEITVEVHFTVHFATDNKTINFTSRFVIRYEGDTFKIVSEHVRSHHT |
| 1074 | PEEDERAVRETDKRRWEAINNGDADKVKSLFHPDATFHQSNGTTYDLDEFREMVERLFKAKVQFQVDSMEIRIDDNGEVTVHAHGTAHRDDEGKTVHFTYTIRYRRNGDEWYIVEYRENRDEKST |
| 1075 | NPEDEEAVRQLIRRFVEAFNNGDLEKLDSLYHDDARYHQQNGQTYNKDQIKELLERLMSQKVKFEVHSVEIRILDNGEVTVHVHGTFHFDESGQTTHATSTFFIVRRGDEYYIVSEELHTDNQST |
| 1076 | PGEENLEEEIRQWVRREIEALNKGDMDTWASLFSPDVTFHDQDTGRDFDREEMIEWWERLLKSAKRWRIELKSLEVNVHGDHIEVHVHITFHWNQDSRTITVHVTSTDHFRLEGDQVKIVERHTSTDEKK |
| 1077 | SSEKELKEIAKEIIERFYEALNRNDADTVASLLDEDWTYTQDGETYKFSRDEIKEWVKQMASQDMQLHIDSIEFQIHGTHTVTVTVHVTVHHRHGDKTHTVEVTDHHFFRFDGDEVKITETRSHRRPRD |
| 1078 | SSEKEIEEAIRDWIRRYIEALNTGDLDTLKSLYDEDVTFTHDGNTYKMDQDQIKEWYSQFAKDGTHIQLDSIHIEIRGQHEVTVTVHITFHHNRGDRTETYSVTAHFHFRLEGDSFKIKEVREHTQPVT |
| 1079 | MSEKELEEAVRDWFRKYFEAFNKGDLDALKSLYAEDATITHSGNTYHLSLDQIKDWYRKLLSQNVQMKVDSLEIRINGTTHVTATVHVTLTMHRGNKTQHVTVTYHFHFEFRGDEVKVKRVQAHHHPRE |
| 1080 | TSEKEMQEIVKEVVERFFEALNKGDWEKVASLYDDDVTFTHDGETYKLSKEQVTEWVKKFHSADIQLQIDEIRIQIHGTNEVTVTVHVTMHQNHGDRTKTVKVTSHIRLEYEGNDFKIKEEHEHRHPVE |
| 1081 | DDSEALERKVREWLRRFAEAFNKGDMDALRSLYAEDFTFQDSRTGQEYDLDELLKMLEEMFKNNVQMHVDHVEFQINDDGTITVHVHVTFTNETTGTHVHVTSTFHFERQDSQIKLKREHATHHQS |
| 1082 | DSTKEQIEKIREIIQRFFDAVDTGNWEEIESLYDEDFRFHNSNTGQTYDLEELLRMLRQLTSQGAHHEVKSIEFQFNDDGTVTVTVHVQVHYESQGRTFTVTSHIHFVFEDKQIKIKEEHMTTDEH |
| 1083 | DEEEAREAIKEIVQRYVEALNTGDMETIKELYHPDARIHFYAHNQTYDYEEFIRWVEEFLRRAKDIRIEIKHLEIKDTSDDSVTVEVHVEFHVGHETYTVTSHIRIKKTDGGWRIVEEHVRWHKQ |
| 1084 | DQPEIQEAAKEILRRLYEALDHNDMDTLESLFHDDWRYTEAATQRTFDKDEFKEWVKEWLSRAKDVRRQIDSIEIKETGDDQVHVTVHVTIHVGTHTVHATDHHTFRKTDGGWRVVSTRAHYNQT |
| 1085 | DTPEIQEEIEKLLRRFYEAYNHGDLDALDEMYHPDWRFTEAATQTTYDKEEFKEWVKQWLETFKDTRLQIDSIEFKEFGKDEVHVTVHVTMHVSHQTFKVTDHHTFRRDDGGWQIVRTQTHWNET |
| 1086 | DDKDAEEEVRKVIRRFYELIDSGDVDALRELLHPDWRFHLASRQETYDKEEFVEWLRQWLSSFKDVRRQLDSLEVKDTSKTEVTVTVHFTMHVRHTTWHTTDHHHLRRYPGGWRIVETEVHMQQT |
| 1087 | DTPEIEEAIKELIQRFFEALDHNDRETLKELFHPDARFHAASEQRTFNYEEFLRWVSEWLSRVKDVRHEIHSIEFKDFTKTEVTVTVHWTQHVGDTTVHGTTHIRFVRTDGGWKILEMRVQFHQT |
| 1088 | DTPEMEEQIREVKQREAEALNNHDMEKIKELIHPDARMHVASENRTYDYEEIVEWFERFLRTFKDIQFQIDSLEFKEFGDDEVTVEVHVTVTAGDTTYTVTSTDRYKRTDGGWKLVETTIHIQPQ |
| 1089 | DTPKDEEDVRQTDRRRIEAWNRGDIDKIRELFHPDARMHISATGETFDYEEIVKWFEEVLKKAKDARMEIESLHIVDFGDDQVQVTAHGTVHVGDTTVHFTYTIFYVRTDGGWKIVRVHVTYHQT |
| 1090 | PSEEEIKEAIKDLLRRLMEALDKGDWETADRLYDDDATIHHEETQTTYNKEEFIRRIQERHRRGIRFQREIKSVEIRVDNGDTVTVTVHFQVHEHRGDKTRTLNITSHIRFVFEGSSVKITEEHVTTNQT |
| 1091 | MSEEELREAIKEVIRRMMEAWDKGDWEKLRELYDDDATFHEISTQRTFDKEEYIRRVQELFSRGFQFHHEIKRIEIQINNGDEATVTVHYQVHTTHGDEHRTFDVTSHIRIRFEDSELKIQEEHVQTDQT |
| 1092 | ASEKEIREAIKELLRQFYEALDKGDWEKLRELMHPDAQFHNGSTQTTYNLEEFVEMVQRFASQNIEVHRQIDEVEIQIDDGDRITVRVRVTVHHHHGDETRTVEIHNTDVFQLVGSRFYIVEHRSTTNQK |
| 1093 | PDEEEQREQIRQTVRRLLEALDKGDWETVARLYAPDATITHISTQQTYDKEEFVRRVKRRHDNNVETHREVKSMEIRIDDGDHVTVTVHYQVHHHHGDKTQTVNVTSHIHVRFEDSELKIKEEHVQTDQT |
| 1094 | VHPEDEEEIRQWVRRFYELLNRGDVDAVASLYHPDATFHHQSGRTFDREEFREWIEKFLSQGFQIQITGIEIHQKDGHITVHVHVTVHNSRTNTTYHFTQTFHFRLIGDSWYLLEDHLTTRQS |
| 1095 | VHPEDETAVRELVKRWAEAFNKGDADAVASLFHPDARYHSQTGETYDRDEYKEWVQKFFSQGYQIHLEEIHIEQKDGEITVTVHWRLQDRRTGRTWQIDTHIHFRRFGDDWKITRMEVHTRQT |
| 1096 | VHPEDEEAVRQALRRYYEAMNRGDWQTLRELMDDDWRYHQHNGQTYDLDEFIDWIRKFLSRGYEIHVTSIHIEQKDGEVTVTVHVTVTERQTNRTVNFQDHHHFRRKGDEWKIVRTETTTRQN |
| 1097 | VHPEDEEEIRQWLRRFYEAFNKGDIEAIRRLFHPDARFHNYRGETYDVDEFAKWVKELISQGVRIEIKSIHIEQKDGRITAEVHVRVHHEQTNETFEVTHHWFFVKFGDRWYVVRVEERTHQT |
| 1098 | DEDQKAAEEVDRRRLEAFNKNDMDTLKELFHPDAQMHVESEQRTYDYEQIIERMERRARHFKIQLDEIHVEVKDDGTVTVRAHVTVTQDDTGRTWEVTYTFFYRRKDGEWKIVEVRIRTEPQE |
| 1099 | DQKKQEDQIREWKKRYFEAFNKKDLDTLEDLFRQTPDVRFHFNGTRYNLEQYKEYLERLLSNDVQIEVKSIEIRFDDDDHVTVHVHFTMHESKTGRTFHYTATDFYRFEGEQLKIVETRYHSDTG |
| 1100 | DEKELEEAAREIVRRYYEALNKKDLETISRLFQSSKDAKYTQDGTTYDIEEFIEMVERMMSHDVQFHVESIEVRNDSKTEVTVTVHVQVTDKRTGRTFTVTHHWFIRKEGDEFKIVRVQAHTDTG |
| 1101 | PSEEQQEKVKDWVRREVEAFNKGDMETWDRLFHPDSTFHMSNISREEFIEWFRRFFRTFKDAQMQVDSLEFKDASDTEITVTVHVTVHDDSTKQTWHFTFHDRFRFENGQLKLVSRQVHSQGS |
| 1102 | PSKEDQEKVREWLKRYYEAFNNKDLDKIRELIHPDAKFHISNVDREQYLEWVERFLKRFPDVHMQITSIEFKDQSKDTVTVTVHVQVHDRSTKTTIHSTEHHHFRKKDGQFYLIETQVHTDGS |
| 1103 | YSDEEVKEEIKKWLERFFKALDNQDLEAVASLFSPDWTYHNTDDNKTYSRDQFKKLVEEFLKTFPDVTHQIDSIEFQKNGDGTYTVHVHVTVHFKTETQTFTETHHFEFTKDGWRIVETHHTTVPT |
| 1104 | MSEEEIRQAIKELVERYVEAFNKGDVDALARLYAEDAKYHHASGQTYNREELKEWYERFMSQQWEMEIKSIEIEVKGRTHATVHVHITMHNQRTNETYHVTQTFHFELRGDEFKITEVHTTTHKT |
| 1105 | MSEEELREAVKDVIRRWVEAFNKGNADEVYELYHDDFRFHHASGQTYSRDEFKKWVKEMFDNNFQVTNQSVEIRVEGTTHVTVHVTMTVTQKSTNHTWTFHSTYHIQFEGDELKITEQHTQTDQD |
| 1106 | PSEEEIKEQIKQLVERMIEAFNKGDADLFYSLVHPDWKYHNNNGETYDRDEFKKWLKEAHDAGIQVTNDSLRVEVKGKDEVTVEVHLTFHDSRSNETYHVTEHHRFRLHGDQWYLTRTHAHTNKT |
| 1107 | MSEEELKRAVEEFVRRFIEAVNKNDLEAIKELYHDDFTFTTSNGETYSKEEFIEWVERMVSANLKFDLQSLEIRVRGTDEVTVHVHVTVHDSRSNTTYHFTSTMHFRFEGDELRILREHLQTDQT |
| 1108 | PDEEQQRKAKETVKRWVDAFNNHDREAFKEMYTPDATNSSYGLNYEQIIEFITRILQTFPRVRVHIDSIRVELQPDGTVVRVTVHLTFHFYKDGKQKTVKITYHFQLVFENGSVKIVNQQEHSDDS |
| 1109 | ASEEDQRKIKEVKQRFIEAFNTGDLDTIRKLVHPDARFAAYQVDLDQFIEWVTEVKRQFPNMQVTVDSIEVRADPSGEEITVTVHVTFTTTHNGETHQFRGTYHDVYRKENGEFKLVNSQESSDNS |
| 1110 | PSPEDQKAIKDVKRRYMEALDKGDMDALSELFHPDFTYAARNLDYDEFLEWVKQWLSRFPTARHKIDSMEFRLHPNGEEVTVTVHVTVHHTHNGRHETVTVTAHDVYRKKDGEFKIVRTREQRDNS |
| 1111 | PDEEEQREIKRILEELAEALDKGDFDTFLSYFDKDWRYSAQNIDYEEFEEWVKEYQRRFPNVQRQIDEIHFHLDPSGEEIRVTVHVTVHHHHNGKTDTQTFTEHHLIRKENGSFKFVETKTQTDNS |
| 1112 | PDEEAQRAIKELLQRYFEALDHGDLDTLRSLFDEDWRDSSRNLNLDQFAEWVEKFFRRFPNARHQIDSIEIEQHPNNRTYRVTVHVTVHFNHNGKTKTVTVTEHHVFVKENGSWKIKHTTTHRDHQ |
| 1113 | ASEEDQKAIKEIVERYMEAFDKGDLDTIRELVAPDATYRGLNIDYEELMEWIKRWAESFPDVHREIEEIRVELDSNNRTFKVTVHWTVHVNHNGQTFTVRGTTHIVLEKQDGSFKIKSMQTHVDNG |
| 1114 | PDKEDQEEIKKTKQRMAEALDKGDRDTLRELFHPDFTFKARNIDYEEFIDWVTEWVKSFPDTKREIKSIHIEQVPEGHTYHVTVHLQVHTNHNGKTTTQRYTSHDRYRKENGSFKLIESQVHTDND |
| 1115 | DSEDEQREAVKEVLKRFYEAVDKGDLDTLEKLLHPGARFHDTSKGQTYDQEQFKERVERQVRNNIQREVHVQRIQIDGTHEVTVTVEVHVHEHHGDETQTHTFQEHHTLRQKDDTFKIIETRITSYQT |
| 1116 | PEKELEEAVREWVEREVEAWNKRDADTWASTLDKDSKYHQTDGPTTYDWEELKEMFERTASSDFHIQITSMEVRIIDDTRVTVTVHVQVHQTHGDQTKTVHITYHLHFEFHNGELRLVEKHSTTDEKE |
| 1117 | DPKELEEKVREWIRQWVEALNKRDLETLKSLYDKDSRVHTTSSPQTFDADQFTEWLKKYASINFQIHITSLEIRIIDDTHVTVEVHVQVHETHGDQTHTIRFTYELHFRFENGQIKLVSQRETTDTSE |
| 1118 | PKDEEQAEQIKDVKRRLIEAFNKGDADALSELFHPGAQFHNSEEPRTYDQEEFREMVERAHRNDVKVQLDEVRIQKVDEDEITVTFHLTVHFTHNGQKVTIKITFHDRYRQTKDGWKVVETHVTEDPKE |
| 1119 | PQEDELREQAKELLERFYRAINQGDLKAVADMYTPGWTYHNSEEPRSYSKEEFIEMLKELLKNDTKHHIEEIEVEVKDENEVEVTVHITVHSTHNGQPYTVRVTDTHRFRYEKDGVKIVETHTTTNPTE |
| 1120 | DKEEMEEAVRQLIKRFYEAFNKGNLDEVASLLDEDWRYHSGQQNQTYSKEEFLEMVEKLIKAGTHLHITSIEIHFDDSRGEVTATVHVEIRTTKDGREKTVKTTDTHHFRQENGSWKIVETRVSRDEKD |
| 1121 | TSEEEIREAVKDWVRRYFEALNHGDVDAVASLYADDARLTDASTGQTFDKEEIRDRIEKFFKQSDFRIQVEDLHIEIDKDGGHATVHVHFTIHVTHKNGETRTVHITSTFHFQFEGKELKLKEEQVHRDNGG |
| 1122 | ASEKELREAVREWLRRYYEALNHKDLDKVVSLFDDDWRVDFSETGKTYDKEEYREMVERFMSNSDFRIEIKSIEIRVDPSGGHVTVHVHVQVHVTDKTGRNHTVDTTDTHHFRMEGDELKIVKTEFRFDHGG |
| 1123 | PMSEEEIKEFVKRFYEAIDRKDAETLRRLLHDDATMHVSNGEQYDSLDSIIERWYKKFVSHAERARHEIKEIRFDGDTVTVHVQVHLTHSSGTQTYNVTDHYVFRGKRITEIRSHWNPQE |
| 1124 | PGEEKLKELVKRYVEAFNNRDMDALKSLFAPGWTFHDQSGVTFNNRDEFVEYWLKTLMSRFKEFHMNIDEIRIDGDTVTVHITVHWHHDGETRTQHFTEHHHVRGDKITETHQHFDQRG |
| 1125 | PSEEEIRQLVRRLVEALNNRDWEKFASLFHPDATIHVNGQTYSNAEEAARQFVEKFAKTFPKARIEIKSLEIDGDTVTVHWTAHVSDEPTTFHGTTHIHVRGKKITRMQIHIDSQP |
| 1126 | MSEEQIRQWVREYVEIFNHKDIDALASLYDEDARIHYNGQTYGNYQEAAKKIYSEIFRTFKNARLEIKRLHIDGDTVTVHVTMHVTDQPTTFHMTVHFHFRGNKLVEVHIHVDQHD |
| 1127 | PTSEEQIREWKKRYIEAWNKGDADKLEELFHPDAQVHFGTEGQTFDTLEQFKEFFKKMMSNNSQIEVKEIHIDKSTGRVTVHFTMHVNTDSQTYTFHATSHDRYENGKITEHRTQFDPQ |
| 1128 | PISEEQIKEWLKRYFEALNKGDLDKLKELLTPDWTFHLYAENKTFTNLEEFLEWVKQMLSLNIRIEVKEIHIDRSTGRVTVHVTVHMHTDSQTYTFHATEHHQFENGRLKETHQHVDPK |
| 1129 | SLQEEVEEWVRRWVEAINRGDLDAVASMYAPDATMHVSAEQVTFNSRESIKKFIEKLVKAGFRFEIKEIRIEVKDGTATVQVHLQVHDESTNRTFHVTSHFHFENGKLKEQRTTWNET |
| 1130 | SLEEQARQIVKRLIEAFNRGDMDEIFELYSDDATVHFTHENVTFHTREAIKKWFTELFSQNWHVQLDSMEVEVKDGTVTVHVHVTQTQQDTKRTRTVTSTFHFKDGQLKEEHVRWHQT |
| 1131 | PDEERAKDIVKKFYEALNTGDWETAVRLMHDDARFTVKSENRTYTTKDEITEWIRRFFSQNIRIEIKRIEVETKDGTVTVTVHVTLQDKRTNRTFDATHHWHFKDGRLVEVRQQVDER |
| 1132 | TSEEQMKELIKRFFEAVDKHDMDKLDELVHDDATYHDYRTNRDYDKEELKELIREWLEKATDVRHEMENPDFRFHGDTVTVHWTVHRTQNGQDFTVKATTHFRFTDGKLVELRVREDDKS |
| 1133 | PADEEQIKEWIKRFFEAFNKKDIDALANLFHPDATFHFYDGTTFHSQEEFRQYMEEFFRQFPRTHVQIDSIEFRGDEVHVHWTMTLHKDGQQYHIRYTTTFRFRNGKITEVHFQEDS |
| 1134 | PSEEEIKEFDKQRFEAFDRGDWEAVAKYVHDDATFTHNNRTYPMSRDEFTEWVKRFLRRFEEMHREWDSNIEVRGHTVHVRFTVHFTQNGKTQTVDARYDFKYRNGRLTRVETQRNTKD |
| 1135 | MSEEEIREWVRRFMEALDKGDWDALKKLLDKDSQIHFPESQKTYDLDQFVEWYKRFKRTTDRTQRHVDRIEIHGDTVHVHWTLHTTHQGDTYTFHGTTTFHFRNGKLVEVRFHVDSHKT |
| 1136 | MSQEEVKELLKRFYEALNQRDADKAASLFTPDATVHFFHGSQQDDITSLDQLKEWLRRWLKKFPDTKIEVKEIRVDGDTATVHYTVHQTQGDQPQTMHATDHYHFTDGRIKEVHSRFDPSAFT |
| 1137 | PQEEINKELIKRFMEALDKGDVDAAAELFHPGWTLHHSSGQTWTTKEEFREWVERLQKSLKDVKREVHIHINGSGTVTVHSQVHVTDDTGQTYTIDFTEHHHFEDNRITETHVQTDNQN |
| 1138 | PDEEKIRELLRRYYEALDKGDFDALREILHPDWTFHDSDGTTITTKDEFEEWVREIQSRWKDIKREVHIHINGNGTVTVHVTVHMTHDNGQRTTMHVTDHHHFRGDKITETHAHTHQEQ |
| 1139 | PKEEEMRRQVKEWVERYFEAWNRKDLDAIMSLFSDDWTYRDSRTGRDFDYEEFREWVEQWLRKFEDAKLQIDSITFHIDGDEITVTVHVTITDSSTGRTWHVTEHHHFRFTKDGLQIVETRVQHDQT |
| 1140 | MSEEEIKEIVKRLYEAFDKGDLDLLRELYDDGAEIHFYGQNITITDKDEFTEYVKRLFDNADTAHQVHIHISGDGTHATVHSHYHRTHNGQTHEQTVTSHFQFRDKRITEEQVHFNQN |
| 1141 | PDDTLEEQIKELKRRFVEAFQSRDPDKLRELFHEDVTFQHRHRSTTLGRDEYKEWVKRFIETFKDFRVEIKDMEIRATENSITVTVHVTFTFHHDSETKHYHATYHDEYEYKDGTVQLVRSQSNMHQEN |
| 1142 | PDSDIEEAIRQWFTHWLRAIDEGDADTLKRLYDDDVQFHHDNSTYKLDREQIAQWWREFFSTFKDVRHELKSMEIQSDKDHVTVTVHVTTTFHGDTSTHHFHATYHFFFRFQNGRLFLTRQQEHTQQQN |
| 1143 | VDKDQQEAIRQWLRRYFEAWDKGDADTVASLFDDDWTIEHSNETKNIDREQYREMFESFFRTFKNVQHHMESVEIRADKDHVTVTVHVTMHVTNDDQKQTYHATEHHHFRFENGELKIKHTQTHTNNSS |
| 1144 | DEQKEKIKDIIRKLIEAIDKGDADTAKSLYAEDATVHFSNGITFHSPDEFREWVEKFNKNTNARREFEIHIDDSTGTVTVHVTMHVNQGDQTYTVHMTRHLQFEGDKLREDRIQMHQQD |
| 1145 | DEHKEKIKEILSRFYEAFNKNDADALAELLHDDAQIHLSNGTTFNNKDEFRKWFEEFQSKFNTQVEWRIHVDDTTGRVTVEVQYHTTHGDQTHTADATEHHRLVGDKITETRTHIDQQD |
| 1146 | DEMEEKIRQILNRYYEALDKGDADAIFELLHDDWRMHSSDGTTFTDRHQFKEWVEKFNKEFNSRREWEIHIDRSRGTVTSHVRVTLNQGDKQTHFTVTETHRFRGDRITETRAHYDTQE |
| 1147 | DDEEEIREKLRQWIREFIRAVNEGDRDALKELYHDGATMHHASGRTWTTKEEIAEWFKQFTDQGFRLEVKSLEIQVDTDGSATVHVHVQVHQENTGRTFDFTSTFHFRFENGQMKLQEERIQTDEQ |
| 1148 | DSEKERQEKAKEWVRRYIEAFNRGDMDALAELFWDGWTFHHSNGQTFTTRDEFREWAEKLFSNDVQVEIESIEVQIDDNGRATVHVHVTFHFRRTGRTVTATETHHMEFHNGQLFVRETRAHWHQR |
| 1149 | DSEEEIKEKIKEFVRREIEIINRGDLDAWKSLFTPGSTMHINNGRTFTTQDELKEFIKELFDNQLQFEIKSLEIEIDSDGTATVHVEVEVHEERTGRTVTVRYTDRIEFHNGQMKIVERHITHNQQ |
| 1150 | DSEDEIKEKIKEFKKQLLEAFNKGDADALAELFTPGVTVHHSSGQTFTDLEELKEWMEKFTASGFQMQIDSLEIHVTEDGSATVHVHVTVHNESTNETYTVTFTDHYRVENGQLKITETHIYVHKQ |
| 1151 | DEKEMKEAAEELIRRFYEALNNGDADAAKSLFDEDKAEMHINGQTFNNADEIKKWIKEVFDNNTQLHVHSIHIQTHDDGTVTVTVHVTVTTNGNTTTVTHHWRFQQEDDKFKIVEVHQQTDQR |
| 1152 | ADEELQEQIKEWLKRYYEALDKGDLDTVLSLYAEDATVHNSNGQQVHYDEWVKRYIESLMKRFDSTRREIDEVRIELRGDTITVTVHVTMHFTHNGKHTTVHVTQHFHFKLETSDLKLKRDQVHNDSNSD |
| 1153 | TDEEAQRDIKEWLERFYESIDRKDLDKIRELVHPDATFHNSDGQRVPADEWVDKFYETVVKAFDQVHHQIDSIEIRQDGNTFTVTVHVTVHTTQNGQTYTFHVTHHWHFKKDSKEFKVTHMEEHRQQNKD |
| 1154 | PDQETREAIKEWKERFIEAINKGDMDTLKELVHPDATFHFNNQTFTDREEFAKWVTKMNRDGFRVEVKEIRISQHGDTATVTVHLTFTNEQTGSTMDVTAHDRYRRSNGSWKLVETHFHIQEKS |
| 1155 | ADKETQEAIKELIDRFAEAFNKGDWETLKSLFHPDARVHVDNQQFTTLDEIAEWAQKMFSQNWEVEVKEIHIELRGDEAHVTVHWEFHQQDTKTTVHVTTHITVRRSNKDWKIIRMEVRRNQQD |
| 1156 | TDPSDQEEIKEWKRRFLEAINKGDLDKVKKLVHPDATFHFNNTTYTTQEEIAKWVKEMFDQKWHIELKSMEIQKDGNEAHVTYHVTFHQEDTKTTVDVQAHDRYRKDNGDLKLIESRFHHNQKD |
| 1157 | TDEEAQRAIKDWFRQHVEAFNKGDMDKLASLYHPDATMHIDDQTFTTREEIKKWFEELFRQKFRIEVKSQEIQQDGDEYHVTTHVTLHQEDTKRTSDVTRHLRFRRSNGDWKLIEDRAHRNTKE |
| 1158 | MSEEEIKEEIKQWKRRFIEALNHGDVDAILSLFHPDATFHHARGQTYNREQFKEWLERFASNSDLRIEVKEIRIEIRGDEIHVTVHFQVTNPQTKTTRTFTQHDRYVLSKDLKLTELQTHTHDG |
| 1159 | MSEEEIKEWLKEFFRALDEGDADTVAELLTDDSRLHIESEQVTYNSKEEIREWVERARSTFDAQREVHIHINDDGTVTVHWTVTTHHNGRTHTFDGTTHFHFKDGKLVEVRITVHQRD |
| 1160 | MGEEAAKEQIKEWVRRYVEAFNKKDLEALESLYTDDAQVTFNETQRTYDKDEVKRWYEKVLKADHQIQIDSIEFRTDDDTTFTVTVHMTVTNDKTGRTFRVTFHFHFQRDSSQLQLTHLQVQIDK |
| 1161 | PSEEAQQAVREIIRRFYEAFDNNDLDTLKSLMHDDWTFHNASQQITFKSREEFLEWVKEFLKTFPDLKHEIKSIEIHQHGSTIQVHVHVTVHFGHSQQTYTVHDTHFFQKHKDDYKIVETRSHVDQA |
| 1162 | PSEEDKERIKEVLRRFYEAFNNGDADALASLLHDDATMHSEYNQITFTSKEEIKEFFKEFLKRFPDTEIEIKSIEIQQHGDYYTVTVHVQVHVKAHQQTYRVTHHWVLQKNGDDFRIVHVRERIDQT |
| 1163 | PSEEDQEAVKEWVRQWIEAWNKRDADKVASLYHPDATFTHNNETYDLSRDEFKEWVERWMRSFKDFSLELKSMEIRKSGDTITVQVHITIHYGDNNQTVDVTYHFFFRKENGSYKLIRQRTHRNERKE |
| 1164 | PSEEDKKAIEEIIRRYYEALNEGDMDAIAELFHPDATFHEDNQTFDLSREEFKKRVKEFKERFPDVEVRIDSIEIRQSGDTITVTVHVTFHFGKNNQTYHVTHHWFFEKQNGDYKITELREHRNNANE |
| 1165 | DREEREKQVRDWLRRFFDAVNKKDWKALMDMLAPDWRFHESNGVTLNNKEEFREWFERLMSNDVKIEIKSMEIRVHGDHVTVTVHVQVTHDSTGRTFRVTEHHHFRFTDGKLRLIETRVHHDGGEG |
| 1166 | DTPEDQEEIKQFKRALVEAFNNKDWEKVVEIIDEGATLHFSNGITLTTKEEITEWVRQFLSNDTKIEVKSIEIQKHGDEARVTVHMDVTFDRTGRTFKVTAHDRYKKTDGQLRLVESRFQFNGGEG |
| 1167 | DEEEMKRAAEEFLRRLYDAFNKGDWEALLRLFADDWTMHISDGETYTTKDEFREWVERMLSNTAHFQIDSLEAEVSGNTVTVTVTVTVTHDHTGRTFTFHDTHHFQFTDGQLRLVETRIHREGGEG |
| 1168 | PEEETRRQIEEWLHRFYELLDRGDADALAELIAPDAQYTDTNSNRTYDQEEFKEMVEEFKSKFPDSQHHIKSITFHQSGDEMTVTVHVEVHYNGSNQTVRFTDHYRFRKTKDSVRVVELRSTTDNTS |
| 1169 | MDKEAEEAIERLVRAFMEAMNKNDLETLKRLLSPDAKYHNSDTNRTYDQEEFFEMMERFMRTFKDFRIEIKSMEFEVHGDQIHVTVHWELHLTGSGQTQEFTTHITVDRTKDSFRIVSWTETTHNKG |
| 1170 | ASEEEAKEWIKQWVRREAEAWQNKDLDAWASLFHPDSQVTHSDEQRTFDKEELREIVERILRRFEDVSVEVKSLEIRFHGDDVQVHVEVTFHFSGSNQTRNVRYTDRFRIENGQFKLVSRTVTTDGT |
| 1171 | TSDEEAKEALRQVVRRFYEALDNHDWEAIARLLDDDWTFHNADEGQTYDKEEFIEWVKEWLKRFDNFRHQIDSLEIEKRGDEYTIHVHVTVTFDKSNTQHRTTDTHVIRQENGRWKIVETHMQTDGT |
| 1172 | MSEEEQREAVKDVVRRYIEALNKKDLDALLSLFDEDWTYTNANENQTYDKEEFREWVERFLKTFPDVKIEIKSQEIEFRGDTITIHTHVQVHFAGENQTVDVTETHHLELENGQFKIKRTQTRTDSS |
| 1173 | SEPEQREKVKEWVKRFYEAFNRRDLDELASLVHPDGTFHHNNQTFHLGKDAFKEWVKEFLERFEDVKIEIKELHVEVNGDTVTVEVHVTFHFHHKDRTQTRHVTDHYHFRFENGELKLVSVQSHTDTRD |
| 1174 | PETEEVKEQIKEWFKRYAEAWNKKDWQTLAELFDPNSEFHFAETQRTYDWEQFVEWVQRLLSRASSARMELKSLRIEQRGDTFHVTTHWEVHYSETKETYHATTHLTFKKENGSWKLAHFQIHTQKT |
| 1175 | PEKDEAEEAIRQWVRRYIEALNKKDWKKVADLFHPDATFRDSREQRDFNWEEFVDRVKKLLEHLSSSRIQVDDLHIEQRGDEFTVHVHWTQHRDETKTTFHGTTTLHFRKENGSFQLKEMQTTTQQT |
| 1176 | PEEKEAREKIREIKRRFAEAFNKKDWKTLADLVHPDSTMEDQSTQSEYNWEQLVRIVRRRLESLKSFRVEIDDMEIRAHGDEFTVHVHMTLHRSDTKTTYHIDATDHYRLKDGQVRLVRSTFRTQPS |
| 1177 | PGEEAMEEAIRRIKRRLVEAINNGDWETVLRLFDPNATVHFSKSPQTATTLEQIRRFVEEFLKANAHFEIKSIHIQFDSENTVTVTVHITVHFDNTKTTVDVTFHDFYVRKDGQVKIVETRIQFDHT |
| 1178 | PGREEQEEQIRQMLRRFAEAFNNGDVEALAEMFDDDWTVHFSQSNTTFHTKDEFKKFIKELLSQAWRMEIRSIEIRFDSDNEVTVTVHVTFQNEKTGETVDVTEHHHFRFENGSVKLVETHIQFDHK |
| 1179 | PEEEEDKRQIEEIDRRRIEALNHGDADAVLSYFHDDAEFHYSATNTTLTTKEEIREWFERFISIPFRIQIHSIRVELDGNGQYTVHATVTVTHRPTGTSVTFTYTIKYRRTKDDWRIQRVQIHYDDGGD |
| 1180 | ADPKAQEDIRQWVRRFYEAFNNNDMDALKSFYDEGATMHLWNGQTFHSQDEIKKWFAEMFSSGVRLEIKSIEIHQNGDEFTVTVHVQAHTSHSTYDVTQHFFFEREGDTWKLKRDEIRMHQT |
| 1181 | PSREEIKELLDRMYESLNRNDVDTFVSLFAPDWTLHIYQGKTFTTLDEVKEWVEKWLSDWTGSRIQIDRMEIHGDTVTVHVTQTLSKTGTTVTITDHHTFQNGKLTETHSHEDST |
| 1182 | MSEEEAKEWVKQFYEALNNGDMDAVASLYTPDATYHVWNGQQFDTRDEVKEWFEKYFKSAKDAQIQIDDLHVDGDTVTVHVTQHEGHSGRTFTITQHFEFTNGRLKRDETRRNQQ |
| 1183 | PSDEEIAETLVKRYIDALNRKDIDELASLYTDGSRIHIHNFRGETINIRDKDEYRKVVEKMLRELEDVHFEIKEIRVDGDTVTVHVTMHVTDSSTGKTTTVNVTQHIQFKDGKIVRHEAHFNQTD |
| 1184 | PSEEEIAETVNKRIADAFNRKDLDKLVEIFTPDWQAHITNWTGNTVTFHSLEEFREWAEEWLKRFQDTHIEVKETRVHGDTVTVHMTVHTTDKSTGKTTTINVTEHHHFENGRLVETHIHIHQTD |
| 1185 | ASEEEIRELIRRLVEAFNKGDMDAIKELYDDGATVHFWNGRTFTSKDEIKKWFASMQKDIRLQITRIEVDGDEGTVHVQVHNNSNPKTFDVTSHIRFKDGRIKSERVHMRETD |
| 1186 | PSEEEIKEIIRRFVEALDKGDADAALSILHDGWQLHVYNGQTFHSREEFKKWVESLSKDTHRQIESIEIRGDTVTVHVTVTHGENPKTFKVTEHHRFKDGKITETQIHMNQTD |
| 1187 | MSEEEIRETVKREAEIWNKKDWEAWASLFHPGATMHHSNGQTWTDLEELTRWFQSIDRSVKVQIDSIEVRGDTVTVHMTFHNNKNPQTFHVTVHIRFKDGKITEKHIHTNQTD |
| 1188 | MSEEEIREVVKRFFEALNKKDWEAIKRLLHPGVTIHHSDGRTFTDLEEFAEWVKSLDSNLRIQVESIHVDGDTVTVHWTVHNGKEPRTFRVTTTFHFKDGKITEIHVHRNQTD |
| 1189 | PGEERIKEFLRRLFEAIDRGDMDAIKELFAPGARMHINNQTFTTLDEIRKWFEEMFKSLKNSRHQIDSIEITGDTVTVHWTLHVTHEDGSTVTMRATTHFTFDKDGRLVEMRIHEDQDDR |
| 1190 | MSEEQIRQIVRRLVEAFDKGDWKTLKELYAEDATVHFSKGQTFTTLDEFAEWIRQFASNWDLQRQIDRIEFNGDTVTVHLTVHRHHGDRTITVHVTSTFHFKDGKIKEEHIRWDERE |
| 1191 | TSEEEIKQWIEEFFRAIDQGDAEAIKRLFHDDARLETNRGEEWTTPDEIAEWFKRVWENWDSNHEIKRIEFNGDTVTVHWTAHRNHGDKTVTLHGTTHIRFKDGKVTEMRIHVDEHD |
| 1192 | PSEEEIKEILRRFYEAFDNGDLDTLKELLHDDWQFHESSGRTFTTLDEFLEWVKKVIDNWNTAHHIERIEINGDTVTVHVTVHRHHGDRTITVHTTDHHTFKDGKITRTEVHWDERE |
| 1193 | TSEEEIRETIRRLVEAFNKNDWEALARIYDDGFTFRWSNGETLTTKEEFVRWVQEMIKNSDTKVEIKSIEIRGDTVTVHVQVHTRHGDKDHTVDVTSHFVFKDGRLKEEQFHTDQRD |
| 1194 | SGEDQERIEEWVRRWIEYMFKTGDADTVASLMHPDWTFHLYNGQTFTSLEEFAEWVRQWWSQFPDAHVEIKSVEIENGTVTVHYTVHVTHNGQPYTFHVTEHHRFKDGRLTETHQHRRNT |
| 1195 | SEEDQERIEEIKRRMIEYLIKTGDVEHFASLLHPDAQLHVDSGTTFNSREEMKEWWARFFKNFPDVRVEIKRIEIQDGHATVHVEMHATHNGQKYTFHQTQHDRYKDGRLTESHIHTETR |
| 1196 | MSEEQIERWIRRMFEAWNRGDVDKLVELYTPDVEVHYFHGNETYTFTTLDEIKEWLQQLFERFEFHIQIHEIRTNGDTVTVHVTVTFTHGDETYHMRVTRHFHFKDGRIKEDHIHFDQKQ |
| 1197 | ARREEIERTIRRMLEEVFNTGDADAAASYYDDDAQLHIWNGITFDTKEEIKKWIEEFWKRFPDSKLEIKHVEFTETHVTFHYTVTFNHDTGQIHVDFTSHFEFTNGKLVSERIHSNQRN |
| 1198 | MDESEIREILREMYERLFNTGDADAFASYFHPDATFHFSEGITFTTLEELKEYLRQWYRKFKDVHVQIDSMHITETHVEVHVTIHVRNKDGKVTIHVRHHWRFSDGYIVELRAHFDRKN |
| 1199 | SSEEEIKDIKKKFFEALDKGDLDKLHDLIHPGATFHFAWYQQTINDRSEFLEWVRSLLENADVQHEIKSIHVSGETVQVHVTFHVTHNGETRQYQATVHDRYKDGKLTESRFTTNQD |
| 1200 | PSEEEVKEWVRRFYEAMNNGDLETLHQLLHPDATMHVSRVQITFHDRSEIIAFIKRTLEEFDLSIEIKSIHVGGDTVTVHYQVHTTHNGQTRTVDVTDHFHFKDGKLTEVRSHFNQT |
| 1201 | SRKEEIEEAVRRFFERFIRAFNQNDADQVAEAFDDGATFHAPDGTQFTTKDEIKKWVEEVASQFDGIKVDDVEIHIHDDTVTVQWHITFHHGDETYHIDVTTHITFKDGKIKEFRIRFDNKE |
| 1202 | SEQEEIKRILKDFYEKYFDALNKGDVDKIIEIIHPGVTYWHTSGQTFTTLEEIKEFIERLLSQIRGIEVKNMEIHVNKDKATVHITQTVNRGDRTYHVDSTHHWQFKDGKITEIREHNDQPT |
| 1203 | SQNEEVEELVRQFFERFLRALNTKDADTFLSLFDDDWKFYHKSGRTFTTKDEFKEWFERFASNFDRVEIKDLEIHVNGDTATAHVTFTYTHGDRDFTNTVTEHHHFKDGKLKETRQQRDNQQ |
| 1204 | PFSEEQVREWVKQYAEAVNTKDLDAIASLYDEDATFHHSNGRTFTTREEIKRWYEEFLRQDWQVQFDEIRIEGDTVQVHVTFTNSHDSKSWTATFHFHFRNGKLKEVRVQENQT |
| 1205 | PLSQEEAKEIIKQYVEAFNTGDMDKLKSLYTDDATMHHSNGQTWTTKDEIKEWYERLFSQQWRVQLDRVEVHGDTVTVHLTFQNETDSKTVKYTFHIRFENGKIKEVQIHTNQT |
| 1206 | PGEELIRRILRRFYEALDNKQDELKEILHPGVTYHDSSGRTFTDWREMADWLLEWFRSHFPDAKREIHSIEIRGDTATVHYTIHVNTDDRTITIHATDHYRFENQRLVEVRSHRDQDNNS |
| 1207 | MDEEEIKQLLKRFYRALDEDWRKFASLVHPGATFHFSGLGITFTTHEEFIRWFTEWFRKRFRRTAHEIKEIHVDGDTVTVHVQVHVTHDDGRTQTYDVTEHHRFRNGKLVETRFHFHSK |
| 1208 | DSEDQQKIKELVKRFFDAINRRDLEALRRILHPDAQYHNNNGQTLHTLDEIIEWFKQLMEEFPRAHIEIKSIHIDSSTKTATVHWTLHVTHPDGQNQTIHATTHFVFKDDKITEIHVHENGG |
| 1209 | PSEEELKEWVKQFFRAWQDWEQAFEFIHPDTTIHEHRNGETYTFTNREEIVDFLQKFLSNHNIQIHIHRIEVHGDTVTVHWTLTVNHDSQQYHYSATTHFVFKDGRVTEVRMHIHQTD |
| 1210 | QGKEEIEKIIRRLYEAFNRGNLDEFISLFHDDAQQHTNHPSGETYTFNSLEEIKEWWKRLFDNDTHVEVKSIEIRKDTVHSTVTVTINHNTQTKTMTVHETHHFKDGRITETHITFDDDG |
| 1211 | PGEEEIRRWLRRYYEAFNRGDADELASLFDKDWTFHYQDHNGQEYTFTTLEEYKEWVERLQSQGAQMEVKSIHFDGDTVTSHVTIHITHGDTDHTFDVTEHHRFKDGKVTETHVHTTDRG |
| 1212 | PLEEQIRQWVKRWVEALNNKDVDALASLYHPGATVHFSKGETLTTLEEIREWFERFAKTFKRMHIEIKDLHIHGNTVTVHMTVHLTQDGRDVTTNVTSTFHFKDGKLTEQHVHIDDEH |
| 1213 | TDKEQQEKIKEVVERFYDALNKKDLDKVASLFHPHAQMHISDGYTAHSRDEIKKWFKEWMEKVKDFRIQIEEIRIEGDTVTVHVTVTTHHNGQQYHIRVTEHHRLDHNGKIVETHQHVDNSE |
| 1214 | ASEEEIKEWVKRFFDAFDKKDMEALKELVDEGATFHFYQGKTFTDKDEFAKWVEEWLKRAKDTAHEIKSIEFRGDTVHVHWTVHVHHDGRTVTIHATTTFHFKDGRLTEVRFNLDNQPT |
| 1215 | TSEEEIKEWLRRFYEAFNKGDLDDLHRLFHDGATMHISNGQTFTTREEIKDWFRRVLESAKDIKIEIKRIEFDGDTVTVHYTVTVNHDGKTVHVHATDHFRFKDGKITEVRSRFDQHDT |
| 1216 | TSEEEIKDVKRRFIEAFNKGDLDALASLVADGATIHFAQGETITTKEQFKEYIDRVLKSVGRVHIELKEVRFDGDTATAHVTMHVHRPGKTVTVQATQHDRYKDGKLKESRTQFDQQDS |
| 1217 | ASEEEIEELLRQFYEAFDKGDADRLFELLHPGATVWLWEGTQFNTREEFKEWVKEWFKQAKRVHREIKSIEFRGDTVTVHYTVHVHHDGKTVTTHATDHFRFKDGKIVEVRSHEDTHDT |
| 1218 | PSEEEIRQWLKRFYEALDKGDMDKLRELIDDGATFHLHQGTTFKDKSEIEEWMERWFKTVGDVRREIERIEFHGDTVTVHYTLHVHHPGKTVTIHATDHFHFKDGKLTRMESHFDRHDT |
| 1219 | PGEETIKELLERYYDALDRKDMDALEELVHPGATMHDSNGYTATDREEIKEFWRRLFETELKDSRHEIKEIHFDGDTVTVHVQAHFTHKTGQHFTFDVTHHWVFKDGKIAHLTYRVHQKD |
| 1220 | PGEEHIREWKKRFVEALNQGDYEKLQELLAPDFTYHNSNGYTATTKEEFIRFYQELHKNGFTDVKMEIKEIHVDGDTVTVHVEVHVTHKTGKTYTFHVTTHDRYKDGKLVETHQQWHQQD |
| 1221 | PGEEAIREWVKRFVEALNKKDMDTFKSLYHPGSQMHDSSGWTGNDVESIVEFFKRLFEETYTDVKIEIKEIRIEGDTVTVHMTMTVTHKDGRTVHIDATRHFRFKDGRLTEDRLHFDQKD |
| 1222 | MSEEQIRDFVKKYLEAINNKDVDKIVSLYAPDATVHFSRGETLTSLEEIKEWYKRFLESVGDIHVELKEQRIDKDTVTSHVEIHVTHKDKTKTVDVTFHFHFRGQRLTEVRVQEDDRD |
| 1223 | MSEEQIREIIHRWYKAWNDKDVDKLAELLHPDWTYENNNGERINSLEEFKKWVEEVMSSNGDFRVELKSIHIDKDTVTVHFEVHVNHKDKTKTMKTTDHHRFRGDRITETQVHVNDKD |
| 1224 | MSEEQIKEWIKRYVEAFNKKDVDTLASLFSPDWQYHDSSGETIHSLEEFKEWVKQFMERAKDVRMEIKSIEITDDTATVHVTVTYNHDGKQIHADVTEHHHFRGDRITETRVTFNPQD |
| 1225 | PSEEEIKEILERYFEAWDKGDWEKLLELLHPDWTQHVSNGQTFTNLEEFREYFERFLSRFERSRHEIKRIEFDGDTVTVHVQAHFRQGHQDYTTDVTEHHRFKDGKIVETRAHYDTKRT |
| 1226 | SDREEIKEFKKQLIEALDRGDADAFAELVSDDVTFTFQQGITHTTKDEFAEWVRRWFSSGFDEVRREIKSMEIDGHTATVHVTVHANHRNGQTFTTHVTYHDRYQDGQLTETRASFDTKDS |
| 1227 | PDERIEEIIRRWFEIFFKTGDADKAADLLHEGATVHFHQGITATTRDEIIKWFREFFSTLKDIKFQIEEIRVDGDTATVHWTATVNMNGKTVHYRGTTHFHFENGKIKEVHIHIHQEDQS |
| 1228 | TSRKEAEDIARRYLEEFYKTGDWHAAADYYDDGATVHFAAYQITFKTLEEIIRFLRRLLSRMQEAHVEIKEVHIEDNHVTVHYQLHFTTDGQTQTYRFTSHIQFTNGKIVSERIHMDSGE |
| 1229 | PDEEEIRKLINRYFEAFDKGDLDTLKSLFDEDWTQHHSSGRTFHSLDEFKDWVKKFLSNVETAHEWEIHIDPSGETVTVHVTVHLTHGNRTTTVHVTEHHHFRNGKIVETHIHEQPDS |
| 1230 | PQEERIKEILRQLFEALDRGDADHFASLYAPDATIHFSQGITLTTREEIKEWVKRMNANSEIHREFEIHIDPSGSTVHVHFTVHRTHGDQTHTFHITSTFHFKDGKIKREQVHIQPDS |
| 1231 | PDKEEIKEWIKRYFEAMDKGNVDELLSLFDDDWQYHNSNGETITSKEEFKEWVERWFSNVDTAHEVHIHVDDNGETVTVHVQVHQTHGDKTQTIDITEHHRFKDGKIVETRVHVQPKS |
| 1232 | PAKEEIKEWKRRYFEAFDNGDIDAMAELVHPDAQFHYSNGITFNSLEELKEFLESTFKNTDSKHDFEVHIDDNNETVTVHVHVHLHHGDKTQTIDVTQHDRYKDGKLTESQIHEQSKS |
| 1233 | PDKEHIRTILERLYEAFDRGDADAVASHLHPDARFEFSKGYTITNLEQFIEWVRRFISNTDVHREVHIHIDESGEHVTVHYTVHFNHGDRTQTVHATDHFRFRNGKITRVQSDVQDDS |
| 1234 | PDEEEIKRLVEQFFEAFDKGDLDALASLYHPHATVHFAQYQVTYHSRDEFRKFFEEFWSNFTGSHHQIHEIRVHGDTVTVHVTTTFTHNGQQVDVRVTRHFTFKNGKLVRDQIQFDHT |
| 1235 | PLEEQIKEWKKRFIEAVNKGDMEKLRQLVHPDAQFHFSASNTTFHNLEEFLKWVEEWLKKAKDARIEIKSIEVRGDTVTVHITVHTTHNGQKVTVRVTVHDRYRNGKLVESRFQFDGS |
| 1236 | PSEEEIKDFVRRLFELLDRHKDADLMKKLYAPDATMHVSNGITFNSQDEIKKWFEEFQKKFGFQAQIDSMEVRGDTVTVHVTITMHKDGQTYHVKVTSHFRFDKEGKLKEEHIHEDQQD |
| 1237 | PDEERLKDWYRRVYEEAINKRDPDALASYYAPDAEVHITHNGNTQTYSREEFKEWFERWLKRFPRVHVQIEEIRVDGDTVTFHVTMHFHHNGKQITVTVTYHVTLQGDKIKEEHVQFDNQRD |
| 1238 | PDQEELKEWKKRVIEAIFNQRDADLFKRYVHPDATIHITQNGNTQRFDLDEFKEWFERFLSQFPRIHVQIDEIRVEGKHVTVHLTFHVHHNGKQVTIKQTQHDRYEGKRLKETHIHTDNDHP |
| 1239 | PGEEDIKEWLKEFYRAIDEGDAETLKQLFHPGATVHFQDGETFHDLEEIVKFFQELFKRSRSRHEIESIEIRGDTVTVHYTAHFTHNGQNYTFHATDHFHFENKRLTEMESHSDQRD |
| 1240 | PGEEKLRQLVRRYIEALNKNDLDKLEELYDDGATMHLSDGRTFTTKEEIKRWFEEMIKKSDARFEIKSIEVRGDTVTVHVTVHHTHNGKTITIHVTSTFHVRGDRIKEERVQFDQQD |
| 1241 | PGEEALREWIRRFIEAFNNRDLDKLRELYDEGATLHISEGTTFTTLDEIIEWFERWMKNADFHIQIKEIRVDGDTVTVHVELHVTHNGKTVTIRVTSHLHFKDKRLKEERVQFDQED |
| 1242 | PGEEKIKEWKKRFFEAFDKGDLDKLRELIHPGATFHFSEGTTFTTIDEFLKWVEEFFSNADFRHEIKEIRVDGDTVTVHVTVHVTHNGKTVTIQVTQHDRYRGDKLVESQIHFQQHD |
| 1243 | PGEEKIKKFVERFFEAINKRDLDKLRELFHEGATMHISEGTTFTTRDEIIEWFERFIKNAEFKFDIESIEIRGDTVTVHWTVHVTHNGQTFTIHATTHFEIRGDKIARVEIHFDQQD |
| 1244 | SSEEEIKQWLERFYRALLETNDPDAIASFFDDDATFHVHRGITYTSRDEIKKWFEEWLRSVSDIRIQIEHIEFRGNTVTVHYTVTVHQGDRTIHLTATDHFHFRNGRLKEMESNLNDQQ |
| 1245 | PDEEEIKEWKKRFFEAMDHNDIDKLLSLFSDNVTIHFSQENITLHTREELKEWIERFLSRFKDVRHEIKSIHIHGNTVTVHVKVTFNTNGRTYHVDVTFHDHYRNGKLAESRVQFDQS |
| 1246 | DSREEIKEWIKRWVEAFQKNDVDELLSLYADHVRVTFEAENQTLTTREEIKEWLERFIKRFKNIRVEIKRIEINGDEVTVHMTITVTHDGEQYHVDVTFHFRFKDGKLTEQRAHFSPQ |
| 1247 | PSEEQQKQVEEFVNRYFEALNKKDVDTAASLFDDDATIHFADNITFTSKEQFRDWVEKFLRHSDIKVEYEIHIDSSTGTVTVHWTMHVHHSHSTHTMHATTHFHFRNGRLKEMRTQIDRED |
| 1248 | ASEEQKEQVRQFVERYYEAIDKGDLDALDELLHPGWTYHNSNGRTFTSKEEFKEWVKRWISNMDTAHTMQVHVDSSHNRATVHGQVHVRHDNSTHTVDVTEHHQFKDGKVTHTRAHVDDRS |
| 1249 | PSEEQQTQIKEIKRRLAEAFNRGDVDALYELYHPGATVHDSSGITFTSLDEIKEWLRQFFENLDVNVEFEVHIDSSTGTVTVHFTFHVTHSDRTKTVHATTHDRYKDGKLTESRTHWDEKS |
| 1250 | PSEEQQSRIKEFLKRLYEAIDKGDLDALHELLHPGWQFHQSKGNTFTDREEFREYIKRIFDNSEVHREVHIHIDKDTGTVTVHVTAHYHHDNKTKEVHYTEHHEFRNGKLTSTQQHVDEKD |
| 1251 | PSDEEKIRELIKQFYRAFAEGDWETLMRLLDENWTVHFARHQVTFHSKEEFREFVERVNRNSDIKFEVHIHIDKSTGTVEVHVTFHVTHGDETRTFHTTDTHHFRGDKITETHFNFNTT |
| 1252 | PSDEEKIREWLRQFYEAFARGDLEALFELLSDDVQFHNSAEQVTFNTREEFKEWVKRFLDNSDVKMEVEIHIDKSRGTVTVHVTFHVTHGDETKTFHATHHWHFRDDKITRVEIHINET |
| 1253 | PSDEEKIKELIKAFYEAFESGDLDRAKEIVHDDATFHITSENVTFHNPDEFKEWMKQVLSNSDIKMEIHIHVDRSTGTATVHYTMHVTHGDETRTRDATDHFRIRGDRITEVRSTWNQR |
| 1254 | PHQREKIREWLERYYEAVARNDLDAVFELVSEDATFHYYWEQVTFHTREELKEWLKRLIDNSDTKMEVHIHIDDRNGTVTVHVTFHTTHGDETRTYHVTHHWHFRGDKLKEVHFRINQT |
| 1255 | PSDEEKIREWVRRWAEAWQKGDIETLASLYTDDSRIHYAAYQITFTSREEWKEWVARHLSNVDVKLELHVHIDRSTGTVTVHITFHFRHGDQDKTVHVTVHFHFRGDRLKEQRVHINET |
| 1256 | PDEEREQAKEWVKRFIETWVKTGDADAVEEFYDDDATMHLSNGQTFTNKDEIKEWFKRWIERFKDMQVEIKSIEVTDDTVTVHVTVHTTHNGEKYTFDMTSTFHFKDGKLKEERIQEDDKG |
| 1257 | HDEDRERAIEWVQRMIEMLVKTGDADAAASLFHPDWTYHHSNGQTFTTLEEWKEWVKEWVKRVKRVHVEIKEIRATDDTVTVHVEVTFHHNGKQVHIDITEHHHFRNGKLTRTQTHEHSRG |
| 1258 | PDEDREEAEEIVRRFYERWVKTGDYRVAQELMHPDATFHVWNGQTFTNLEEIAEWFKRWVERFKDFRFEIKSIEFTDKTVTFHGEVHFTHNGQQVTQKVTHHWTFRNGRIVEVRVRRHSTG |
| 1259 | PSEDQRQIEEFVRRWIEIVDKTKDVDAAKSMLHPDWQFHDSNGQTFTDLDEFREWLERWLKRFKDFRVQIDEVRFDENTVTFHVTFTFTHNGEEYHFDVPEHHTFKDGRLVETQVHENDVG |
| 1260 | TSEEEMERVLRRYYEAFNRGDLDALVELMTPDWTYHDETHGITITTLEEHRDWVRKFMERFKDYRFEIERIEFTGDKVTVHVTLHFTDKRTGQTYTVRVTDHHRFKDGKLKETRSRFNEVK |
| 1261 | QDDHQAAEEIIRRYVEAYNKGDMEKIKSLYHPGSTYHQDSGQTFDHEEFLEMVEKRARNTHVQLDHIEIHVKDGTITVHATMTVTEKGSGRTYEFHSTFHMEKKGDTLYIVEEHLHSDST |
| 1262 | PHDQQAAKEAVRRYLEALNRRDVEAVLRLFDDDWTYYDSSGQPYDKEEFKEWLEKYASLNFRIEITTTIHAEVKDGEVTVTIHVTVTWEQTGQTTHMTEHHHLRRDSTGWKIQRTQTHTDNTPT |
| 1263 | PDDHEEIKELVERFLEAFDKGDADTLDKLLHDDWRYHHESGRTFDKEEFKEMVERFASVRVKREIETTIEIHVKDGHATVTVHVTVTFEDTNTTTHATETHHLRKTKDGWKFVRTQQHTDNQPT |
| 1264 | PVDREQAAEVIERYYRAWQNKDIDALRSLLHPGATFYDSTGQRYDREEWLEMVKELLRHTDAQVQIHFDHFEQDGDEIRVTVTVHITYQATQQTYTATHHWRLRLFNGSVKILEVREHTRTS |
| 1265 | MSEEHIRRLVKRYYEAFNRKDMDEIKSLLTDDWRYHNDKTKRTYDVEEFLDMVRKALSTDIEIRIESLEIEIRGDHATVHVRVHITHRQTGQTFTTRDTHHLERGDGSGKFKETRVQFDDGH |
| 1266 | PGSEEEIKELLQRFYELLNNGDADALAELFHPDATFTFEGQNYDLEQFREWVERLFRANVDIQINGNIEIRFDGDEATATVQVTATNKQTGETYNFTHHWFFRQIGNQVFVIRLEIHSDQS |
| 1267 | AGSKEEIKEIIKRLVEAFNKGDLEAIKSLYHPDATFEFDGQTYDLEQYIEMVEELHKNDAKLHINGNIEIRISGDEATVHVNFTITNSQTGETYTYRRTFRFRQQGNDLRIVRDEVHSDQS |
| 1268 | AGSEEEVKDIVRRLIEAFNNGDLDALESLFHDDWTFHLNGQTYDKEQFKKMVEEMFRAGVKIQIEGNIEIEFDGDEVTVHVTFTVHDDRTGTTYTFHETHHLRLEGNRWRIIETRVTTDQG |
| 1269 | DAEEAIKQWVRRYVEAWNKKDMDALESLFDDDWTFHDSDGQRFNREEWKERVKRIQDNTDSQMEVRNSIHIEIRGDTAHVTVYFTVTFHHSHSTHTFSVTEHHTFERENGDWKLKETRTRMHHT |
| 1270 | DDQEAIKEWLKQFYEALDKGDMDALRELLHPDVQFHNQNGEQYDLDEFLKMVEEQNRNSNSKHDIINSIEVRINGDTITVHVTVQVHFHHSNRTQTTNFHETHHFEKKDGDFKLLRTETHSEQT |
| 1271 | AGEEAVRETVRRFYEAWQNRDMDKLRELLDEDWKYHNTATQTTYDAEEWLEWVKQFASFNFRVHIHSMEVRQDGDHYTVTVHVTFTDDHTGRTVKVTDHHHFRRGDGRLKVVETRTHYHTS |
| 1272 | SDQEEIRQWLRRFFEAMNNNDIDALVELFSPDWRFTHSSGTTDNSLEAFRKWVEEFMRRFDDVKIEVKEIEINGDTVHVHVQIHTTHGDKTQTFRVTETHHFRNGKLTETRVHVYDSG |
| 1273 | ASEKQIRDAVREFYRAFNENDLDALMELIHPHAEVTFSAEGRTFTNREEYEEWFRRYMSRFDAKVEIKDMHVQDDGTVTVHVTITFTHNGQEFHVDVTDHYHFQDGKLTRVHSQFDQQ |
| 1274 | TSEEEIREILKRYYESFNKKDVDKAASLLAPDWTQHNAAHQRTFTTRDEFREYFEKFAKRFDAHVEIKSIRIWSDGTATVHVTMTFTHNGQEYHVDVTEHHHFKDGKIKETRVNFDQR |
| 1275 | TSEEEIKELLRRFYEAFDKGDWEKLHELLHPDWRLHVAEEQQTWTTREEFIEWVKEFFKRFNTAHRLEEIRVHDDGTVTVHVQMHRTHNGQTQTFDITEHHTFRNGKMVETRVSFQPT |
| 1276 | TSREEIKQLVERFYEAFDRGDWKALAELLHPDWQMHIGRLNITLHDLEELIEFAKKFLANFDIQRHIESIHISDDGTVTVHVTVHMTHNGETRTFRTTDTHRFKDGKITETHADFDQT |
| 1277 | DQEEEIKEWVKRFVEAFNRGDLDALAELFHDDWTFHHSNGQTFNSKEEFKKWVEELLSFDTKIETKSIEIRGDTAHVHVTMTTNHPGQQRHVQVTETHHFSDGKVTETRVHFGDTQTT |
| 1278 | PSEEEIKEWLRRFFESLDNKDDEFAELLDDNWQYHDSSGNTGRDAKEMIEWLKKWFSQFDTAHHIDRIEIDKSRQTVTVHVTVHQTHKNGQTYTVHVTEHHHFRGKKLTETRVHRNQD |
| 1279 | ASEEQIRQWVKRFVELMNNNDVDALMSIFHPDWTMHVQQWNGQQYDFTTREQLKEWFKRLASLPFRIEIKSLEIRGNTVEVHITIHVNHKSGQTHTHDVTETHHFKDGKLTETHQRWDQK |
| 1280 | MSSEAIEELKRRYIQTAFNTGDADKFKSFFSDDSTMHVYRGITYTTLDEIKEWFERMVKEMQDYHVEIKSIEIRGDTVTVHLTTTVHRGDQTYQMTVTVHDVYKNGRLAHSEFRVDNKE |
| 1281 | SSEENKEFVKRLIEIFFNTRDVKQFLDIVDEDWTFHDSDGSTLTTREEVKKWLEDFLEHFKDAKVKIDEIRAEGDTVTVHMTMHVRHGDQTHTYHITEHHRFEGKKVTETRVRTHPTS |
| 1282 | ASEEEIRDWARRFYEALNNRDRETLHKLFSPDVQMHLADGRTFNSQDEITEWFNQWLEKAKDFRIEIEEIRVHGDTVTIHYTVEFHHKDSTVTIHFRDTFHFKNGKLTEVRSTWDERQQ |
| 1283 | PSEEELKEFLKEFYERLWNTGDWRAMSEYMHDDVTVHYYRSSGETYTATTIEQVVKYFQEFASRFPDVKVTIDSIEVHGNHVTVHITIHATTDNQDFQFHVTHHWRFRNGKIVEVQAHWNQKE |
| 1284 | MSEEEIREWLKRFYEAFNKRDLDKLLELVHPGARFHFSAHGETITDLEEFRKWFKRLLEELQDFRIQIDEIRVDGDTVTVHVTVHVTHEDGRTQTVHVTEHHHFVKDGKLVETRFHFNGS |
| 1285 | PSQEQIEELVRRFFEAINHKDVDKIRELLDDDVRVHFSESNTTFTTKEEVIRWLEEFAKRFKDIRFEIKEIRVHGNTVTVHWTAHFTRDDGRTFTIHATTHFVFKKDGRIVEIRFHLNTG |
| 1286 | ASQEEIKQWIERFFEAFNRRDVDKLISLLAPGWTFHNASNNQTWTNPEEFKEWVERWLKKAKRFRFQIDSIEIRGDTATVHVTVHVTRDDGRDFTYHITEHHHFNKDGKITETRQHINGG |
| 1287 | PDEEIKRWVKEFIEAFQHKDLDALVSMLTPGWTYHDTDGTTYNDPEEFREWVERFMSNADFRVEVHEIRIDKRSQTVTIHLTMHVTHPGSHHTYTATEHHHFRDGRLVSTKEDRDENR |
| 1288 | DQREEIKTWLDRFYESINQKDLDAIKRLYHPDATMHITKEQRTFTTQDEIKEWFERIFRTYPNMRVEIKSIEIRGDTVTVHVTITFTGDGKTFQVTDHYRFKDGKLIEQHSHFNDT |
| 1289 | PEEEELKELAQRFIETVARAMNNGDADAIISLFDDDSTVHFSSEGRTFNSKEEIKEWFEKFASRFEIHIEIKRIEVHDGRVTVHWQLHYTHNGQPYTTNVTTHFVFKDGRFKHFRFQADHS |
| 1290 | SSISEEQIREWLKRFYEAIDKRDRDTFRELIHDDAEFTVDGRTFTNADEFVEQFLRQWISNAQRTQRHIESIEIDDSTVHVHYTIHVTNGHERYQIHVTDTFHFRDGRLVRVQSHRDTKKT |
| 1291 | PISEEQIKEWVKRWVEAFNKGDMDKLMSLYSEGSRFHDSRTGKTYDREEIERLIRQHLSQGIKVQLDSLEIRGDTVTVHVTFHQDQTGRTFHVTRHLQFKDGKLTEDRATWNET |
| 1292 | TSEEQIKQLVERLIEALNRRDIDAFLSLVHPDWTDHQYHKNRSETYDKEEYKELLERMLSKAKDVKFEIKSIHIDGDTVTVHVEFHHTTTENGSEKHRTFRVTEHHRFRNGRLVETHSRFHDE |
| 1293 | ASEEEIKDWLKKWYEAFNNKDLEKLAKMIHPDATFHLTQGNQTRTYDYEEMIEWIERFLKHFKRFHLEIKSIEIRGDTATVHVQLHLHEDKNGSTETHTFRVTHHWVFNNGKVTEVRAQEDPI |
| 1294 | ASEEEAKEWVRRFIEAINRRDLDAIASLYADDVQFHFYHGDRTETYSKEEFREWFETMLRRWQEIHVEIKEIRIDGDTVTVHVQFHFTRTKDGSNETRTYRVTSHIHMRNGRVKEEHIHFHNT |
| 1295 | PMSEEEIKDVKRRLIEALNNRDADKFASLFHPDATLHFSKGETLTTREEIKEWVERFLKQFPTATFQVDSIHIDQSTGRVEVHVTVTVHHDHKTVHIHVTFHDRYDPNGRLKETRANYDEGQ |
| 1296 | PISREEIKEVIRRLVEAFNRKDVDELVELYAPDATVHFYQGETLNNLESIKEWLKQFFRTFKDVKVQIDSIHIDQSTHRVTVHITMHFHHDDRTKTVKVTRHFQFNSDGKLVEDRIHEDHGD |
| 1297 | LSEEQEKAARELARRYVEAFNKDMDELERLYTDDSQMHVSEGITFDTKDEIKDWLTKLAKNARLEIKDMHVEYNPDGTVTVHVTVTVHHPNGTVTVHSTLHFEQRNGQFYLKREHIRWDSN |
| 1298 | DAEEEIKEFLKRFYESLDNGDLEALARLIHDDAEMHVDGGRTFKTKDEIKKWFEEWLKKAKDSKHDIEEIRVDGDTATVHVTVHVNHKDGTVTIHVTEHHEFRNGKLVRTQAHEDPQPT |
| 1299 | TSEEEIREAVQRYYEAWNHKDMDKLLSLIHDDWQNDTYNLTTKEEYKEFVKKFISRFPDFRIEIEEIRVSDGTVTVHVTFHFNHKDRTQTIRTTDHHHFKDGKITRTRQHRNN |
| 1300 | TSEEEIKQWLERYYEAWNKKDLETLASLFHPDARIHSLGITTKEEFKEWVKRFFEQFKDVTVEIEEIRFSDGTVTVHYTVHQTTKDETRTTRYTDHYHFKDGRLTELRSQTDN |
| 1301 | PSEEEIRDWFRRYFEAVNNGDIDALKSLYHPGATVHFSGGRTFKTQDEIREWYERFMRNFNAKIEFEVHVHDNGTATVHFTMHVTHPDGKNQTIDITVHVRFKDGKVTEVRFHEDQS |
| 1302 | PSKESIKDWKRRYLEAIFRSGDADVAKSLVDDDATFHIAETQRTYDADEYREMWRQLMEKIQRVHVKIDRIEIDGDTVTVHVQIHVHHGDQTVTIDVTYHDKYRNGKLTSSQSHNDQKD |
| 1303 | PQQEEIKEVLKRFYESFNRKDLDAIKRILHPDATIHLTHNTTTHTYDLDQYIEWVKRMLENVKRMKIDIHSIHVEGDRATVEYTVHQTHGDKDITMEAHDHVEMRNGKIVEMRSHIDERPT |
| 1304 | DMEEQAKDTIRRLIEAFNHGDLEALKELYDDDATIHINKGDQTYTFSKEEFIEWVEEFLRRAQRARIQLDDLHIDGDTVTVHMTLHVTHGDKDRTIRITSHFRLKDGKIVEEHMHVDDTPQ |
| 1305 | PDEEEIRRWVEEFYEAFNRRDVEALASYLDDDWTYHIQNGSQPQTYSKDDFKRWVEEFMRRAKDFHLEFKDLHIDGKTVHVHGTLTTRHGDQTFHMHTTDTHHFKDGKVKETRATYDEQPT |
| 1306 | PDQEEIKEWLRQFYEALDKGDWEKVKRLLHPDATVHLTHGDQTETYSADEFIEWYKEWKKSASQVHREIHEIRIDGDTATVHYTVHVRFGDKTQTIHATDHFVFKDGKVTELRSHVDERPT |
| 1307 | PSEEEIREWLRRFFEAINKKDLDAIASLFHDDATMHIYRGRTFTTKEEIKEWFERWLRTFKDIQIQIESIEINGDTVTVHWTAHVRDDEPRTITIRATTHVHFKDGKVTEMHIHLNQDDNNG |
| 1308 | GGEEEFKEQIKQFFERYVEATLKGDLDTVKSLFHEDWQYHDNSNQITFNNREEFVKWVEEWQSNVSSTQFEIKSIEVRGDTVTVHVEVHFESNNQTFRVTEHHTFDKDGKLVSTEQTWHQHS |
| 1309 | GGEEELREWVRQFFERLAEAINKGDTDAIRELYTPDSRYHNAATGRTATTLDEIVRFFQQMIKNLPQTTIEIKRLEVRGNTVHVHVEVHHSSTNRTFRVTSTFHFDHDGKLKEEEVHWHEHS |
| 1310 | ASEEEIRQVVRRFFEAFNKKDIDTLVDLFHPGATIHFSKGRTFTTREEIREWFERVFRTFPDAQIEVKSIHVDGDTVTVHWEMHVHHNGKTVTVRATTTFHFKDGKLTEMHVQIHEHD |
| 1311 | MSEEQIRQWLRRFYEALDKKDVDKVVSLFHPGATVHFSKGITFTDLEEIREWFEKWLKTYKDTAHHLESIHIDGDTVTVHYTVTFTTDGKQVHVRVTDHFHFKDGKLTRMESHEDERD |
| 1312 | MSEEQIREVIRQFYEALNKGDLDAVMSYYDDGATMHVYRGITFNDKEEIKRWFEEFLKEFKDIQFHIESIHIHGDTVTVHVTVHMHRNGKTVTIRITQHFVFRNGKIVEDHIHVNEHD |
| 1313 | LDQETAKELVRRFFKALNDGDLDALKELLHDDWTMHYNNQTFNSLDEFKEWVREWAQGWDVEIKSIEVHENPDGTVTVHVHLTTHNKKTGQTYHQTETHHFNKDGKIVETRAHMQD |
| 1314 | SDQEEAKEIKKELIRALNEGDMDAFKELVHPDAQMHVDNQTFNSLEEIVEWFAQFISGTGIEVERIEVQENPDGTITVTVHFTFTDSRTGQTYRATSHDRYSQDGKIVETHFHRQS |
| 1315 | PDEEQQEQIKEWVERWVRLFNTDVDKAASLYDKDSQVHINGQTFNDREEIIKWMRELKQTHIQVKSIEIRLHPDGTITVHVHLEVHYDHQTFRVTTTLHFRKEGDRWKLTHHHTTIDQVR |
| 1316 | TDESEQREIVRQLIRRFYEALNKNWEEAAELFHEGVTMHADGRTFTTADEITEYIKQLFDQDIKIEIKSIEIRFHDDGSVTVTVHVELHNKDGTVSATHHWFFEQRNGQFRLVEMREHRQPQ |
| 1317 | SDEEEIEEDARQVVRRMAEAFNTDMDELKRLYHPGATVHIDGQTFTNLEEIKDWATKLYSNDVKVQIDSIEVQVNPDGSVTVHVHFTVHTKDGTYKVTSTLFIELHNGKWYIVEERVRTQPT |
| 1318 | TSEEEIKEAIKELVERFFEAFNKDWREAASLMHPGSTMHVDGQTFTTKDEITEWIRQLFSNDIKIEIKSIEIQINPDGSVTAHVHWTAHNSTGTVDATTTFFFTQHNGSFRLVEVRFRNQPT |
| 1319 | TSEEEQREIIKEIVRREAEAFNNDRDAWYSLFHPDARFHYDNQTYDLDEVKEMLERAKDQKIQIDSMEVQVNDDGTATVEVHWTATRQGQTYHFTSTLRIEVGEGGWRIVEKHIHTIQS |
| 1320 | PSEEQKEAIKELVHRFVEAVDKDPDAAFEFVHDDWTFHTDNQTFTDREEFREWVARLPEFRREIKSIEIRLNPDGTITVHVHVEVHYPGSTIKGTETHRFRQEGDEWKIVETHAHFQPT |
| 1321 | SSEKEAEEEIKDFKQKLIEALNDGDMDKFESLLDKDQSQYHYNGQTFHTMEEIKEWFTQWLPGWKMQLDDLEVEIKPGGEATVHSTVTMHGPGKTITIHTTDRYRYEGDSWKLVETRTHTDEGE |
| 1322 | TSRKEFEEEIRKLVKRFFEAFNKNDLDKLAELLDKDHATVHYNGRTFTTKDEIIDWFRNYAPGWNIEIKDLEVELKPGGEATVHVTWHATTNGQTYTGRTTIHVRKEGDTVKIIEVRIHIDPGS |
| 1323 | PHPETESQVRDWIRRWVEIFNRGDSFDNLYDDDAQVHFNNRTFNDKEEIEEWFERLLKTFPSHIQIESLEIRQGDGEVTVHVTFTVTHENTNRDIRFHATFHFQKDDSSLRLVEQRVHTEG |
| 1324 | THPSVEEAIRDIKRRMLEAMDNGDSVDNLFHPDAQMHVEDQTFHNKEEIEEWVKRMLESTSMHHQITGMEVRQGDGEVEVQVHVRVTTPETGTTYSFTFHDRYRREDDSWKLLETQIHTDG |
| 1325 | PKPEDEKAARDLVRAMYEAFNRRDSMDGLMHDDAQMTVNTQDFHSKESVEEWFRRVARTFDSTVQIDSMEVHIGDGEVTVHVHVTVTRNETNRDFHFTHTWFIRRDDDSWRIVRVEFHRTD |
| 1326 | PEDEEAAKETAKRYYTAMNTDIDAVRELFHPDFTMHIYNGQQFDTLEEVVEFLKQFIENTTIEIKSIEVRTNPDGTVTVTVHVTVHRKDGTIHVTHHWFIEKRDSRWQIVHLEERRQE |
| 1327 | PSEEQIREMLRQFFRAINEGDLDTIKSLFDDDATVHFARFSVTVHTKDEIKKFFEEMQKRFDMKIEVKEIRIDGDTVTVHWEVHVTHNGQPYTFSATTTFFFVLKNGQLYISRMEIHINPT |
| 1328 | PSEEQIKEMLRRFFEAINRKDLDAIAELIHPDATVHFASHNVTFKDLEEIKEYFEEMMRKFDFHIEVKEIEIRGDTVTVHWEVHTTHNGQQYTYSVTTHFFFRLQDGQLYITEVRVHMNPT |
| 1329 | MGEEAVRDWIRRFYEAFNTDIDKLSQMYAPDAQFHVNGGQQFTTLEEITKWFRELASTTRIQIHHIEVHPNDDGTYTATVHATMHQDHRTVKVTQHFHFQVRGDDLQLIRDEVHFQPT |
| 1330 | PEDEEQIRRFKERVIRAIQTDWEAVRELFHDDSTFELKKTQREYDLDEFTEWVRKWAENASVQLDSMEIHKHDDGTYTVHVTLTTHTRTKTFTIHATDFYRKIGNQWYIIRTTFDFTDD |
| 1331 | PPPEDEKVLRDLVRQFVESMNKGGDRAKELYDDDATMHVDGQTFKTREEIAQWFKEWASTWEIHIREIRIQSNKDTGTYTVEVHVEVRTKDTTVNVTSTFFFQKHPNGQWRLKSEHIHRNQHD |
| 1332 | SEREEQQKQIEEFLRRYYELLDRGDLEAIYELFHPDVRFTDARTGQTFDLDEYKEMLEEFMRRFRHSRHQIDSLRIEQKSETHATVHVTVTVQLEGNSHTQQFHDTYFFEFRGDQMYIVEVRSQTDDT |
| 1333 | SEEEEAREQIKEWFRRYVEAFNKNDVDALASLYTPDARFQDSRTGRTFDKEEIREWYREILERFPDLQLQVESMRIQVDDETHLTAEVHVTIHLPGNETTVHVTYHVRFERRGDQVYITSMHARFHST |
| 1334 | SENEEMERQIREFLKRYYEAWNKKDLDKIYELMHPDVTFVDARTGKEFNLEEFREMVERILKTFPELRFEIDSIRFQFDSKTSVTVEVHVTVHHSGNKKTVRVTHHWFFRFEGDELKVTELRERTHDT |
| 1335 | SEEKELRERAREFLERFYTALQNKDLDAIAEMMSPDVTFQDSRTGKEYNKEQIREMIERWLRTFEDAQVEIKHMEAEIKDKNHITVRVTVQFTTKGNKQTYQFHHEWHFELRGDRLFVVRVQVHIHST |
| 1336 | DLDPEQEKAVREFVKRFYELLNNGDADAVASLLHPDARFHHSSGRTFDREEFKEMVKELKKQGWEVQIESIEIRVESSTTVTVTVHVTVHNQSTGRTFHVTNHDRLVFEGDEVKLVEHRSHMEERST |
| 1337 | DVDPTQQEAVKQLVERFFEAVNKGDLDALRELIHPDARFHHSSGQTYNLDEFLEMVEELFKQGWHIHIHSIEIRVVDKDTITVHVHWTAHNDSTGRQFDATTTFFVTQYGDEWKIVEVRIHIDTKTN |
| 1338 | DADPEQEKQVKEIIERFFEAFNKGDADAVASLLHPDWTYHHSNGQTYDKEEFKEMVKELFKAGAEIRVHKIDFEVKDKDEIEVTVHYQMHNKSTGRTFDVTEHHRFVLTGDKWQIVETRVHIDEKTN |
| 1339 | PDEMEEAVRKTDRKRLEAFNNRDADKFMEVVHPDATFYENGQPQQLSRDEIREWFEQWAKRAKDVHIQLHELHIQIGDDHHATATGHITFTFPGTNRHVQVTYTVHYQFQDGRLFLTEMREHRDSKQS |
| 1340 | DDEQKEKIEEWIRRFYEALNNNDVDAVASLYTPGVKFYHNGQPRDIDRDEFREWVKEMLERLKDVKIEIKSIEIRVGDDTEITVTVHVQMHQPGTGITVDITQHFRFRFEDGRLRILEDREHSDSKQK |
| 1341 | DDEMKEKAEEWKRRFVEAWNKNDLDAFDELFHDGAKIVVDGQEFPLSKEEIKEFIERLIRSAQDAQIHIKSLEIRIDDKDEATVHVHLEMHIRGTNQTVHITQTDRYRFEDGRIKLLESRSHRDHRNS |
| 1342 | DDEAKEEAEKLVRRYYEAFNNNDWKAIKELLHPGATMHSDGQTYPRDQDELVEFWRRTVESLKDVHVQIDSIEIRVGDKDEVHVTVHVTFTQPGTNEHTHVTHHWRFVNTDGRWFIVSVREHRDERKS |
| 1343 | PDEMQEAAKEADRRRIEAFNKGDLDKIASLIHPGATFYHNGQQFDIDRDEFKDWVREMRRRFQDVQIQIHEIRAQVGDDTEVTVTVHVTFTIPGTNIHIDVTYTVRYRRKDGRWFIIEVREHSDSKNS |
| 1344 | DSEKEIQDTIKTIVKREIEAFNNGDVEAWASLYHPDATFTSGNRTENRSKDDIKRFVKQLLSQQWRVELESLEVRVDSKDTVTVHIHVTWKNERTGRTWKATQTIRLEKHNGQWYIVEKKSDRNQT |
| 1345 | DSEKQIQEAIRTIVKREIEALNNGDADTWASLFHPGATFTSGNETQNRDQEDLKEWVERFFSQDWRIKLDSLEVRVDTEDEVTVHVHITFTQDRTKRTYHATFTIFVRKKDGEWKIVRKKSDRRQT |
| 1346 | PSEEDERQLREVVRRYYEAINKGDMDTLRELLHPHATFVHDDQQFDISRDEYVEWVEKFVKNNDLKFDIDSIEVRKIDDTNFTVTVHVTVTERRTGRTYHVTDHYFFRRTEDGRWFIVRVESHTNQNN |
| 1347 | PSKEDEEAIREIVRRLVEALNHGDMETLKELYDEHATIHHDDQTYPLSRDEIAEWIEKWLRNNDTRIHIKSIEIRVRSDKDYTVHVHVQVTNNKTGRTVKVTSTIHLKKRDDGRWYIQEEREHRNPET |
| 1348 | PSPEDEKAIREVMKRFYDAINRNDWDELAKLFHPDARFHHDNQTFDLSRDEYTEWWRKQFESFDLEVRVESIEVRKISEKEFTVHVHVTVTDRSTGRTYRVDETHFLRKDDSGQWRIVETRSHRHQEN |
| 1349 | NPYEEEARDLKRKYVEALNKNPDEFIKLVHPDATFHIEANNTTYKSLEEIRDYVEKQVKNSHVQVHSVRVEVKPNGEVTVEVHATVHHKDGTVDVTQHDRYRMQDGQLYLVETHERMQPR |
| 1350 | MGEENLKEEVKKVLKQMYEAFNKGDWEALLRIMHPDATFHHSNGQTYNSLEEFREMVEELQKHDTQIEIKEIRIQIDSDHTVTVTVHATDTTRSDPTTVTVTHHWFMRFEGDTVKILEVREQFDRS |
| 1351 | MGEEEAKRQIRETIKRFYDAFNNGDWEKLLRLLHPDATFHHSNGQTYNTKDEFRKWVEELLKNNVQIEIHSIEIQMHDETNYTVRVHATMKNSKDSRTIHVTHHWHFRFHGDQLRIVRVQVQFDQD |
| 1352 | KGDEEVEKQIREIKRRLVEAFNKGDWEALARIFHDDFTFHDSNGQTITSKEEFREWFERWQSTNQQIEVKEIRIETKSKDEYTVEVHFKVTNSRSSTTVDVTAHDHYRVIGDNVYIIRSQVHFHQS |
| 1353 | MGEEQLKEQIKEWKKRYIEAFNNKDWEKLLRLLHPDVTFHYSNGQTFNSLDEFREFFERLAKNDAQIEVHSIEIQVHDETEVTVHVRVTDRTGTDPTEVEVRSTDRYTFHGDELKLTETTAQFDQS |
| 1354 | AGEEQIKEQVKEVLRQFYEAFNNKDVDKLLELLHPDFQYHIWNGQTLNSLEEFREYFEELAKNDVQLEIHEIRIEVKDETRVTVTVHVTDHTGQDPTTYHVTEHHFMVREGDQMYIAETRVQVDQT |
| 1355 | PDEERIKEWLKRFYELFDRGDLDALASLLHPDWTFHHSSGTTLTTREEFKEWVKQFLERFENTAHQIDQIDIDGNTVTVHVTVHFHTDGRTVTIHVTDHHRFDKDGKLTETRSHFNQTD |
| 1356 | PDEEEIKEILERFYEAFDRGDMDALFELLHPDATMHISNGETFHSAEEIKEWFERVLKTFGDVRHEIERIEFDGDTVTVHYTTHWHTKDSTTTVRATDHFVFDKDKKIKEVRSHWNPTG |
| 1357 | PSEEEIREIDKQRLDAFDNGDLDALMSYFHPGATFHDSSGQTYHTLEEIKRWFKEFLDKFDVKRTEVRIHVDGHGTVTVHVTAHGTHNGKKIQFHYTYTVHYRNGKITEIHVHIDTQD |
| 1358 | DSEEEIKEIIRRLIEAFDRGDLDALAELYDDGATYHDASGYTATDREDIKEFFKQFLEKFHEQFHHTEIRIHIDPNGETVTVHVTVQVHTKDQTYTYRVTSTVHVKDGKVVEERVHINQEN |
| 1359 | PFEEEVKDLKTKFLEAINRGDVDALVELLTDDSTVHFTMYQITITSKEEFRKWFERFWKTYNAHFDISDVKIQIHGNEEATVHFTVHFRHGDRDITVTVTVHDRYVNGRFKRTRIHFNPHE |
| 1360 | PTEERIKELLKRFYEALNKKDLDEIASLFHDDVRMHNQSNNITYNSKEEIREFIERLLSADFKIEIKEIRIDRDRQRVTVHVTMHRNHNGQTYTFDVTHHWQFKDGRIVEVRHHTRQRT |
| 1361 | PDQEQIKEWLKRYVEAWNKGDLDALVELFHPGWTMHLSNGQTFHTLEEFREWVKELLDKFQDTQVEIDSIHIDQSKGRVTVHVTYHHNHPGQTRTAHVTEHHQFEGDRLVETHFHVDQKG |
| 1362 | PEKEIEERIREWIKRWYKSWNDRDVDKIISLLHPGWTYHHSSGQTFTTLEQFREWVERLMKNADFRIEIKKIDISGGTVTVHVQMHWTHGDKTTTIDVTEHHHFRNGRLTETRAHYTSSPKD |
| 1363 | PEEEEKQKIKEWLKQFYDALNNKDADAIKELVHPGATMHIADGITFTSRDEVKEYVKRIADNADIRLEIKEMHIQDGHVTVHVEVHEHHGDTTYTVRVTHHWRFKDGKLTEVREQTDRQPHS |
| 1364 | PEPEEQEKIKEWVKRYVEALNNKDVDALASLYDDGATVHFANGTTFTTREQVREWYERMAKNSDMRVNLESLEVRNGHVTVHVTVTFHHGDKTTHIRITVHFRFENGRLKEVRFHWDSQPQS |
| 1365 | PGEERIRELAKRFYTAINNNDLDTIARLLTPDVEVTFASEQRTFHNLEEVKEWFQRFIESNPRFRMQIEEIRVDGDTVTVRYTVHVNDKRTDSTYTTHAQDHFHVKDGKITRVTSNRDTHQT |
| 1366 | PGEETIREWVKRYIEAWNKKDLKTLLDLVTDDWTLHLGSYQITFTTREEYKEWVERWMKTFPDFRIEIKSIEIRGDTVTVHVQIHGTNKDEPKTFTWDVTEHHHFKDGRITETHMDFDQKET |
| 1367 | PGEEEIRKWVKRFVEAFNKRDADTLFELVTEDWTYHAAGQQITFTNAEEFKKWVEELLRTLSDFQMDITSIEVRGDTVTVHVTVHMRDDRTPKTVTTHITEHHHFRNGKITETRATWDTKDT |
| 1368 | PSQEEIEEIDRQRIEAFNRRDADAVASYFAPGAEIHLSNGQTFTDLEEFKEWVERFLRRFKDIHIHIHEIHVNKDTVTVHMTVHFTHDNGQTYTMTVTYTIRYDKDGRITRMQVHVDEKR |
| 1369 | PSEEQIKEWLRQWFEAFNNNDWETLASLYDDGATMTHYRGETFKDKEEIIEYIKRFLESMKRMRMEVKEIRFNDNTVTINVEVHVTHETGQDVTVHIQFHFRFDKDGKLVEQHTRTDQQN |
| 1370 | PDESQIREWLRRFFEALDNRDLEELMRLFDDDWTMHIYNGQTFTDRDEVKKWFKEILDRMKDFRHQIESIHFTEETVTINVTVTVNHDNGQTFHIRVQEHHRFDKDGRLKETQFREDQQD |
| 1371 | PPELEEQIKEFVRRFIEALNRNDLDAVAEILHDGWTMHINNQTFTTKEEVRKWVEEFLKKNKDLRIQIDSLEIRGNTVTVHVTVHFTHNGRTYDVSVTEHHRFKDGKLVETHVQRDQIH |
| 1372 | MSEEEIRDWLQKFFEAIQNKDLDTFKRLITEGVTFHFSAEQKTFHDPEEVVEWFERWMRTFPDHRVEIHSIEIRGDTVTVHWTMHVTHNGQTYTIDVTTTFHFRDKKLVEVRFHFNERR |
| 1373 | MSREQIERFVRKLVEAFNRKDRDAFEEVYSEGSTLTSDSTQQTFHNKDEIVEYFKRLADAFERFRVEIKEIRIHGDTVHVHLTFHFTHNGKDVTFDVTSTFHFRGNRLVEEKITVHERR |
| 1374 | PTEERIRELLKRFYEAWNKKDLDKLVSLFHPGATIHSSEGITLTTQEELREYIERVMRRWKDTRIEIEEIRIDGDTATVHVTTHFHTNSQTKTVTATHHWVFQDNFVVELEERSNEH |
| 1375 | PDQEDITEWLKRFFEAWNKKDLDTLVELFADGSTYHHADGITVTTKEEYKEFLRERFERAKDTQIEVKSIEIHGKTVTVHWTMHLTSNSQRHTVDVTTHFVFRGKRVTELRQHFNQK |
| 1376 | DLREEIERFVRRLFEAINKKDLDALKEIFHPGWTFHLSQGTTFTTLDEFREWVREVQERFKDTQIQIHEIRIDGYTVTIHVTVHFHSNSQTKEVTVTEHHHFKDHKLTETHQHHNEK |
| 1377 | ASEEEIKQWVERWAEAFNKKDLDELARLFHPDSQYHDQNGETLTTKEEYREWYERLMRSLKNFHIEVKSVEIHGDTATVHWQMTTHRPDGQQYHYTATTHFQFKDGRLTEVRQHTDGT |
| 1378 | SSKDEIKEAVERFYDAFNKGDVDLLASLVHPGATFHDSSGETLHSKEEFREWVKRFLDEMEDTKFEIKSIEIRGQTVTVHFRLTVTHSNGETRDGTYTDHYHFKDGKLVEIRSHEHGE |
| 1379 | PSEEEIKEFANRFYEAINQGDLDAIMRLFHPGATFTVNEGTTYHSKEEIRKWFKEFFERVKQIHIQIHSIEIRGDTATVHGTVTVHHEDGRQQDFTVTHHWHFKDGKLVRVEERRHGS |
| 1380 | PSKDEIKEWLERFYEAIDKNDWEALERIIWDGWTFTDSSGQTWTTRDEFREWFETMVERFKDMRHQIDSIEIRGDTVTVHVTATFHHESGETRTQHVTDTHHFKDGRITRTRSHRHSS |
| 1381 | PSKDEIKEWLERFYEAFNQKDLDALDSLVDDGWTFTHSSGETLTTREEFRKWVEQFLREVQDIHIQIESIEIRGDTVTVHVTVTVHHKDGRQQHMRVTETHHFKDGRITETRQHIHGS |
| 1382 | PSEEEIKDIKRRFAEALDNNDVDTAASLFHDDATVHFSKGETLTTKDEIREWIERWASRFKDTAHEIKEIRIDGDTVTVHVEVHFHHKDETKTVRVTVHDKYKDGKITRSQFNYDEGG |
| 1383 | PSEEEIRDFIRRLVELLDSGDADAVASLYDDDSRVHFSEGETLTSREEIKEWVERYFKSLGNVHREIKRIEFDGDTVTVHVTVHIHHNGKTVTVHVTRTFHFKDGKLKEDHVHIDNGG |
| 1384 | SDEEEVKEIKRRLVEAMDKGDWETLARLFHDDAQFHFEATGTTFHDKEEFIRWFQQFVSNAEIHRQVHIHFDGDTVTVHLHVTVTHGDQTHHVTATVHDRYKDGRLTESRFTFDPRE |
| 1385 | SVSEEELREWLKRFYEAFQKGDIDALKELIHDDWTYHDSSQGITFTTKDEFREWIKELFEKFDFQIDSMEIHVHGNTVTVHVTVTVHTKDKTKTIHVTDTHRFKDGKLTETHSHVDEKET |
| 1386 | SISEEEVKEFIRRFLEAFEKGDLDALAELFTDDWTVHFEYSGITFHTLEEFKKWVKEVLEKWEYRVDSMKIDVHGDHVTVHVQVTRHHPGETQEFTYTEHHRFKDGKITETRFRRHQHDT |
| 1387 | QISEEEVKEWVRRFAEAFQRGDVDALISLYTDDATIHFESEGRTFTTKEELKEWLTQLFRRAEFHVDSIEIHVDGNTATVHVTITATQPGRTYTYHVTSHFRFKDGKLKSERVTIETRGS |
| 1388 | QISEEEAKEVVRHLIEAFQNGDMDALMSIYTDDATFHMASEGRTYKNKEEIKDWFRRLRESADYQFDEVEIHVDGNTVTVHMTVTVHHKDKTVQQHYTSHFRFENGKLKEEHVHVDTRET |
| 1389 | SISEEEIREFLKRFFEAFQRGDMDALKRLMHDDATVHFSSEGRTYHTLDEIKDWFRRVWESAEYHFDEIEIHVHGDTVTVHVHVTVHHPGETDHITYTVHDHYKDGKLTRRQVHIERRRT |
| 1390 | PISEEEVKEWIKRYAEAFQNKDLDAIMSLYTDDATVHFASTGTTFETKEEIRDFYRKVLENADYQFDEIEIHIHGSRVTVHVHVTVHHPGKTVKITITMHFQFKDGKLKEVQIHVETKQT |
| 1391 | PEQQEKIKEFLKRFYEAFERGDLDAMFEMLHDDWRFHYEYENITFHNREEFKEWAERFMKRFEFQVHMTIHVDTDNTVTVHVTVHTTSPDGQPYTFHTTDHHRFRGDRLIETRVHVRQET |
| 1392 | PEDREKIKEIVKREVEAFQRRDLDTWAELVHDDARITFQYENRTFTNKEEYKEWLRRFMETWEFRVEVTIHITSSHTVHVHLTFHVRHPSGQDHTFHVTVTIHFRGDKITEKHFQWHQED |
| 1393 | PEDRERIKEWLERFFRAFANGDLDALKELLTPDWQFHYEPQNITAHTLEEFKEWVERFIKQFDMRVEIHIHVSDSDRVTVHVTLHVTHPTGQPQTYHITEHHRFKDDKITETRVHVDQQE |
| 1394 | PEQREKAKQTLERFFEAFQRGDMEKLERLLHDDWTFHWEQQNITFTTKEEYKEWVRQLLSHFDVQFEIEIHISDSNTATVHVTIHWTHRSGQKRTIHVTEHHHLRGDKITETRAHIEQKE |
| 1395 | MSEEELREWLRRFYEALDRGDADALASLYDEGVTIHISEENRTFKNKDEFREWFRHWFSNTDTAHEVHIEIKDDTTVTVHVHVTLHHKDGKTVHITVTQHFHFRGKKITEDRAHIDTKET |
| 1396 | TSEEQIREFLKRAYEALNRKDLDAFAELFTPGWRYYHAEENRTFTDKSEVREWIRQYLEKTDAKIEVHIHVDDDTRVTVRVTVHFTTKDGKKQTVHTRDHHHFEGDRITETRAHYDTKDT |
| 1397 | AHEEKIREWLKRYIEAVNRKDLDAVISLFHEGWQMHHANGITFHTRDEVRKWFEEFLRRFPRVRVEIKSIEVTTDTVTIHVTVTVHHDGQTIHIDVTEHHTFKGDRITETRIHIDEKPT |
| 1398 | PDEEEIKRILKAFYDAIDNGDMEKIKELMHPDAQLHNSSQGITWHDLEEIAEWFARFASLRNTAHEIKRIEFDQDHVTVHYTTTVNKNDQTYHFNVTDHFRFKDGKITEVRSHIQGE |
| 1399 | PSEEEIKEIKKRLLRSVDDNPNDMDWFKKVFDKDHVTMHSDNTTMTTADEIVEWFQRQFATNNSKRELKSLEIRGDTVTVHVEVHETRTGRTFTVTYHDQYKDGKIVSTTAHRDST |
| 1400 | SSEEEIRQWLKRFYEAFDRGDADALKEIVHDGWTLRSYNITDKEEFKEWVERWFSQFPQMHRQLDRLEVEGGTVTAHFTVHFTHNGRTFTMHTTDHHHFKDGKLTSTQQHTDEQPT |
| 1401 | PSEEEIKEIDKRRLEALDRGDMETFKELVHPHAQMHVSEGKTFDTRDEIAEWFERFWKSFKDMRRHIEEIRVENGTVEVHVTVHFTHDNKTTTVHITYHVRYDKDGKILSMQFHRNQTSD |
| 1402 | PSEEIARQIKRRFIEALDRGDADAIAELFHPGATFHDSSGRTFTTKEEIKEFVERWQSQFQDSRHQIDNVHFDKDHVTWHVTQHFTHNGQTFTVHVTVHDRYKDNRLVETHFHHNHS |
| 1403 | SSKEEIKEWLKRFYELINRHDLDKIAEFLDEGWTMHIESEQYTATNKEQVRKWFEEFMKKVERIRIEIEEIRIDDNTVTVHVTMHVTHNGQTQTYHVTEHHHFKDGKITETRQRVHTG |
| 1404 | MSEEQIKEWLRRMYEAIDNNDLEALMRLFHDGWTMHIESQQVTFKDKSEVKDWFRRYLESVKDTAHQIDSIHISDDTVEVHVTTHENHNGQTYTYRVTETHHIKNGQFKETRQTVDTG |
| 1405 | MSEEEIKDWVRKWAEALDNNDLDTLASLYDDGATVHFQSEQRTFNNKEEIKEWVERWLKTVKDSQHDIDDLHIDKDTVHVHFTVHLTHNGQDHTYHVTSTFHFKDGRLKEERIQVHTG |
| 1406 | MSKEEIKEWIKRYFEAINRKDADKIVSLFHPGWTFTNESQQITFEDQSEFKEWVERWLKSVKDIHVEIDRIEIDKDTVTVHFTVHVTRNGQDHTFHATEHHHFRNGQLKETQLQIDTG |
| 1407 | DREEAEKAIEEWVRRFLTALNNKDLDKVLSLLDDDWTYHDSSGNTYTTREEVEEWVREFMSQNWQITYQDLHIQQHGTHTYTVTVHVQYHDKRTGRTFQVTEHHHIERQPDGSWRVVETRVHTDNE |
| 1408 | DELEEFKRRAFKAIIDTNDWDKMAKYYHEGATITVNNETRDLGREQLVEWFRRFLSRVGEMHWQIDRVEVDQDHVTVHLTVTFTHDGRRVTVRVTEHDRYKDGKLTSSNQHRDQDDS |
| 1409 | DELRELKKKFYEYIVNTRDLDAVKEYFHPGATITEGNRTETKDQDQIVEWFERWWSRVGQAHHEIKSIHVSDNHVTVHVEVHTTHNGKKITFRVTQHDRYENGKLVEMHVHVDQDDT |
| 1410 | PGDESKIKDIVRKMYEEAFNSNDWKAFEEAVTPDSTMTVQNGQTFTTLEEIVRWFQEFIKTFKDIRVEVDSIEVRGNTVTIHVTVHAHHDNRTITFHATHHWHIENDRFTEVHSRWNQKD |
| 1411 | SSEEEIRQLVKRFYESINNNDVDAAASLFHPDATMHISQTGQTFTTKEEIRKWFEEFLRSVSDIKVEIKSMEVRGQTVTVHVTITVNHGDRTVHMHATHHWTVKDGKITEMREHVDRQPD |
| 1412 | ASEEEIREIIRRLLEAMDKGDMDALRELYHPDARFEASETGQQFDTLDEIMEWFEKWHKSVGEVHREIKSLEIRGDTATVHVTVHTTHGDKTYTTDFTSHFRFKDGKITEEQLHVRTQPT |
| 1413 | PSEEEIEEILRRLYEAFDKGDWEKIASYMHPDAQMHISQTGTTFDTLEEIIEFVERFFKEAGDVKRQIHSIEIRGNTATVHSTVTHNRGDQTFHMRATHHWHFKDGRITSVKEDVDTQPS |
| 1414 | PSEEELKEWVKRHLERIFNSGDWNAAKAMFHPGVTVHVYNGQTFKNPEEIAEFIRRFIESFKDVRLEIERIEVEKDTVHVHWTAEFHTDTETVHIHGTTTFHFENGRLKEVHIHLRQTN |
| 1415 | PEFEEEVEEWVRRMFQAFFRTGDWRALEEAFHPDAKVTWARGEQTYDQEEFIRWFTKMMSRFTNAHFEIKEIRVDGNHVTIHWTVTFHSPTQTFTMHGTTHFRFKDGKLTHVRFEIHQDDGS |
| 1416 | PISEEQIKEIVKRFYDAWNNNDVDKLVSLFHPGATFHFWNGITATDLEEFKEYVERFLSNFNTQAQIHNMEIRGDTVHVTYTMHRTHNGKTVTLEATDTFHFKDGKIAEYKSDVDDKD |
| 1417 | PISEEEVKEFKKRFIEALNKNDLDELKELFHPGSTFHFADGETVTTLDEFKEWVERMFKNFNFEFHVKEIRVHGRHVTVHVEVHFTHNGQTFTIHVTVHDTYKDGKITHSQFDIDQTD |
| 1418 | PVSEEQIKEVLKRFYDAINNGDIDAIVELIHPGATLHFSKGITLTTLEEIRKFFERTQKRFNTKIEIHSIEIRGDTVTVHVTMHATHNGQPYTYRVTHHWRLQNGKVAEVHHQENDTS |
| 1419 | PEPEEIAEELVRTIEEAMNKGDVDKMLSLYAPDATVHFSNGQTLTTREEIKRWFEEFFSKFKDVQVEVKSVEVNGDTVTIHFTVHTNHENKTTTFHITSHFHFKDGKLKEERVHIDQEET |
| 1420 | PEAEQAVRDFVRTFLDAFNRGDVDAIASMYHPDATMHLSNGQTFTNLEEIKEWFRRWFSNAKDVHIELKSVEINGDKVTFHFEIHVNHEDRTVTFDVTSHVEFKDGKIVSEQIHTDQKQS |
| 1421 | PEDQERIEEILRRFYELFDRGDMDRAVDEAFDDGATMHLAEGQTFTTKDEIKEWFRRLWSTIKRLHRQIERIEIRGDTVTIHVTVTVHRDNEQIHMRATHHWTFKDGRITEVHSHTDQHN |
| 1422 | PKERQDRITEIDRQRAEAWNRKDLDAVMSYVHPDATFHFAETNTTYDREEFREWVERVLKTFPDMHVQIEEIEVRGEHVTVHSTVHVNTPTKTRTIRVTYTVRYKDNRLQEMHVHTDTEDQ |
| 1423 | PEEDERRIKDLKKKEVEAFQNKDLDKVMSLFHPDATFHFADRGETYDYEEFREWVERVLKTFKSIQVEIKEIRIHKDEATVHLQFHVHTDTKTHTVDVTVHDRYEGDKLTETQFHTDQKHS |
| 1424 | TSEEEIREIKRQYIERFDKTGDARVFEDMFHPDAQYHNDSEKRTFDTMEEIKRWFNESWSNHDSQRHIHEVHFENGTATWHVTVHRTDKSTGQNYTIEFTYHDEYKDGKIVRTQEHSRQT |
| 1425 | MSEEEIRDLDRRRIEAFNKNDVDKVASHYHDDATVHYNEGITITNKEQLKEFVRKFIESAKRVKLDIDGMEIRGDTVTAHGTMHVTHGNKDTTIHFTYTITYRDNRIQEEHVHRNQQD |
| 1426 | MSEEQIKEFIKRLLEALDRHDVDKFVSMFHPDAEVHFSSGITFTNREEFEEWVRRWMSQFKDSQHDVHSIEVRGDTATVHWTQHANHGDKTTTFHGTTHFVFRGNKLVSMHVHIDQQD |
| 1427 | PDRERIADFVRRMLEEFFKKRDVDAAMSYYDDDATLHIYQGKTFTTKDEIKEWLERFLKTYKDAHVEIKSIHVSDGTVTVHIELHLNHKDQTRTYTITSTFRFRNDRIVEERVHEHQQ |
| 1428 | PVSEEEEKRIRDILRRLIEAFNKGDLDALASLYHDDWTFHLSEGYTAHSKDEFKKWVEEWLKKAQQIHLEIKSIEIRGDTVTMHVRITVTHNGKQVDMDVTEHHTFRNGRLTETRMHTDTKDK |
| 1429 | MDEEQIKEIIERFFKAMDTGDLDTIRELFHPDARVHFEAYQITLTTLDEFIDFIRRWLEQFPDVRHEIESIHIDGDEVTVHWTAHFNHNGETQTFRYTTHIRFKDGKIVEMQIHENDER |
| 1430 | TDESQIRELVQQLFEAMNNRDRDTFKSLYHPDATMHIEEEQITFTTREEIAEWFTRFLKRFPNARFEIESMEIRGDTVTVHVTVHFTHNGQQYTYRYTRHFRFKDGKLTRDQMHVDNQE |
| 1431 | TDESQIRELKKRFIEAFNNKDWDKLRKLVHPDATFHFEKEGVTLTTLDEFVEWFKRFLKRFPDVQIEIKSVHIDGDTVTVHMTMTFTHNGETRHVNVTVHDRYKDGKLVESRFHFDDTY |
| 1432 | TDQEQEEHIREVLRQFYEALDKGDADKVASLIHPDATIHIARDQITFTNKDELKEWAERWFSQFPDIQRHIESIEFRGDTVTVHFTVHVTHNGKTYTVRVTDHYHFRNGKLIRVESHVQPE |
| 1433 | ADKEQQEEIKDILRKLYEALDRGDMDKFKSFFHPDATYHNIETQIKFDDLDEMKEFLARWLSQFPDTAHEIKSIHISGDTVTVHVQTHLTHNGKTYTYDVTEHHRFKDGKIVETHTQFDEP |
| 1434 | DTDRQEHIKELKKRFLEAFDRGDAEALAQMFHDDATITFASVQITFQDKEEFKEFVKRFASTFKGAQREIKSIEIRGDTVTVHVTLHVTHDGKTVTIQQTTHDRYKDGKLTETHTTTDSKDT |
| 1435 | DTEIREQIKQFLRRLFEAFDKGDWEKLARLFHDDWQFTSAAEGKTAHTKEEFIRWFREWVSRVPGAQREIKEIRISGHTVTVHVTQHRHHPGSTVTTHVTEHHRFKDGKLTETRQHMSTEQQ |
| 1436 | DTELKEQIKEILQRLFEALDKGDLDAVASFYSDDSTVHVSYEQITFKNKDELKKWLEEFKKHFPGSQREMESIEINGDTVTVHVTVHHTHKDSTVTTHQTRHLRFKDGQLKEDHIQTHEHQQ |
| 1437 | PQEEQIRQWLRRFFEAMDKGDWQTLHDLFHPGATIHFYNGVTLTTREEIARFFKKMTESFSDVKRQIESIEIRGDEVTVHWTMHTTQGDETYTYHATTHFRFKDGKVVEVHVHINQQD |
| 1438 | MSREEIKELIERFYAAFNTNDPDAVREALHPGAEVHSSKGITFTDLEEIKEWVERWAKTFQNTRIQIDRIEFHGDTVTVHVTIHTNHKDHTVTTHATHHWVFRNGRITHLQEHTDDQQ |
| 1439 | PEKDERTIEELKRRFIEAVAKGDDPDKLKKLMHPDAQFHDSNGQTFNSLDEIAEWFAEVQSKFKDMQVEIKRIQFDGDTVTVHVTVHATHNGRTVTYTVTSHDTYKDNRIVETHVHIDSND |
| 1440 | QDEEEETIKELLERFYRAIANGQSWEWIEEFLHPDFTMHLSNGQTFNDREEVARWFQEVMKRTKDTRIEIKSIEFKGDTVTVHVTVHTTHNNQKRTITVTHHWTFKDGKIVRLEERRDSND |
| 1441 | SDEEEEKIKELIHRFVELYAKGDDPSAFKSLLSDDWTFHDSSGRTFHSKDEFVEWVERALSQFKDSKIDIDSIEFKGDTVTVHVTQHATTNNQTYTFTITEHHRFTKDQIVETRQHRDSND |
| 1442 | DREEELKEALRRYYEALDRGDMDAAASLFDKDWTMHQTHSDGSQQDFTTKDEFKEYFREQFEKSDIKRGEVRIHVNDDGTVTVQIEVHVTHDNSTRTRDTHDTHVVKDGKFKETHIHWDYG |
| 1443 | PDEEIIKELLKRFYEALDKGDWKALKDLLDKDWQYHQTHSDGSSRTFTTLDEFVEWYKEQVKNSEVHRGDIEIRINPDGTVTVESEVHVHHKDGTKTVNTHDTHVFKDGKIVETHVHWDDG |
| 1444 | PDESEIRETIKREAEAFNKGDVDAWASLFHPDATMHIYNGQTFTNLEEIKEWFERLIKRFKDAKVEVKSIRFEGDTVTVHVTFHFHHDNETKTFHVTQHITFKDGKITEKRQQMDEER |
| 1445 | TDEEEIRQWLRRFYEALNNKDWEKVKSLYHPHATMHIWNGRTFTDREEIAEWFRRILERFSRVRFEIKEIRIDGNTVTVHFRVTFHHDNQTRHATVTQHFHFKDGKLTEDRIHLNQED |
| 1446 | PSEEEIKEWLKQFYEAMDKGDWQKLSEMLHPDVTITYEAFQITFHTLEEFIRWYRRFMSTVEIHRQITKIDIDGDTVTVHFTTHATHGDETYTMNFTDHYHFKDGKVTEIRSHSDQS |
| 1447 | PSQEELREWVERWYEIVLKTRDYETAAQLFSDDWTYHVYNGTTFTTKEEFKEWFKRFMDRFKDVRIEIDSIHVEGDTVHVHLTMTTHHGDQQYHFRTTDTHRFENGRIKETRTREHDTR |
| 1448 | PSQEELEEWVRRFIEEMDKTKDVDTVASYYSDDAQYHAYQGITFNDREEFKKWVEEFLKKFKDVHIEIKEIRVEGDTVTVHVEVHTTHGDQTYTFDVTRHFEFRNGKLVRDEERMRDTS |
| 1449 | MDEEQIKEFIKRWVEAFDSGDADAVLSFYTPDSRVHFYNGETLTTLEEIKEWVERWQKSVKDAKREIKKIDVENGTATVHMTLHVTHDTHTITVDQTRHLKFKDGKLKEDHLHEDQKP |
| 1450 | MSEEELKEWKKRFFEAFNKGDLDKIAELFTDDSTVHFSEGITLNDREEIKEWFERFMKRFERFRIEIKSIHIDGDRVTVHVEVHFTQNGQDFTTEVTAHDQYSDGKIKESRFHFRTR |
| 1451 | MSEEEIKEFKKRLVEALNKGDLDKVAELLDDNSRFEDSEGTELRDREEIREWFKQMFENIQQTHIEIKEIRIDGDTATFHVEQHTTHNGKQVTVEVTSHDRYSDGKLTETQVHRRQR |
| 1452 | PRDPSQILEWLLKALYEIFQNNDADAFAEMFSDDWTLHWVEGITYTTKRRVKEFFERFVKRYPDTQFQVDRVEVDGNRITVHVTVHTRHGDQTYTIHQTEHHQFDHDGKIKETRIHSDEKPT |
| 1453 | PRDPEQIAQTVVRRFYEVFNKGDADAFLEMISDDWTFHDSNNRTYTTRDEVKTWFEEFLKRFPDSKFEFKRVEIDGDTVTFHVTVHVNHGDKTVTYHVTEHHHFDKDGKIKETRAHWETREV |
| 1454 | PRDPEQIKEWVVKAFAEAFNSNDWELFMSIITPDWTMHDSSGTTYTTMDEVREWYERFLKRYPDAKFQVDRVEAEGDTVTVHVTITVNHGDKTYTFHQTEHHKFDHDGKIRETRFHIEQRPT |
| 1455 | SRDPSQIAEWLAKRLLEALNRGDVDAVMSLYTPDATMHIYRGETFTTLDEIRKWFETFLRTFPDTHFQIDSVEVDGNTVTIHVTVHTHHGDKTVTFHITSHFTFDKDGRLKEEHIHTEQRDT |
| 1456 | PGEEAIKELKKRMLEALDKGDMDKFKSLFHPDARVTFSRHQRTYHNLEEVVKFFKEVTEKFKDMRHEVKEIRIDGNKVTVHVTVHVTHDSGQTVTIDVTEHDRYDHNGKLKETDSHFNQKN |
| 1457 | PGEETVKEVLRRFYEAFNHHDLDKLMSHYAPDATVTFSREQRTFKTKDEIRKWFEEVLKKFKDARREIKEIRIDGRQVTVHVTMHVTHDNGQTITIDVTQHFRFDDDGRIVRDDSHFNQRN |
| 1458 | MSQEEIKETLARYIQEAINKGDADLLRELYHDDATVTFNGRTFNSRDEFKEWFERWFKSVDDTKAEIKSIEVDGDTATVHVTIHVTHGDQPVTIHVTSHFVFDHDGKIVREEINAQST |
| 1459 | PSQEEIKEFVRRIIEEAFQQKRVDLAARFYTDDVRMTYNGQTFHSKDEIREFLERFLRSVDDTKVEVKSIEVDGDKVTVHVEVHQNHGDQTQTYHITRHITFDKDNRIKEDELNQQST |
| 1460 | PEDEEKLKRWKKRFVEEVFRTGDADKFLSYVHDDATVHFSSTQYTTKDQLRKWMEEWSKFEDVKVKIDEIRVDGNTVTVHVTVHATNSTTGKTVTFHMTSHDRYKDGKLVESRIRIDPQPQE |
| 1461 | PDQEEIKRLLREYYRYLDTGDAADELFDEDWEFHNNGRTFTTKEQIKDYLRELQSFKDMRREIKEIRVEGDHVTVHGEVTRNETDKTTTVHFTDTHEVHNGKFTRTQAHYDEKDT |
| 1462 | TDEEQIREILHRFYRAIDNGDWETVSRLVHDDATFHDSGSKRTFTTREEVAEWVKQVASFDTAHHLESIEIRGDTATVHVQTHETHNGRKVTVDATHHWRFKDGKVVEVQGHYDST |
| 1463 | MSEEQAREFVERVLRRMSTQLDDDWRAHVHTKDSTITFENPREFLEWWKKWARSRSRDIKFEIKRVEVDGDTVTVHVTVTMHHNGQRFTYHVTEHHHFRNGKLVETHFHIHQEDG |
| 1464 | PEEEIREWVKRFIEAFDKGDWEKLVKLLHPGVTIHFDGDNVTITSQEEFREFVERWLSRFDEMHRTPNEVHISNGTVTVHWELHVTQGDKTYTIRVTTTFHFKDHKLTEIRIHFDTQPQ |
| 1465 | ASEEEIKELDTQRLEAIDKGDMDRVREYFHPDAEIHISMHNKTFTSRDEFVEKFMREFLSQFEDVHRQIDQMHVDGHTVTAHVTVTFHHNGRQYHYRVTYTVTYSSKKLTRMEIHVDSGG |
| 1466 | PSEEEIKEFKERLVEALNNGDLDTFISLVTPDATFHYSEGITFTDLEELREWVERFLKTFKDLRIEIKDMHIDGDEVTVHIQFHVTQNGKQVTIDVTYHDRYDKNGRLVSSRAHFNQQ |
| 1467 | PSEEEIRDTLRRFYEAFDNGDIETLQRLLHPDVEVHFWEGITAHSLEEFTEFVRRFLSRFKDVRREIKEIRIHGDTVTVTYTMTVTRNGKDYTYTGTDHFHLDKDGKIKRVQSQVHQT |
| 1468 | PSEEEIRDWLRQFYEALDRGDADKAADLFHPDAELHFSEGITLTTKEEIKEFVERWLKRFPDTAHHLESIHIHGKTVTVHVTVHVNRNGQTFTFHVTDHYHFDDDGQVKRMESHINQS |
| 1469 | PSEEEIKELLRRFYEAFNNNDLDKLVSYFHPDAQVHIFQGITFNNLEEFKEWLERVLKTFKDVQIEIKRIEIDGKTVTVHVTVTFTRNGQTFHVDVTHHWHFDDDGQIVRVEEHSNER |
| 1470 | PSEEEIKQLLKRYYDAFDNNDLDTLLSLLHDDWTYHNNSGNTITDREEFKEWVKRWLEKFQDTAHHMESIHIHGDTVTVHVTLHVTHNGKDITFHTTDHHTFDHDGKIVRTEAHVNQS |
| 1471 | PEPDKEKIKQFVERFAEAFQKKDVDAIASLFHDDATMHVYNGQTFHSKDEIKEWFKRFVDNNLDTKVEIKEIRIDDSRGQVTVHWTATVHHNGKQVDIHATTHFVFKDDKIVRVHIHEDQHTD |
| 1472 | PSDEEIEEWVRRFYTAFNNNDWKKLADLISDDATFHDSRGEEITDREEFREWFERVIKTFKDIQLEIKSIEFDKKTPRVTVHVTMHVTHGDQDTTIDVTHHWHFNKDGQIVEVRAHFNPR |
| 1473 | PSEEEIRQWVRRWFEAFNNRDVDALASLYSEDATIHFSEGVTVTNREQFKEWLERFTKRFKDYRLEIKEIRVDKSDPTVTVHVTIHFTHGNEDRTIDVTQHFKFDKDGKLKEQRVQIDER |
| 1474 | TSEEQIKEWVRQFYEALNNNDWEKVLRLLDDDWQFHNANGETYTTKEQVREFIERFLKKFSDSRIEIDDMHIQDGTVQVHVTIHVTHPDGSTQTVHIQEHHRFKDGKITETHQHTDEST |
| 1475 | TSEEEIRDVIRRLIEAFNNNDWEAILRLLDEDWQMHISKGETYTTADEVREWVERFLKSFPDTRVEIDELRIENGTVEVHVTFHFTDHNGQDFTIRVTEHHHFKDGKLTETHFHFDESR |
| 1476 | MSEEELKEFVRRLVEAFNKGDLDALAELYHPDFTYHHDSGSTIRDKEEFRQWVREWVEKAKDYRIEIKSLEIRGDTVTVHVQVHVHDDSRTTTVDVTSHFRFQGKRITEEHAHSDQKD |
| 1477 | MSEEEIRDFVRRFIEAFNNGDLDTLAELVHDDATFHFPDGSDIKDREEFKKYLEELFRRYGDFRIEIHSIEVRGDTGTVHWTLHVTKPDKHHTFHATTTFHFRGKRIVEVHFHVDQHD |
| 1478 | DPREEIKEIKRRFVEAFDRGDIDALISLFHPGFTFHWNNRTFNNPEEVRKFLEELIKRYKDSRHEIKSLEIRGDTATVHVQAHFHHEDKTRTITVRVDDRYKDGKITESRFHEDQDG |
| 1479 | PDQEEIKEIDKQRFEAIDNGDLDAVMSFFHPGATMHWNNRTFTTPEEIRRFFEEWLKRVKRSRHEIKEMHIEGQTVTAHVDVELHHDNETKTVTVTYTITYKDGKIVEFRSQEDQKD |
| 1480 | ASEEEIRDWLRKFYEALNKRDADTVASLFHPDATYQDSSTGKEYSYEEFKRWVERWLKSARNFHIQIEEIRVDDNTVTVHYTVTFNHPGETQHYRFTDHFHFRNGKLTRMQSTTDNR |
| 1481 | PGEEEIREIVKQFFEALDTGDMDKLRELVHPDAQFHFTHDGKTITMHSQEEFLEYVKQLQRKFKDVKRQIDHIEFNKDTVHVHWTMHVTDEDGTTVTFRGTTTFHFRNGKIVEVRFHVDTEQ |
| 1482 | PGEEEIRRILKEFYEALDKGDLDRIAELVHPDAQFHYTQNGNTVTFDSKDEFKKWVEEWKSQHPDVKRQIHRIEVNGNTATVHYTVHVNHPNGRTFTFHATDHFHFRNGKIVEVRSHVEQRD |
| 1483 | PEFEERVKDWKKKFVEAYQNNDLDKLVSLLHDDATIHHSSGETITNKEEIREFLKKLLDTNKDARVEIKSIEIRGNTVTFHVEMHTTHNGQKVTFRVTVHDRYENGRLKESRFQFHQEE |
| 1484 | SLSEEEIRQWVTRFIEAMFNTGDWKQAADLLHPDAKITFNSHQRTFDMEEMIQWLREWKSRVSDFKIQIESIEVRGDEVTVHWTATVTHNGKTYTVHGTTTFRFRGNRVTEVRIQLHTSDT |
| 1485 | MSEEEIKEFYVRFFTDAFNRNDVDALLEIYHDDVTFHHADGSTITDKEEFKEWVERFIRRFKDIRIQWDRIEVDGDRVTFHITIHFRQGDQDYTVHVTVHVTFSDGRITEEHEQSDES |
| 1486 | PSEEEIREWLKRFYEALDKGDLEAVKRLLSDDATIEISATQKEYDVEEFIEWVERVQKRAKRIHREIKEIEIRGDTVTVHVDVTFHHKDKTVTVSFTETHHFRGKRLVSTHIHVDERE |
| 1487 | TSEEEIRQILRRLYEAFNNKDLDTLEELFHPDATMHVEAENRTFTNIEEIKEWLERFMKRFDVKLDITRIEVTEDKATVHVKVHMHTKDSTHTFDVTEHHHFKDGRLIETRIHIRQVD |
| 1488 | TSEEEVRRIKKRLVEAFNNHDWEALKRIFHPDATIHFYNSDGSTITFTNPEEFEKWFREWAKTFPDVKIQVDSLEVRGDTVTVHVTMHVTHNTQDYTFKVTTHDRYKNGKLTETRGHYNTT |
| 1489 | MSEEEIKQVLKRFYEAFNNKDLDALSELFHPDATIHFTNHDGSTTTFEDPSEFRRWVEQMLRTFEDMKIEIKEIRVDGDTVTSHVTVHVRHGDQPQTYTVTEHHRFKDGKITETRTTWHQQ |
| 1490 | ASEEELRQAMRRFYEAFNNKDIDKLSELFHPDATMHVENPDGSRITFHDPDEIRKWIKQTADTNPDVKIQIDEIRVDGDTVTVHVTVHVTHNTQPQTYTVTEHHHFKDGKITETRVTFNQQ |
| 1491 | DEDKEEIKDIIRRWVEAFNRRDVDALLSLYHPDAQVHFSNGETLHNLEDIKKFLARLFSNMQEFQVEVKSIHIEPNHVTVHMQLHTTHNGEKYTYDITVHFQIKDGKIVRQHVHTNEKK |
| 1492 | DEEQEEIKEILRQYYDSFNKNDLDKAASLMHPDATVHYANGETLHSKEEIREFLERLMKTWQEFHVTIEEIRVEPNHATVHYTVHVTHNGKKITYHGTDHYRFENGKIKEVHSHRNQRD |
| 1493 | PEQEKEIKEILERYYEALDRRDVDAVLSLLHPDVQVHHADGTTLNSLESVKEFIERLLKRYQEMQHTMEEIHFEPDHATVTYTMHTTHNGQKYTFHGTDHFRFENGKIKRIESHVNQRD |
| 1494 | PNPEQITELLRRFLEEAWERGDLDALEEFYAPGAQLHIWNGITFNDREEIKRWFKEMFEKFPDTHVEIKHVEVRGDTVTVHMTFHVRHGDQKYTYHITSHFHFDKDGKLRREQAHEQQQR |
| 1495 | PNQEQIVDTVIKWFEEAFNKGDADAFASFYSDGAQYHNWQGITAHSREEIKELFERFLKKFPDAQFEVKSVEVDGDTVTFHVQVHVRHGDQKVTYHVTSHMHFDKDGRLVEEHVHLQEQK |
| 1496 | MSEEEIKEILRQLYEAFDRGDWEKLARLLHDDARMHVSEEGRTFTNKEEIIRWYQQWWSRHETQRGEIDIHIHGDTATVHVTVHHHNGDQTHTVDYTHHWVFKDGKVVEVRIHTDSK |
| 1497 | TDESQIREWKKRYMEALDKKDLDAIEELIHPGATFHFADGRTFRDKDEYKEWVRQWLSKYDPKSREVHIHVDDSRTVTVHFTVTVNQNGKEVHIHGTVHDRYENGKLVESQYHHEQRD |
| 1498 | MSEEEIREFIRQLLEALDRGADEAASYYHPGSTMHVHHKDGTITWNSKDEIREWLKRFQSQFEFRHEVEDMHIDGNTVTVHVTVHVTHNGETRTYTVTRHFHFEGKRLTEDHNNWDQA |
| 1499 | ASEEQIRQIVRRLFEAMNNNDVEKLKELYHPDARIHFAGEGYTATTREEFAEWFKKFWSRNQFTIEIEDMHIDGDRVTVHFTVHFTHGDQPFTTRITSHFQFKDGKLVSEHIHMNTEQ |
| 1500 | MSEEEIKETLKRFFEAFDKGDADKLRELIHPDATFHFTFSNTTITTLEEFVEWLKEFLKTFQIHLRHIESIEIRKDEVTVHWTMTFTHNGETYDIRVTTTFRFKDGKIVEVRFHFNQT |
| 1501 | MSEEEIREWLKQFFEAFDKGDLDKLEELFHPHARIHDESSNTTFTSREEFKRWVKEFIEKFQIHLVTIHSIHIHKDTVTVHWTLQVHHNGQTYHVNATTTFHFRDGKLVEVRFSYNDT |
| 1502 | PSEEEIREILRQFYEAFDKRDWKKLAELWHPDGTMHVTATGTTATTLEEVIRMIKEFASKFDFQVIQIDSIEFNGDYVTVHVTITFTHNGQNYTVTVTETHHMKDGKIVETRMQFDQQ |
| 1503 | SDEEEIEEIVRRLFEAMSKTGDADAAAKLLHPDAQYTNSDEQRTYDREEYIEMIERRVKNDLKVHIQIESIEIRGDTVTVHWTATTTQGDEDQTYRYTTHIRFKDGKIVELRETTIT |
| 1504 | SDAEEIEEILRRMFQRLARQKDPEAFAQIVHPDAQFHDSSTNKQYDREEYKEWWERLIKNGIDTQIHIESIEIRGDTVHVHWTAHHQQGDTTHTVRGTTTFEFKDGKIVRIQFTHRT |
| 1505 | DSEQQQRIKEWLQRFFEAFNNNDLDTLKSLFDDDWQYHDQSGETLHSKEDFEEWVKKFLSTFPNVQIEIKSIHIDGDTVTVHVQMHVTHDGKTYTFHVTEHHRFENGKLKETRITFDPHP |
| 1506 | PSQEEQIREALRRLYEALNRGDIDTLVSLFHPDWQFHHSSGQTFHDLEEFREWAERWARSNKEYRVQIDEIEFRGDTVTVHVTIHVTHDTGTRTIHVTDTHHFRNGKLTETRARYDEKQT |
| 1507 | PGEDTEKIKEFIQRFIEAVNHNDADALAELFHPGATVHFSDGVTITSKEEFKEWLEEWIKTFKDVRIEIKSIHIDGKTVTVHIQVHVTDKSTGQTFTFRVTVHDRYENGKLKERRIHIDKSG |
| 1508 | PDFQEEIKEIDTRRVEYVWRTNDADALFEIFHPGAEIHYYSEQVTLTDLEELKEWFKQLVERFPDVKVEIKSIEVRGDRVTVHVTFHFNQSEGTTTYQVTYTVEYKNGKITRYEVHIHQT |
| 1509 | PSSEEIKEFIKRLLEAWNKDADEFYEIVTEDWQYQDARTGQTYDAEEFREMVKQWQETFGDVKVEVHIDARLENDTVTVHYTVHVNTTTKTTTTDFTEHHTFKNGRLVRTRQHADTKG |
| 1510 | ASEEEVEDFKRKFVEAFAKDWREVRKIMTPDSTFIDARTGTEYSLDEFADWVRRFQEKFSDIKFEIDFNLEFHGNRVTVHVTFHANTTNRTYTFHYTFHDEYRNGKLTRSQIHTRQEG |
| 1511 | PEDEEKIRELVKRFYEAFNRRDLDALEELFHDDGRYHNSSDNNSYDKEEFKEWVETFLRQFPRARMEIKSIEVRGKTVEVTVQVHLYHPNRTQTFTVHDTYHVRGDKIVEIRSHVDDG |
| 1512 | DESTEKIRQWLKRYIEALNNKDLDALESLFDKDWRYHDARENNSYDRDEFKKRVETFLRQFPNVRFEIKEIHFDGNTVQVHVQVHVHHDDQTRTYDVTEHHRFEGDKITETRVHHDST |
| 1513 | PREDEMKEKIKELKRRIVEAFNKGDLDALASLFDPGATFHLSNGQTFTTKEEFREWVKQFIDANIKIQVTSMEIQVTDDTHVTVTVHVTIHQTGTGKTVNITQHDHYRFENGQLKIVEVRTQVDEG |
| 1514 | PKDEKAKEDIKEWFKREAELFNNGDMDAWASLFHPGSTFTDASTNQTYNRDQLKEWFTEMFKNDVKIEIKELRIEKKSDKTFTVTVHVTFHMNHNGETRTVHITFHDHFELNGSEWRLVHRRIQTDGS |
| 1515 | PDQEKAKEDIKELVERFYRAFNNNDLDALKSFLAPDWRYHDSRTGQTYNAEQFLEMVEKMLRQDTHLQIDSIRIEQKDENEFTVTIHVTMTRTHNGQKQDIHVTDTHRVRKHGDGFKFVETRTRFDNG |
| 1516 | PREDEIKEAIKETLKRLYEAINRGDADAVAELFAPDWRYHNKSENQTYNKEQFREMIKKMFSNDTRLQIDQIDIEIKSKDTATVTVHVTFHTNHNGQTYTVHVTDTHHFEYQDSRLLLRRTESQFDGG |
| 1517 | PSEEDQEALRQTKRRYVEALNTGDLDTLSELLTDDSTFHHDDQTFQISKEEFKEWVKKMNSNDTKIQIHSIEVHLDSDGTTFTVTVHVTVHHNHGDKTKTVHVTTHDHYKKTDNDFKLKETRTHTDERS |
| 1518 | ADPSDQEAIRQVVERYYEALNKGDLDTLEKLLTDDWTLTTDNSTQPVSRDEFKERVKELLKNNTQIQIHSIEVRQHDNGTEFTVTVHVTVHHNHGDRTKTVHVEDHHTLRKDDSDFKIVHTQSHRDERS |
| 1519 | PDPNDEEAIREIVRQMFEAFNKGDLDAIASYYDDDVTFHHDNQTFQISKEEWKKWVEEVLRRDVKLEIKSIEVRQHPDGTHFTVTVHVTVHQNRGDKTHTVHVTRHFFFRKDDSDFKLKEDREHTDNRP |
| 1520 | PGEKTLQEAIEETDRRRVEAINKGDWKKIKELFTDDATFHHQSGQTYDHEDFLRWVKTMHSQQWQFEIKSIEIHIHPNGRTVTVHVTVTATNQRTKQDVQFHYTIVYVFENGDVKVKEMHLSTDH |
| 1521 | PGEETAEEQIREWKKRFIEAVNNNDRDKVKELLTDDATFHDANGQTYDADEFADMVEEFARQDWKIQLDSLEIRFQPDNEHVTVHVHITVHNDSTGTQIHFDATDFYRFENGSLKLVSTQYHTNN |
| 1522 | PGEEEAREKIRQVKRRLIEALDKGDWETVKRLFDDDFTFHDASGQTYDADEFRRMVEKMASHDVRREIKSLEIRRDSDNHNFTAHVHLTVQDSRTGQEVKFTATDFYRFENGSVKIVETHLHTDS |
| 1523 | DVDPSQIEQLEELKRRFVEALNKQDLDKMKSLMDDDSTIHSNPMNRDQFKEWVERLAKATKDARIEIKEIHFQLRSKDTVTVTVHITFHATHNGKTKTVHITYHDHYVQTKDGWKLKSTEERSDEP |
| 1524 | DMSKEDQEQIEEWVRRLIEAINKQDRDKVLSLYHDDSTNDSNPMDAEELKEWIEKMLRDLKDSRIQIEKIDFEQLDKDHVRVTVHVTIHRTHNGRTQTVTVTSHFIFEKDKDGWKLKSEEERRTNQ |
| 1525 | SQNEEIKKAVRELLKRLFEAFDKGDADAMKSLMHPDWRYTDASTQTTYDLDEFREWVERWQRNFSTRHEIKSIEIEIKPDGTVTAHVHVTTHTTHRNGEKRTFDVTETHHFDIGPWKVKETRVQYDNKG |
| 1526 | PDDEEAKEAVKQFLERYFEAINKKDVDALRSLFDDDWRYYDARTNQTYDADEFAEWVERFQSNFDSQIHIHSIEIRIHSDGTVTVTVHVTVHMTHPDGQTQTVKVTEHHRLDRREWKILETRAHWDNGG |
| 1527 | SDEEEIKEAVKEVIRRYMELMDNKDADAVASLYAPDARLHNADRNETYNFEELKEWYRRFLSNFDSKHDIKSIEIRIHPDGTITVHVHVEVHVTHKNGQNHTVRYTYTFVLDQSEWRIKEVHVHTDNGG |
| 1528 | MSDDEMREAAKDWLRRYFEAWNKGDLDKLRKLYHPDATFHDSSGQTYDKEEWVKRAEELLKTFDFTLQVDEVRAQVHPNKTHITVEVHVTAHVHHQSGETRTQTVTSEFHFQFENGQIYLLEEHVTTEQK |
| 1529 | SVGEETIERLIRRFYDAMNRGDLDEFISLLHDDWRWHDTDRGETYDKEEWREMWERRFRNSDFRVQIDSMEIRQVDENEYRVTVHVTVTHHDGNRTRHIHIEDHHRFRQTDGELKLVETHTHTDPG |
| 1530 | PMSEEAVREVIRRWFEAWNNKDLDKLASLYHDDATFHFKSHNQTYDKEEWLEWVKQLLRHSDIKVQVDSLEVRQVSKDEYTVTVHFTVTHHDGNETRHIHITAHFHLRYSKDSVKIVEQRDDYNSG |
| 1531 | PMGEEELKKFLKRLYEIMDSGDADKFASMFHDDVTFHNADTNQTYDKEEMLELLRRFASNAETHRQIDSIEIRYVDKDEIRVTVHVTVQVHQGNKTEHYHITHHWVFRYTDNRVQIVEVHESRDPG |
| 1532 | SSDREAVERMVRRYFEAWDKKDWEKLASLFDEDWRYHDAETNQTYDREEFIEMFRRFASNAQVHHEIHSLEVRQVTPDTFTVTVHFTVHEHHGNETHTFRVTEHHHFKDTKDGVKVQETHVHFDSE |
| 1533 | PASQEQVERLIRREAEALNKNDLDAWASLFHDDATFHDPDRNQTYDKEQMRKMIEEFIRNTDFRVHIDSIRIEQTDKDEYTATVHWTIHEHRGNETHTYHATTHDRFRYTDGDLKIIERHTTTDPG |
| 1534 | DTEELEEAARQIVRRLIEAFNRGDMDELSELYAKDEFTFTQYQGQTFSYEEFITMLERRFSNDTKVQLDSIRIEIKSKNEVTVEVHVTFHTHHNDETQTYHVTSEMHIRKEGDSWKILEEHERTNST |
| 1535 | SDEEAEEAARELVRRYYEAINKGDLDALQSLIDKDTWRYHTSSGTTYDYEEFVKMIEELFKAGVHVEIKGIEVRIVDKDEATVTVNVQFHQNHGDRTKTVDVEDHHHFRKKGDSWKLTRTETHHRST |
| 1536 | DQPEDDEEIKELIERMYRSLNENDIDKAASLLDPENFRYHTDSNTEYDYEEFLEMIERMASQGFRIQIDSLEIRWVDKNEVRVTVHVTIHVRHGDKTTTFHATHHWEFVKEGDSWKIKRLETHRNST |
| 1537 | DEEEIKSAIEELKRRLIEALNRNDMDTIRSIFDPTNFRFHQWQGQTFDFEEFITMIERRASAGFEIQLDSLEIRVQSSNSATATVHITVHFHHGDKTQTYHYTFHDHYRKEGDSWKITHTQEHRHST |
| 1538 | PDPSERQEQIKRIKERYIEAVNKGDLDEIRELIHDDARVQAEPLDKDQIIKMVEEITRNDLKLQLDSVEIHVDENDQVHATFHVTVTDGRTGQTTHVTYHDVYEASTELKIKSTTSRNDS |
| 1539 | ADPKEIIEQAEEVLRRIYQAFNEGDVDAVADLFHPDARIQSEPLNRDQFRDWVEKFFKAGFEMHVESIEFRVDHNDEATVHVHVTVTDRRTGSSIRVQETHRVRYSNGLRIVSTRTHNDS |
| 1540 | PDPSEIISQAREILTQFYEAINKGDLDRIEELLDDDVRIHSSPQDKEQFKEWVEKLTRNNFHLEIKSMEFRVDHSDEITVTVHVDITDQRTGRTITVTNHDFLRYKTELRIVSHTWQNNS |
| 1541 | IDPKEAEEQIRDLVRRFYEALNKGDAEAVARLLHPDVRVHTNPLDREQIKEWVEKFFRNNFEVKIDDLEIRFDSSERVTVTVHVTITDRRTGTTFHVTHHWFIDQSTELKIKHVREHNTS |
| 1542 | PSEEEQREEIKQLKERFAEALNKGDVDAVMRMYHDDARVTANPINKEQIKKWVKEWHDQGWEVEVKSLEIHVDENEEVHATAHVTVTDRRTGQTFHVTYHDVYVLSNGLKIKSTQSHNDS |
| 1543 | PGEESAREIIKEIKRRYVEAMNNGDADAAAAFFTDDATFWHNGQQQPLSREEFKKMIEEFFSKIDVKVEIKSIEIRIYDDGRIEVTVHVTFTYSETGTTADVDQHDVYREKDGTMKIIESRTHTRRKQS |
| 1544 | PGEKTMEEQVREVIQRFYEAMNNNDLDTLAELFTDDATFYHNGQQYDLSKEEFKKWMEEFLRHMDVKFEVDSIEIHIHDDGTVTAHVHVTLKHDKTGRTFEVTETHFFVVRNGSVKIKETREHTDSTKD |
| 1545 | PGEKTQQEQVREFVRRLITAFNTGDADAIKSLYTPDATFTHNGETYQLDQDEFKEWVEKFIKNLDFRLEIKSIEIRVDHDGTVTVTVHVQQTEDKTGRHFHFTRHFFFRFKDGEIRLKRDEEHTEERKD |
| 1546 | PGKKTIQEQVEEIVRRYYEALQNRDADKIASLFDKDATFTENGETHPLDKEEFKKRVEETLRKFDFRVHITGIEVEVKDDGTVTVTVHVEFHFKETGRTFKVTEHHQLRVENGSVKIVRTETHRDSTKD |
| 1547 | PGKETIEEQIRQWVHRYYDAFDKGDADTLASLIDDDWRYTTDGQTFQVSREEFKEAMEKIAKSVQTAHQIESIEIRVDDSGTATVHVHVTVHDSKTGRTFRVTDTHFFQLKDGELRLVETRTTTRSTSD |
| 1548 | SSEKEIREAVKEMIKRYIEAFNKGDLDALKDLYDKDARFHMGGYNLEQYTEWVRQLLESGTKVSLDSVEIHVNDDGTATATIHVTFHTHTPTSTHTVHVTRHLHFRVQDGRYKIVRDEIRENH |
| 1549 | MSEEERQSAVKEVLRQLYEAINKGDMDKVRSLVHPHATFHDSSRNQDYNREEFIEMAEKLTKNGVHFEIKEIRVQFTDKDEATVTVHVTVHHHHGDKTKTVQVEEHHRFRFKDGDVKLVETRAHFDDT |
| 1550 | MSEEELKKAIKEFDEERAKAFNTGDWEKLRSLMHDDATVHNYAEGQTYNRDQFLEWVKKMHSQGTRIQITEIRVQVTDENTVTVEVHGTVHTQRGDQTYTVHATYTMFYRFNNGDIKVIRMEIRLDNS |
| 1551 | PSEEELKKQIEETLRRLFEALNKGDLDAFLSFVDDDWRFHNTATNRTFDREEFRKWLKEFKENGAQIDIHEIRVQVTDQTNVTVTVHVTVHHNRGDRTTTIRVTEHHHFRFENGSVKLVRTQVHVDNT |
| 1552 | ASEEELRTQIKEYKRRLVEAFNNGNWEEFKRLVHPDATFHISATQRTYNADEFVEMLKELWKNGTRIEIKQIDIQFGERTHVTVTVHFTVHTNHGDKTRTIHATAHDRYRFKDGQIYLIESQFHFDNS |
| 1553 | MSEEEQKKAVEELVRRLVEAFNKGDLDELKEIYAPDATFHYNSTQRTYSKEEFIKMVEELTKAGTQIEIKEIRVEIKDDTTITVHVHVQVHTRRGDRTTTFDATRTFHFKLENGSFKLKHDQVTIDNT |
| 1554 | MSEEEMERQIREFKRRFIEAFNKGDMEKLKSMFHPDAQFHFANTQRTYNAEQFIEWLKRVLDNDTQIQIDSMEFRFGDSTHVTVHVHVTVHTNHGDRTYTVRVTATDHYRFENGSLKLVRSQFHHDNT |
| 1555 | PSKKDEEDIREFKRRYIEAFNKGDWEKLKRLIHPDATFHQNNTGRTFDVDEWVEMLKQLADQGVKIQLDSLEVQQRSSETVTVTAHVTVHDSQTGRTRHAQQHDRYRKIGDTWYLIETRVTRHGH |
| 1556 | PSKEQEEAVREILRRYVEAVNSGDADQLKELYHPDATFHQDDTGRTYDLEELREWYERMFRNNVRMQVDQIDIQVDDKDQVTVTVHITFHNASTGTTTHAQYHFTIRKEGDSWKIVEVHVRTHGE |
| 1557 | PSKKQEEDIKEILKHYIRAINEGDVDAVASLYHPDARLHQDGTGRTFDYEEIREWYERMVRNDVKVQLDSVEIRVDDKDQVTVHVHVTYHNGKTGSTRHATQTFTVRLEGDTWKIVEVHVTTHGE |
| 1558 | DEEEAQEQIKEMVEREVELLNKGDMDTWKSLVHPDATIHFSNGQTLTDRDEMKEWYKRFFETTNIQITIKSIEIQYGDKGEATVTVHFEMHRHTKDGTITAHFTAHIRVRLDNGGLKILSKEVHLDNRGG |
| 1559 | DESEAEEQIREFIRRFIEALNKNDADTLASLFDDDWRLHVADGETITTREEFREWVRRFYSEFDFQIQVDSLEVQKSHEGHYTVTVHVTVHFHHKDGTVTAHVTEHHHFRRDDGGMKVVETRVTIDRRGG |
| 1560 | DESESEEQIREFIKRYYTAFNNNDLDALAELLAPDWTFHHSNGSTITNLEEFKEWVRRLIERFNVQMEIKSLEIRKGDEGTATVHVTVQVTFHTKDGTYHATVHDTHHFQLDDGGLKLKETHSRWDHSGS |
| 1561 | SESEAEEQIKEWLKRFYEAINKGDLDAAASLLTDDWRYEDSSGEEITSREEFKDWLRRVMERFNFQIEIKSIEVRYGSPDFYTVTVHLEVHFTHKDGQYTRDTTDHHVFRRGNGGMKIVETRVHWDDKGG |
| 1562 | DQSETEEQVRETLRRFYEAVDKGDAETAARLLHDDWQFHNSKGETIHTLEEFKEWVERFFKNTDFQRQIDKQDFQPGPEGTMTVTVHVTIHFHHKDGTVTVHVTEHHHVRRDEGRLRIVRTQVHIDERGG |
| 1563 | PSEEDEQAIREIVERFYEAINQGDLETIDRLVHDDATIHDSSTNTDYDKEQFIRMIEKLFKNDFKLEIKSIEIHQDGNNDFTVTVHVQIHVTHNGENQTYHVTHHWFIRKKDGRYKIVEVRIHWKGNPS |
| 1564 | PSEEDQRAIEEMLRQFYRAFNTGDFEQIDSLVHDDATFEDKSTGQQYDKEQFREMFERLVKNNTKIQIHGIEFRSNGNNTYTVHVHVTFHTTHNGENHTQRVTETHVFEKRNGEWYIVETKFQYDGAPS |
| 1565 | PSEEQERKLREWKQRYVEAFNKGDLETIKSLFHDDATFTDSSNNQSYDKEQFIERIRRLQSNDTKMQLDSMEIRLNGNNTATVRVEFTFTTNHNGKTQDQRFTYTDFYRFQDGSLKLVRTEIHRDGAPK |
| 1566 | PSEEEEKEIRRLVKEFYDAFNNKDLEALASFYAEDAKFHDARTNQTYDREEIKRMIEQMLRNDVKMQVHSIHVQSNGNNTITVTVHVQVHETHNGKNQSFTFTQHYFFVKQNGSWKLVEDRLHLDGAPQ |
| 1567 | PDPSQEEEIKRLLKEYYEALDNGDLDTAASLMTDDWRYHDARTGQTYDKEEFKKMVGERFKRSDFQRHIHGIQIDDAKGNTVTVTVHVTVTHHHNGKQFHTHYTDTHHLTFFNGELKIVETRSQWDDKPD |
| 1568 | VDPSQEEKVRQFIRRYVEAWNNKDVDALASLYADDARYQDSRTGQEFDKEEMRERYEKFAKNSDATMHIDSIEIKDAKGNHVTVTVNMTVHVTHNGKTITIKVQYHFFFKFENGELKLKRLTSTHDEKPD |
| 1569 | ADPSQQEAVRQVDKERVEAFNRNDVDRMASLFHPDARIHFAPTGQTYDRDEFREWVERFLKNFDVKVQIDSIEIKDAKGNEVTVTATFTMHVTHNGKTITVHATYTIRYRLKDGQWKIIEVRIHRDEQKD |
| 1570 | MSEKELQEALKEWVEREIEAFNKGDMEKWKELFHPGSTFQNESTQQTYDREEVIKMIEELLSNGVKIQLDRLEVQFDHSNHITVRVEVQVHQHHGDQTHTMNVERHDHFRLKDGQFRLVRRQVRIDNT |
| 1571 | MSEEEMKEAVKDWVRRYFEAINNNDLDALRRLYTPGATMHIARESQTYDYEQIVKWYEEMLRSDLKMEVKSLEIQIDHTNEATVTVHFTVHENHGDQTHTFRAEAHFRFRFENGQLKLLEVHFHFDQT |
| 1572 | MSEEELREAIKDWFRRYVEAFNKNDLDALDSLYHPGVTMHIKENNKDYNKDQIREWYERLLSQDTKMQLDRLDIQIDHTNEVHVRVEMTFHTNRGDQTHTVHVQAHFRFRFENGSLKLVEVHFQFDHS |
| 1573 | PGEEEAEKQIRELVRRFFEAIDKGDLDQLLEMLDDDFRFHDASKGETYDKEEFAEWAERLASQDSKHDIHELHIEFNGDTITVRVHWTLHLTHGDQQYTFHGTTELHFERNNGTLYLKEMRVEIHGE |
| 1574 | PGEEEAERAIREWVRRFVEAINKGDMDTVDELFTPDWRFHHRSTQTTYDKEEFREWVEKLFRNDTKIEIKSLEIEIRGHTITVHVTITVHTNRGDQTYTFEVHETHHFERREGEFKLVRTEAETHGE |
| 1575 | PGEEENTEVIKELLKRFYEAIDKGDWQAIHDLYHPDARLHNRSHNQTYNREQIVRMAEKQASQDTRRQIDEMHIEVRGNTITVHVHVTVHVNRGDRTETYTFTQTFHFEYSNGEFKLIEDHVQTHTN |
| 1576 | PGEETQKEQVKEWLTQYFDLINKGDADAVASLYHPDARVHNSSHNQTYTYEELKEWYERLASADFQIQVDSIEIEINGDTVTVHVHYTMHVNRGDKTYTFHGTTTFHFENQDGSMYLVEVHTTTHSS |
| 1577 | PEDEEAIKEIKRQLIEALDKGDMETLRQLFHPDVTFHHKDQTWNLNIEEWLERVARTLSNVEVHRHMKSIHIQPTTETTWTAEVHLEVHETHNGQKHTFRVTQHDRYVKLGDEWKILESESHRDST |
| 1578 | VDESEQREAIKEWLQRYYEALNNKDWEAVASLYTPDSQYYQPNGQQYDREQFIDMIRKMLSRNDVQFHVKQIDIEVKPDGTVTVTVHVEVHETHNGQQYTFRITQEFRFRYEDSEFKLKEDHVRRDDG |
| 1579 | PKEEEAKEAIKEWLKQFYEALQRHDVDAVASLYDKDSRFHDSRDPRTYDRDEFREWVERMLSSIDDFKVEIKEIRIQFNGEDRVTVEVHGHVTFNHGDSQRHATVTQTFVFNLKDWKLVEDHATFHDHT |
| 1580 | PEKEIREQIKEIKERLVEALNKGDMDTFASLFHPDAQMHISRGRTFNSRDEIKKWFKDYKETFDVRIQITSIEIRISDSNQATVTVHITFHYNEDNKTVDYTFHDRYVIKDGDMRIVRSQIHSHER |
| 1581 | PEEDEMREQIKEFVKREFEAINKGDLKTWEELVHPDATFHNSSEGRTYDVEEMKERIKKFVEVPTQIEVKSIEIQINKNGEATVTVHVQVHEHHGDQTHTFNITQHDHLRRINGQWYFIERHVTTDST |
| 1582 | PSEEDEKEIRETKRQYVEAWNNNDLDKLADIVHPDAKYASRNLDYEEYIEWVKRWMERFKNVQIQIHSIHIQQDGSDNITVTVHFTVTEHHNGQQQHHRYTAHDHYRKSNGKWFIVRMETRRHGS |
| 1583 | PSEEDERAVRDVVRRFYEAINHNDLDKLLELMHPDATFRARNLNYEQIKEWFREWMERFKNARLEIKSIEVRQHGSDTFTVTVHVTMHVTHNGQDQTYHITHHWVLVKFNGEWKIVSVEERTNGG |
| 1584 | ADKEQQEDIRRIIQRLVEAWNNNDLDTWLSLFDKDWRYSSRNIDKEEFREWMERVIRTFQDLKVQLDSIRIELDGSDTITVHVHITFHHYHNGQQHTHHVTETHHFVRKEGRWYIVRTTTRRTDG |
| 1585 | PDEEEQEKVKKFIQRYVEAFNNNDLEELESLYHPGAQLDINGVDLKQFKEIVERLIRNGFEFRLEIKSIEIRKDGDEITAHVHVQVHTTHNGKTDTVDVTQTFHFDTRHDQLKNHQVHHQGS |
| 1586 | SNKETEELVRELIKQWFEAVDTDPDKAASYYDEDAQVHSSTGETLHDLQSIKDYIRNHSKDYQHELKSIEIRIHPDGTVTVHVTFQVHDRTTGQTYTVHTTFFIVQKGDTVYIKEQRTTIHTG |
| 1587 | LSEEEIKEWLKRFYEAVDRGDRDKIRKLLHPDVQMHSASGRTFNNIDEIIKWLSEFTSRGFKRHIREIHIEITKDEVHVTVHAEIHFEKTKQTIKFTHHWTFDLRHKKIVSVQFHETG |
| 1588 | PSDEELKEWKERFIEAINKGDLDKLRELLHPDVTFHIWNGRTFNNLDEVLKWFEEFTSQNWHMEIKDMRIEHKDNEIRVTVHITVHNDSTGQTFHFTFHDRYDKRHKKLTESRIQETQ |
| 1589 | LSKEEARELVERFFEAFNRGDMDAIREIVHPDAQFHDSNGEQLTNIEEIVEWAKKLFDQGVEIQITEIHVEVTETHVHVTVHWNGTDRRTGRTFRGTTHFVFDKRTKKIVRLEFRHTG |
| 1590 | DTPEDEEAIKELVKREVEAFNKGDMDALASFYDDDAQMHLSNGQTFDTKEEIKKWLEELSKNSQIHVESLHIQKDDDNTFHVEVHAQMRNKTTGQDVTVTRHVTVKREDTGLKIKHDQVRIDSN |
| 1591 | KDEELEKAAREVVRRLIEAINTGDLDTLKELYAPDAQFHVWEGRTFDTLEEIAKWFKEFAEKFDLEIKSIEIRIDDDDRVTVHVHVKVTNRETKQDFTFRSTMHLKFDHGGVKIKREQVDVDPN |
| 1592 | DKEEDETDIKEILKRLFRAMDEGDMDTMKSLFDEDWQYHNTSGRTFTTLEEFAEWISQFSSDTAHHIKSIEIRKDDNNQLEVHVHVEVHRSSTGQTFRYTETHRFEKKDGSWKIVETRQHVDPS |
| 1593 | DDEEMEEEVRQLVRRLAEALNKGDRDAIRELYHPDARMELWNGRQFDTLDEIVEWFMKWASHFDIEIKSIEVRVDDNQTVTVHVHFEATDRRTGKTVTFTSTIHFVKNDTGMKIKEEQIHADSN |
| 1594 | PETEEQVKDWKKKFVEAVNNGDMDKLRELFHPDARMHLSNGRTFTTLDEILEWFKKWASTYTIHIHSLEIRENDDGSVTVHVHGTVHNRRTGKQVQVTATDHYDRSRNKLIRSQFNFDPQ |
| 1595 | PDLQEEIRELVKRFYAAMNTGDADTLRDLFHPDATMHLSNGQTFTTLDEILEWLIKFASHFDFQITSIHIQVNHDGTITVTVHVTVRDRRTGQEYRVTDHYHVDSRHGKIIEVRSHVDST |
| 1596 | PDFQEEAKEIVRRLAEAFNKNDRDKLKDLFHPDAKMHVYNGQTFETRDEIVEWFSRLASHAQIHIHSIETHLHDDGSVTTHVHWTMTDKRTGQNFTGTTTFHVDRRTGKITEVRIHIDSN |
| 1597 | SDESEIEEAIRQIVRRLVEAFNKNDMDKLAEIYDKDATFHINGQTFSDLEEFLEWVKQVVSRGVQVTFTVEIHINGDTVTVHVRATFHFESHNQTFTGHSTIHIRLEGDQWKIVEEHLQIDQN |
| 1598 | SDRKEFEEEIREIIRRMFEAFNKGDRDTLRELYDKDATIHINGQTFTSWEEFLKWVTDVNSRGVRVEFRVHVEVHGDTVTVRVEAHLHQESTGETYTVRSTIHFRLENNRLKIVEEHVQVDKQ |
| 1599 | ADESEWQEQVKQWVERFVEAFNKNDRDALRELYDDDSTVHINGQTFTDWQEILEYISQLASTNAQVTFTIEIHVSGNTVTVHVTLTVTQENTGRTFTVHSTFHFELQDSQLRLVEEHVQTDQN |
| 1600 | NDESEFRQVIKEWVERFFEAFNKNDRDELRKLLDDDATFHINGQTYSTIEEFLEWIERVWSRGVQVTFTIEVHVDGDEATVRVEWTAHFKSTGETYHGTTTFHFRVEGRQFKIVEVRIQMDKS |
| 1601 | TSEDEFKEQVKEVLNQFYEAINRGDRDTLRELFSKGGTVHFQNGRTFTDQEEVVDWFSKFASSGFRIEWHIEIKEITEEHVTVTVHIRITDQNNNTQNVTDHYFMRLENGTWKITEVRSQYDN |
| 1602 | MSEKEIREAIKEFLERFYELLDRGDLDAIASMYADGATMHLENGQTFNSKEEIKQWFEKQISSDMRREVEVHIREISETTATVTAHVTLHGKDGTTIHITQEMHIEFQNGSIKIKEDRITTDS |
| 1603 | PSPEDQEKIRQIIRRYYEAFNKGDADKMAELFHDDWTFHHANGQTFTTKDEFREWATRLLSQNLTIEVEVHIHSGSKDEVTVTVHFHVRQKDTTFTEHHRFRKDKDGRLFIVETRVRQDGS |
| 1604 | MSKEQEDQIREWFERYYEAINKKDLETIVRLVHDDVTFHHDNGRTFTTKEEFKEWVKQILDQDWTLRFSIRIDSGSEKEVTVTVHVTYEQEPRTVREEHHFRFNDDGSLRVVETRFDSHGG |
| 1605 | PDPHVLEAIKDFVRRYAEAFNKKDLDTLVSLFHEDWTYHHSNGQTFTTRDEYREWVKRLLEQDWTIDVEVHFTSGSEKEYTVEVHFTFRQQPMTVTEHHRFRRHDNGQVRIVETRARWHGG |
| 1606 | PSKEQEEKVREWLERFYEAFNKKDLDTMASLVHDDVTLHLSNGQTFTTKDEIIKFLKELLSQDLTLEFEIHIDSGTEEEITVTVHVTIKQIDQTVTHHWFFRFDKDGNIYLVEVRENLTGK |
| 1607 | PSEEQEKAAREVIRRFYDAFNKGDIDKIAELIHDDATLHIWNGQTFTTKEEVIEFIQRLLSQNIQIDFEIHVKGGSKDEVTVEVEVHYRQSDMTVTHHWFLRRLPDGSWKVVELRARSDGK |
| 1608 | MSEEEIRQLKRRLAEAFNKKDADAIAELFHPDSRVTFASDNRTFTDKDEIKEWFTQYASRDIQVEIKSIEIRGKTVTVHMTFHNKRTGTTTHATFHDHYDDNGRLASSEIHFNTQN |
| 1609 | PSEEEIREWARRYYEALNNNDADTAVSLMDDDWTVHFSEGRTFHSKEEFKKWVEEMLSTDVKIEIKDLHIEGDTVTVHGTVTRTHNGQTRDLRTTDHHTFDKNGRITETHFHHDQQ |
| 1610 | PISEEQIKEFIKRFFEALNKGDVDLLAELVHDDATFTHNNQTFRMSKEEFREWVKKWLSSDTQIEIKSIEIRGDTVTVHWTAHTNKGDRTQTVHGTTHFEFDHDGKLVRVQTHNNPTS |
| 1611 | PVSEEQIKEFKKREVDAFNTGDMDKLADLYHDDATLTHGDQTFPMSKEEIKKWFEELFRNDTKFEIKSIEIRGDTVTVHVTLHTNHGDQTTTIHYTYHDRYQPDGKLTESRESEHPTS |
| 1612 | PMSEEQIEELVRRFYEAFNNGDLDKLREIMHDDATWTNGDQTFPMSKDELIEMFQKMLSTDTKLEIKSIEVRGDTVTVHVTVHTNHGDRTHTFHVTHHWTFNKDGKIVEVRQHMQPTR |
| 1613 | STSQEQNKEIDTRRVEAFNKGDWEKLSDLFHDDATFTHNNQTQPRSKEEFIRWVKKVHSNDTKFEIERMEAHGDTVHVEVTVHHTHGDKTTTVKVRYTVRYHSNGKILEYHEHRHPTS |
| 1614 | PISQEQIKEFLRQFYEALDKGDLDKLRELVHDDWTYVNNDQRYHMSKEEFKEWVKKFASSDTKREIKSMEIRGDTVTVHVTLHTNHGSETKTIQYTEHHRFDHDGKLVETQQHEQPTS |
| 1615 | PASEEEIREWLKQFYEAFNNKDWKKLADLMAPDARMEVTQSNGQNYEFTTKEEIIRWFQQLMSQFDSIEIKEIRIDQDRQTVTSHVTVHFTHNGQRFTVRVTEHHHFDHDGKARETKTTFDHGG |

### REFERENCES

1. Jiang, L. et al. De novo computational design of retro-aldol enzymes. Science 319, 1387-1391 (2008).

2. Rothlisberger, D. et al. Kemp elimination catalysts by computational enzyme design. Nature 453, 190-195 (2008).

3. Yeh, H. W. et al. Red-shifted luciferase-luciferin pairs for enhanced bioluminescence imaging. Nat. Methods 14, 971-974 (2017).

4. Love, A. C. & Prescher, J. A. Seeing (and Using) the Light: Recent Developments in Bioluminescence Technology. Cell Chemical Biology 27, 904-920 (2020).

5. Syed, A. J. & Anderson, J. C. Applications of bioluminescence in biotechnology and beyond. Chem. Soc. Rev. 50, 5668-5705 (2021).

6. Yeh, H.-W. & Ai, H.-W. Development and Applications of Bioluminescent and Chemiluminescent Reporters and Biosensors. Annu. Rev. Anal. Chem. 12, 129-150 (2019).

7. Zambito, G., Chawda, C. & Mezzanotte, L. Emerging tools for bioluminescence imaging. Curr. Opin. Chem. Biol. 63, 86-94 (2021).

8. Markova, S. V., Larionova, M. D. & Vysotski, E. S. Shining Light on the Secreted Luciferases of Marine Copepods: Current Knowledge and Applications. Photochem. Photobiol. 95, 705-721 (2019).

9. Wu, N. et al. Solution structure of Gaussia Luciferase with five disulfide bonds and identification of a putative coelenterazine binding cavity by heteronuclear NMR. Sci. Rep. 10, (2020).

10. Jiang, T. Y., Du, L. P. & Li, M. Y. Lighting up bioluminescence with coelenterazine: strategies and applications. Photochem. Photobiol. Sci. 15, 466-480 (2016).

11. Shakhmin, A. et al. Coelenterazine analogues emit red-shifted bioluminescence with NanoLuc. Org. Biomol. Chem. 15, 8559-8567 (2017).

12. Michelini, E. et al. Spectral-resolved gene technology for multiplexed bioluminescence and high-content screening. Anal. Chem. 80, 260-267 (2008).

13. Rathbun, C. M. et al. Parallel screening for rapid identification of orthogonal bioluminescent tools. ACS Cent. Sci. 3, 1254-1261 (2017).

14. Yeh, H.-W., Wu, T., Chen, M. & Ai, H.-W. Identification of Factors Complicating Bioluminescence Imaging. Biochemistry 58, 1689-1697 (2019).

15. Su, Y. C. et al. Novel NanoLuc substrates enable bright two-population bioluminescence imaging in animals. Nat. Methods 17, 852-860 (2020).

16. Lombardi, A., Pirro, F., Maglio, O., Chino, M. & DeGrado, W. F. De Novo design of four-helix bundle metalloproteins: One scaffold, diverse reactivities. Acc. Chem. Res. 52, 1148-1159 (2019).

17. Chino, M. et al. Artificial diiron enzymes with a DE Novo designed four-helix bundle structure. Eur. J. Inorg. Chem. 2015, 3352-3352 (2015).

18. Basler, S. et al. Efficient Lewis acid catalysis of an abiological reaction in a de novo protein scaffold. Nat. Chem. 13, 231-235 (2021).

19. Anishchenko, I. et al. De novo protein design by deep network hallucination. Nature (2021) doi:10.1038/s41586-021-04184-w.

20. Wang, J. et al. Scaffolding protein functional sites using deep learning. Science 377, 387-394 (2022).

21. Norn, C. et al. Protein sequence design by conformational landscape optimization. Proc. Natl. Acad. Sci. U. S. A. 118, (2021).

22. Yang, J. Y. et al. Improved protein structure prediction using predicted interresidue orientations. Proc. Natl. Acad. Sci. U. S. A. 117, 1496-1503 (2020).

23. Basanta, B. et al. An enumerative algorithm for de novo design of proteins with diverse pocket structures. Proc. Natl. Acad. Sci. U. S. A. 117, 22135-22145 (2020).

24. Loening, A. M., Fenn, T. D. & Gambhir, S. S. Crystal structures of the luciferase and green fluorescent protein from Renilla reniformis. J. Mol. Biol. 374, 1017-1028 (2007).

25. Tomabechi, Y. et al. Crystal structure of nanoKAZ: The mutated 19 kDa component of Oplophorus luciferase catalyzing the bioluminescent reaction with coelenterazine. Biochem. Biophys. Res. Commun. 470, 88-93 (2016).

26. Ding, B. W. & Liu, Y. J. Bioluminescence of Firefly Squid via Mechanism of Single Electron-Transfer Oxygenation and Charge-Transfer-Induced Luminescence. J. Am. Chem. Soc. 139, 1106-1119 (2017).

27. Isobe, H., Yamanaka, S., Kuramitsu, S. & Yamaguchi, K. Regulation mechanism of spin-orbit coupling in charge-transfer-induced luminescence of imidazopyrazinone derivatives. J. Am. Chem. Soc. 130, 132-149 (2008).

28. Kondo, H. et al. Substituent effects on the kinetics for the chemiluminescence reaction of 6-arylimidazo[1,2-a]pyrazin-3(7H)-ones (Cypridina luciferin analogues): support for the single electron transfer (SET)-oxygenation mechanism with triplet molecular oxygen. Tetrahedron Lett. 46, 7701-7704 (2005).

29. Branchini, B. R. et al. Experimental Support for a Single Electron-Transfer Oxidation Mechanism in Firefly Bioluminescence. J. Am. Chem. Soc. 137, 7592-7595 (2015).

30. Zubatyuk, R., Smith, J. S., Leszczynski, J. & Isayev, O. Accurate and transferable multitask prediction of chemical properties with an atoms-in-molecules neural network. Science Advances 5, (2019).

31. Dou, J. Y. et al. De novo design of a fluorescence-activating beta-barrel. Nature 561, 485-491 (2018).

32. Cao, L. et al. Design of protein-binding proteins from the target structure alone. Nature 605, 551-560 (2022).

33. Jumper, J. et al. Highly accurate protein structure prediction with AlphaFold. Nature 596, 583-+ (2021).

34. Dauparas, J. et al. Robust deep learning based protein sequence design using ProteinMPNN. bioRxiv (2022) doi:10.1101/2022.06.03.494563.

35. Yeh, H.-W. et al. ATP-Independent Bioluminescent Reporter Variants To Improve in Vivo Imaging. ACS Chem. Biol. 14, 959-965 (2019).

36. Bhaumik, S. & Gambhir, S. S. Optical imaging of Renilla luciferase reporter gene expression in living mice. Proc. Natl. Acad. Sci. U. S. A. 99, 377-382 (2002).

37. Szent-Gyorgyi, C., Ballou, B. T., Dagnal, E. & Bryan, B. Cloning and characterization of new bioluminescent proteins. in Biomedical Imaging: Reporters, Dyes, and Instrumentation (eds. Bornhop, D. J., Contag, C. H. & Sevick-Muraca, E. M.) (SPIE, 1999). doi:10.1117/12.351015.

38. Hall, M. P. et al. Engineered luciferase reporter from a deep sea shrimp utilizing a novel imidazopyrazinone substrate. ACS Chem. Biol. 7, 1848-1857 (2012).

39. Baek, M. et al. Accurate prediction of protein structures and interactions using a three-track neural network. Science 373, 871-+ (2021).

40. Giger, L. et al. Evolution of a designed retro-aldolase leads to complete active site remodeling. Nat. Chem. Biol. 9, 494-498 (2013).

41. Yao, Z. et al. Multiplexed bioluminescence microscopy via phasor analysis. Nat. Methods 19, 893-898 (2022).

42. Loening, A. M., Dragulescu-Andrasi, A. & Gambhir, S. S. A red-shifted Renilla luciferase for transient reporter-gene expression. Nat. Methods 7, 5-6 (2010).

43. Dijkema, F. M. et al. Flash properties of Gaussia luciferase are the result of covalent inhibition after a limited number of cycles. Protein Sci. 30, 638-649 (2021).

44. Schenkmayerova, A. et al. Engineering the protein dynamics of an ancestral luciferase. Nat. Commun. 12, (2021).

45. Ando, Y. et al. Development of a quantitative bio/chemiluminescence spectrometer determining quantum yields: Re-examination of the aqueous luminol chemiluminescence standard. Photochem. Photobiol. 83, 1205-1210 (2007).

46. Li, W. & Godzik, A. Cd-hit: a fast program for clustering and comparing large sets of protein or nucleotide sequences. Bioinformatics 22, 1658-1659 (2006).

47. Farrell, D. P. et al. Deep learning enables the atomic structure determination of the Fanconi Anemia core complex from cryoEM. IUCrJ7, 881-892 (2020).

48. Zhang, Y. & Skolnick, J. TM-align: a protein structure alignment algorithm based on the TM-score. Nucleic Acids Res. 33, 2302-2309 (2005).

49. Needleman, S. B. & Wunsch, C. D. A general method applicable to the search for similarities in the amino acid sequence of two proteins. J. Mol. Biol. 48, 443-453 (1970).

50. Oda, T., Lim, K. & Tomii, K. Simple adjustment of the sequence weight algorithm remarkably enhances PSI-BLAST performance. BMC Bioinformatics 18, 288 (2017).

51. Altschul, S. F. et al. Gapped BLAST and PSI-BLAST: a new generation of protein database search programs. Nucleic Acids Res. 25, 3389-3402 (1997).

52. Coventry, B. & Baker, D. Protein sequence optimization with a pairwise decomposable penalty for buried unsatisfied hydrogen bonds. PLoS Comput. Biol. 17, (2021).

53. Smith, A. J. T. et al. Structural Reorganization and Preorganization in Enzyme Active Sites: Comparisons of Experimental and Theoretically Ideal Active Site Geometries in the Multistep Serine Esterase Reaction Cycle. J. Am. Chem. Soc. 130, 15361-15373 (2008).

54. Salomon-Ferrer, R., Case, D. A. & Walker, R. C. An overview of the Amber biomolecular simulation package. Wiley Interdiscip. Rev. Comput. Mol. Sci. 3, 198-210 (2013).

55. Kellogg, E. H., Leaver-Fay, A. & Baker, D. Role of conformational sampling in computing mutation-induced changes in protein structure and stability. Proteins 79, 830-838 (2011).

56. Park, H. et al. Simultaneous optimization of biomolecular energy functions on features from small molecules and macromolecules. J. Chem. Theory Comput. 12, 6201-6212 (2016).

57. Klein, J. C. et al. Multiplex pairwise assembly of array-derived DNA oligonucleotides. Nucleic Acids Res. 44, (2016).

58. Loening, A. M., Wu, A. M. & Gambhir, S. S. Red-shifted Renilla reniformis luciferase variants for imaging in living subjects. Nat. Methods 4, 641-643 (2007).

59. Liang, J., Feng, X., Hait, D. & Head-Gordon, M. Revisiting the performance of time-dependent density functional theory for electronic excitations: Assessment of 43 popular and recently developed functionals from rungs one to four. J. Chem. Theory Comput. 18, 3460-3473 (2022).

60. Chai, J.-D. & Head-Gordon, M. Long-range corrected hybrid density functionals with damped atom-atom dispersion corrections. Phys. Chem. Chem. Phys. 10, 6615-6620 (2008).

61. Ditchfield, R., Hehre, W. J. & Pople, J. A. Self-consistent molecular-orbital methods. IX. An extended Gaussian-type basis for molecular-orbital studies of organic molecules. J. Chem. Phys. 54, 724-728 (1971).

62. Grimme, S. Exploration of chemical compound, conformer, and reaction space with meta-dynamics simulations based on tight-binding quantum chemical calculations. J. Chem. Theory Comput. 15, 2847-2862 (2019).

63. Pracht, P., Bohle, F. & Grimme, S. Automated exploration of the low-energy chemical space with fast quantum chemical methods. Phys. Chem. Chem. Phys. 22, 7169-7192 (2020).

64. Luchini, G., Alegre-Requena, J. V., Funes-Ardoiz, I. & Paton, R. S. GoodVibes: automated thermochemistry for heterogeneous computational chemistry data. F1000Res. 9, 291 (2020).

65. Li, Y.-P., Gomes, J., Mallikarjun Sharada, S., Bell, A. T. & Head-Gordon, M. Improved force-field parameters for QM/MM simulations of the energies of adsorption for molecules in zeolites and a free rotor correction to the rigid rotor harmonic oscillator model for adsorption enthalpies. J. Phys. Chem. C Nanomater. Interfaces 119, 1840-1850 (2015).

66. Götz, A. W. et al. Routine microsecond molecular dynamics simulations with AMBER on GPUs. 1. Generalized Born. J. Chem. Theory Comput. 8, 1542-1555 (2012).

67. Becke, A. D. Density-functional thermochemistry. III. The role of exact exchange. J. Chem. Phys. 98, 5648-5652 (1993).

68. Grimme, S., Antony, J., Ehrlich, S. & Krieg, H. A consistent and accurate ab initio parametrization of density functional dispersion correction (DFT-D) for the 94 elements H-Pu. J. Chem. Phys. 132, 154104 (2010).

69. Grimme, S., Ehrlich, S. & Goerigk, L. Effect of the damping function in dispersion corrected density functional theory. J. Comput. Chem. 32, 1456-1465 (2011).

70. Meiler, J. & Baker, D. ROSETTALIGAND: Protein-small molecule docking with full side-chain flexibility. Proteins 65, 538-548 (2006).

71. Davis, I. W. & Baker, D. RosettaLigand docking with full ligand and receptor flexibility. J. Mol. Biol. 385, 381-392 (2009).

72. Davis, I. W., Raha, K., Head, M. S. & Baker, D. Blind docking of pharmaceutically relevant compounds using RosettaLigand. Protein Sci. 18, 1998-2002 (2009).

73. Wang, J., Wolf, R. M., Caldwell, J. W., Kollman, P. A. & Case, D. A. Development and testing of a general amber force field. J. Comput. Chem. 25, 1157-1174 (2004).

74. Bayly, C. I., Cieplak, P., Cornell, W. & Kollman, P. A. A well-behaved electrostatic potential based method using charge restraints for deriving atomic charges: the RESP model. J. Phys. Chem. 97, 10269-10280 (1993).

75. Besler, B. H., Merz, K. M. & Kollman, P. A. Atomic charges derived from semiempirical methods. J. Comput. Chem. 11, 431-439 (1990).

76. Singh, U. C. & Kollman, P. A. An approach to computing electrostatic charges for molecules. J. Comput. Chem. 5, 129-145 (1984).

77. Jorgensen, W. L., Chandrasekhar, J., Madura, J. D., Impey, R. W. & Klein, M. L. Comparison of simple potential functions for simulating liquid water. J. Chem. Phys. 79, 926-935 (1983).

78. Maier, J. A. et al. Ffl4SB: Improving the accuracy of protein side chain and backbone parameters from ff99SB. J. Chem. Theory Comput. 11, 3696-3713 (2015).

79. Darden, T., York, D. & Pedersen, L. Particle mesh Ewald: AnN·log(N) method for Ewald sums in large systems. J. Chem. Phys. 98, 10089-10092 (1993).

80. Roe, D. R. & Cheatham, T. E., III. PTRAJ and CPPTRAJ: Software for processing and analysis of molecular dynamics trajectory data. J. Chem. Theory Comput. 9, 3084-3095 (2013).

We claim:

1. A polypeptide comprising an amino acid sequence at least 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-1615, not including any functional domains added fused to the protein (whether N-terminal, C-terminal, or internal), and wherein the 1, 2, 3, 4, or 5 N-terminal and/or C-terminal amino acid residues may be present or absent when considering the percent identity.

2. The polypeptide of claim 1, wherein 1, 2, 3, 4, or 5 N-terminal and/or C-terminal amino acid residues are absent and are not included when determining the percent identity relative to the reference amino acid sequence.

3. The polypeptide of claim 1, wherein all amino acid residues in the reference sequences are included when determining the percent identity relative to the reference protein.

4. The polypeptide of claim 1, wherein the polypeptide comprises the secondary structure arrangement H1-L1-H2-L2-E1-L3-E2-L4-H3-L5-E3-L6-E4-L7-E5-L8-E6, wherein “H” is a helical domain, “L” is a loop domain, and “E” is a beta strand domain.

5. The polypeptide of claim 4, wherein domain H1 is 19 amino acids in length, domain H2 is 7 amino acids in length, domain H3 is 14 amino acids in length, domain E1 is 4 amino acids in length, domain E2 is 4 amino acids in length, domain E3 is 10 amino acids in length, domain E4 is 12 amino acids in length, domain E5 is 14 amino acids in length, and domain E6 is 13 amino acids in length.

6. A fusion protein comprising the polypeptide of claim 1 fused to one or more functional polypeptides.

7. The fusion protein of claim 6, wherein the one or more functional polypeptides are fused to the N-terminus and/or C-terminus of the polypeptide.

8. The fusion protein of claim 5, wherein one or more functional polypeptides are inserted internally into the protein.

9. The fusion protein of claim 8, wherein the polypeptide comprises the secondary structure arrangement H1-L1-H2-L2-E1-L3-E2-L4-H3-L5-E3-L6-E4-L7-E5-L8-E6, wherein “H” is a helical domain, “L” is a loop domain, and “E” is a beta strand domain, and wherein each of the one or more functional polypeptide inserted internally into the polypeptide is inserted into a loop domain.

10. A nucleic acid encoding the protein or fusion protein of claim 1.

11. An expression vector comprising the nucleic acid of claim 10 operatively linked to a suitable control sequence.

12. A host cell comprising the expression vector of claim 11.

13. A library comprising 5, 10, 35, 50, 100, 250, 500, 750, 1000, or more different polypeptides according to claim 1.

14. A method for using the library of claim 1 for identifying or designing enzymes or small molecule binders.
